(19) United States
(12) Patent Application Publication
(10) Pub. No.: US 2024/0409964 A1
(43) Pub. Date: Dec. 12, 2024

(54) RECOMBINANT YEAST CELL HAVING INCREASED PYRUVATE DECARBOXYLASE ACTIVITY

(71) Applicant: DANSTAR FERMENT AG, Zug (CH)

(72) Inventors: Aaron Argyros, Lebanon, NH (US); Johannes Pieter Van Dijken, Lebanon, NH (US); Bailey Morgan Carignan, Lebanon, NH (US); Trisha Barrett, Lebanon, NH (US); Emily Stonehouse, Lebanon, NH (US)

(21) Appl. No.: 18/699,829

(22) PCT Filed: Oct. 11, 2022
(86) PCT No.: PCT/IB2022/059754

§ 371 Date: Apr. 9, 2024

Related U.S. Application Data
(60) Provisional application No. 63/254,366, filed on Oct. 11, 2021.

(51) Int. Cl. C12P 7/06; C12N 9/04; C12N 9/10; C12N 9/88

(52) U.S. Cl. CPC C12P 7/06; C12N 9/0006; C12N 9/1029; C12N 9/88; C12Y 101/01001; C12Y 203/01054; C12Y 401/01001

(57) ABSTRACT

The present disclosure provides a recombinant yeast cell for making ethanol. The recombinant yeast cell comprises a first genetic modification to increase an ethanol yield in the recombinant yeast cell when compared to a parental yeast cell. The recombinant yeast cell also comprises a second genetic modification capable of increasing pyruvate decarboxylase activity in the recombinant yeast cell when compared to the parental yeast cell. The parental yeast cell lacks the first genetic modification and the second genetic modification. The present disclosure also provides methods for making the recombinant yeast cell as well processes for using the recombinant yeast cell to make ethanol.

### CROSS-REFERENCE TO RELATED APPLICATIONS AND DOCUMENTS

This application claims priority from U.S. provisional patent application 63/254,366 filed on Oct. 11, 2021 and herewith incorporated in its entirety. This application also comprises a sequence listing in electronic form which is also incorporated in its entirety.

### TECHNOLOGICAL FIELD

The present disclosure concerns recombinant yeast cells for making ethanol and which are capable of exhibiting increased pyruvate decarboxylase activity.

### BACKGROUND

In the ethanol yeast bioconversion industry, improving yield often requires successive genetic engineering events which can be detrimental to the specific growth rate of the yeast and ultimately reduce the fermentation kinetic. It may thus be desirable to provide further genetic engineering events which would restore, at least in part the yeast specific growth rate while maintaining ethanol yield improvement.

### BRIEF SUMMARY

The present disclosure concerns a recombinant yeast host cell designed to improve its ethanol yield, while substantially maintaining its growth/fermentation rate. The recombinant yeast host cell comprises increased pyruvate decarboxylase activity.

In a first aspect, the present disclosure concerns a recombinant yeast cell for making ethanol. The recombinant yeast cell comprises one or more first genetic modifications to increase a yield of ethanol in the recombinant yeast cell as compared to a parental yeast cell. The recombinant yeast cell comprises a second genetic modification to increase pyruvate decarboxylase activity in the recombinant yeast cell when compared to the parental yeast cell. The parental yeast cell lacks the first genetic modification and the second genetic modification. In an embodiment, the one or more first genetic modification is capable of causing a reduction in a specific cell growth rate in an intermediate yeast cell as compared to the parental strain, wherein the intermediate yeast cell comprises the one or more first genetic modifications and lacks the second genetic modification. In an embodiment, the one or more first genetic modification is capable of causing a reduction in an ethanol production rate in an intermediate yeast cell as compared to the parental strain, wherein the intermediate yeast cell comprises the one or more first genetic modifications and lacks the second genetic modification. In another embodiment, the one or more first genetic modification is for, when compared to the parental yeast cell: reducing the production of glycerol, downregulating glycerol synthesis, decreasing the activity or production of one or more enzymes that facilitate glycerol synthesis; and/or facilitating glycerol transport. In some embodiments, the one or more first genetic modification comprises a genetic modification for reducing the expression or inactivating one ore more of the following native genes: gpd1, gpd2, gpp1 and/or gpp2, when compared to the parental yeast cell. In yet another embodiment, the one or more first genetic modification comprises a genetic modification for overexpressing a native polypeptide having glycerol proton symporter activity, and/or expressing a heterologous polypeptide having glycerol proton symporter activity. In some further embodiments, the native or the heterologous polypeptide having glycerol proton symporter activity is stl1. In still another embodiment, the one or more first genetic modification comprises a genetic modification for increasing formate/acetyl-CoA production, when compared to the parental yeast cell. In some embodiments, the one or more first genetic modification comprises a genetic modification for overexpressing a native polypeptide having pyruvate formate lyase activity and/or expressing a heterologous polypeptide having pyruvate formate lyase activity. In further embodiments, the native or the heterologous polypeptide having pyruvate formate lyase activity comprises pflA and/or pflB. In yet another embodiment, the first genetic modification comprises a genetic modification for increasing acetaldehyde/alcohol dehydrogenase activity, when compared to the parental yeast cell. In some embodiments, the first genetic modification comprises a genetic modification for overexpressing a native polypeptide having acetaldehyde/alcohol dehydrogenase activity and/or expressing a heterologous polypeptide having acetaldehyde/alcohol dehydrogenase activity. In some further embodiments, the native or the heterologous polypeptide having acetaldehyde/alcohol dehydrogenase activity comprises an acetaldehyde/alcohol dehydrogenase, such as, for example, adhE. In yet additional embodiments, the second genetic modification is for expressing a heterologous polypeptide having pyruvate decarboxylase activity. In some embodiments, the heterologous polypeptide having pyruvate decarboxylase activity has a lower Km than a native polypeptide having pyruvate decarboxylase activity. In yet additional embodiments, the heterologous polypeptide having pyruvate decarboxylase activity has the amino acid sequence of SEQ ID NO: 12, 14, 16, 17, 34, 35, 36 or 69, is a variant of the amino acid sequence of SEQ ID NO: 12, 14, 16, 17, 34, 35, 36 or 69 having pyruvate decarboxylase activity or is a fragment of the amino acid sequence of SEQ ID NO: 12, 14, 16, 17, 34, 35, 36 or 69 having pyruvate decarboxylase activity. In yet another embodiment, the recombinant yeast cell has at least one inactivated copy of a native gene encoding a native polypeptide having pyruvate decarboxylase activity. In still another embodiment, the recombinant yeast cell comprises a third genetic modification. In an embodiment, the third genetic modification comprises a genetic modification for overexpressing a native enzyme belonging to EC 1.2.1.9 or 1.2.1.90 and/or expressing a heterologous enzyme belonging to EC 1.2.1.9 or 1.2.1.90, such as, for example gapN. In another embodiment, the third genetic modification comprises a genetic modification for overexpressing a native polypeptide having alcohol dehydrogenase activity and/or expressing a heterologous polypeptide having alcohol dehydrogenase activity, such as, for example, adhB or adhA. In yet another embodiment, the recombinant yeast cell has at least one inactivated copy of a native gene encoding a native polypeptide having glucose-6-phosphate dehydrogenase activity. In still yet another embodiment, the recombinant yeast cell comprises at least one inactivated copy of a native gene encoding a native polypeptide having butanediol dehydrogenase activity. In some embodiments, the recombinant yeast cell is from the genus Saccharomyces sp., such as, for example, from the species Saccharomyces cerevisiae.

According to a second aspect, the present disclosure provides a method of making a recombinant yeast cell for producing ethanol. The method comprises introducing, in a parental yeast cell, one or more first genetic modification and a second genetic modification to obtain the recombinant yeast cell. The first genetic modification is for increasing a yield of ethanol in the recombinant yeast cell when compared to the parental yeast. The second genetic modification is for increasing pyruvate decarboxylase activity in the recombinant yeast cell when compared to the parental yeast cell. The parental yeast cell lacks the one or more first genetic modification and the second genetic modification. In some embodiments, the method is for increasing the yield in ethanol in the recombinant yeast cell when compared to the parental yeast cell, decreasing a yield in a fusel alcohol in the recombinant yeast cell when compared to the parental yeast cell, decreasing a yield in glycerol in the recombinant yeast cell when compared to the parental yeast cell and/or for providing tolerance in a stressful fermentation (e.g., in conditions of nitrogen scarcity, in the presence of a bacterial contamination, in the presence of a plurality of fermentation cycles and/or in the presence of a high temperature) in the recombinant yeast cell, when compared to the parental yeast cell. In an embodiment, the one or more first genetic modifications are defined as described herein. In another embodiment, the second genetic modification is defined as described herein. In another embodiment, the method further comprises inactivating a copy of a native gene encoding a native polypeptide having pyruvate decarboxylase activity to obtain the recombinant yeast cell. In still another embodiment, the method further comprises introducing a third genetic modification in the parental yeast cell to obtain the recombinant yeast cell, wherein the third genetic modification is as described herein. In yet another embodiment, the method further comprises inactivating a copy of a native gene encoding a native polypeptide having glucose-6-phosphate dehydrogenase activity to obtain the recombinant yeast cell. In still another embodiment, the method further comprises inactivating a copy of a native gene encoding a native butanediol dehydrogenase. In some embodiments, the recombinant yeast cell is defined as described herein.

According to a third aspect, the present disclosure provides a process for making ethanol. The process comprises contacting the recombinant yeast cell described herein, obtainable or obtained by the method described herein with a substrate under a condition allowing the conversion of at least part of the substrate into ethanol. In an embodiment, the process comprises contacting a dose of an exogenous enzyme with the recombinant yeast cell and the substrate. In another embodiment, the process comprises contacting a dose of a nitrogen source with the recombinant yeast cell and the substrate. In yet another embodiment, the process comprises a plurality of fermentation cycles. In some embodiments, the substrate is or comprises corn or a product derived from corn. In yet a further embodiments, the substrate is a corn mash. In some embodiments, the substrate is or comprises sugarcane or a product derived from sugarcane. In yet additional embodiments, the substrate is a sugarcane must. In an embodiment, the process is for increasing the yield in ethanol in the recombinant yeast cell when compared to the parental yeast cell. In another embodiment, the process is for decreasing a yield in a fusel alcohol in the recombinant yeast cell when compared to the parental yeast cell. In another embodiment, the process is for decreasing a yield in glycerol in the recombinant yeast cell when compared to the parental yeast cell. In a further embodiments, the process is for providing tolerance (e.g., in conditions of nitrogen scarcity, in the presence of a bacterial contamination, in the presence of a plurality of fermentation cycles, and/or in the presence of a high temperature) in the recombinant yeast cell, when compared to the parental yeast cell.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 provides the schematic representation of endogenous fermentation pathway in Saccharomyces cerevisiae. Illustration of fermentation products, secondary metabolites, enzymes, and redox co-factors produced during fermentation. S. cerevisiae contains several enzymes involved in the conversion of pyruvate to acetaldehyde or acetoin: pdc1, pdc5, and pdc6. pdc1 and is also responsible for converting pyruvate to acetaldehyde or acetoin. 2,3-butanediol, formed through a reaction catalyzed by bdh1 and bdh2, is considered a dead-end secondary metabolite, as butanediol cannot be further converted to ethanol anaerobically.

FIGS. 2A and 2B provide a time lapse of the cell counts associated with strains M2390 and M28047 during the fermentation.

(FIG. 2A) provides the total number of cells (per mL, left axis) in function of time (in h) for strain M2390 (dashed line) and M28047 (complete line). Viable cells (right axis) for M2390 (X) and M28047 (▴) is also provided.

(FIG. 2B) provides the total number of living cells (per mL, left axis) in function of time (in h) for strain M2390 (dashed line) and M28047 (complete line). Viable cells (right axis) for M2390 (X) and M28047 (▴) is also provided.

FIGS. 3A to 3D provide the CO₂ profiles obtained during fermentations using different yeast strains. Results are provided as CO₂ measured (in mL/min) in function of time (hours) and of the strain used.

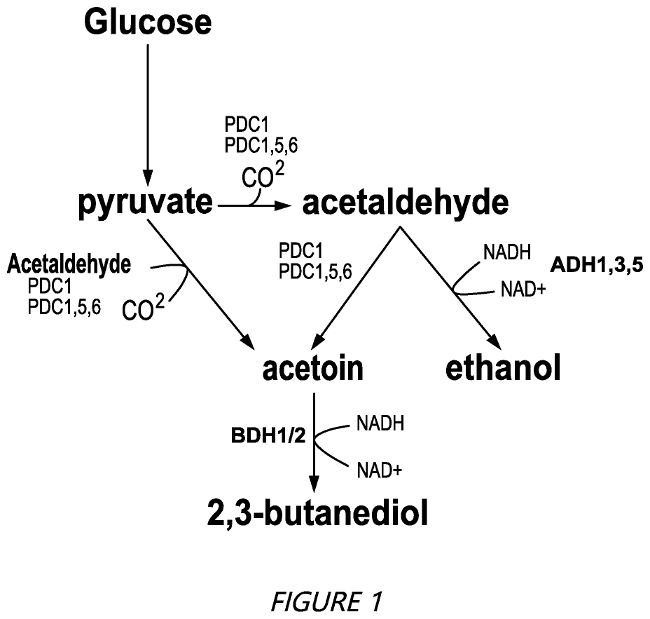

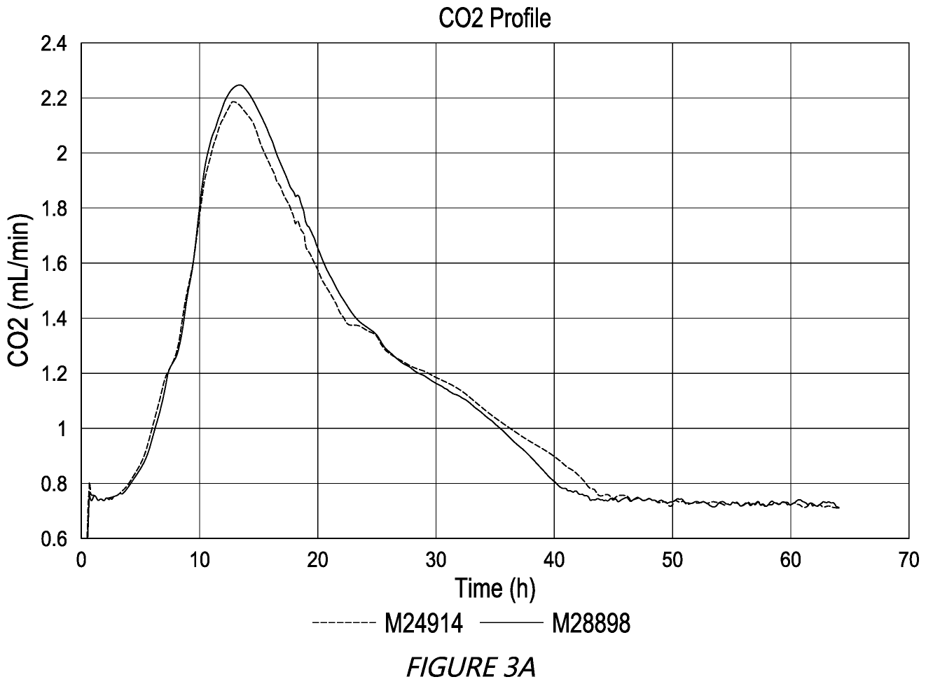

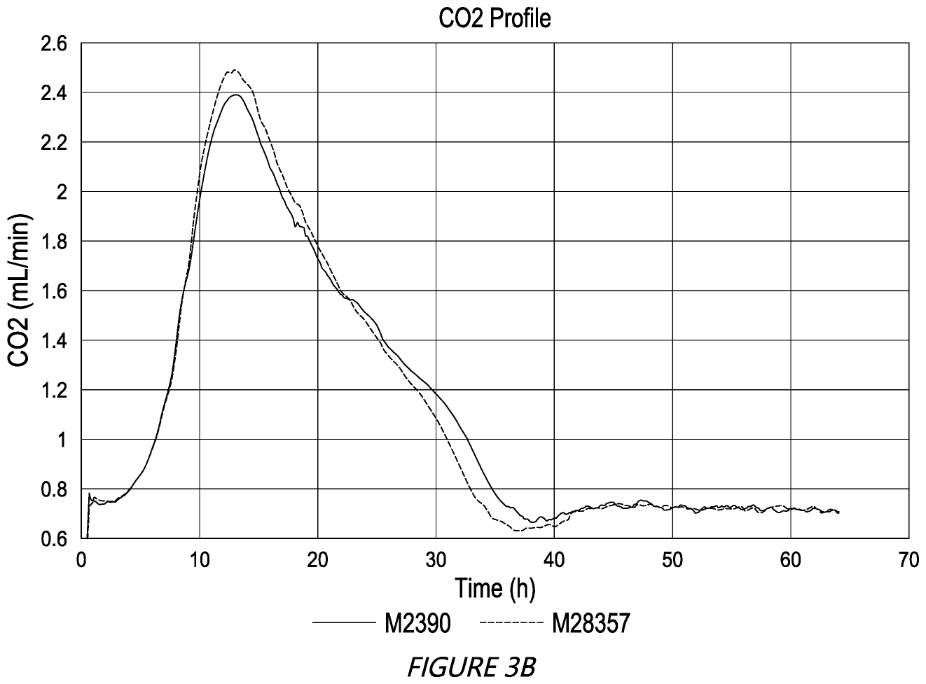

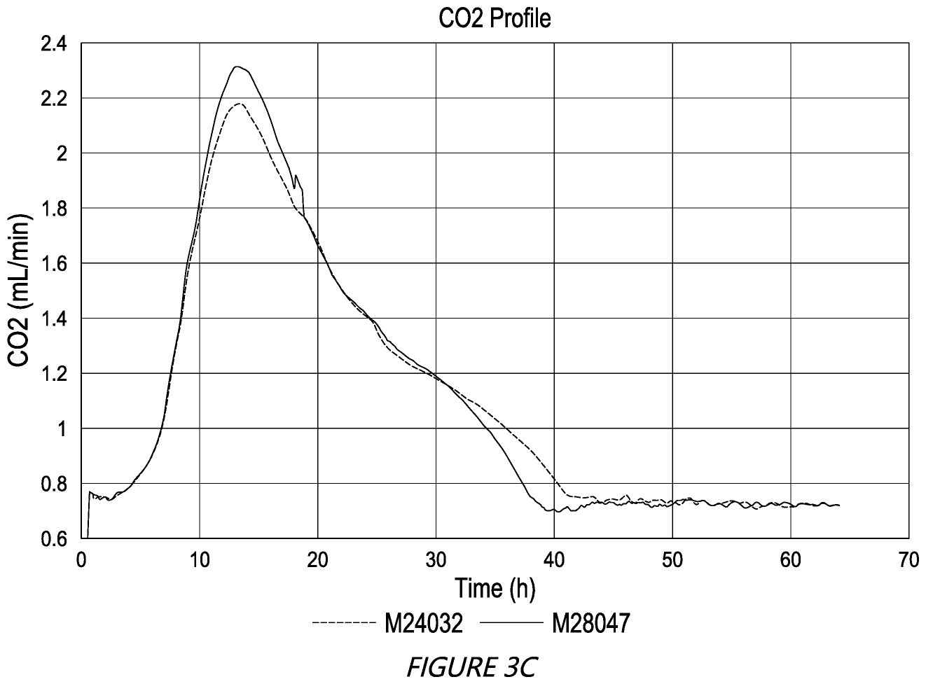

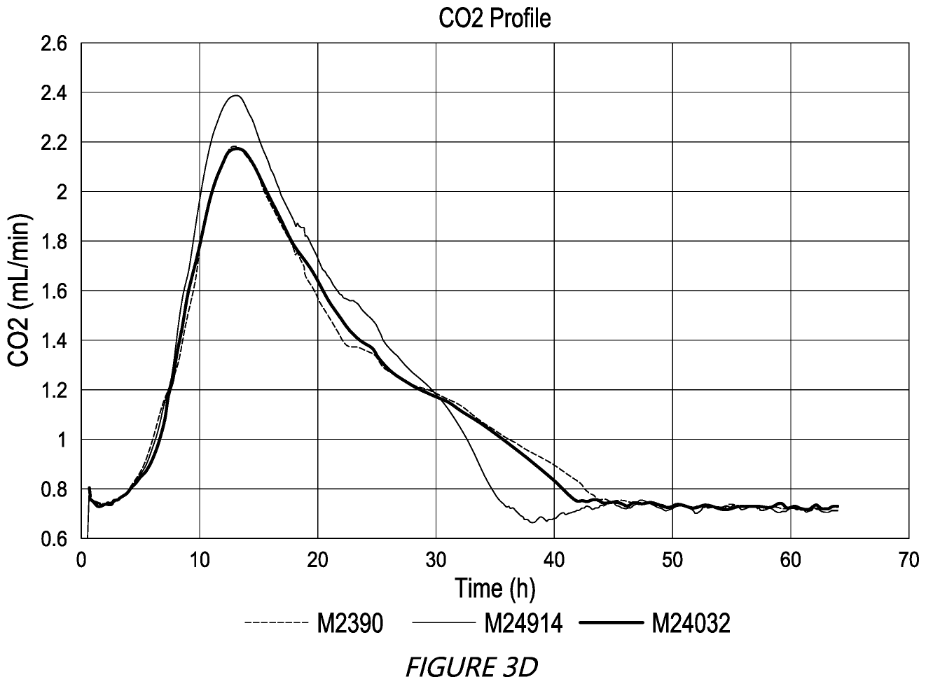

(FIG. 3A) provides the data obtained with strains M24914 (dashed line) and M28898 (complete line).

(FIG. 3B) provides the data obtained with strains M2390 (complete line) and M28357 (dotted line).

(FIG. 3C) provides the data obtained with strains M24032 (dashed line) and M28047 (complete line).

(FIG. 3D) provides the data obtained with strains M2390 (complete line), M24914 (dashed line) and M24032 (dotted line).

### DETAILED DESCRIPTION

The present disclosure provides a recombinant yeast cell including a genetic modification allowing it to increase its overall pyruvate decarboxylase activity (when compared to corresponding parental yeast cells lacking such genetic modification). The recombinant yeast cell can, in some embodiments, overexpress one or more native polypeptide having pyruvate decarboxylase activity and/or express one or more heterologous polypeptide having pyruvate decarboxylase activity. In some embodiments, the recombinant yeast cells of the present disclosure include a heterologous nucleic acid encoding a heterologous polypeptide having pyruvate decarboxylase activity. In additional embodiments, the heterologous polypeptide having pyruvate decarboxylase activity has a higher affinity (e.g., and thus a lower Km) towards pyruvate that the native polypeptide(s) having pyruvate decarboxylase activity that may be expressed by the parental yeast cell (and optionally in the recombinant yeast cell as well). The increase in the pyruvate decarboxylase activity in the recombinant yeast cell can advantageously be used to increase its specific growth rate, to increase its fermentation rate, to provide tolerance in stressful fermentations (e.g., for example, in conditions of nitrogen scarcity, in the presence of a bacterial contamination, in the presence of a plurality of fermentation cycles, and/or in the presence of a high temperature) to increase a yield of ethanol and/or to decrease a yield of one or more fermentation by-product (such as, for example, glycerol and/or a fusel alcohol).

In the context of the present disclosure, the recombinant yeast cell can be, for example, from the genus Saccharomyces, Kluyveromyces, Arxula, Debaryomyces, Candida, Pichia, Phaffia, Schizosaccharomyces, Hansenula, Kloeckera, Schwanniomyces or Yarrowia. Suitable yeast species can include, for example, Saccharomyces cerevisiae, Saccharomyces bulderi, Saccharomyces barnetti, Saccharomyces exiguus, Saccharomyces uvarum, Saccharomyces diastaticus, Kluyveromyces lactis, Kluyveromyces marxianus or Kluyveromyces fragilis. In some embodiments, the recombinant yeast cell is selected from the group consisting of Saccharomyces cerevisiae, Schizzosaccharomyces pombe, Candida albicans, Pichia pastoris, Pichia stipitis (Komagatella phaffi), Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In some embodiments, the recombinant yeast cell can be an oleaginous yeast cell. For example, the oleaginous yeast cell can be from the genus Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiments, the recombinant yeast cell can be an oleaginous microalgae host cell (e.g., for example, from the genus Thraustochytrium or Schizochytrium). In an embodiment, the recombinant yeast cell is from the genus Saccharomyces and, in some additional embodiments, from the species Saccharomyces cerevisiae.

In some embodiments, the present disclosure concerns the expression of one or more polypeptides (including enzymes), a variant thereof or a fragment thereof in a yeast cell. A variant comprises at least one amino acid difference when compared to the amino acid sequence of the wild-type polypeptide. The polypeptide “variants” have at least 50%, 55%, 60%, 65%, 70%, 75%, 80%, 85%, 90%, 95%, 96%, 97%, 98% or 99% identity to the wild-type polypeptides described herein. The term “percent identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The polypeptide variants exhibit the biological activity associated with the wild-type polypeptide. In an embodiment, the variant polypeptide exhibits at least 50%, 55%, 60%, 65%, 70%, 75%, 80%, 85%, 90%, 95%, 96%, 97%, 98% or 99% of the biological activity of the wild-type polypeptide. The biological activity of the polypeptides and variants can be determined by methods and assays known in the art.

The variant polypeptides described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide.

A “variant” of the polypeptide can be a conservative variant or an allelic variant. As used herein, a conservative variant refers to alterations in the amino acid sequence that do not adversely affect the biological function(s) of the polypeptide. A substitution, insertion or deletion is said to adversely affect the polypeptide when the altered sequence prevents or disrupts a biological function associated with the polypeptide. For example, the overall charge, structure or hydrophobic-hydrophilic properties of the polypeptide can be altered without adversely affecting a biological activity. Accordingly, the amino acid sequence can be altered, for example to render the polypeptide more hydrophobic or hydrophilic, without adversely affecting the biological activitie(s) of the polypeptide.

The polypeptides (including enzymes) can be a fragment of wild-type polypeptide or fragment of a variant polypeptide. Polypeptide “fragments” have at least at least 100, 200, 300, 400, 500 or more consecutive amino acids of the wild-type polypeptide or the polypeptide variant. A fragment comprises at least one less amino acid residue when compared to the amino acid sequence of the wild-type polypeptide. In some embodiments, the fragment corresponds to the wild-type polypeptide to which the signal sequence was removed. In some embodiments, the “fragments” have at least 50%, 55%, 60%, 65%, 70%, 75%, 80%, 85%, 90%, 95%, 96%, 97%, 98% or 99% identity to the wild-type polypeptides described herein. In some embodiments, fragments of the polypeptides can be employed for producing the corresponding full-length enzyme by peptide synthesis. Therefore, the fragments can be employed as intermediates for producing the full-length polypeptides.

The fragments of wild-type polypeptide or of variants the polypeptides exhibit the biological activity of the wild-type polypeptide or the variant polypeptide In an embodiment, the fragment polypeptide exhibits at least 50%, 55%, 60%, 65%, 70%, 75%, 80%, 85%, 90%, 95%, 96%, 97%, 98% or 99% of the biological activity of the wild-type polypeptide or variant thereof. The biological activity of the wild-type polypeptide and variants can be determined by methods and assays known in the art.

In some additional embodiments, the present disclosure also provides reducing the expression of or inactivating a gene or a gene ortholog of a gene known to encode a polypeptide. A “gene ortholog” is understood to be a gene in a different species that evolved from a common ancestral gene by speciation. In the context of the present invention, a gene ortholog encodes a polypeptide exhibiting the same biological function than the wild-type polypeptide.

In some further embodiments, the present disclosure also provides reducing the expression or inactivating a gene or a gene paralog of a gene known to encode polypeptide. A “gene paralog” is understood to be a gene related by duplication within the genome. In the context of the present invention, a gene paralog encodes a polypeptide that exhibit a similar biological function and could exhibit an additional biological function when compared to the wild-type polypeptide.

### Methods for Making the Recombinant Yeast Cell

The present disclosure provides methods for making the recombinant yeast cell. Broadly, the method comprises introducing the one or more first and the one or more second genetic modifications, in any order or at the same time, in a parental yeast cell to obtain the recombinant yeast cell of the present disclosure. In the context of the present disclosure, the expression “first” genetic modification does not mean that it is necessarily introduced before the “second” genetic modification in the yeast cell. The expression “first” genetic modification refers to a class of genetic modifications capable of causing an increase in a yield of ethanol. In an embodiment, the first genetic modification can include increasing the native expression of a first polypeptide capable of increasing a yield in ethanol. In another embodiment, the first genetic modification can include providing the heterologous expression of a first polypeptide capable of increasing a yield in ethanol. By the same token, the expression “second” genetic modification does not mean that it is necessarily introduced after the “first” genetic modification in the yeast cell. The expression “second” genetic modification refers to a class of genetic modifications capable of increasing pyruvate decarboxylase activity. In an embodiment, the second genetic modification can include increasing the native expression of a second polypeptide capable of increasing pyruvate carboxylase activity. In another embodiment, the second genetic modification can include providing the heterologous expression of a second polypeptide capable of increasing pyruvate decarboxylase activity. In some embodiments, the method can include introducing one or more heterologous nucleic acid molecules (which comprises, for example, includes at least one of the first or the second genetic modification and optionally additional the third genetic modition and/or further genetic modifications) in the parental yeast cell to obtain the recombinant yeast cell. The one or more heterologous nucleic acid molecules can include, for example, a promoter to increase the expression of one or more first native polypeptide and/or one or more first second native polypeptide. The one or more heterologous nucleic acid molecules can include, for example, a gene encoding for one or more first heterologous polypeptide and/or one or more second heterologous polypeptides. The heterologous nucleic acid molecules can be introduced in the genome of the recombinant yeast cell by any known genetic engineering methods, such as, for example, by a double strand break mechanism, Cre-LoxP mediated recombination, delitto perfetto, meganuclease-mediated double strand break, MAD7 and/or CRISPR/Cas9. In some embodiments, the method can include determining if the genetic modifications have been correctly integrated in the recombinant yeast cell genome.

The present disclosure also provides methods for making the intermediate yeast cell. In some embodiments, the intermediate yeast cell can be used to make the recombinant yeast cell of the present disclosure. In additional embodiments, the intermediate yeast cell can be used for comparison with the recombinant yeast cell or the parental yeast cell. Broadly, the method comprises introducing the first genetic modification(s) in a parental yeast cell to obtain the intermediate yeast cell. The method specifically excludes introducing the second genetic modification(s) in the intermediate yeast cell because it does not include (e.g., excludes) the second genetic modification(s). For example, the method can include introducing one or more heterologous nucleic acid molecules in the parental yeast cell to obtain the intermediate yeast cell. The heterologous nucleic acid molecules can be introduced in the genome of the intermediate yeast cell by any known genetic engineering methods, such as, for example, by a double strand break mechanism, Cre-LoxP mediated recombination, delitto perfetto, meganuclease-mediated double strand break, MAD7 and/or CRISPR/Cas9. In some embodiments, the methods also include introducing one or more third genetic modifications and, in some further embodiments, additional genetic modifications (but not the second genetic modification) to obtain the intermediate yeast cell. In some embodiments, the method can include determining if the genetic modifications have been correctly integrated in the recombinant yeast cell genome.

In additional embodiments, the recombinant yeast cell of the present disclosure can include a third genetic modification for overexpressing a native enzyme belonging to EC 1.2.1.9 or 1.2.1.90 and/or expressing a heterologous enzyme belonging to EC 1.2.1.9 or 1.2.1.90. As such, in some embodiments, the method described herein can include introducing a third genetic modification for overexpressing a native enzyme belonging to EC 1.2.1.9 or 1.2.1.90 and/or expressing a heterologous enzyme belonging to EC 1.2.1.9 or 1.2.1.90 in the recombinant yeast cell.

In some embodiments, the recombinant yeast cell of the present disclosure can include additional further genetic modifications for reducing the expression or inactivating one or more native genes. The reduction in the expression or the inactivation can be observed in at least one inactivated copy of a native gene encoding a native polypeptide having glucose-6-phosphate dehydrogenase activity. In such embodiment, the method comprises introducing a further genetic modification for reducing the expression or inactivating one or more native genes encoding one or more native polypeptides having glucose-6-phosphate dehydrogenase activity in the recombinant yeast cell. The reduction in the expression or the inactivation can be observed in at least one inactivated copy of a native gene encoding a native polypeptide having butanediol dehydrogenase activity. In such embodiment, the method comprises introducing a further genetic modification for reducing the expression or inactivating one or more native genes encoding one or more native polypeptides having having butanediol dehydrogenase activity in the recombinant yeast cell. This further genetic modification can include for example, removing at least one nucleic acid residue from the codon region (and in some embodiments the entire codon region) of the gene whose is intended to be inactivated or whose expression is intended to be reduced. The further genetic modification can also include, for example, adding at least one nucleic acid residue in the coding region (e.g., interrupting the codon region) of the the gene whose is intended to be inactivated or whose expression is intended to be reduced.

When the genetic modification is aimed at increasing the expression of a specific targeted gene (which may native or heterologous), the genetic modification can be made in one or multiple genetic locations. When the genetic modification is aimed at reducing or inhibiting the expression of a specific targeted gene (which is endogenous to the host cell), the genetic modifications can be made in one or all copies of the targeted gene(s). In the context of the present disclosure, when recombinant yeast cells and intermediate yeast cells are qualified as being “genetically engineered”, it is understood to mean that they have been manipulated to either add at least one or more heterologous nucleic acid residue and/or remove at least one endogenous (or native) nucleic acid residue in order to reduce or inhibit the expression of the targeted gene(s). In some embodiments, the one or more nucleic acid residues that are added can be derived from a heterologous cell or the recombinant/intermediate yeast cell itself. In the latter scenario, the nucleic acid residue(s) can (are) added at a genomic location which is different than the native genomic location or one or more additional copies can be knocked-in at the genomic location of a native gene (to introduce additional heterologous copies of the native gene). The genetic manipulations did not occur in nature and are the results of in vitro manipulations of the parental yeast cell.

In some embodiments, each genetic modification can be encoded on one or more heterologous or native nucleic acid molecules. In some embodiments, the heterologous or native nucleic acid molecule can encode one or more polypeptide (which may be additional copies of a native gene). In other embodiments, the heterologous nucleic acid molecules can encode a promoter or other regulatory sequence for upregulating or downregulating native polypeptide expression. In some embodiments, the heterologous nucleic acid molecules of the present disclosure can include a signal sequence to favor the secretion of the heterologous polypeptide or the native polypeptide.

The term “heterologous” when used in reference to a nucleic acid molecule (such as a promoter, a terminator or a coding sequence) or a polypeptide/polypeptide refers to a nucleic acid molecule or a polypeptide/polypeptide that is not natively found in the recombinant host cell. “Heterologous” also includes a native coding region/promoter/terminator, or portion thereof, that was removed from the source organism and subsequently reintroduced into the source organism in a form that is different from the corresponding native gene. In one embodiment, the native coding region/promoter/terminator, or portion thereof, that was removed from the source organism and was reintroduced into the source organism in a different location than its natural location in the parental yeast cell. “Heterologous” also includes a native coding region/promoter/terminator, or portion thereof, that was introduced into the source organism is introduced in additional copies not present in the parental yeast cell. “Heterologous” further includes replacing a native coding region/promoter/terminator with another combination of a native coding region/promoter/terminator that are not present in the source organism. Such replacement can be made, in some embodiments, at the natural location of the native coding region/promoter/terminator. In a specific example, the native coding region/promoter regions of a target gene can be removed and replaced by another coding region of the target gene (which, in some embodiments, maybe the identical to the native coding region) but combined with another promoter than the native promoter of the target gene. In yet another example, the native coding region/terminator regions of a target gene can be removed and replaced by another coding region of the target gene (which, in some embodiments, maybe the identical to the native coding region) but combined with another terminator than the native terminator of the target gene.

The heterologous nucleic acid molecule is purposively introduced into the recombinant yeast cell. For example, a heterologous element could be derived from a different strain of host cell, or from an organism of a different taxonomic group (e.g., different kingdom, phylum, class, order, family genus, or species, or any subgroup within one of these classifications). As used herein, the term “native” when used in inference to a gene, polypeptide, enzymatic activity, or pathway refers to an unmodified gene, polypeptide, enzymatic activity, or pathway originally found in the recombinant host cell. In some embodiments, heterologous polypeptides derived from a different strain of host cell, or from an organism of a different taxonomic group (e.g., different kingdom, phylum, class, order, family, genus, or species, or any subgroup within one of these classifications) can be used in the context of the present disclosure.

The heterologous nucleic acid molecules of the present disclosure can comprise a coding region for the heterologous polypeptide. A DNA or RNA “coding region” is a DNA or RNA molecule (preferably a DNA molecule) which is transcribed and/or translated into a heterologous polypeptide in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, transcription terminators, translation leader sequences, RNA processing site, effector binding site and stem-loop structure. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell (such as the recombinant yeast cell of the present disclosure), a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The heterologous nucleic acid molecules described herein can comprise transcriptional and/or translational control regions. “Transcriptional and translational control regions” are DNA regulatory regions, such as promoters, enhancers, terminators, and the like, that provide for the expression of a coding region in a recombinant host cell. In eukaryotic cells, polyadenylation signals are considered control regions.

In some embodiments, the heterologous nucleic acid molecules of the present disclosure include a coding sequence for a heterologous polypeptide, optionally in combination with a promoter and/or a terminator. In some embodiments, the heterologous nucleic acid molecules of the present disclosure include a nucleic acid sequence encoding a promoter for overexpressing a native gene encoding a native polypeptide. In the heterologous nucleic acid molecules of the present disclosure, the promoter and the terminator (when present) are operatively linked to the nucleic acid coding sequence of the heterologous or native polypeptide, e.g., they control the expression and the termination of expression of the nucleic acid sequence of the heterologous or the native polypeptide. The heterologous nucleic acid molecules of the present disclosure can also include a nucleic acid sequence coding for a signal sequence, e.g., a short peptide sequence for exporting the heterologous polypeptide outside the host cell. When present, the nucleic acid sequence coding for the signal sequence is directly located upstream and in frame of the nucleic acid sequence coding for the heterologous polypeptide.

In the recombinant yeast cell described herein, the nucleic acid molecule coding for the promoter and the nucleic acid molecule coding for the heterologous or the native polypeptide are operatively linked to one another. In the context of the present disclosure, the expressions “operatively linked” or “operatively associated” refers to fact that the promoter is physically associated to the nucleic acid molecule coding for the heterologous or the native polypeptide in a manner that allows, under certain conditions, for expression of the heterologous polypeptide from the nucleic acid molecule. In an embodiment, the promoter can be located upstream (5′) of the nucleic acid sequence coding for the heterologous polypeptide. In still another embodiment, the promoter can be located downstream (3′) of the nucleic acid sequence coding for the heterologous polypeptide. In the context of the present disclosure, one or more than one promoter can be included in the heterologous nucleic acid molecule. When more than one promoter is included in the heterologous nucleic acid molecule, each of the promoters is operatively linked to the nucleic acid sequence coding for the heterologous or native polypeptide. The promoters can be located, in view of the nucleic acid molecule coding for the heterologous or native polypeptide, upstream, downstream as well as both upstream and downstream.

The term “promoter” refers to a DNA fragment capable of controlling the expression of a coding sequence or functional RNA. The term “expression,” as used herein, refers to the transcription and stable accumulation of sense mRNA from the heterologous nucleic acid molecule or the native gene described herein. Expression may also refer to translation of mRNA into a polypeptide. Promoters may be derived in their entirety from the promoter of a native gene, or be composed of different elements derived from different promoters found in nature, or even comprise synthetic DNA segments. It is understood by those skilled in the art that different promoters may direct the expression at different stages of development, or in response to different environmental or physiological conditions. Promoters which cause a gene to be expressed in most cells at most times at a substantial similar level are commonly referred to as “constitutive promoters”. Promoters which cause a gene to be expressed during the propagation phase of a yeast cell are herein referred to as “propagation promoters”. Propagation promoters include both constitutive and inducible promoters, such as, for example, glucose-regulated, molasses-regulated, stress-response promoters (including osmotic stress response promoters) and aerobic-regulated promoters. It is further recognized that since in most cases the exact boundaries of regulatory sequences have not been completely defined, DNA fragments of different lengths may have identical promoter activity. A promoter is generally bounded at its 3 terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as polypeptide binding domains (consensus sequences) responsible for the binding of the polymerase.

The promoter can be native or heterologous to the nucleic acid molecule encoding the native or the heterologous polypeptide. The promoter can be heterologous to the native gene encoding the native polypeptide to be overexpressed. The promoter can be heterologous or derived from a strain being from the same genus or species as the recombinant host cell. In an embodiment, the promoter is derived from the same genus or species of the yeast cell and the heterologous polypeptide is derived from a different genus than the host cell. The promoter can be a single promotor or a combination of different promoters. In some embodiments, the promoter is a propagation promoter. In some embodiments, the promoter is an aerobic promoter.

In the context of the present disclosure, the promoter controlling the expression of the heterologous polypeptide or the native polypeptide can be a constitutive promoter (such as, for example, tef1p (e.g., the promoter of the tef1 gene), tef2p (e.g., the promoter of the tef2 gene), cwp2p (e.g., the promoter of the cwp2 gene), ssa1p (e.g., the promoter of the ssa1 gene), eno1p (e.g., the promoter of the eno1 gene), eno2p (e.g., the promoter of the eno2 gene), hxk1p (e.g., the promoter of the hxk1 gene), pgk1p (e.g., the promoter of the pgk1 gene), ydr524c-bp (e.g., the promoter of the ydr524c-b gene), gpm1p (e.g., the promoter of the gpm1 gene), and/or tpi1p (e.g., the promoter of the tpi1 gene). However, in some embodiments, it is preferable to limit the expression of the polypeptide. As such, the promoter controlling the expression of the heterologous polypeptide or the native polypeptide can be an inducible or modulated promoters such as, for example, a glucose-regulated promoter (e.g., the promoter of the hxt3 gene (referred to as hxt3p), the promoter of the hxt7 gene (referred to as hxt7p), or the promoter of the cyc1 gene (referred to as the cyc1p)). In still another embodiment, the promoter can be a sulfite-regulated promoter (e.g., the promoter of the gpd2 gene (referred to as gpd2p or the promoter of the fzf1 gene (referred to as the fzf1p)), the promoter of the ssu1 gene (referred to as ssu1p), the promoter of the ssu1-r gene (referred to as ssur1-rp). In yet another embodiment, the promoter is a ribosomal promoter (e.g., the promoter of the rp/3 gene (referred to as the rpl3p) or the promoter of the qcr8 gene (referred to as qcr8p)) In an embodiment, the promoter is an anaerobic-regulated promoter, such as, for example tdh1p (e.g., the promoter of the tdh1 gene), pau5p (e.g., the promoter of the pau5 gene), hor7p (e.g., the promoter of the hor7 gene), adh1p (e.g., the promoter of the adh1 gene), tdh2p (e.g., the promoter of the tdh2 gene), tdh3p (e.g., the promoter of the tdh3 gene), gpd1p (e.g., the promoter of the gpd1 gene), cdc19p (e.g., the promoter of the cdc19 gene), pdc1p (e.g., the promoter of the pdc1 gene), hxt3p (e.g., the promoter of the hxt3 gene), dan1p (e.g., the promoter of the dan1 gene), tir1p (e.g., the promoter of the tir1 gene) and tpi1p (e.g., the promoter of the tpi1 gene). In another embodiment, the promoter is a stress-regulated promoter such as, for example, hor7p (e.g., the promoter of the hor7 gene). In still another embodiment, the promoter is a glycolytic-regulated promoter such as, for example, adh1p (e.g., the promoter of the adh1 gene), eno2p (e.g., the promoter of the eno2 gene), pgk1p (e.g., the promoter of the pgk1 gene), tef1p (e.g., the promoter of the tef1 gene), tef2p (e.g., the promoter of the tef2 gene), gpm1p (e.g., the promoter of the gpm1 gene) and/or tpi1p (e.g., the promoter of the tpi1 gene). One or more promoters can be used to allow the expression of each heterologous polypeptides in the recombinant yeast cell.

One or more promoters can be used to allow the expression of each heterologous/native polypeptides in the recombinant yeast cell. In the context of the present disclosure, the expression “functional fragment of a promoter” when used in combination to a promoter refers to a shorter nucleic acid sequence than the native promoter which retain the ability to control the expression of the nucleic acid sequence encoding the heterologous polypeptide. Usually, functional fragments are either 5′ and/or 3′ truncation of one or more nucleic acid residue from the native promoter nucleic acid sequence.

The heterologous nucleic acid molecule of the present disclosure can be integrated in the chromosome(s) of the yeast's genome. The term “integrated” as used herein refers to genetic elements that are placed, through molecular biology techniques, into the genome of the recombinant yeast cell. For example, genetic elements can be placed into the chromosomes of the recombinant yeast cell as opposed to in a vector such as a plasmid carried by the recombinant yeast cell. Methods for integrating genetic elements into the chromosome of a host cell are well known in the art and include homologous recombination. The heterologous nucleic acid molecule can be present in one or more copies in the recombinant yeast cell's chromosome. Alternatively, the heterologous nucleic acid molecule can be independently replicating from the recombinant yeast cell's chromosome. In such embodiment, the nucleic acid molecule can be stable and self-replicating. The heterologous nucleic acid molecules can be present in one or more copies in the recombinant yeast cell. For example, each heterologous nucleic acid molecules can be present in 1, 2, 3, 4, 5, 6, 7, 8 copies or more per genome or chromosome.

The present disclosure also provides nucleic acid molecules for modifying the yeast cell so as to allow the expression of the one or more heterologous polypeptide, variants or fragments thereof or the overexpression of one or more native polypeptide. The nucleic acid molecule may be DNA (such as complementary DNA, synthetic DNA or genomic DNA) or RNA (which includes synthetic RNA) and can be provided in a single stranded (in either the sense or the antisense strand) or a double stranded form. The contemplated nucleic acid molecules can include alterations in the coding regions, non-coding regions, or both. Examples are nucleic acid molecule variants containing alterations which produce silent substitutions, additions, or deletions, but do not alter the properties or activities of the encoded polypeptide, variants or fragments.

In some embodiments, the heterologous nucleic acid molecules which can be introduced into the recombinant host cells are codon-optimized with respect to the intended recipient recombinant yeast cell. As used herein the term “codon-optimized coding region” means a nucleic acid coding region that has been adapted for expression in the cells of a given organism by replacing at least one, or more than one, codons with one or more codons to optimize expression levels. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism.

The heterologous nucleic acid molecules can be introduced in the yeast cell using a vector. A “vector,” e.g., a “plasmid”, “cosmid” or “artificial chromosome” (such as, for example, a yeast artificial chromosome) refers to an extra chromosomal element and is usually in the form of a circular double-stranded DNA molecule. Such vectors may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

### Methods for Propagating and Formulating the Recombinant Yeast Cell

The present disclosure allows for the propagation of recombinant yeast cell of the present disclosure and ultimately the formulation of propagated recombinant yeast cells. In the propagation process, the recombinant yeast cell is placed in a culture medium under suitable condition for cell growth. The culture medium can comprise a carbon source (such as, for example, molasses, sucrose, glucose, dextrose syrup, ethanol, corn, glycerol, corn steep liquor and/or a lignocellulosic biomass), a nitrogen source (such as, for example, ammonia or another inorganic source of nitrogen) and a phosphorous source (such as, for example, phosphoric acid or another inorganic source of phosphorous). The culture medium can further comprise additional micronutrients such as vitamins and/or minerals to support the propagation of the recombinant yeast cell.

The propagation can be conducted under conditions to allow cell growth and the accumulation of yeast biomass as well as to limit fermentation product production (e.g., ethanol production). The propagation process can be conducted in aerobic conditions. The propagation process can be conducted at a specific pH and/or a specific temperature which is optimal for the expression of the heterologous polypeptide or for the over-expression of the native polypeptide. In embodiments in which the recombinant yeast cell is from the genus Saccharomyces, the process can comprise controlling the pH of the culture medium to between about 3.0 to about 6.0, about 3.5 to about 5.5 or about 4.0 to about 5.5. In a specific embodiment, the pH is controlled at about 4.5. In another example, in embodiments in which the recombinant yeast cell is from the genus Saccharomyces, the process can comprise controlling the temperature of the culture medium between about about 20° C. to about 40° C., about 25° C. to about 30° C. or about 30° C. to about 35° C. In a specific embodiment, the temperature is controlled at between about about 30° C. to about 35° C. (32° C. for example).

The formulation step can also include a step of removing some or the majority of the water used during the propagation process. For example, the formulation step can include a step of dehydrating, filtering (including ultra-filtrating) and/or centrifuging the propagated recombinant yeast cell. The formulation step can include providing the recombinant yeast cells in the form of a cream. The formulation can optionally include drying the propagated recombinant yeast cell to provide it in a dried form. The drying step, when present, can include, for example, with spray-drying and/or fluid-bed drying.

### First Genetic Modification(s)

In embodiments, the recombinant yeast cell of the present disclosure includes one or more first genetic modifications for increasing a yield of ethanol in the recombinant yeast cell when compared to the parental yeast cell (lacking the first genetic modification). When submitted to comparable/similar fermentation conditions, the recombinant yeast cell and the parental yeast cell will generate, respectively, a first yield of ethanol and a second yield of ethanol. It is understood that, at the end of the fermentation, the first yield of ethanol obtained from using the recombinant yeast cell will be higher than the second yield of ethanol obtained using the parental yeast cell. This increase in ethanol yield is due in part to the presence of the one or more genetic modifications.

It is possible that, in some embodiments, the presence of the one or more first genetic modifications (in the absence of the second genetic modification) is incapable of causing and/or does not cause a reduction in the specific growth rate in a yeast cell. It is also possible that, in some embodiments, the presence of the one or more first genetic modifications (in the absence of the second genetic modification) is capable of causing and/or causes a reduction in a specific cell growth rate in a yeast cell. As it is known in the art, the specific growth rate is known as the rate of increase of biomass of a cell population (e.g., a yeast population) per unit of biomass concentration. The specific growth rate can be calculated by evaluating biomass formation from samples collected during exponential growth phase of a fermentation. In some embodiments, the specific growth rate (μ) can be determined by optical density (OD) measurements using the following formula:

$$μ = ln ( N ( t ) / N 0 ) / t = [ ln ( Nt ) - ln ( No ) ] / t$$

wherein μ (h⁻¹) is the specific growth rate and Nt final cell density, NO is the original cell density, t is the time (in h) between samples. The maximum specific growth rate (μmax) is the the point in the fermentation, at which the cells are growing at the highest specific growth rate. In some embodiments, the presence of the one or more first genetic modifications (in the absence of the second genetic modification) is capable of causing and/or causes a reduction in a maximum specific cell growth rate in a yeast cell. As it is known in the art, the specific growth rate as well as the maximum specific growth rate can be determined during the exponential growth phase of the yeast cell. In some embodiments, the second genetic modification increases the specific growth rate and, in some further embodiments, increases the maximal specific growth rate of the recombinant yeast cell when compared to the intermediate yeast cell.

In some embodiments, the presence of the one or more first genetic modifications is capable of causing and/or causes an increase in the time to complete a fermentation in the intermediate yeast cell when compared to the parental yeast cell. The time to complete a fermentation can be calculated as the time elapsed between the start of the fermentation and the end of the fermentation. In rapid fermentations (such as those using sugar cane must as a substrate), the start of the fermentation can be determined as the time at which CO₂ starts to be generated by the population of yeasts. The end of the fermentation can be determined as the time at which no more CO₂ is produced/detected above a certain threshold by the population of yeasts cells. In other fermentations (such as those using corn or corn mash as a substrate), the start of the fermentation can be determined as the time at which the carbohydrates start being consumed by the population yeast cells. The end of the fermentation can be determined as the time at which at least 95% of the carbohydrates have been consumed by the population of yeasts. In some embodiments, the second genetic modification is capable of decreasing and/or decreases the time to complete the fermentation of the recombinant yeast cell when compared to the intermediate yeast cell.

In some embodiments, the presence of the one or more first genetic modifications is capable of causing and/or causes a reduction in the glucose consumption rate (q-, which can, in some embodiments, be provided as g glucose/g cells/h) in the intermediate yeast cell when compared to the parental yeast cell. In some embodiments, the second genetic modification is capable of increasing and/or increases the glucose consumption rate of the recombinant yeast cell when compared to the intermediate yeast cell.

In some embodiments, the presence of the one or more first genetic modifications is capable of causing and/or cases a reduction in the yield of glycerol (Y_{glycerol}) per amount of glucose consumed (S) (Y_{glycerol}/S which can, in some embodiments, be provided as g of glycerol/g of glucose consumed), in the intermediate yeast cell when compared to the parental yeast cell. In some embodiments, the second genetic modification is capable of increasing and/or increases the yield of glycerol (Y_{glycerol}) per amount of glucose consumed (S) of the recombinant yeast cell when compared to the intermediate yeast cell.

In some embodiments, the presence of the one or more first genetic modifications is capable of causing and/or cases a reduction in the rate of carbon dioxide production in the intermediate yeast cell when compared to the parental yeast cell. In some embodiments, the second genetic modification is capable of increasing and/or increases the rate of carbon dioxide production of the recombinant yeast cell when compared to the intermediate yeast cell.

In some embodiments, the presence of the one or more first genetic modifications is capable of causing and/or cases a a reduction in the rate of ethanol production in the intermediate yeast cell when compared to the parental yeast cell. In some embodiments, the second genetic modification is capable of increasing and/or increases the rate of ethanol production of the recombinant yeast cell when compared to the intermediate yeast cell.

The reduction in specific growth rate, in the glucose consumption rate, in the yield of glycerol (Y_{glycerol}) per amount of glucose consumed (S), in the rate of carbon dioxide production and/or in the rate of ethanol production as well as the increase in the time to complete a fermentation can be observed, for example, when a yeast cell (referred to as an intermediate yeast cell) comprises the one or more first genetic modifications and lacks the second genetic modification is being submitted to fermentation. For example, when submitted to comparable/similar fermentation conditions, the recombinant yeast cell, the parental yeast cell and the intermediate yeast cell will exhibit, respectively, a first specific growth rate, a second specific growth rate and a third specific growth rate. In some embodiments, the third specific growth rate of the intermediate cell can be reduced when compared to the second specific growth rate of the parental yeast cell, therefore highlighting the impact of the one or more first genetic modifications on the specific growth rate of the intermediate yeast cell. Also, in further embodiments, the first specific growth rate of the recombinant yeast cell can be increased when compared to the third specific growth rate of the intermediate yeast cell, therefore highlighting that, in some embodiments, the second genetic modification can restore, at least in part, the specific growth rate in the recombinant yeast cell. In still additional embodiments, the first specific growth rate of the recombinant yeast cell can be substantially similar or increased with respect to the second specific growth rate of the parental yeast cell. In some embodiments, the second genetic modification can restore, at least in part, the specific growth rate in the recombinant yeast cell without increasing the yield of glycerol (when compared to the intermediate yeast cell for example).

In another example, when submitted to comparable/similar fermentation conditions, the recombinant yeast cell, the parental yeast cell and the intermediate yeast cell will exhibit, respectively, a first time to complete a fermentation, a second time to complete a fermentation and a third time to complete a fermentation. In some embodiments, the third time to complete a fermentation of the intermediate cell can be increased when compared to the second time to complete a fermentation of the parental yeast cell, therefore highlighting the impact of the one or more first genetic modifications on the time to complete a fermentation the intermediate yeast cell. Also, in further embodiments, the first time to complete a fermentation of the recombinant yeast cell can be decreased when compared to the third time to complete the fermentation of the intermediate yeast cell, therefore highlighting that, in some embodiments, the second genetic modification can decrease, at least in part, the time to complete a fermentation in the recombinant yeast cell. In still additional embodiments, the first time to complete a fermentation of the recombinant yeast cell can be substantially similar or decreased with respect to the second time to complete a fermentation of the parental yeast cell. In some embodiments, the second genetic modification can decrease, at least in part, the time to complete a fermentation in the recombinant yeast cell without increasing the yield of glycerol (when compared to the intermediate yeast cell for example).

In yet another example, when submitted to comparable/similar fermentation conditions, the recombinant yeast cell, the parental yeast cell and the intermediate yeast cell will exhibit, respectively, a first glucose consumption rate, a second glucose consumption rate and a third glucose consumption rate. In some embodiments, the third glucose consumption rate of the intermediate cell can be reduced when compared to the second glucose consumption rate of the parental yeast cell, therefore highlighting the impact of the one or more first genetic modifications on the glucose consumption rate in the intermediate yeast cell. Also, in further embodiments, the first glucose consumption rate of the recombinant yeast cell can be increased when compared to the third glucose consumption rate of the intermediate yeast cell, therefore highlighting that, in some embodiments, the second genetic modification can restore, at least in part, the glucose consumption rate in the recombinant yeast cell. In still additional embodiments, the first glucose consumption rate of the recombinant yeast cell can be substantially similar or increased with respect to the second glucose consumption rate of the parental yeast cell.

In yet another example, when submitted to comparable/similar fermentation conditions, the recombinant yeast cell, the parental yeast cell and the intermediate yeast cell will exhibit, respectively, a first yield of glycerol (Y_{glycerol}) per amount of glucose consumed (S), a second yield of glycerol (Y_{glycerol}) per amount of glucose consumed (S) and a third yield of glycerol (Y_{glycerol}) per amount of glucose consumed (S). In some embodiments, the third yield of glycerol (Y_{glycerol}) per amount of glucose consumed (S) of the intermediate cell can be reduced when compared to the second yield of glycerol (Y_{glycerol}) per amount of glucose consumed (S) of the parental yeast cell, therefore highlighting the impact of the one or more first genetic modifications on the yield of glycerol (Y_{glycerol}) per amount of glucose consumed (S) of the intermediate yeast cell. Also, in further embodiments, the first yield of glycerol (Y_{glycerol}) per amount of glucose consumed (S) of the recombinant yeast cell can be increased when compared to the third yield of glycerol (Y_{glycerol}) per amount of glucose consumed (S) of the intermediate yeast cell, therefore highlighting that, in some embodiments, the second genetic modification can restore, at least in part, the yield of glycerol (Y_{glycerol}) per amount of glucose consumed (S) in the recombinant yeast cell. In still additional embodiments, the first yield of glycerol (Y_{glycerol}) per amount of glucose consumed (S) of the recombinant yeast cell can be substantially similar or increased with respect to the second yield of glycerol (Y_{glycerol}) per amount of glucose consumed (S) of the parental yeast cell.

In yet a further example, when submitted to comparable/similar fermentation conditions, the recombinant yeast cell, the parental yeast cell and the intermediate yeast cell will exhibit, respectively, a first rate of carbon dioxide production, a second rate of carbon dioxide production and a third rate of carbon dioxide production. In some embodiments, the third rate of carbon dioxide production of the intermediate cell can be reduced when compared to the second rate of carbon dioxide production of the parental yeast cell, therefore highlighting the impact of the one or more first genetic modifications on the rate of carbon dioxide production in the intermediate yeast cell. Also, in further embodiments, the first rate of carbon dioxide production of the recombinant yeast cell can be increased when compared to the third rate of carbon dioxide production of the intermediate yeast cell, therefore highlighting that, in some embodiments, the second genetic modification can restore, at least in part, the rate of carbon dioxide production in the recombinant yeast cell. In still additional embodiments, the first rate of carbon dioxide production of the recombinant yeast cell can be substantially similar or increased with respect to the third rate of carbon dioxide production of the parental yeast cell.

In still another example, when submitted to comparable/similar fermentation conditions, the recombination yeast cell, the parental yeast cell and the intermediate yeast cell will exhibit, respectively, a first rate of ethanol production, a second rate of ethanol production and a third rate of ethanol production. In some embodiments, the third rate of ethanol production of the intermediate cell can be reduced when compared to the second rate of ethanol production of the parental yeast cell, therefore highlighting the impact of the one or more first genetic modifications on the rate of ethanol production in the intermediate yeast cell. Also, in further embodiments, the first rate of ethanol production of the recombinant yeast cell can be increased when compared to the third rate of ethanol production of the intermediate yeast cell, therefore highlighting that, in some embodiments, the second genetic modification can restore, at least in part, the rate of ethanol production in the recombinant yeast cell. In still additional embodiments, the first rate of ethanol production of the recombinant yeast cell can be substantially similar or increased with respect to the second rate of ethanol production of the parental yeast cell.

As indicated above, in some embodiments, the one or more first genetic modification are intended to reduce the yield of glycerol per amount of glucose consumed, downregulate glycerol synthesis, decrease the activity or production of one or more enzymes that facilitates glycerol synthesis and/or facilitate glycerol transport (in the recombinant yeast cell when compared to the parental yeast cell). In some further embodiments, a first genetic modifications can exhibit one or more of a reduction in the production of glycerol, a downregulation in glycerol synthesis, a decrease the activity or production of one or more enzymes that facilitates glycerol synthesis or a facilitation glycerol transport.

In some embodiments, the one or more first genetic modifications include a genetic modification capable of causing or which causes a reduction in the expression and/or an inactivation of a native gene encoding an enzyme for producing glycerol, an ortholog encoding an enzyme for producing glycerol or a paralog encoding an enzyme for producing glycerol. Enzymes involved in glycerol production include, without limitation, polypeptides having glycerol-3-phosphate dehydrogenase activity and/or polypeptides having glycerol-3-phosphate phosphatase activity. The reduction in expression and/or the inactivation of one or more genes encoding a polypeptide having glycerol-3-phosphate dehydrogenase activity can be introduced in the recombinant yeast cell. The reduction in expression and/or the inactivation of one or more genes encoding a polypeptide having glycerol-3-phosphate phosphatase activity can be introduced in the recombinant yeast cell. The reduction in expression and/or the inactivation of one or more genes encoding a polypeptide having glycerol-3-phosphate dehydrogenase activity as well as the reduction in expression and/or the inactivation of one or more genes encoding a polypeptide having glycerol-3-phosphate phosphatase activity can be introduced in the recombinant yeast cell.

Polypeptides having glycerol-3-phosphate dehydrogenase activity include, without limitation, glycerol-3-phosphate dehydrogenases (E.C. Number 1.1.1.8) such as glycerol-3-phosphate dehydrogenase 1 (referred to as gpd1) and glycerol-3-phosphate dehydrogenase 2 (referred to as gpd2). The recombinant yeast cell and/or the intermediate yeast cell of the present disclosure can include a reduction in the expression or an inactivation of gpd1, gpd2 or both. Polypeptides having glycerol-3-phosphate phosphatase activity include, without limitation glycerol-3-phosphate phosphatases (E.C. Number 3.1.3.21) such as glycerol-3-phosphate phosphatase 1 (referred to gpp1) and glycerol-3-phosphate phosphatase 2 (gpp2). The recombinant yeast cell and/or the intermediate yeast cell of the present disclosure can include a reduction in the expression or an inactivation of gpp1, gpp2 or both. In yet another embodiment, the recombinant yeast cell and/or the intermediate yeast cell does not bear a genetic modification in its native genes for producing glycerol and includes its native genes coding for the gpp/gpd polypeptides.

Gpd1 genes encoding the gpd1 polypeptide include, but are not limited to Saccharomyces cerevisiae Gene ID: 851539, Schizosaccharomyces pombe Gene ID: 2540547, Schizosaccharomyces pombe Gene ID: 2540455, Neurospora crassa Gene ID: 3873099, Candida albicans Gene ID: 3643924, Scheffersomyces stipitis Gene ID: 4840320, Spathaspora passalidarum Gene ID: 18874668, Trichoderma reesei Gene ID: 18482691, Nectria haematococca Gene ID: 9668637, Candida dubliniensis Gene ID: 8046432, Chlamydomonas reinhardtii Gene ID: 5716580, Brassica napus Gene ID: 106365675, Chlorella variabilis Gene ID: 17355036, Brassica napus Gene ID: 106352802, Mus musculus Gene ID: 14555, Homo sapiens Gene ID: 2819, Rattus norvegicus Gene ID: 60666, Sus scrofa Gene ID: 100153250, Gallus gallus Gene ID: 426881, Bos taurus Gene ID: 525042, Xenopus tropicalis Gene ID: 448519, Pan troglodytes Gene ID: 741054, Canis lupus familiaris Gene ID: 607942, Callorhinchus milii Gene ID: 103188923, Columba livia Gene ID: 102088900, Macaca fascicularis Gene ID: 101865501, Myotis brandtii Gene ID: 102257341, Heterocephalus glaber Gene ID: 101702723, Nannospalax galili Gene ID: 103746543, Mustela putorius furo Gene ID: 101681348, Callithrix jacchus Gene ID: 100414900, Labrus bergylta Gene ID: 109980872, Monopterus albus Gene ID: 109969143, Castor canadensis Gene ID: 109695417, Paralichthys olivaceus Gene ID: 109635348, Bos indicus Gene ID: 109559120, Hippocampus comes Gene ID: 109507993, Rhinolophus sinicus Gene ID: 109443801, Hipposideros armiger Gene ID: 109393253, Crocodylus porosus Gene ID: 109324424, Gavialis gangeticus Gene ID: 109293349, Panthera pardus Gene ID: 109249099, Cyprinus carpio Gene ID: 109094445, Scleropages formosus Gene ID: 108931403, Nanorana parkeri Gene ID: 108789981, Rhinopithecus bieti Gene ID: 108543924, Lepidothrix coronata Gene ID: 108509436, Pygocentrus nattereri Gene ID: 108444060, Manis javanica Gene ID: 108406536, Cebus capucinus imitator Gene ID: 108316082, Ictalurus punctatus Gene ID: 108255083, Kryptolebias marmoratus Gene ID: 108231479, Miniopterus natalensis Gene ID: 107528262, Rousettus aegyptiacus Gene ID: 107514265, Coturnix japonica Gene ID: 107325705, Protobothrops mucrosquamatus Gene ID: 107302714, Parus major Gene ID: 107215690, Marmota marmota marmota Gene ID: 107148619, Gekko japonicus Gene ID: 107122513, Cyprinodon variegatus Gene ID: 107101128, Acinonyxjubatus Gene ID: 106969233, Poecilia latipinna Gene ID: 106959529, Poecilia mexicana Gene ID: 106929022, Calidris pugnax Gene ID: 106891167, Sturnus vulgaris Gene ID: 106863139, Equus asinus Gene ID: 106845052, Thamnophis sirtalis Gene ID: 106545289, Apteryx australis mantelli Gene ID: 106499434, Anser cygnoides domesticus Gene ID: 106047703, Dipodomys ordii Gene ID: 105987539, Clupea harengus Gene ID: 105897935, Microcebus murinus Gene ID: 105869862, Propithecus coquereli Gene ID: 105818148, Aotus nancymaae Gene ID: 105709449, Cercocebus atys Gene ID: 105580359, Mandrillus leucophaeus Gene ID: 105527974, Colobus angolensis palliatus Gene ID: 105507602, Macaca nemestrina Gene ID: 105492851, Aquila chrysaetos canadensis Gene ID: 105414064, Pteropus vampyrus Gene ID: 105297559, Camelus dromedarius Gene ID: 105097186, Camelus bactrianus Gene ID: 105076223, Esox lucius Gene ID: 105016698, Bison bison bison Gene ID: 105001494, Notothenia coriiceps Gene ID: 104967388, Larimichthys crocea Gene ID: 104928374, Fukomys damarensis Gene ID: 04861981, Haliaeetus leucocephalus Gene ID: 104831135, Corvus cornix cornix Gene ID: 104683744, Rhinopithecus roxellana Gene ID: 104679694, Balearica regulorum gibbericeps Gene ID: 104630128, Tinamus guttatus Gene ID: 104575187, Mesitornis unicolor Gene ID: 104539793, Antrostomus carolinensis Gene ID: 104532747, Buceros rhinoceros silvestris Gene ID: 104501599, Chaetura pelagica Gene ID: 104385595, Leptosomus discolor Gene ID: 104353902, Opisthocomus hoazin Gene ID: 104326607, Charadrius vociferus Gene ID: 104284804, Struthio camelus australis Gene ID: 104144034, Egretta garzetta Gene ID: 104132778, Cuculus canorus Gene ID: 104055090, Nipponia nippon Gene ID: 104011969, Pygoscelis adeliae Gene ID: 103914601, Aptenodytes forsteri Gene ID: 103894920, Serinus canaria Gene ID: 103823858, Manacus vitellinus Gene ID: 103760593, Ursus maritimus Gene ID: 103675473, Corvus brachyrhynchos Gene ID: 103613218, Galeopterus variegatus Gene ID: 103598969, Equus przewalskii Gene ID: 103546083, Calypte anna Gene ID: 103536440, Poecilia reticulata Gene ID: 103464660, Cynoglossus semilaevis Gene ID: 103386748, Stegastes partitus Gene ID: 103355454, Eptesicus fuscus Gene ID: 103285288, Chlorocebus sabaeus Gene ID: 103238296, Orycteropus afer afer Gene ID: 103194426, Poecilia formosa Gene ID: 103134553, Erinaceus europaeus Gene ID: 103118279, Lipotes vexillifer Gene ID: 103087725, Python bivittatus Gene ID: 103049416, Astyanax mexicanus Gene ID: 103021315, Balaenoptera acutorostrata scammoni Gene ID: 103006680, Physeter catodon Gene ID: 102996836, Panthera tigris altaica Gene ID: 102961238, Chelonia mydas Gene ID: 102939076, Peromyscus maniculatus bairdii Gene ID: 102922332, Pteropus alecto Gene ID: 102880604, Elephantulus edwardii Gene ID: 102844587, Chrysochloris asiatica Gene ID: 102825902, Myotis davidii Gene ID: 102754955, Leptonychotes weddellii Gene ID: 102730427, Lepisosteus oculatus Gene ID: 102692130, Alligator mississippiensis Gene ID: 102576126, Vicugna pacos Gene ID: 102542115, Camelus ferus Gene ID: 102507052, Tupaia chinensis Gene ID: 102482961, Pelodiscus sinensis Gene ID: 102446147, Myotis lucifugus Gene ID: 102420239, Bubalus bubalis Gene ID: 102395827, Alligator sinensis Gene ID: 102383307, Latimeria chalumnae Gene ID: 102345318, Pantholops hodgsonii Gene ID: 102326635, Haplochromis burtoni Gene ID: 102295539, Bos mutus Gene ID: 102267392, Xiphophorus maculatus Gene ID: 102228568, Pundamilia nyererei Gene ID: 102192578, Capra hircus Gene ID: 102171407, Pseudopodoces humilis Gene ID: 102106269, Zonotrichia albicollis Gene ID: 102070144, Falco cherrug Gene ID: 102047785, Geospiza fortis Gene ID: 102037409, Chinchilla lanigera Gene ID: 102014610, Microtus ochrogaster Gene ID: 101990242, Ictidomys tridecemlineatus Gene ID: 101955193, Chrysemys picta Gene ID: 101939497, Falco peregrinus Gene ID: 101911770, Mesocricetus auratus Gene ID: 101824509, Ficedula albicollis Gene ID: 101814000, Anas platyrhynchos Gene ID: 101789855, Echinops telfairi Gene ID: 101641551, Condylura cristata Gene ID: 101622847, Jaculus jaculus Gene ID: 101609219, Octodon degus Gene ID: 101563150, Sorex araneus Gene ID: 101556310, Ochotona princeps Gene ID: 101532015, Maylandia zebra Gene ID: 101478751, Dasypus novemcinctus Gene ID: 101446993, Odobenus rosmarus divergens Gene ID: 101385499, Tursiops truncatus Gene ID: 101318662, Orcinus orca Gene ID: 101284095, Oryzias latipes Gene ID: 101154943, Gorilla gorilla Gene ID: 101131184, Ovis aries Gene ID: 101119894, Felis catus Gene ID: 101086577, Takifugu rubripes Gene ID: 101079539, Saimiriboliviensis Gene ID: 101030263, Papio anubis Gene ID: 101004942, Pan paniscus Gene ID: 100981359, Otolemur garnettii Gene ID: 100946205, Sarcophilus harrisii Gene ID: 100928054, Cricetulus griseus Gene ID: 100772179, Cavia porcellus Gene ID: 100720368, Oreochromis niloticus Gene ID: 100712149, Loxodonta africana Gene ID: 100660074, Nomascus leucogenys Gene ID: 100594138, Anolis carolinensis Gene ID: 100552972, Meleagris gallopavo Gene ID: 100542199, Ailuropoda melanoleuca Gene ID: 100473892, Oryctolagus cuniculus Gene ID: 100339469, Taeniopygia guttata Gene ID: 100225600, Pongo abelii Gene ID: 100172201, Ornithorhynchus anatinus Gene ID: 100085954, Equus caballus Gene ID: 100052204, Mus musculus Gene ID: 100198, Xenopus laevis Gene ID: 399227, Danio rerio Gene ID: 325181, Danio rerio Gene ID: 406615, Melopsittacus undulatus Gene ID: 101872435, Ceratotherium simum simum Gene ID: 101408813, Trichechus manatus latirostris Gene ID: 101359849 and Takifugu rubripes Gene ID: 101071719).

The gpd2 genes encoding the gpd2 polypeptide include, but are not limited to Mus musculus Gene ID: 14571, Homo sapiens Gene ID: 2820, Saccharomyces cerevisiae Gene ID: 854095, Rattus norvegicus Gene ID: 25062, Schizosaccharomyces pombe Gene ID: 2541502, Mus musculus Gene ID: 14380, Danio rerio Gene ID: 751628, Caenorhabditis elegans Gene ID: 3565504, Mesocricetus auratus Gene ID: 101825992, Xenopus tropicalis Gene ID: 779615, Macaca mulatta Gene ID: 697192, Bos taurus Gene ID: 504948, Canis lupus familiaris Gene ID: 478755, Cavia porcellus Gene ID: 100721200, Gallus gallus Gene ID: 424321, Pan troglodytes Gene ID: 459670, Oryctolagus cuniculus Gene ID: 100101571, Candida albicans Gene ID: 3644563, Xenopus laevis Gene ID: 444438, Macaca fascicularis Gene ID: 102127260, Ailuropoda melanoleuca Gene ID: 100482626, Cricetulus griseus Gene ID: 100766128, Heterocephalus glaber Gene ID: 101715967, Scheffersomyces stipitis Gene ID: 4838862, Ictalurus punctatus Gene ID: 108273160, Mustela putorius furo Gene ID: 101681209, Nannospalax galili Gene ID: 103741048, Callithrix jacchus Gene ID: 100409379, Lates calcarifer Gene ID: 108873068, Nothobranchius furzeri Gene ID: 07384696, Acanthisitta chloris Gene ID: 103808746, Acinonyxjubatus Gene ID: 106978985, Alligatormississippiensis Gene ID: 102562563, Alligator sinensis Gene ID: 102380394, Anas platyrhynchos, Anolis carolinensis Gene ID: 100551888, Anser cygnoides domesticus Gene ID: 106043902, Aotus nancymaae Gene ID: 105719012, Apaloderma vittatum Gene ID: 104281080, Aptenodytes forsteri Gene ID: 103893867, Apteryx australis mantelli Gene ID: 106486554, Aquila chrysaetos canadensis Gene ID: 105412526, Astyanax mexicanus Gene ID: 103029081, Austrofundulus limnaeus Gene ID: 106535816, Balaenoptera acutorostrata scammoni Gene ID: 103019768, Balearica regulorum gibbericeps, Bison bison bison Gene ID: 104988636, Bos indicus Gene ID: 109567519, Bos mutus Gene ID: 102277350, Bubalus bubalis Gene ID: 102404879, Buceros rhinoceros silvestris Gene ID: 104497001, Calidris pugnax Gene ID: 106902763, Callorhinchus milii Gene ID: 103176409, Calypte anna Gene ID: 103535222, Camelus bactrianus Gene ID: 105081921, Camelus dromedarius Gene ID: 105093713, Camelus ferus Gene ID: 102519983, Capra hircus Gene ID: 102176370, Cariama cristata Gene ID: 104154548, Castor canadensis Gene ID: 109700730, Cebus capucinus imitator Gene ID: 108316996, Cercocebus atys Gene ID: 105576003, Chaetura pelagica Gene ID: 104391744, Charadrius vociferus Gene ID: 104286830, Chelonia mydas Gene ID: 102930483, Chinchilla lanigera Gene ID: 102017931, Chlamydotis macqueenii Gene ID: 104476789, Chlorocebus sabaeus Gene ID: 103217126, Chrysemys picta Gene ID: 101939831, Chrysochloris asiatica Gene ID: 102831540, Clupea harengus Gene ID: 105902648, Colius striatus Gene ID: 104549356, Colobus angolensis palliatus Gene ID: 105516852, Columba livia Gene ID: 102090265, Condylura cristata Gene ID: 101619970, Corvus brachyrhynchos, Coturnix japonica Gene ID: 107316969, Crocodylus porosus Gene ID: 109322895, Cuculus canorus Gene ID: 104056187, Cynoglossus semilaevis Gene ID: 103389593, Dasypus novemcinctus Gene ID: 101428842, Dipodomys ordii Gene ID: 105996090, Echinops telfairi Gene ID: 101656272, Egretta garzetta Gene ID: 104135263, Elephantulus edwardii Gene ID: 102858276, Eptesicus fuscus Gene ID: 103283396, Equus asinus Gene ID: 106841969, Equus caballus Gene ID: 100050747, Equus przewalskii Gene ID: 103558835, Erinaceus europaeus Gene ID: 103114599, Eurypyga helias Gene ID: 104502666, Falco cherrug Gene ID: 102054715, Falco peregrinus Gene ID: 101912742, Felis catus Gene ID: 101089953, Ficedula albicollis Gene ID: 101816901, Fukomys damarensis Gene ID: 104850054, Fundulus heteroclitus Gene ID: 105936523, Galeopterus variegatus Gene ID: 103586331, Gavia stellata Gene ID: 104250365, Gavialis gangeticus Gene ID: 109301301, Gekko japonicus Gene ID: 107110762, Geospiza fortis Gene ID: 102042095, Gorilla gorilla Gene ID: 101150526, Haliaeetus albicilla Gene ID: 104323154, Haliaeetus leucocephalus Gene ID: 104829038, Haplochromis burtoni Gene ID: 102309478, Hippocampus comes Gene ID: 109528375, Hipposideros armiger Gene ID: 109379867, Ictidomys tridecemlineatus Gene ID: 101965668, Jaculus jaculus Gene ID: 101616184, Kryptolebias marmoratus Gene ID: 108251075, Labrus bergylta Gene ID: 109984158, Larimichthys crocea Gene ID: 104929094, Latimeria chalumnae Gene ID: 102361446, Lepidothrix coronata Gene ID: 108501660, Lepisosteus oculatus Gene ID: 102691231, Leptonychotes weddellii Gene ID: 102739068, Leptosomus discolor Gene ID: 104340644, Lipotes vexillifer Gene ID: 103074004, Loxodonta africana Gene ID: 100654953, Macaca nemestrina Gene ID: 105493221, Manacus vitellinus Gene ID: 103757091, Mandrillus leucophaeus Gene ID: 105548063, Manis javanica Gene ID: 108392571, Marmota marmota marmota Gene ID: 107136866, Maylandia zebra Gene ID: 101487556, Mesitornis unicolor Gene ID: 104545943, Microcebus murinus Gene ID: 105859136, Microtus ochrogaster Gene ID: 101999389, Miniopterus natalensis Gene ID: 107525674, Monodelphis domestica Gene ID: 100014779, Monopterus albus Gene ID: 109957085, Myotis brandtii Gene ID: 102239648, Myotis davidii Gene ID: 102770109, Myotis lucifugus Gene ID: 102438522, Nanorana parkeri Gene ID: 108784354, Nestor notabilis Gene ID: 104399051, Nipponia nippon Gene ID: 104012349, Nomascus leucogenys Gene ID: 100590527, Notothenia coriiceps Gene ID: 104964156, Ochotona princeps Gene ID: 101530736, Octodon degus Gene ID: 101591628, Odobenus rosmarus divergens Gene ID: 101385453, Oncorhynchus kisutch Gene ID: 109870627, Opisthocomus hoazin Gene ID: 104338567, Orcinus orca Gene ID: 101287409, Oreochromis niloticus Gene ID: 100694147, Ornithorhynchus anatinus Gene ID: 100081433, Orycteropus afer afer Gene ID: 103197834, Oryzias latipes Gene ID: 101167020, Otolemurgar nettii Gene ID: 100966064, Ovis aries Gene ID: 443090, Pan paniscus Gene ID: 100970779, Panthera pardus Gene ID: 109271431, Panthera tigris altaica Gene ID: 102957949, Pantholops hodgsonii Gene ID: 102323478, Papio anubis Gene ID: 101002517, Paralichthys olivaceus Gene ID: 109631046, Pelodiscus sinensis Gene ID: 102454304, Peromyscus maniculatus bairdii Gene ID: 102924185, Phaethon lepturus Gene ID: 104624271, Phalacrocorax carbo Gene ID: 104049388, Physeter catodon Gene ID: 102978831, Picoides pubescens Gene ID: 104296936, Poecilia latipinna Gene ID: 106958025, Poecilia mexicana Gene ID: 106920534, Poecilia reticulata Gene ID: 103473778, Pongo abelii Gene ID: 100452414, Propithecus coquereli Gene ID: 105807399, Protobothrops mucrosquamatus Gene ID: 107289584, Pseudopodoces humilis Gene ID: 102109711, Pterocles gutturalis Gene ID: 104461236, Pteropus alecto Gene ID: 102879110, Pteropus vampyrus Gene ID: 105291402, Pundamilia nyererei Gene ID: 102200268, Pygocentrus nattereri Gene ID: 108411786, Pygoscelis adeliae Gene ID: 103925329, Python bivittatus Gene ID: 103059167, Rhincodon typus Gene ID: 109920450, Rhinolophus sinicus Gene ID: 109445137, Rhinopithecus bieti Gene ID: 108538766, Rhinopithecus roxellana Gene ID: 104654108, Rousettus aegyptiacus Gene ID: 107513424, Saimiri boliviensis Gene ID: 101027702, Salmo salar Gene ID: 106581822, Sarcophilus harrisii Gene ID: 100927498, Scleropages formosus Gene ID: 108927961, Serinus canaria Gene ID: 103814246, Sinocyclocheilus grahami Gene ID: 107555436, Sorex araneus Gene ID: 101543025, Stegastes partitus Gene ID: 103360018, Struthio camelus australis Gene ID: 104138752, Sturnus vulgaris Gene ID: 106861926, Sugiyamaella lignohabitans Gene ID: 30033324, Sus scrofa Gene ID: 397348, Taeniopygia guttata Gene ID: 100222867, Takifugu rubripes Gene ID: 101062218, Tarsius syrichta Gene ID: 103254049, Tauraco erythrolophus Gene ID: 104378162, Thamnophis sirtalis Gene ID: 106538827, Tinamus guttatus Gene ID: 104572349, Tupaia chinensis Gene ID: 102471148, Tursiops truncatus Gene ID: 101330605, Ursus maritimus Gene ID: 103659477, Vicugna pacos Gene ID: 102533941, Xiphophorus maculatus Gene ID: 102225536, Zonotrichia albicollis Gene ID: 102073261, Ciona intestinalis Gene ID: 100183886, Meleagris gallopavo Gene ID: 100546408, Trichechus manatus latirostris Gene ID: 101355771, Ceratotherium simum simum Gene ID: 101400784, Melopsittacus undulatus Gene ID: 101871704, Esox lucius Gene ID: 10502249 and Pygocentrus nattereri Gene ID: 108411786. In an embodiment, the gpd2 polypeptide is encoded by Saccharomyces cerevisiae Gene ID: 854095.

The gpp1 genes encoding the gpp1 polypeptide include, but are not limited to Saccharomyces cerevisiae Gene ID: 854758, Arabidopsis thaliana Gene ID: 828690, Scheffersomyces stipitis Gene ID: 4836794, Chlorella variabilis Gene ID: 17352997, Solanum tuberosum Gene ID: 102585195, Homo sapiens Gene ID: 7316, Millerozyma farinosa Gene ID: 14521241, 14520178, 1451927 and 14518181, Sugiyamaella lignohabitans Gene ID: 30035078, Candida dubliniensis Gene ID: 8046759.

The gpp2 genes encoding the the gpp2 polypeptide include, but are not limited to Saccharomyces cerevisiae Gene ID: 856791, Sugiyamaella lignohabitans Gene ID: 30035078, Arabidopsis thaliana Gene ID: 835849, Nicotiana attenuata Gene ID: 109234217, Candida albicans Gene ID: 3640236, Candida glabrata Gene ID: 2891433, 2891243 and 2889223.

In some embodiments, the one or more first genetic modifications comprise a genetic modification for facilitating glycerol transport which may, in further embodiments, reduce the production of glycerol (in some specific embodiments, by downregulating the expression of one or more enzymes that facilitate glycerol synthesis). In some additional embodiments, the one or more first genetic modification can include a genetic modification for overexpressing a native polypeptide facilitating glycerol transport and/or expressing a heterologous polypeptide facilitating glycerol transport. The recombinant yeast cell of the present disclosure can include a genetic modification for overexpressing a native polypeptide facilitating glycerol transport. The recombinant yeast cell of the present disclosure can include a genetic modification for expressing a heterologous polypeptide facilitating glycerol transport. The recombinant yeast cell of the present disclosure can include a genetic modification for overexpressing a native polypeptide facilitating glycerol transport and another one for expressing a heterologous polypeptide facilitating glycerol transport.

Polypeptides facilitating glycerol transport include but are not limited to polypeptides having glycerol proton symporter activity. An embodiment of a polypeptide having glycerol proton symporter activity is stl1, a polypeptide encoded by a stl1 gene ortholog and/or a polypeptide encoded by a stl1 gene paralog. stl1 can be natively expressed in yeasts and fungi. stl1 genes encoding the stl1 polypeptide include, but are not limited to, Saccharomyces cerevisiae Gene ID: 852149, Candida albicans, Kluyveromyces lactis Gene ID: 2896463, Ashbya gossypii Gene ID: 4620396, Eremothecium sinecaudum Gene ID: 28724161, Torulaspora delbrueckii Gene ID: 11505245, Lachancea thermotolerans Gene ID: 8290820, Phialophora attae Gene ID: 28742143, Penicillium digitatum Gene ID: 26229435, Aspergillus oryzae Gene ID: 5997623, Aspergillus fumigatus Gene ID: 3504696, Talaromyces atroroseus Gene ID: 31007540, Rasamsonia emersonii Gene ID: 25315795, Aspergillus flavus Gene ID: 7910112, Aspergillus terreus Gene ID: 4322759, Penicillium chrysogenum Gene ID: 8310605, Alternaria alternata Gene ID: 29120952, Paraphaeosphaeria sporulosa Gene ID: 28767590, Pyrenophora tritici-repentis Gene ID: 6350281, Metarhizium robertsii Gene ID: 19259252, Isaria fumosorosea Gene ID: 30023973, Cordyceps militaris Gene ID: 18171218, Pochonia chlamydosporia Gene ID: 28856912, Metarhizium majus Gene ID: 26274087, Neofusicoccum parvum Gene ID: 19029314, Diplodia corticola Gene ID: 31017281, Verticillium dahliae Gene ID: 20711921, Colletotrichum gloeosporioides Gene ID: 18740172, Verticillium albo-atrum Gene ID: 9537052, Paracoccidioides lutzii Gene ID: 9094964, Trichophyton rubrum Gene ID: 10373998, Nannizzia gypsea Gene ID: 10032882, Trichophyton verrucosum Gene ID: 9577427, Arthroderma benhamiae Gene ID: 9523991, Magnaporthe oryzae Gene ID: 2678012, Gaeumannomyces graminis var. tritici Gene ID: 20349750, Togninia minima Gene ID: 19329524, Eutypa lata Gene ID: 19232829, Scedosporium apiospermum Gene ID: 27721841, Aureobasidium namibiae Gene ID: 25414329, Sphaerulina musiva Gene ID: 27905328 as well as Pachysolen tannophilus GenBank Accession Numbers JQ481633 and JQ481634, Saccharomyces paradoxus stl1 and Millerozyma farinose GenBank Accession Number CCE78002. In an embodiment, the stl1 polypeptide is encoded by Saccharomyces cerevisiae Gene ID: 852149. In a specific embodiment, the stl1 polypeptide is derived from Saccharomyces sp. and in further embodiments from Saccharomyces cerevisiae. In yet additional embodiment, the stl1 polypeptide has the amino acid sequence of SEQ ID NO: 8, is a variant of the amino acid sequence of SEQ ID NO: 8 having glycerol proton symporter activity or is a fragment of the amino acid sequence of SEQ ID NO: 8 having glycerol proton symporter activity. In additional embodiment, the stl1 polypeptide can be encoded by a nucleic acid molecule comprising the nucleic acid sequence of SEQ ID NO: 7 or can comprise a degenerate sequence encoding the amino acid sequence of SEQ ID NO: 8. In some specific embodiments, the heterologous nucleic acid molecule encoding the stl1 polypeptide, its variants or its fragments is knocked-in at the native position at which the gene of the native stl1 polypeptide is located.

In some embodiments, the one or more first genetic modifications comprise a genetic modification are intended to increase formate and/or acetyl-CoA production (in the recombinant yeast cell when compared to the parental yeast cell). In some further embodiments, the first genetic modifications can exhibit one or more of an increase in formate production or an increase in acetyl-CoA production. In some specific embodiments, the one or more first genetic modification comprises a genetic modification for overexpressing a native polypeptide having pyruvate formate lyase activity and/or expressing a heterologous polypeptide having pyruvate formate lyase activity. The recombinant yeast cell of the present disclosure can include a genetic modification for overexpressing a native polypeptide having pyruvate formate lyase activity. The recombinant yeast cell of the present disclosure can include a genetic modification for expressing a heterologous polypeptide having pyruvate formate lyase activity. The recombinant yeast cell of the present disclosure can include a genetic modification for overexpressing a native polypeptide having pyruvate formate lyase activity and another one for expressing a heterologous polypeptide having pyruvate formate lyase activity. In some embodiments, the recombinant yeast cell of the present disclosure comprises a further genetic modification for reducing the expression or inactivating in one or more native genes encloding for a native polypeptide having pyruvate formate lyase activity (optionally in combination with the expression of one or more heterologous polypeptides having pyruvate formate lyase activity). In another embodiment, the recombinant yeast cell of the present disclosure lacks a genetic modification for reducing the expression and/or inactivating one or more native genes encloding for a native polypeptide having pyruvate formate lyase activity and comprises its native genes encloding for native polypeptides having pyruvate formate lyase activity (optionally in combination with the expression of one or more heterologous polypeptides having pyruvate formate lyase activity).

Polypeptides having formate lyase activity include, without limitations, pflA, pflB, a polypeptide encoded by a pfla gene ortholog or paralog, as well as a polypeptide encoded by a pflb gene ortholog or paralog. In some embodiments, the yeast cell comprises a genetic modification for expressing pflA. In some additional embodiments, the yeast cell comprises a genetic modification for expressing a pflB. In a specific embodiment, the yeast cell comprises a genetic modification for expressing pflA and pflB.

Embodiments of pflA can be derived, without limitation, from the following (the number in brackets correspond to the Gene ID number): Escherichia coli (MG1655945517), Shewanella oneidensis (1706020), Bifidobacterium longum (1022452), Mycobacterium bovis (32287203), Haemophilus parasuis (7277998), Mannheimia haemolytica (15341817), Vibrio vulnificus (33955434), Cronobacter sakazakii (29456271), Vibrio alginolyticus (31649536), Pasteurella multocida (29388611), Aggregatibacter actinomycetemcomitans (31673701), Actinobacillus suis (34291363), Finegoldia magna (34165045), Zymomonas mobilis subsp. mobilis (3073423), Vibrio tubiashii (23444968), Gallibacterium anatis (10563639), Actinobacillus pleuropneumoniae serovar (4849949), Ruminiclostridium thermocellum (35805539), Cylindrospermopsis raciborskii (34474378), Lactococcus garvieae (34204939), Bacillus cytotoxicus (33895780), Providencia stuartii (31518098), Pantoea ananatis (31510290), Teredinibacter turnerae (29648846), Morganella morganii subsp. morganii (14670737), Vibrio anguillarum (77510775106), Dickeya dadantii (39379733484), Xenorhabdus bovienii (8830449), Edwardsiella ictaluri (7959196), Proteus mirabilis (6801040), Rahnella aquatilis (34350771), Bacillus pseudomycoides (34214771), Vibrio alginolyticus (29867350), Vibrio nigripulchritudo (29462895), Vibrio orientalis (25689084), Kosakonia sacchari (23844195), Serratia marcescens subsp. marcescens (23387394), Shewanella baltica (11772864), Vibrio vulnificus (2625152), Streptomyces acidiscabies (33082227), Streptomyces davaonensis (31227069), Streptomyces scabiei (24308152), Volvox carteri f. nagariensis (9616877), Vibrio breoganii (35839746), Vibrio mediterranei (34766273), Fibrobacter succinogenes subsp. succinogenes (34755395), Enterococcus gilvus (34360882), Akkermansia muciniphila (34173806), Enterobacter hormaechei subsp. steigerwaltii (34153767), Dickeya zeae (33924935), Enterobacter sp. (32442159), Serratia odorifera (31794665), Vibrio crassostreae (31641425), Selenomonas ruminantium subsp. lactilytica (31522409), Fusobacterium necrophorum subsp. funduliforme (31520833), Bacteroides uniformis (31507008), Haemophilus somnus (233631487328), Rodentibacter pneumotropicus (31211548), Pectobacterium carotovorum subsp. carotovorum (29706463), Eikenella corrodens (29689753), Bacillus thuringiensis (29685036), Streptomyces rimosus subsp. Rimosus (29531909), Vibrio fluvialis (29387180), Klebsiella oxytoca (29377541), Parageobacillus thermoglucosidans (29237437), Aeromonas veronii (28678409), Clostridium innocuum (26150741), Neisseria mucosa (25047077), Citrobacter freundii (23337507), Clostridium bolteae (23114831), Vibrio tasmaniensis (7160642), Aeromonas salmonicida subsp. salmonicida (4995006), Escherichia coli O157:H7 str. Sakai (917728), Escherichia coli O83:H1 str. (12877392), Yersinia pestis (11742220), Clostridioides difficile (4915332), Vibrio fischeri (3278678), Vibrio parahaemolyticus (1188496), Vibrio coralliilyticus (29561946), Kosakonia cowanii (35808238), Yersinia ruckeri (29469535), Gardnerella vaginalis (99041930), Listeria fleischmannii subsp. Coloradonensis (34329629), Photobacterium kishitanii (31588205), Aggregatibacter actinomycetemcomitans (29932581), Bacteroides caccae (36116123), Vibrio toranzoniae (34373279), Providencia alcalifaciens (34346411), Edwardsiella anguillarum (33937991), Lonsdalea quercina subsp. Quercina (33074607), Pantoea septica (32455521), Butyrivibrio proteoclasticus (31781353), Photorhabdus temperata subsp. Thracensis (29598129), Dickeya solani (23246485), Aeromonas hydrophila subsp. hydrophila (4489195), Vibrio cholerae O1 biovar El Tor str. (2613623), Serratia rubidaea (32372861), Vibrio bivalvicida (32079218), Serratia liquefaciens (29904481), Gilliamella apicola (29851437), Pluralibacter gergoviae (29488654), Escherichia coli O104:H4 (13701423), Enterobacter aerogenes (10793245), Escherichia coli (7152373), Vibrio campbellii (5555486), Shigella dysenteriae (3795967), Bacillus thuringiensis serovar konkukian (2854507), Salmonella enterica subsp. enterica serovar Typhimurium (1252488), Bacillus anthracis (1087733), Shigella flexneri (1023839), Streptomyces griseoruber (32320335), Ruminococcus gnavus (35895414), Aeromonas fluvialis (35843699), Streptomyces ossamyceticus (35815915), Xenorhabdus doucetiae (34866557), Lactococcus piscium (34864314), Bacillus glycinifermentans (34773640), Photobacterium damselae subsp. Damselae 34509297, Streptomyces venezuelae 34035779, Shewanella algae (34011413), Neisseria sicca (33952518), Chania multitudinisentens (32575347), Kitasatospora purpeofusca (32375714), Serratia fonticola (32345867), Aeromonas enteropelogenes (32325051), Micromonospora aurantiaca (32162988), Moritella viscosa (31933483), Yersinia aldovae (31912331), Leclercia adecarboxylata (31868528), Salinivibrio costicola subsp. costicola (31850688), Aggregatibacter aphrophilus (31611082), Photobacterium leiognathi (31590325), Streptomyces canus (31293262), Pantoea dispersa (29923491), Pantoea rwandensis (29806428), Paenibacillus borealis (29548601), Aliivibrio wodanis (28541257), Streptomyces virginiae (23221817), Escherichia coli (7158493), Mycobacterium tuberculosis (887973), Streptococcus mutans (1028925), Streptococcus cristatus (29901602), Enterococcus hirae (13176624), Bacillus licheniformis (3031413), Chromobacterium violaceum (24949178), Parabacteroides distasonis (5308542), Bacteroides vulgatus (5303840), Faecalibacterium prausnitzii (34753201), Melissococcus plutonius (34410474), Streptococcus gallolyticus subsp. gallolyticus (34397064), Enterococcus malodoratus (34355146), Bacteroides oleiciplenus (32503668), Listeria monocytogenes (985766), Enterococcus faecalis (1200510), Campylobacter jejuni subsp. jejuni (905864), Lactobacillus plantarum (1063963), Yersinia enterocolitica subsp. enterocolitica (4713333), Streptococcus equinus (33961143), Macrococcus canis (35294771), Streptococcus sanguinis (4807186), Lactobacillus salivarius (3978441), Lactococcus lactis subsp. lactis (1115478), Enterococcus faecium (12999835), Clostridium botulinum A (5184387), Clostridium acetobutylicum (1117164), Bacillus thuringiensis serovar konkukian (2857050), Cryobacterium flavum (35899117), Enterovibrio norvegicus (35871749), Bacillus acidiceler (34874556), Prevotella intermedia (34516987), Pseudobutyrivibrio ruminis (34419801), Pseudovibrio ascidiaceicola (34149433), Corynebacterium coyleae (34026109), Lactobacillus curvatus (33994172), Cellulosimicrobium cellulans (33980622), Lactobacillus agilis (33975995), Lactobacillus sakei (33973512), Staphylococcus simulans (32051953), Obesumbacterium proteus (29501324), Salmonella enterica subsp. enterica serovar Typhi (1247402), Streptococcus agalactiae (1014207), Streptococcus agalactiae (1013114), Legionella pneumophila subsp. pneumophila str. Philadelphia (119832735), Pyrococcus furiosus (1468475), Mannheimia haemolytica (15340992), Thalassiosira pseudonana (7444511), Thalassiosira pseudonana (7444510), Streptococcus thermophilus (31940129), Sulfolobus solfataricus (1454925), Streptococcus iniae (35765828), Streptococcus iniae (35764800), Bifidobacterium thermophilum (31839084), Bifidobacterium animalis subsp. lactis (29695452), Streptobacillus moniliformis (29673299), Thermogladius calderae (13013001), Streptococcus oralis subsp. tigurinus (31538096), Lactobacillus ruminis (29802671), Streptococcus parauberis (29752557), Bacteroides ovatus (29454036), Streptococcus gordonii str. Challis substr. CH1 (25052319), Clostridium botulinum B str. Eklund 17B (19963260), Thermococcus litoralis (16548368), Archaeoglobus sulfaticallidus (15392443), Ferroglobus placidus (8778929), Archaeoglobus profundus (8739370), Listeria seeligeri serovar 1/2b (32488230), Bacillus thuringiensis (31632063), Rhodobacter capsulatus (31491679), Clostridium botulinum (29749009), Clostridium perfringens (29571530), Lactococcus garvieae (12478921), Proteus mirabilis (6799920), Lactobacillus animalis (32012274), Vibrio alginolyticus (29869205), Bacteroides thetaiotaomicron (31617701), Bacteroides thetaiotaomicron (31617140), Bacteroides cellulosilyticus (29608790), Bacteroides ovatus (29453452), Bacillus mycoides (29402181), Chlamydomonas reinhardtii (5726206), Fusobacterium periodonticum (35833538), Selenomonas flueggei (32477557), Selenomonas noxia (32475880), Anaerococcus hydrogenalis (32462628), Centipeda periodontii (32173931), Centipeda periodontii (32173899), Streptococcus thermophilus (31938326), Enterococcus durans (31916360), Fusobacterium nucleatum (31730399), Anaerostipes hadrus (31625694), Anaerostipes hadrus (31623667), Enterococcus haemoperoxidus (29838940), Gardnerella vaginalis (29692621), Streptococcus salivarius (29397526), Klebsiella oxytoca (29379245), Bifidobacterium breve (29241363), Actinomyces odontolyticus (25045153), Haemophilus ducreyi (24944624), Archaeoglobus fulgidus (24793671), Streptococcus uberis (24161511), Fusobacterium nucleatum subsp. animalis (23369066), Corynebacterium accolens (23249616), Archaeoglobus veneficus (10394332), Prevotella melaninogenica (9497682), Aeromonas salmonicida subsp. salmonicida (4997325), Pyrobaculum islandicum (4616932), Thermofilum pendens (4600420), Bifidobacterium adolescentis (4556560), Listeria monocytogenes (986485), Bifidobacterium thermophilum (35776852), Methanothermobacter sp. CaT2 (24854111), Streptococcus pyogenes (901706), Exiguobacterium sibiricum (31768748), Clostridioides difficile (4916015), Clostridioides difficile (4913022), Vibrio parahaemolyticus (1192264), Yersinia enterocolitica subsp. enterocolitica (4712948), Enterococcus cecorum (29475065), Bifidobacterium pseudolongum (34879480), Methanothermus fervidus (9962832), Methanothermus fervidus (9962056), Corynebacterium simulans (29536891), Thermoproteus uzoniensis (10359872), Vulcanisaeta distributa (9752274), Streptococcus mitis (8799048), Ferroglobus placidus (8778420), Streptococcus suis (8153745), Clostridium novyi (4541619), Streptococcus mutans (1029528), Thermosynechococcus elongatus (1010568), Chlorobium tepidum (1007539), Fusobacterium nucleatum subsp. nucleatum (993139), Streptococcus pneumoniae (933787), Clostridium baratii (31579258), Enterococcus mundtii (31547246), Prevotella ruminicola (31500814), Aeromonas hydrophila subsp. hydrophila (4490168), Aeromonas hydrophila subsp. hydrophila (4487541), Clostridium acetobutylicum (1117604), Chromobacterium subtsugae (31604683), Gilliamella apicola (29849369), Klebsiella pneumoniae subsp. pneumoniae (11846825), Enterobacter cloacae subsp. cloacae (9125235), Escherichia coli (7150298), Salmonella enterica subsp. enterica serovar Typhimurium (1252363), Salmonella enterica subsp. enterica serovar Typhi (1247322), Bacillus cereus (1202845), Bacteroides thetaiotaomicron (1074343), Bacteroides thetaiotaomicron (1071815), Bacillus coagulans (29814250), Bacteroides cellulosilyticus (29610027), Bacillus anthracis (2850719), Monoraphidium neglectum (25735215), Monoraphidium neglectum (25727595), Alloscardovia omnicolens (35868062), Actinomyces neuii subsp. neuii (35867196), Acetoanaerobium sticklandii (35557713), Exiguobacterium undae (32084128), Paenibacillus pabuli (32034589), Paenibacillus etheri (32019864), Actinomyces oris (31655321), Vibrio alginolyticus (31651465), Brochothrix thermosphacta (29820407), Lactobacillus sakei subsp. sakei (29638315), Anoxybacillus gonensis (29574914), variants thereof as well as fragments thereof. In an embodiment, pflA is derived from the genus Bifidobacterium and in some embodiments from the species Bifidobacterium adolescentis. In still another embodiment, pflA has the amino acid sequence of SEQ ID NO: 2, is a variant of the amino acid sequence of SEQ ID NO: 2 having pyruvate formate lyase activity or is a variant of the amino acid sequence of SEQ ID NO: 2 having having pyruvate formate lyase activity. In yet another embodiment, pflA is encoded by a nucleic acid molecule having the nucleic acid sequence of SEQ ID NO: 1 or comprising a degenerate sequence encoding the amino acid sequence of SEQ ID NO: 2.

Embodiments of pflB can be derived, without limitation, from the following (the number in brackets correspond to the Gene ID number): Escherichia coli (945514), Shewanella oneidensis (1170601), Actinobacillus suis (34292499), Finegoldia magna (34165044), Streptococcus cristatus (29901775), Enterococcus hirae (13176625), Bacillus (3031414), Providencia alcalifaciens (34345353), Lactococcus garvieae (34203444), Butyrivibrio proteoclasticus (31781354), Teredinibacter turnerae (29651613), Chromobacterium violaceum (24945652), Vibrio campbellii (5554880), Vibrio campbellii (5554796), Rahnella aquatilis HX2 (34351700), Serratia rubidaea (32375076), Kosakonia sacchari SP1 (23845740), Shewanella baltica (11772863), Streptomyces acidiscabies (33082309), Streptomyces davaonensis (31227068), Parabacteroides distasonis (5308541), Bacteroides vulgatus (5303841), Fibrobacter succinogenes subsp. succinogenes (34755392), Photobacterium damselae subsp. Damselae (34512678), Enterococcus gilvus (34361749), Enterococcus gilvus (34360863), Enterococcus malodoratus (34355213), Enterococcus malodoratus (34354022), Akkermansia muciniphila (34174913), Lactobacillus curvatus (33995135), Dickeya zeae (33924934), Bacteroides oleiciplenus (32502326), Micromonospora aurantiaca (32162989), Selenomonas ruminantium subsp. lactilytica (31522408), Fusobacterium necrophorum subsp. funduliforme (31520832), Bacteroides uniformis (31507007), Streptomyces rimosus subsp. Rimosus (29531908), Clostridium innocuum (26150740), Haemophilus] ducreyi (24944556), Clostridium bolteae (23114829), Vibrio tasmaniensis (7160644), Aeromonas salmonicida subsp. salmonicida (4997718), Listeria monocytogenes (986171), Enterococcus faecalis (1200511), Lactobacillus plantarum (1064019), Vibrio fischeri (3278780), Lactobacillus sakei (33973511), Gardnerella vaginalis (9904192), Vibrio vulnificus (33954428), Vibrio toranzoniae (34373229), Anaerostipes hadrus (34240161), Edwardsiella anguillarum (33940299), Edwardsiella anguillarum (33937990), Lonsdalea quercina subsp. Quercina (33074710), Enterococcus faecium (12999834), Aeromonas hydrophila subsp. hydrophila (4489100), Clostridium acetobutylicum (1117163), Escherichia coli (7151395), Shigella dysenteriae (3795966), Bacillus thuringiensis serovar konkukian (2856201), Salmonella enterica subsp. enterica serovar Typhimurium (1252491), Shigella flexneri (1023824), Streptomyces griseoruber (32320336), Cryobacterium flavum (35898977), Ruminococcus gnavus (35895748), Bacillus acidiceler (34874555), Lactococcus piscium (34864362), Vibrio mediterranei (34766270), Faecalibacterium prausnitzii (34753200), Prevotella intermedia (34516966), Photobacterium damselae subsp. Damselae (34509286), Pseudobutyrivibrio ruminis (34419894), Melissococcus plutonius (34408953), Streptococcus gallolyticus subsp. gallolyticus (34398704), Enterobacter hormaechei subsp. steigerwaltii (34155981), Enterobacter hormaechei subsp. steigerwaltii (34152298), Streptomyces venezuelae (34036549), Shewanella algae (34009243), Lactobacillus agilis (33976013), Streptococcus equinus (33961013), Neisseria sicca (33952517), Kitasatospora purpeofusca (32375782), Paenibacillus borealis (29549449), Vibrio fluvialis (29387150), Aliivibrio wodanis (28542465), Aliivibrio wodanis (28541256), Escherichia coli (7157421), Salmonella enterica subsp. enterica serovar Typhi (1247405), Yersinia pestis (1174224), Yersinia enterocolitica subsp. enterocolitica (4713334), Streptococcus suis (8155093), Escherichia coli (947854), Escherichia coli (946315), Escherichia coli (945513), Escherichia coli (948904), Escherichia coli (917731), Yersinia enterocolitica subsp. enterocolitica (4714349), variants thereof as well as fragments thereof. In an embodiment, the pflB polypeptide is derived from the genus Bifidobacterium and in some embodiments from the species Bifidobacterium adolescentis. In still another embodiment, pflB has the amino acid sequence of SEQ ID NO: 4, is a variant of the amino acid sequence of SEQ ID NO: 4 having pyruvate formate lyase activity or is a variant of the amino acid sequence of SEQ ID NO: 4 having having pyruvate formate lyase activity. In yet another embodiment, pflB is encoded by a nucleic acid molecule having the nucleic acid sequence of SEQ ID NO: 3 or comprising a degenerate sequence encoding the amino acid sequence of SEQ ID NO: 4.

In some embodiments, the one or more first genetic modifications include a genetic modification capable of causing or which causes a modulation (and is some embodiments a decrease) in aldehyde dehydrogenase (NADP(+)) activity. Aldehyde dehydrogenase (NADP(+)) are classified in EC number 1.2.1.4 and catalyze the conversion of an aldehyde with NADP(+) in carboxylate with NADPH. In some specific embodiments, the one or more first genetic modifications comprise a genetic modification for reducing the expression and/or inactivativating at least one copy of a native gene encoding a polypeptide having aldehyde dehydrogenase (NADP(+)) activity. In some specific embodiments, the one or more first genetic modifications comprise a genetic modification for reducing the expression and/or inactivativating at least one copy of a native gene encoding an ald6 polypeptide.

In some embodiments, the one or more first genetic modifications include a genetic modification capable of causing or which causes a modulation (and in some embodiments an increase) in acetaldehyde dehydrogenase (acetylating) activity. Acetaldehyde dehydrogenases (acetylating) are classified in EC number 1.2.1.10 and catalyze the conversion of an acetaldehyde, CoA and NAD(+) in acetyl-CoA and NADH. In some specific embodiments, the one or more first genetic modifications comprise a genetic modification for overexpressing a native polypeptide having acetaldehyde dehydrogenase (acetylating) activity and/or expressing a heterologous polypeptide having acetaldehyde dehydrogenase (acetylating) activity. The recombinant yeast cell of the present disclosure can include a genetic modification for overexpressing a native polypeptide having acetaldehyde dehydrogenase (acetylating) activity. The recombinant yeast cell of the present disclosure can include a genetic modification for expressing a heterologous polypeptide having acetaldehyde dehydrogenase (acetylating).

In some embodiments, the one or more first genetic modifications include a genetic modification capable of causing or which causes a modulation (and is some embodiments an increase) in both alcohol dehydrogenase and acetaldehyde dehydrogenase (acetylating) activity. This can be achieved, for example, when the one or more first genetic modifications are for expression a heterologous polypeptide having both alcohol dehydrogenase and acetaldehyde dehydrogenase (acetylating) activity, referred herein as a polypeptide having acetaldehyde/alcohol dehydrogenase activity. Polypeptides having acetaldehyde/alcohol dehydrogenase activity are described in U.S. Pat. No. 8,956,851 and WO 2015/023989, incorporated herewith in their entirety. Polypeptides having acetaldehyde/alcohol dehydrogenase activity of the present disclosure include, but are not limited to, the adhE polypeptides or a polypeptide encoded by an adhe gene ortholog or gene paralog. In an embodiment, the adhE polypeptide is derived from a Bifidobacterium genus and, in specific embodiments, from Bifidobacterium adolescentis. In still another embodiment, the adhE polypeptide having the amino acid sequence of SEQ ID NO: 6, is a variant of the amino acid sequence of SEQ ID NO: 6 having acetaldehyde/alcohol dehydrogenase activity or is a fragment of the amino acid sequence of SEQ ID NO: 6 having acetaldehyde/alcohol dehydrogenase activity. In yet further embodiments, the adhE polypeptide is encoded by a nucleic acid molecule having the nucleic acid sequence of SEQ ID NO: 5 or comprising a degenerate sequence encoding SEQ ID NO: 6.

### Second Genetic Modification(s)

In embodiments, the recombinant yeast cells of the present disclosure comprises one or more second genetic modifications for increasing pyruvate decarboxylase activity in the recombinant yeast cell when compared to the parental yeast cell (lacking the second genetic modification). Pyruvate decarboxylase (E.C. 4.1.1.1) are capable of converting 2-oxo carboxylate into aldehyde and CO₂. In some embodiments, the recombinant yeast cells exhibit an increased ability in converting pyruvate to acetaldehyde (when compared to the parental yeast cell lacking the second genetic modification). Alternatively or in combination, the recombinant yeast cells exhibit a decreased ability in converting substrates other than pyruvate to aldehyde (when compared to the parental yeast cell lacking the second genetic modification). When submitted to comparable conditions, the recombinant yeast cell and the parental yeast cell will exhibit, respectively, a first level of pyruvate decarboxylase activity and a second level of pyruvate decarboxylase activity. It is understood that the first level of pyruvate decarboxylase activity associated with the recombinant yeast cell will be higher than the second level of pyruvate decarboxylase activity associated with the parental yeast cell. This increase in the level of activity of polypeptides having pyruvate decarboxylase activity is due in part to the presence of the second genetic modification in the recombinant yeast cell.

The increased pyruvate decarboxylase activity associated with the recombinant yeast cell can be used to further increase the yield in ethanol (when compared to yield in ethanol obtained with the parental yeast cell and in some embodiments, when compared to the yield in ethanol obtained with the intermediate yeast cell during comparable fermentations).

The increased pyruvate decarboxylase activity associated with the recombinant yeast cell can be used to increase a rate of production of ethanol (when compared to rate of production of ethanol obtained with the intermediate yeast cell and in some embodiments, when compared to the rate of production of ethanol obtained with the parental yeast cell during comparable fermentations). The increased pyruvate decarboxylase activity associated with the recombinant yeast cell can be used to increase the specific ethanol production rate. As used in the context of the present disclosure, the specific ethanol production rate (referred to as qₑₜₕₐₙₒₗ, which can be provided, in some embodiments, to the g ethanol/g cells/h) refers to an amount of ethanol produced/amount of yeast/unit of time

The increased pyruvate decarboxylase activity associated with the recombinant yeast cell can be used to increase the specific growth rate (when compared to specific growth rate obtained with the intermediate yeast cell during comparable fermentations). The increased pyruvate decarboxylase activity associated with the recombinant yeast cell can be used to increase the specific growth rate while at least maintaining (or in some embodiments increasing) its ethanol yield (when compared to specific growth rate and ethanol yield obtained with the intermediate yeast cell during comparable fermentations).

The increased pyruvate decarboxylase activity associated with the recombinant yeast cell can be used to at least maintaining (or in some embodiments decreasing) its glycerol production yield (when compared to the glycerol production obtained with the intermediate yeast cell during comparable fermentations). In some embodiments, the increased pyruvate decarboxylase activity reduces the specific glycerol production. As used in the context of the present disclosure, the specific glycerol production rate” (referred to as q_{glycerol}, which can, in some embodiments, be provided as g glycerol/g cells/h) refers to an amount of glycerol produced/amount of yeast/unit of time. The increased pyruvate decarboxylase activity associated with the recombinant yeast cell can be used to increase the specific growth rate while at least maintaining (or in some embodiments decreasing) its glycerol production (when compared to specific growth rate and the glycerol production obtained with the intermediate yeast cell during comparable fermentations). The increased pyruvate decarboxylase activity associated with the recombinant yeast cell can be used to increase the specific growth rate while at least maintaining (or in some embodiments increasing) its ethanol yield and maintaining (or in some embodiments decreasing) its glycerol production (when compared to specific growth rate, the ethanol yield and the glycerol production obtained with the intermediate yeast cell during comparable fermentations).

The increased pyruvate decarboxylase activity associated with the recombinant yeast cell can be used to at least maintaining (or in some embodiments decreasing) its fusel alcohol production (when compared to the fusel alcohol production obtained with the intermediate yeast cell during comparable fermentations). The increased pyruvate decarboxylase activity associated with the recombinant yeast cell can be used to increase the specific growth rate while at least maintaining (or in some embodiments decreasing) its fusel alcohol production (when compared to specific growth rate and the fusel alcohol production obtained with the intermediate yeast cell during comparable fermentations). The increased pyruvate decarboxylase activity associated with the recombinant yeast cell can be used to increase the specific growth rate while at least maintaining (or in some embodiments increasing) its ethanol yield and maintaining (or in some embodiments decreasing) its fusel alcohol production (when compared to specific growth rate, the ethanol yield and the fusel alcohol production obtained with the intermediate yeast cell during comparable fermentations).

The increased pyruvate decarboxylase activity associated with the recombinant yeast cell can be used to provide tolerance in stressful fermentations (when compared to the tolerance of the parental yeast cell and/or the intermediate yeast cell during comparable fermentations). As used in the context of the present disclosure, the expression “tolerance” refer to the ability of the recombinant yeast host cell to maintain or even improve its fermentation performances when compared to the parental yeast cell or the intermediate yeast cell in similar stressful conditions. In an embodiment, the fermentation is considered stressful because of low nitrogen availability (e.g., nitrogen scarcity which can, in some embodiments, correspond to non-protein nitrogen source available in a biomass fermentation supplemented with less than 500 ppm or less than 450 ppm urea). Conditions of nitrogen scarcity can refer, in some embodiments, to the amount of nitrogen available in a biomass fermentation supplemented with 200 ppm or less of urea. Fermentation performances includes, without limitation, the fermentation rate, the yield of ethanol, glycerol production, the rate of glycerol production, fusel alcohol production, the rate of fusel alcohol production, specific growth rate, etc. In another embodiment, the fermentation is considered stressful because of the presence of a bacterial contamination which can lead, in some additional embodiments, in a pH decrease of the substrate being fermented. In yet another embodiment, the fermentation is considered stressful because it includes a plurality of fermentation cycles and/or the use of an acid washing step between fermentation cycles. In still another embodiment, the fermentation is considered stressful because of the presence of a heat temperature being applied during the fermentation process.

The second genetic modification can, in some embodiments, cause the overexpression of one or more native polypeptides having pyruvate decarboxylase activity and/or the expression of one or more heterologous polypeptides having pyruvate decarboxylase activity. In some embodiments, the recombinant yeast cells of the present disclosure include, as the second genetic modification, a heterologous nucleic acid encoding a heterologous polypeptide having pyruvate decarboxylase activity. In additional embodiments, the heterologous polypeptide having pyruvate decarboxylase activity capable of being expressed or expressed by the recombinant yeast cell has a higher affinity (e.g., and thus a lower Km) towards pyruvate than the native polypeptides having pyruvate decarboxylase activity that may be expressed by the parental yeast cell (and optionally in the recombinant yeast cell as well). In specific embodiments, the Km of the heterologous polypeptide having pyruvate decarboxylase activity expressed by the recombinant yeast cell is equal to or less than 0.4 mM, 0.3 mM, 0.2 mM, 0.1 mM, 0.09 mM, 0.08 mM, 0.07 mM, 0.06 mM or even lower.

Polypeptides having pyruvate decarboxylase activity include pyruvate decarboxylases (EC 4.1.1.1). Pyruvate decarboxylases are involved in the conversion of pyruvate and NADH into ethanol and NAD+. The pyruvate decarboxylase can be of prokaryotic or eukaryotic origin. Pyruvate decarboxylases can be derived, for example, from Lactobacillus florum (Accession Number WP_009166425.1), Lactobacillus fructivorans (Accession Number WP_039145143.1), Lactobacillus lindneri (Accession Number WP_065866149.1), Lactococcus lactis (Accession Number WP_104141789.1), Carnobacterium gallinarum (Accession Number WP_034563038.1), Enterococcus plantarum (Accession Number WP_069654378.1), Clostridium acetobutylicum (Accession Number NP_149189.1), Bacillus megaterium (Accession Number WP_075420723.1), Kluyveromyces lactis (Accession Number CAA61155) and/or Bacillus thuringiensis (Accession Number WP_052587756.1).

In an embodiment, the pyruvate decarboxylase is derived from the genus Zymomomas, and in some further embodiments, from Zymomomas mobilis. In some further embodiments, the puryvate decarboxylase can be pdc1 from Zymomomas mobilis. In yet further embodiments, the pyruvate decarboxylase can have the amino acid sequence of SEQ ID NO:12, be a variant of the amino acid sequence of SEQ ID NO: 12 having pyruvate decarboxylase activity or be a fragment of the amino acid sequence of SEQ ID NO: 12 having pyruvate decarboxylase activity. In yet additional embodiments, the pyruvate decarboxylase can be encoded by a nucleic acid molecule having the nucleic acid sequence of SEQ ID NO: 11 or SEQ ID NO: 70 or can comprise a degenerate sequence encoding SEQ ID NO: 12, a variant thereof or a fragment thereof.

In an embodiment, the pyruvate decarboxylase is derived from the genus Zymobacter, and in some further embodiments, from Zymobacter palmae. In some further embodiments, the pyruvate decarboxylase can be pdc1 from Zymobacter palmae. In yet further embodiments, the pyruvate decarboxylase can have the amino acid sequence of SEQ ID NO:14, be a variant of the amino acid sequence of SEQ ID NO: 14 having pyruvate decarboxylase activity or be a fragment of the amino acid sequence of SEQ ID NO: 14 having pyruvate decarboxylase activity. In yet additional embodiments, the pyruvate decarboxylase can be encoded by a nucleic acid molecule having the nucleic acid sequence of SEQ ID NO: 13 or can comprise a degenerate sequence encoding SEQ ID NO: 14, a variant thereof or a fragment thereof.

In an embodiment, the pyruvate decarboxylase is derived from the genus Pisum and in some further embodiments, from Pisum sativum. In some further embodiments, the puryvate decarboxylase can be pdc1 from Pisum sativum. In yet further embodiments, the pyruvate decarboxylase can have the amino acid sequence of SEQ ID NO:16 or 18, be a variant of the amino acid sequence of SEQ ID NO: 16 having pyruvate decarboxylase activity or be a fragment of the amino acid sequence of SEQ ID NO: 16 having pyruvate decarboxylase activity. In yet additional embodiments, the pyruvate decarboxylase can be encoded by a nucleic acid molecule having the nucleic acid sequence of SEQ ID NO: 15 or comprising a degenerate sequence encoding SEQ ID NO: 16, a variant thereof or a fragment thereof. In some further embodiments, the puryvate decarboxylase can be pdc2 from Pisum sativum. In yet further embodiments, the pyruvate decarboxylase can have the amino acid sequence of SEQ ID NO:17, be a variant of the amino acid sequence of SEQ ID NO: 17 having pyruvate decarboxylase activity or be a fragment of the amino acid sequence of SEQ ID NO: 17 having pyruvate decarboxylase activity. In yet additional embodiments, the pyruvate decarboxylase can be encoded by a nucleic acid molecule comprising a degenerate sequence encoding SEQ ID NO: 17, a variant thereof or a fragment thereof.

In an embodiment, the pyruvate decarboxylase is derived from the genus Saccharomyces and in some further embodiments, from Saccharomyces cerevisiae. In some further embodiments, the puryvate decarboxylase can be pdc1 from Saccharomyces cerevisiae. In yet further embodiments, the pyruvate decarboxylase can have the amino acid sequence of SEQ ID NO: 34, be a variant of the amino acid sequence of SEQ ID NO: 34 having pyruvate decarboxylase activity or be a fragment of the amino acid sequence of SEQ ID NO: 34 having pyruvate decarboxylase activity. In yet additional embodiments, the pyruvate decarboxylase can be encoded by a nucleic acid molecule having the nucleic acid sequence of SEQ ID NO: 33 or comprising a degenerate sequence encoding SEQ ID NO: 34, a variant thereof or a fragment thereof. In some further embodiments, the puryvate decarboxylase can be pdc5 from Saccharomyces cerevisiae. In yet further embodiments, the pyruvate decarboxylase can have the amino acid sequence of SEQ ID NO: 35, be a variant of the amino acid sequence of SEQ ID NO: 35 having pyruvate decarboxylase activity or be a fragment of the amino acid sequence of SEQ ID NO: 35 having pyruvate decarboxylase activity. In yet additional embodiments, the pyruvate decarboxylase can be encoded by a nucleic acid molecule comprising a degenerate sequence encoding SEQ ID NO: 35, a variant thereof or a fragment thereof. In some further embodiments, the puryvate decarboxylase can be pdc6 from Saccharomyces cerevisiae. In yet further embodiments, the pyruvate decarboxylase can have the amino acid sequence of SEQ ID NO: 36, be a variant of the amino acid sequence of SEQ ID NO: 36 having pyruvate decarboxylase activity or be a fragment of the amino acid sequence of SEQ ID NO: 36 having pyruvate decarboxylase activity. In yet additional embodiments, the pyruvate decarboxylase can be encoded by a nucleic acid molecule comprising a degenerate sequence encoding SEQ ID NO: 36, a variant thereof or a fragment thereof.

In an embodiment, the pyruvate decarboxylase is derived from the genus Gluconacetobacter and in some further embodiments, from Gluconacetobacter diazotrophicus. In some further embodiments, the puryvate decarboxylase can be pdc1 from Gluconacetobacter diazotrophicus. In yet further embodiments, the pyruvate decarboxylase can have the amino acid sequence of SEQ ID NO: 69, be a variant of the amino acid sequence of SEQ ID NO: 69 having pyruvate decarboxylase activity or be a fragment of the amino acid sequence of SEQ ID NO: 69 having pyruvate decarboxylase activity. In yet additional embodiments, the pyruvate decarboxylase can be encoded by a nucleic acid molecule comprising a degenerate sequence encoding SEQ ID NO: 69, a variant thereof or a fragment thereof.

In an embodiment, the pyruvate decarboxylase is derived from the genus Kluyveromyces and in some further embodiments, from Kluyveromyces lactis. In some further embodiments, the puryvate decarboxylase can be pdc1 from Kluyveromyces lactis. In yet further embodiments, the pyruvate decarboxylase can have the amino acid sequence of SEQ ID NO: 30, be a variant of the amino acid sequence of SEQ ID NO: 30 having pyruvate decarboxylase activity or be a fragment of the amino acid sequence of SEQ ID NO: 30 having pyruvate decarboxylase activity. In yet additional embodiments, the pyruvate decarboxylase can be encoded by a nucleic acid molecule having the nucleic acid sequence of SEQ ID NO: 29 or can comprise a degenerate sequence encoding SEQ ID NO: 30, a variant thereof or a fragment thereof.

### Additional Genetic Modifications

In some embodiments, the recombinant yeast cell of the present disclosure can include one or more third genetic modifications for increasing the glyceraldehyde-3-phosphate dehydrogenase lacking phosphorylating activity in the recombinant yeast cell (when compared to the parental yeast cell). Polypeptides exhibiting glyceraldehyde-3-phosphate dehydrogenase lacking phosphorylating activity are known to belong to EC 1.2.1.9 or 1.2.1.90.

Glyceraldehyde-3-phosphate dehydrogenases from EC 1.2.1.9 are also known as triosephosphate dehydrogenases catalyze the following reaction:

D-glyceraldehyde 3-phosphate+NADP⁺+H₂O<=>3-phospho-D-glycerate+NADPH

Glyceraldehyde-3-phosphate dehydrogenase from EC 1.2.1.90 are also known as non-phosphorylating glyceraldehyde-3-phosphate dehydrogenase and catalyze the following reaction:

D-glyceraldehyde 3-phosphate+NAD(P)⁺+H₂O<=>3-phospho-D-glycerate+NAD(P)H

For example, the third genetic modification is capable of causing or causes the overexpression of a native enzyme belonging to EC 1.2.1.9 or 1.2.1.90 and/or the expression of a heterologous enzyme belonging to EC 1.2.1.9 or 1.2.1.90. In some embodiments, the recombinant yeast cell of the present disclosure comprises a genetic modification for overexpressing a native enzyme belonging to EC 1.2.1.9 or 1.2.1.90. In some embodiments, the recombinant yeast cell of the present disclosure comprises a genetic modification for expressing a heterologous enzyme belonging to EC 1.2.1.9 or 1.2.1.90. In some embodiments, the recombinant yeast cell of the present disclosure comprises a genetic modification for overexpressing a native enzyme belonging to EC 1.2.1.9 or 1.2.1.90 and another one for expressing a heterologous enzyme belonging to EC 1.2.1.9 or 1.2.1.90.

In some embodiments, the glyceraldehyde-3-phosphate dehydrogenase can be derived from a bacteria, for example, from the genus Streptococcus and, in some instances, from the species Streptococcus mutans. The glyceraldehyde-3-phosphate dehydrogenase can be encoded by the gapN gene from Streptococcus mutans, or a gapN gene ortholog, or a gapN gene paralog. In an embodiment, the gapN has the amino acid sequence of SEQ ID NO: 23, is a variant of the amino acid of SEQ ID NO: 23 having glyceraldehyde-3-phosphate dehydrogenase lacking phosphorylating activity or is a fragment of SEQ ID NO: 23 having glyceraldehyde-3-phosphate dehydrogenase lacking phosphorylating activity. In additional embodiments, the gapN is encoded by a nucleic acid molecule having the nucleic acid sequence of SEQ ID NO: 22 or comprising a degenerate sequence encoding SEQ ID NO: 23, a variant thereof or a fragment thereof.

In some embodiments, the glyceraldehyde-3-phosphate dehydrogenase can be derived from a bacteria, for example, from the genus Lactobacillus and, in some instances, from the species Lactobacillus delbrueckii. The glyceraldehyde-3-phosphate dehydrogenase can be encoded by the gapN gene from Lactobacillus delbrueckii, or a gapN gene ortholog, or a gapN gene paralog. In an embodiment, the gapN has the amino acid sequence of SEQ ID NO: 38, is a variant of the amino acid of SEQ ID NO: 38 having glyceraldehyde-3-phosphate dehydrogenase lacking phosphorylating activity or is a fragment of SEQ ID NO: 38 having glyceraldehyde-3-phosphate dehydrogenase lacking phosphorylating activity. In additional embodiments, the gapN is encoded by a nucleic acid molecule having the nucleic acid sequence of SEQ ID NO: 37 or comprising a degenerate sequence encoding SEQ ID NO: 38, a variant thereof or a fragment thereof.

In some embodiments, the glyceraldehyde-3-phosphate dehydrogenase can be derived from a bacteria, for example, from the genus Streptococcus and, in some instances, from the species Streptococcus thermophilus. The glyceraldehyde-3-phosphate dehydrogenase can be encoded by the gapN gene from Streptococcus thermophilus, or a gapN gene ortholog, or a gapN gene paralog. In an embodiment, the gapN has the amino acid sequence of SEQ ID NO: 40, is a variant of the amino acid of SEQ ID NO: 40 having glyceraldehyde-3-phosphate dehydrogenase lacking phosphorylating activity or is a fragment of SEQ ID NO: 40 having glyceraldehyde-3-phosphate dehydrogenase lacking phosphorylating activity. In additional embodiments, the gapN is encoded by a nucleic acid molecule having the nucleic acid sequence of SEQ ID NO: 39 or comprising a degenerate sequence encoding SEQ ID NO: 40, a variant thereof or a fragment thereof.

In some embodiments, the glyceraldehyde-3-phosphate dehydrogenase can be derived from a bacteria, for example, from the genus Streptococcus and, in some instances, from the species Streptococcus macacae. The glyceraldehyde-3-phosphate dehydrogenase can be encoded by the gapN gene from Streptococcus macacae, or a gapN gene ortholog, or a gapN gene paralog. In an embodiment, the gapN has the amino acid sequence of SEQ ID NO: 42, is a variant of the amino acid of SEQ ID NO: 42 having glyceraldehyde-3-phosphate dehydrogenase lacking phosphorylating activity or is a fragment of SEQ ID NO: 42 having glyceraldehyde-3-phosphate dehydrogenase lacking phosphorylating activity. In additional embodiments, the gapN is encoded by a nucleic acid molecule having the nucleic acid sequence of SEQ ID NO: 41 or comprising a degenerate sequence encoding SEQ ID NO: 42, a variant thereof or a fragment thereof.

In some embodiments, the glyceraldehyde-3-phosphate dehydrogenase can be derived from a bacteria, for example, from the genus Streptococcus and, in some instances, from the species Streptococcus hyointestinalis. The glyceraldehyde-3-phosphate dehydrogenase can be encoded by the gapN gene from Streptococcus hyointestinalis, or a gapN gene ortholog, or a gapN gene paralog. In an embodiment, the gapN has the amino acid sequence of SEQ ID NO: 44, is a variant of the amino acid of SEQ ID NO: 44 having glyceraldehyde-3-phosphate dehydrogenase lacking phosphorylating activity or is a fragment of SEQ ID NO: 44 having glyceraldehyde-3-phosphate dehydrogenase lacking phosphorylating activity. In additional embodiments, the gapN is encoded by a nucleic acid molecule having the nucleic acid sequence of SEQ ID NO: 43 or comprising a degenerate sequence encoding SEQ ID NO: 44, a variant thereof or a fragment thereof.

In some embodiments, the glyceraldehyde-3-phosphate dehydrogenase can be derived from a bacteria, for example, from the genus Streptococcus and, in some instances, from the species Streptococcus urinalis. The glyceraldehyde-3-phosphate dehydrogenase can be encoded by the gapN gene from Streptococcus urinalis, or a gapN gene ortholog, or a gapN gene paralog. In an embodiment, the gapN has the amino acid sequence of SEQ ID NO: 46, is a variant of the amino acid of SEQ ID NO: 46 having glyceraldehyde-3-phosphate dehydrogenase lacking phosphorylating activity or is a fragment of SEQ ID NO: 46 having glyceraldehyde-3-phosphate dehydrogenase lacking phosphorylating activity. In additional embodiments, the gapN is encoded by a nucleic acid molecule having the nucleic acid sequence of SEQ ID NO: 45 or comprising a degenerate sequence encoding SEQ ID NO: 46, a variant thereof or a fragment thereof.

In some embodiments, the glyceraldehyde-3-phosphate dehydrogenase can be derived from a bacteria, for example, from the genus Streptococcus and, in some instances, from the species Streptococcus canis. The glyceraldehyde-3-phosphate dehydrogenase can be encoded by the gapN gene from Streptococcus canis, or a gapN gene ortholog, or a gapN gene paralog. In an embodiment, the gapN has the amino acid sequence of SEQ ID NO: 48, is a variant of the amino acid of SEQ ID NO: 48 having glyceraldehyde-3-phosphate dehydrogenase lacking phosphorylating activity or is a fragment of SEQ ID NO: 48 having glyceraldehyde-3-phosphate dehydrogenase lacking phosphorylating activity. In additional embodiments, the gapN is encoded by a nucleic acid molecule having the nucleic acid sequence of SEQ ID NO: 47 or comprising a degenerate sequence encoding SEQ ID NO: 48, a variant thereof or a fragment thereof.

In some embodiments, the glyceraldehyde-3-phosphate dehydrogenase can be derived from a bacteria, for example, from the genus Streptococcus and, in some instances, from the species Streptococcus thoraltensis. The glyceraldehyde-3-phosphate dehydrogenase can be encoded by the gapN gene from Streptococcus thoraltensis, or a gapN gene ortholog, or a gapN gene paralog. In an embodiment, the gapN has the amino acid sequence of SEQ ID NO: 50, is a variant of the amino acid of SEQ ID NO: 50 having glyceraldehyde-3-phosphate dehydrogenase lacking phosphorylating activity or is a fragment of SEQ ID NO: 50 having glyceraldehyde-3-phosphate dehydrogenase lacking phosphorylating activity. In additional embodiments, the gapN is encoded by a nucleic acid molecule having the nucleic acid sequence of SEQ ID NO: 49 or comprising a degenerate sequence encoding SEQ ID NO: 50, a variant thereof or a fragment thereof.

In some embodiments, the glyceraldehyde-3-phosphate dehydrogenase can be derived from a bacteria, for example, from the genus Streptococcus and, in some instances, from the species Streptococcus dysgalactiae. The glyceraldehyde-3-phosphate dehydrogenase can be encoded by the gapN gene from Streptococcus dysgalactiae, or a gapN gene ortholog, or a gapN gene paralog. In an embodiment, the gapN has the amino acid sequence of SEQ ID NO: 52, is a variant of the amino acid of SEQ ID NO: 52 having glyceraldehyde-3-phosphate dehydrogenase lacking phosphorylating activity or is a fragment of SEQ ID NO: 52 having glyceraldehyde-3-phosphate dehydrogenase lacking phosphorylating activity. In additional embodiments, the gapN is encoded by a nucleic acid molecule having the nucleic acid sequence of SEQ ID NO: 51 or comprising a degenerate sequence encoding SEQ ID NO: 52, a variant thereof or a fragment thereof.

In some embodiments, the glyceraldehyde-3-phosphate dehydrogenase can be derived from a bacteria, for example, from the genus Streptococcus and, in some instances, from the species Streptococcus pyogenes. The glyceraldehyde-3-phosphate dehydrogenase can be encoded by the gapN gene from Streptococcus pyogenes, or a gapN gene ortholog, or a gapN gene paralog. In an embodiment, the gapN has the amino acid sequence of SEQ ID NO: 54, is a variant of the amino acid of SEQ ID NO: 54 having glyceraldehyde-3-phosphate dehydrogenase lacking phosphorylating activity or is a fragment of SEQ ID NO: 54 having glyceraldehyde-3-phosphate dehydrogenase lacking phosphorylating activity. In additional embodiments, the gapN is encoded by a nucleic acid molecule having the nucleic acid sequence of SEQ ID NO: 53 or comprising a degenerate sequence encoding SEQ ID NO: 54, a variant thereof or a fragment thereof.

In some embodiments, the glyceraldehyde-3-phosphate dehydrogenase can be derived from a bacteria, for example, from the genus Streptococcus and, in some instances, from the species Streptococcus ictaluri. The glyceraldehyde-3-phosphate dehydrogenase can be encoded by the gapN gene from Streptococcus ictaluri, or a gapN gene ortholog, or a gapN gene paralog. In an embodiment, the gapN has the amino acid sequence of SEQ ID NO: 56, is a variant of the amino acid of SEQ ID NO: 56 having glyceraldehyde-3-phosphate dehydrogenase lacking phosphorylating activity or is a fragment of SEQ ID NO: 56 having glyceraldehyde-3-phosphate dehydrogenase lacking phosphorylating activity. In additional embodiments, the gapN is encoded by a nucleic acid molecule having the nucleic acid sequence of SEQ ID NO: 55 or comprising a degenerate sequence encoding SEQ ID NO: 56, a variant thereof or a fragment thereof.

In some embodiments, the glyceraldehyde-3-phosphate dehydrogenase can be derived from a bacteria, for example, from the genus Clostridium and, in some instances, from the species Clostridium perfringens. The glyceraldehyde-3-phosphate dehydrogenase can be encoded by the gapN gene from Clostridium perfringens, or a gapN gene ortholog, or a gapN gene paralog. In an embodiment, the gapN has the amino acid sequence of SEQ ID NO: 58, is a variant of the amino acid of SEQ ID NO: 58 having glyceraldehyde-3-phosphate dehydrogenase lacking phosphorylating activity or is a fragment of SEQ ID NO: 58 having glyceraldehyde-3-phosphate dehydrogenase lacking phosphorylating activity. In additional embodiments, the gapN is encoded by a nucleic acid molecule having the nucleic acid sequence of SEQ ID NO: 57 or comprising a degenerate sequence encoding SEQ ID NO: 58, a variant thereof or a fragment thereof.

In some embodiments, the glyceraldehyde-3-phosphate dehydrogenase can be derived from a bacteria, for example, from the genus Clostridium and, in some instances, from the species Clostridium chromiireducens. The glyceraldehyde-3-phosphate dehydrogenase can be encoded by the gapN gene from Clostridium chromiireducens, or a gapN gene ortholog, or a gapN gene paralog. In an embodiment, the gapN has the amino acid sequence of SEQ ID NO: 60, is a variant of the amino acid of SEQ ID NO: 60 having glyceraldehyde-3-phosphate dehydrogenase lacking phosphorylating activity or is a fragment of SEQ ID NO: 60 having glyceraldehyde-3-phosphate dehydrogenase lacking phosphorylating activity. In additional embodiments, the gapN is encoded by a nucleic acid molecule having the nucleic acid sequence of SEQ ID NO: 59 or comprising a degenerate sequence encoding SEQ ID NO: 60, a variant thereof or a fragment thereof.

In some embodiments, the glyceraldehyde-3-phosphate dehydrogenase can be derived from a bacteria, for example, from the genus Clostridium and, in some instances, from the species Clostridium botulinum. The glyceraldehyde-3-phosphate dehydrogenase can be encoded by the gapN gene from Clostridium botulinum, or a gapN gene ortholog, or a gapN gene paralog. In an embodiment, the gapN has the amino acid sequence of SEQ ID NO: 62, is a variant of the amino acid of SEQ ID NO: 62 having glyceraldehyde-3-phosphate dehydrogenase lacking phosphorylating activity or is a fragment of SEQ ID NO: 62 having glyceraldehyde-3-phosphate dehydrogenase lacking phosphorylating activity. In additional embodiments, the gapN is encoded by a nucleic acid molecule having the nucleic acid sequence of SEQ ID NO: 61 or comprising a degenerate sequence encoding SEQ ID NO: 62, a variant thereof or a fragment thereof.

In some embodiments, the glyceraldehyde-3-phosphate dehydrogenase can be derived from a bacteria, for example, from the genus Bacillus and, in some instances, from the species Bacillus cereus. The glyceraldehyde-3-phosphate dehydrogenase can be encoded by the gapN gene from Bacillus cereus, or a gapN gene ortholog, or a gapN gene paralog. In an embodiment, the gapN has the amino acid sequence of SEQ ID NO: 64, is a variant of the amino acid of SEQ ID NO: 64 having glyceraldehyde-3-phosphate dehydrogenase lacking phosphorylating activity or is a fragment of SEQ ID NO: 64 having glyceraldehyde-3-phosphate dehydrogenase lacking phosphorylating activity. In additional embodiments, the gapN is encoded by a nucleic acid molecule having the nucleic acid sequence of SEQ ID NO: 63 or comprising a degenerate sequence encoding SEQ ID NO: 64, a variant thereof or a fragment thereof.

In some embodiments, the glyceraldehyde-3-phosphate dehydrogenase can be derived from a bacteria, for example, from the genus Bacillus and, in some instances, from the species Bacillus anthracis. The glyceraldehyde-3-phosphate dehydrogenase can be encoded by the gapN gene from Bacillus anthracis, or a gapN gene ortholog, or a gapN gene paralog. In an embodiment, the gapN has the amino acid sequence of SEQ ID NO: 66, is a variant of the amino acid of SEQ ID NO: 66 having glyceraldehyde-3-phosphate dehydrogenase lacking phosphorylating activity or is a fragment of SEQ ID NO: 66 having glyceraldehyde-3-phosphate dehydrogenase lacking phosphorylating activity. In additional embodiments, the gapN is encoded by a nucleic acid molecule having the nucleic acid sequence of SEQ ID NO: 65 or comprising a degenerate sequence encoding SEQ ID NO: 66, a variant thereof or a fragment thereof.

In some embodiments, the glyceraldehyde-3-phosphate dehydrogenase can be derived from a bacteria, for example, from the genus Bacillus and, in some instances, from the species Bacillus thuringiensis. The glyceraldehyde-3-phosphate dehydrogenase can be encoded by the gapN gene from Bacillus thuringiensis, or a gapN gene ortholog, or a gapN gene paralog. In an embodiment, the gapN has the amino acid sequence of SEQ ID NO: 68, is a variant of the amino acid of SEQ ID NO: 68 having glyceraldehyde-3-phosphate dehydrogenase lacking phosphorylating activity or is a fragment of SEQ ID NO: 68 having glyceraldehyde-3-phosphate dehydrogenase lacking phosphorylating activity. In additional embodiments, the gapN is encoded by a nucleic acid molecule having the nucleic acid sequence of SEQ ID NO: 67 or comprising a degenerate sequence encoding SEQ ID NO: 68, a variant thereof or a fragment thereof.

In some embodiments, the glyceraldehyde-3-phosphate dehydrogenase can be derived from a bacteria, for example, from the genus Pyrococcus and, in some instances, from the species Pyrococcus furiosus. The glyceraldehyde-3-phosphate dehydrogenase can be encoded by the gapN gene from Pyrococcus furiosus, or a gapN gene ortholog, or a gapN gene paralog. In an embodiment, the gapN has the amino acid sequence of SEQ ID NO: 32, is a variant of the amino acid of SEQ ID NO: 32 having glyceraldehyde-3-phosphate dehydrogenase lacking phosphorylating activity or is a fragment of SEQ ID NO: 32 having glyceraldehyde-3-phosphate dehydrogenase lacking phosphorylating activity. In additional embodiments, the gapN is encoded by a nucleic acid molecule having the nucleic acid sequence of SEQ ID NO: 31 or comprising a degenerate sequence encoding SEQ ID NO: 32, a variant thereof or a fragment thereof.

In some embodiments, the glyceraldehyde-3-phosphate dehydrogenase can be derived from a bacteria, for example, from the genus Populus and, in some instances, from the species Populus deltoides. The glyceraldehyde-3-phosphate dehydrogenase can be encoded by the gapN gene from Populus deltoides, or a gapN gene ortholog, or a gapN gene paralog.

Embodiments of glyceraldehyde-3-phosphate dehydrogenase can also be derived, without limitation, from the following (the number in brackets correspond to the Gene ID number): Triticum aestivum (543435); Streptococcus mutans (1028095); Streptococcus agalactiae (1013627); Streptococcus pyogenes (901445); Clostridioides difficile (4913365); Mycoplasma mycoides subsp. mycoides SC str. (2744894); Streptococcus pneumoniae (933338); Streptococcus sanguinis (4807521); Acinetobacter pittii (11638070); Clostridium botulinum A str. (5185508); Bacillus thuringiensis serovar konkukian str. (2857794); Bacillus anthracis str. Ames (1088724); Phaeodactylum tricornutum (7199937); Emiliania huxleyi (17251102); Zea mays (542583); Helianthus annuus (110928814); Streptomyces coelicolor (1101118); Burkholderia pseudomallei (U.S. Pat. Nos. 3,097,058, 3,095,849); variants thereof as well as fragments thereof.

Additional embodiments of glyceraldehyde-3-phosphate dehydrogenase can also be derived, without limitation, from the following (the number in brackets correspond to the Pubmed Accession number): Streptococcus macacae (WP_003081126.1), Streptococcus hyointestinalis (WP_115269374.1), Streptococcus urinalis (WP_006739074.1), Streptococcus canis (WP_003044111.1), Streptococcus pluranimalium (WP_104967491.1), Streptococcus equi (WP_012678132.1), Streptococcus thoraltensis (WP_018380938.1), Streptococcus dysgalactiae (WP_138125971.1), Streptococcus halotolerans (WP_062707672.1), Streptococcus pyogenes (WP_136058687.1), Streptococcus ictaluri (WP_008090774.1), Clostridium perfringens (WP_142691612.1), Clostridium chromiireducens (WP_079442081.1), Clostridium botulinum (WP_012422907.1), Bacillus cereus (WP_000213623.1), Bacillus anthracis (WP_098340670.1), Bacillus thuringiensis (WP_087951472.1), Pyrococcus furiosus (WP_011013013.1) as well as variants thereof and fragments thereof.

In some embodiments, the one or more third genetic modifications include a genetic modification capable of causing or which causes a modulation (and is some embodiments an increase) in alcohol dehydrogenase activity. Alcohol dehydrogenases are classified in EC number 1.1.1.1 and catalyze the conversion of a primary or a secondary alcohol with NAD(+) in an aldehyde or a ketone with NADH. In some specific embodiments, the one or more third genetic modifications comprise a genetic modification for overexpressing a native polypeptide having alcohol dehydrogenase activity and/or expressing a heterologous polypeptide having alcohol dehydrogenase activity. The recombinant yeast cell of the present disclosure can include a genetic modification for overexpressing a native polypeptide having alcohol dehydrogenase activity. The recombinant yeast cell of the present disclosure can include a genetic modification for expressing a heterologous polypeptide having alcohol dehydrogenase activity. In some embodiments, recombinant yeast cell comprising the one or more third genetic modification for increasing alcohol dehydrogenase activity can be further modified to reduce the expression and/or inactivate at least one copy of a native gene encoding a polypeptide having alcohol dehydrogenase activity. In some embodiments, the Km of the alcohol dehydrogenase(s) that is (are) being expressed in the recombinant yeast host cell is equal to or below 0.22, and is some embodiments, between 0.008 and 0.22. In some additional embodiments, the Km of the alcohol dehydrogenase(s) that is (are) being expressed in the recombinant yeast host cell is below 0.22, and is some embodiments, between 0.008 and below 0.22. Embodiments of alcohol dehydrogenases are disclosed in WO 92/16615 A1 and are herewith incorporated in their entirety.

Heterologous alcohol dehydrogenases includes, but are not limited to the adhA polypeptide (also known as the adhl polypeptide), a polypeptide encoded by an adha gene ortholog or gene paralog, the adhB polypeptide (also known as the adhll polypeptide) or a polypeptide encoded by an adhb gene ortholog or gene paralog. In an embodiment, the polypeptide having alcohol dehydrogenase activity is derived from a Zymomonas genus and, in specific embodiments, from Zymomonas mobilis. In still another embodiment, the adhA polypeptide having the amino acid sequence of SEQ ID NO: 19, is a variant of the amino acid sequence of SEQ ID NO: 19 having alcohol dehydrogenase activity or is a fragment of the amino acid sequence of SEQ ID NO: 19 having alcohol dehydrogenase activity. In yet further embodiments, the adhA polypeptide is encoded by a nucleic acid molecule having the nucleic acid sequence of SEQ ID NO: 18 or comprising a degenerate sequence encoding SEQ ID NO: 19. In still another embodiment, the adhB polypeptide has the amino acid sequence of SEQ ID NO: 21, is a variant of the amino acid sequence of SEQ ID NO: 21 having alcohol dehydrogenase activity or is a fragment of the amino acid sequence of SEQ ID NO: 21 having alcohol dehydrogenase activity. In yet further embodiments, the adhB polypeptide is encoded by a nucleic acid molecule having the nucleic acid sequence of SEQ ID NO: 20 or comprising a degenerate sequence encoding the polypeptide having the amino acid sequence of SEQ ID NO: 21, a variant thereof or a fragment thereof.

In additional embodiments, the recombinant yeast cell of the present disclosure can include one or more third genetic modification for increasing alcohol dehydrogenase activity in the recombinant yeast cell (when compared to the parental yeast cell). Heterologous alcohol dehydrogenases includes, but are not limited to the adh polypeptide and a polypeptide encoded by an adh gene ortholog or gene paralog. In an embodiment, heterologous alcohol dehydrogenase do not have acetaldehyde dehydrogenase activity. In an embodiment, the polypeptide having alcohol dehydrogenase activity is derived from a Sporotrichum genus and, in specific embodiments, from Sporotrichum pulverulentum. In such embodiment, the adh polypeptide has the amino acid sequence of SEQ ID NO: 73, is a variant of the amino acid sequence of SEQ ID NO: 73 or is a fragment of the amino acid sequence of SEQ ID NO: 73 having alcohol dehydrogenase activity. In a further embodiment, the adh polypeptide is enclosed by a nucleic acid molecule having the nucleic acid sequence of SEQ ID NO: 72 or comprising a degenerate sequence encoding the adh polypeptide having the amino acid sequence of SEQ ID NO: 73, a variant thereof or a fragment thereof. In an embodiment, the polypeptide having alcohol dehydrogenase activity is derived from a Saccharomyces genus and, in specific embodiments, from Saccharomyces cerevisiae (which corresponds to GenBank Accession number CAA99098.1 or Uniprot P00330). In a specific embodiment, the polypeptide having alcohol dehydrogenase activity has the amino acid sequence of SEQ ID NO: 89, is a variant of the amino acid sequence of SEQ ID NO: 89 having alcohol dehydrogenase activity or is a fragment of the amino acid sequence of SEQ ID NO: 89 having alcohol dehydrogenase activity. In such embodiment, the alcohol dehydrogenase can be referred to as ADH1. In an embodiment, the polypeptide having alcohol dehydrogenase activity is derived from a Aspergillus genus and, in specific embodiments, from Aspergillus nidulans. In such embodiment, the adh polypeptide has the amino acid sequence of SEQ ID NO: 75, is a variant of the amino acid sequence of SEQ ID NO: 75 or is a fragment of the amino acid sequence of SEQ ID NO: 75 having alcohol dehydrogenase activity. In a further embodiment, the adh polypeptide is enclosed by a nucleic acid molecule having the nucleic acid sequence of SEQ ID NO: 74 or comprising a degenerate sequence encoding the adh polypeptide having the amino acid sequence of SEQ ID NO: 75, a variant thereof or a fragment thereof. In an embodiment, the polypeptide having alcohol dehydrogenase activity is derived from a Natronomonas genus and, in specific embodiments, from Natronomonas pharaonis. In such embodiment, the adh polypeptide has the amino acid sequence of SEQ ID NO: 77, is a variant of the amino acid sequence of SEQ ID NO: 77 or is a fragment of the amino acid sequence of SEQ ID NO: 77 having alcohol dehydrogenase activity. In a further embodiment, the adh polypeptide is enclosed by a nucleic acid molecule having the nucleic acid sequence of SEQ ID NO: 76 or comprising a degenerate sequence encoding the adh polypeptide having the amino acid sequence of SEQ ID NO: 77, a variant thereof or a fragment thereof. In an embodiment, the polypeptide having alcohol dehydrogenase activity is derived from a Homo genus and, in specific embodiments, from Homo sapiens. In such embodiments, the alcohol dehydrogenase can be referred to as isoenzyme beta 1 or ADH2. In such embodiment, the adh2 polypeptide has the amino acid sequence of SEQ ID NO: 79, is a variant of the amino acid sequence of SEQ ID NO: 79 or is a fragment of the amino acid sequence of SEQ ID NO: 79 having alcohol dehydrogenase activity. In a further embodiment, the adh polypeptide is enclosed by a nucleic acid molecule having the nucleic acid sequence of SEQ ID NO: 78 or comprising a degenerate sequence encoding the adh polypeptide having the amino acid sequence of SEQ ID NO: 79, a variant thereof or a fragment thereof. In an embodiment, the polypeptide having alcohol dehydrogenase activity is derived from a Saimiri genus and, in specific embodiments, from Saimiri sciureus. In such embodiment, the adh polypeptide has the amino acid sequence of SEQ ID NO: 81, is a variant of the amino acid sequence of SEQ ID NO: 81 or is a fragment of the amino acid sequence of SEQ ID NO: 81 having alcohol dehydrogenase activity. In a further embodiment, the adh polypeptide is enclosed by a nucleic acid molecule having the nucleic acid sequence of SEQ ID NO: 80 or comprising a degenerate sequence encoding the adh polypeptide having the amino acid sequence of SEQ ID NO: 81, a variant thereof or a fragment thereof. In an embodiment, the polypeptide having alcohol dehydrogenase activity is derived from a Meyerozyma genus and, in specific embodiments, from Meyerozyma guilliermondii. In such embodiment, the alcohol dehydrogenase can be referred to as ADH1. In such embodiment, the adh1 polypeptide has the amino acid sequence of SEQ ID NO: 83, is a variant of the amino acid sequence of SEQ ID NO: 83 or is a fragment of the amino acid sequence of SEQ ID NO: 83 having alcohol dehydrogenase activity. In a further embodiment, the adh polypeptide is enclosed by a nucleic acid molecule having the nucleic acid sequence of SEQ ID NO: 82 or comprising a degenerate sequence encoding the adh polypeptide having the amino acid sequence of SEQ ID NO: 83, a variant thereof or a fragment thereof. In an embodiment, the polypeptide having alcohol dehydrogenase activity is derived from a Rattus genus and, in specific embodiments, from Rattus norvegicus. In such embodiment, the alcohol dehydrogenase can be referred to as isoenzyme 3. In such embodiment, the isoenzyme 3 polypeptide has the amino acid sequence of SEQ ID NO: 85, is a variant of the amino acid sequence of SEQ ID NO: 85 or is a fragment of the amino acid sequence of SEQ ID NO: 85 having alcohol dehydrogenase activity. In a further embodiment, the adh polypeptide is enclosed by a nucleic acid molecule having the nucleic acid sequence of SEQ ID NO: 84 or comprising a degenerate sequence encoding the adh polypeptide having the amino acid sequence of SEQ ID NO: 85, a variant thereof or a fragment thereof.

In some embodiments, the recombinant yeast cell of the present disclosure includes a further additional genetic modification to reduced the native pyruvate decarboxylase activity (in the recombinant yeast cell when compared to the parental cell). In some embodiments, this further genetic modification is capable of reducing the expression or inactivating at least one or more native gene encoding a native polypeptide having pyruvate decarboxylase activity. This further additional genetic modification can be done, for example, to reduce the cell's ability to convert substrates other than pyruvate into fusel alcohols (such as acetoin and/or butanediol). This further additional genetic modification can be made in one or all copies of a native gene encoding a native polypeptide having pyruvate decarboxylase activity. In some embodiments, the recombinant yeast cell include the inactivation of at least one copy (and in some embodiments all copies) of a native gene encoding a native pdc1 polypeptide, an ortholog thereof or a paralog thereof. In some embodiments, the recombinant yeast cell include the inactivation of at least one copy (and in some embodiments all copies) of a native gene encoding a native pdc5 polypeptide, an ortholog thereof or a paralog thereof. In some embodiments, the recombinant yeast cell include the inactivation of at least one copy (and in some embodiments all copies) of a native gene encoding a native pdc6 polypeptide, an ortholog thereof or a paralog thereof.

In some embodiments, the recombinant yeast cell of the present disclosure includes a further additional genetic modification to reduce a native butanediol dehydrogenase activity (in the recombinant yeast cell when compared to the parental cell). In some embodiments, the further additional genetic modification is capable of reducing the expression or inactivating at least one or more native gene encoding a native polypeptide having butanediol dehydrogenase activity. This further additional genetic modification can be done, for example, to reduce butanediol accumulation. This further additional genetic modification can be made in one or all copies of a native gene encoding a native polypeptide having butanedial dehydrogenase activity. In some embodiments, the recombinant yeast cell include the inactivation of at least one copy (and in some embodiments all copies) of a native gene encoding a native bdh1 polypeptide, an ortholog thereof or a paralog thereof. In some embodiments, the recombinant yeast cell include the inactivation of at least one copy (and in some embodiments all copies) of a native gene encoding a native bdh2 polypeptide, an ortholog thereof or a paralog thereof.

In some embodiments, the recombinant yeast can optionally include one or more further genetic modification allowing the expression of a heterologous saccharolytic enzyme. As used in the context of the present disclosure, a “saccharolytic enzyme” can be any enzyme involved in carbohydrate digestion, metabolism and/or hydrolysis, including amylases, cellulases, hemicellulases, cellulolytic and amylolytic accessory enzymes, inulinases, levanases, and pentose sugar utilizing enzymes. In an embodiment, the saccharolytic enzyme is an amylolytic enzyme. As used herein, the expression “amylolytic enzyme” refers to a class of enzymes capable of hydrolyzing starch or hydrolyzed starch. Amylolytic enzymes include, but are not limited to alpha-amylases (EC 3.2.1.1, sometimes referred to fungal alpha-amylase, see below), maltogenic amylase (EC 3.2.1.133), glucoamylase (EC 3.2.1.3), glucan 1,4-alpha-maltotetraohydrolase (EC 3.2.1.60), pullulanase (EC 3.2.1.41), iso-amylase (EC 3.2.1.68) and amylomaltase (EC 2.4.1.25). In an embodiment, the one or more amylolytic enzymes can be an alpha-amylase from Aspergillus oryzae, a maltogenic alpha-amylase from Geobacillus stearothermophilus, a glucoamylase from Saccharomycopsis fibuligera, a glucan 1,4-alpha-maltotetraohydrolase from Pseudomonas saccharophila, a pullulanase from Bacillus naganoensis, a pullulanase from Bacillus acidopullulyticus, an iso-amylase from Pseudomonas amyloderamosa, and/or amylomaltase from Thermus thermophilus. Some amylolytic enzymes have been described in US Patent Application published under US/2022/0127564, incorporated herewith incorporated by reference.

In some embodiments, the recombinant yeast cell can bear one or more genetic modifications allowing for the production of a heterologous glucoamylase. Many microbes produce an amylase to degrade extracellular starches. In addition to cleaving the last α(1-4) glycosidic linkages at the non-reducing end of amylose and amylopectin, yielding glucose, γ-amylase will cleave α(1-6) glycosidic linkages. The heterologous glucoamylase can be derived from any organism. In an embodiment, the heterologous polypeptide is derived from a γ-amylase, such as, for example, the glucoamylase of Saccharomycopsis fibuligera (e.g., encoded by the glu 0111 gene). Examples of yeast host cells bearing such second genetic modifications are described in U.S. patent Ser. Nos. 10/385,345 and 11/332,728 both herewith incorporated in their entirety.

In another embodiment, the recombinant yeast cell can optionally include one or more further genetic modification allowing the expression of a glucoside hydrolase capable of hydrolyzing an unfermentable carbohydrate source that is present in the storage medium (e.g., trehalose for example). The glucoside hydrolase can have trehalase activity and can be a trehalase. Trehalases are glycoside hydrolases capable of converting trehalose into glucose. Trehalases have been classified under EC number 3.2.1.28. Trehalases can be classified into two broad categories based on their optimal pH: neutral trehalases (having an optimum pH of about 7) and acid trehalases (having an optimum pH of about 4.5). The heterologous trehalases that can be used in the context of the present disclosure can be of various origins such as bacterial, fungal or plant origin. In a specific embodiment, the trehalase is from fungal origin. In such embodiment, the substrate or cellular component can be trehalose or a trehalose-containing biological product. Various embodiments of heterologous trehalases that can be used in the recombinant yeast cell of the present description are disclosed in US Patent Application published under US/2021/0348145, incorporated herewith in its entirety.

Promoter-Optimized Recombinant Yeast Cell Expressing a Stl1 or a gapN Polypeptide

The present disclosure also provides a recombinant yeast cell expressing a stl1 polypeptide under the control of a promoter (e.g., referred herewith as a promoter-optimized recombinant yeast cell). The promoter-optimized recombinant yeast cell can be used, for example, to improve a yield in ethanol (when compared to its corresponding parental yeast cell not expressing the stl1 polypeptide or not expressing a promoter-optimized stl1 polypeptide). Such promoter-optimized recombinant yeast cell comprises one or more heterologous nucleic acid molecules, comprising a first polynucleotide (comprising one or more promoters). In some embodiments, the first polynucleotide of the one or more heterologous nucleic molecules is operatively associated with a second polynucleotide (encoding the stl1 polypeptide). In some embodiments, the heterologous nucleic acid molecules may include the same or different promoter(s). In additional embodiments, the heterologous nucleic acid molecules may include the same or different second polynucleotides (which may encode the same or different stl1 polypeptides). One or more copies of the heterologous nucleic acid molecules may be integrated in the recombinant yeast cell's genome (and in some embodiments, in the recombinant yeast cell's chromosome). In some embodiments, the heterologous nucleic acid molecules may be knocked-in at the genomic location of the native promoter of the native gene encoding the native stl1 polypeptide. In some embodiments, the heterologous nucleic acid molecules may be knocked-in at the genomic location of the native gene encoding the native stl1 polypeptide. In additional embodiments, the promoter-optimized recombinant yeast cell can include a further genetic modification to reduced the expression or inactivate at least one copy (and in some embodiments all copies) of the native gene encoding the native stl1 polypeptide.

The first polynucleotide includes one or more promoters capable of controlling the expression of a downstream polynucleotide encoding a native or a heterologous stl1 polypeptide. The promoter or combination of promoters present in the first polynucleotide can include one or more of constitutive promoters (such as, for example, tef1p (e.g., the promoter of the tef1 gene), tef2p (e.g., the promoter of the tef2 gene), cwp2p (e.g., the promoter of the cwp2 gene), ssa1p (e.g., the promoter of the ssa1 gene), eno1p (e.g., the promoter of the eno1 gene), eno2p (e.g., the promoter of the eno2 gene), hxk1p (e.g., the promoter of the hxk1 gene), pgk1p (e.g., the promoter of the pgk1 gene), ydr524c-bp (e.g., the promoter of the ydr524c-b gene), gpm1p (e.g., the promoter of the gpm1 gene), and/or tpi1p (e.g., the promoter of the tpi1 gene). The promoter or combination of promoters present in the first polynucleotide can include one or more of inducible promoters. Inducible promoters include, without limitation, glucose-regulated promoters (e.g., the promoter of the hxt3 gene (referred to as hxt3p), the promoter of the hxt7 gene (referred to as hxt7p), or the promoter of the cyc1 gene (referred to as the cyc1p)), sulfite-regulated promoters (e.g., the promoter of the gpd2 gene (referred to as gpd2p), the promoter of the fzf1 gene (referred to as the fzf1p), the promoter of the ssu1 gene (referred to as ssu1p), the promoter of the ssu1-r gene (referred to as ssur1-rp), ribosomal promoters (e.g., the promoter of the rp/3 gene (referred to as the rpl3p) or the promoter of the qcr8 gene (referred to as qcr8p)), anaerobic-regulated promoters (e.g., tdh1p (e.g., the promoter of the tdh1 gene), pau5p (e.g., the promoter of the pau5 gene), hor7p (e.g., the promoter of the hor7 gene), adh1p (e.g., the promoter of the adh1 gene), tdh2p (e.g., the promoter of the tdh2 gene), tdh3p (e.g., the promoter of the tdh3 gene), gpd1p (e.g., the promoter of the gpd1 gene), cdc19p (e.g., the promoter of the cdc19 gene), pdc1p (e.g., the promoter of the pdc1 gene), hxt3p (e.g., the promoter of the hxt3 gene), dan1p (e.g., the promoter of the dan1 gene), tir1p (e.g., the promoter of the tir1 gene) and tpi1p (e.g., the promoter of the tpi1 gene)), stress-regulated promoters (e.g., hor7p (e.g., the promoter of the hor7 gene), glycolytic-regulated promoters (e.g., adh1p (e.g., the promoter of the adh1 gene), eno2p (e.g., the promoter of the eno2 gene), pgk1p (e.g., the promoter of the pgk1 gene), tef1p (e.g., the promoter of the tef1 gene), tef2p (e.g., the promoter of the tef2 gene), gpm1p (e.g., the promoter of the gpm1 gene) and/or tpi1p (e.g., the promoter of the tpi1 gene)). In a specific embodiment, the promoter is one or more of adh1p (e.g., the promoter of the adh1 gene), eno2p (e.g., the promoter of the eno2 gene), pgk1p (e.g., the promoter of the pgk1 gene), ydr524c-bp (e.g., the promoter of the ydr524c-b gene), tef1p (e.g., the promoter of the tef1 gene), tef2p (e.g., the promoter of the tef2 gene), tpi1p (e.g., the promoter of the tpi1 gene), gpm1p (e.g., the promoter of the gpm1 gene), rpl3p (e.g., the promoter of the rp/3 gene), cyc1p (e.g., the promoter of the cyc1p gene), tdh1p (e.g., the promoter of the tdh1 gene), qcr8p (e.g., the promoter of the qcr8 gene), tir1p (e.g., the promoter of the tir1 gene) or hor7p (e.g., the promoter of the hor7 gene).

The second polynucleotide encodes a stl1 polypeptide, a polypeptide encoded by a stl1 gene ortholog and/or a polypeptide encoded by a stl1 gene paralog. The stl1 genes encoding the stl1 polypeptide include, but are not limited to, Saccharomyces cerevisiae Gene ID: 852149, Candida albicans, Kluyveromyces lactis Gene ID: 2896463, Ashbya gossypii Gene ID: 4620396, Eremothecium sinecaudum Gene ID: 28724161, Torulaspora delbrueckii Gene ID: 11505245, Lachancea thermotolerans Gene ID: 8290820, Phialophora attae Gene ID: 28742143, Penicillium digitatum Gene ID: 26229435, Aspergillus oryzae Gene ID: 5997623, Aspergillus fumigatus Gene ID: 3504696, Talaromyces atroroseus Gene ID: 31007540, Rasamsonia emersonii Gene ID: 25315795, Aspergillus flavus Gene ID: 7910112, Aspergillus terreus Gene ID: 4322759, Penicillium chrysogenum Gene ID: 8310605, Alternaria alternata Gene ID: 29120952, Paraphaeosphaeria sporulosa Gene ID: 28767590, Pyrenophora tritici-repentis Gene ID: 6350281, Metarhizium robertsii Gene ID: 19259252, Isaria fumosorosea Gene ID: 30023973, Cordyceps militaris Gene ID: 18171218, Pochonia chlamydosporia Gene ID: 28856912, Metarhizium majus Gene ID: 26274087, Neofusicoccum parvum Gene ID: 19029314, Diplodia corticola Gene ID: 31017281, Verticillium dahliae Gene ID: 20711921, Colletotrichum gloeosporioides Gene ID: 18740172, Verticillium albo-atrum Gene ID: 9537052, Paracoccidioides lutzii Gene ID: 9094964, Trichophyton rubrum Gene ID: 10373998, Nannizzia gypsea Gene ID: 10032882, Trichophyton verrucosum Gene ID: 9577427, Arthroderma benhamiae Gene ID: 9523991, Magnaporthe oryzae Gene ID: 2678012, Gaeumannomyces graminis var. tritici Gene ID: 20349750, Togninia minima Gene ID: 19329524, Eutypa lata Gene ID: 19232829, Scedosporium apiospermum Gene ID: 27721841, Aureobasidium namibiae Gene ID: 25414329, Sphaerulina musiva Gene ID: 27905328 as well as Pachysolen tannophilus GenBank Accession Numbers JQ481633 and JQ481634, Saccharomyces paradoxus stl1 and Millerozyma farinose GenBank accession number CCE78002. In an embodiment, the stl1 polypeptide is encoded by Saccharomyces cerevisiae Gene ID: 852149. In a specific embodiment, the stl1 polypeptide is derived from Saccharomyces sp. and in further embodiments from Saccharomyces cerevisiae. In yet additional embodiment, the stl1 polypeptide has the amino acid sequence of SEQ ID NO: 8, is a variant of the amino acid sequence of SEQ ID NO: 8 having glycerol proton symporter activity or is a fragment of the amino acid sequence of SEQ ID NO: 8 having glycerol proton symporter activity. In additional embodiment, the stl1 polypeptide can be encoded by a nucleic acid molecule comprising the nucleic acid sequence of SEQ ID NO: 7 or SEQ ID NO: 71 or can comprise a degenerate sequence encoding the amino acid sequence of SEQ ID NO: 8, a variant thereof or a fragment thereof. In some specific embodiments, the heterologous nucleic acid molecule encoding the stl1 polypeptide, its variants or its fragments is knocked-in at the native position at which the gene of the native stl1 polypeptide is located.

The present disclosure also provides a recombinant yeast cell expressing a gapN polypeptide under the control of a promoter (e.g., referred herewith as a promoter-optimized recombinant yeast cell), including those described in PCT/IB2019/060527, incorporated herewith in its entirety. The promoter-optimized recombinant yeast cell can be used, for example, to improve a yield in ethanol (when compared to its corresponding parental yeast cell not expressing the gapN polypeptide or not expressing a promoter-optimized gapN polypeptide). Such promoter-optimized recombinant yeast cell comprises one or more heterologous nucleic acid molecules, comprising a first polynucleotide (comprising one or more promoters). In some embodiments, the first polynucleotide of the one or more heterologous nucleic molecules is operatively associated with a second polynucleotide (encoding the gapN polypeptide). In some embodiments, the heterologous nucleic acid molecules may include the same or different promoter(s). In additional embodiments, the heterologous nucleic acid molecules may include the same or different second polynucleotides (which may encode the same or different gapN polypeptides). One or more copies of the heterologous nucleic acid molecules may be integrated in the recombinant yeast cell's genome (and in some embodiments, in the recombinant yeast cell's chromosome). In some embodiments, the heterologous nucleic acid molecules may be knocked-in at the genomic location of the native promoter of the native gene encoding the native gapN polypeptide. In some embodiments, the heterologous nucleic acid molecules may be knocked-in at the genomic location of the native gene encoding the native gapN polypeptide. In additional embodiments, the promoter-optimized recombinant yeast cell can include a further genetic modification to reduced the expression or inactivate at least one copy (and in some embodiments all copies) of the native gene encoding the native gapN polypeptide.

The first polynucleotide includes one or more promoters capable of controlling the expression of a downstream polynucleotide encoding a native or a heterologous gapN polypeptide. The promoter or combination of promoters present in the first polynucleotide can include one or more of constitutive promoters (such as, for example, tef1p (e.g., the promoter of the tef1 gene), tef2p (e.g., the promoter of the tef2 gene), cwp2p (e.g., the promoter of the cwp2 gene), ssa1p (e.g., the promoter of the ssa1 gene), eno1p (e.g., the promoter of the eno1 gene), eno2p (e.g., the promoter of the eno2 gene), hxk1p (e.g., the promoter of the hxk1 gene), pgk1p (e.g., the promoter of the pgk1 gene), ydr524c-bp (e.g., the promoter of the ydr524c-b gene), gpm1p (e.g., the promoter of the gpm1 gene), and/or tpi1p (e.g., the promoter of the tpi1 gene). The promoter or combination of promoters present in the first polynucleotide can include one or more of inducible promoters. Inducible promoters include, without limitation, glucose-regulated promoters (e.g., the promoter of the hxt3 gene (referred to as hxt3p), the promoter of the hxt7 gene (referred to as hxt7p), or the promoter of the cyc1 gene (referred to as the cyc1p)), sulfite-regulated promoters (e.g., the promoter of the gpd2 gene (referred to as gpd2p), the promoter of the fzf1 gene (referred to as the fzf1p), the promoter of the ssu1 gene (referred to as ssu1p), the promoter of the ssu1-r gene (referred to as ssur1-rp), ribosomal promoters (e.g., the promoter of the rp/3 gene (referred to as the rpl3p) or the promoter of the qcr8 gene (referred to as qcr8p)), anaerobic-regulated promoters (e.g., tdh1p (e.g., the promoter of the tdh1 gene), pau5p (e.g., the promoter of the pau5 gene), hor7p (e.g., the promoter of the hor7 gene), adh1p (e.g., the promoter of the adh1 gene), tdh2p (e.g., the promoter of the tdh2 gene), tdh3p (e.g., the promoter of the tdh3 gene), gpd1p (e.g., the promoter of the gpd1 gene), cdc19p (e.g., the promoter of the cdc19 gene), pdc1p (e.g., the promoter of the pdc1 gene), hxt3p (e.g., the promoter of the hxt3 gene), dan1p (e.g., the promoter of the dan1 gene), tir1p (e.g., the promoter of the tir1 gene) and tpi1p (e.g., the promoter of the tpi1 gene)), stress-regulated promoters (e.g., hor7p (e.g., the promoter of the hor7 gene), glycolytic-regulated promoters (e.g., adh1p (e.g., the promoter of the adh1 gene), eno2p (e.g., the promoter of the eno2 gene), pgk1p (e.g., the promoter of the pgk1 gene), tef1p (e.g., the promoter of the tef1 gene), tef2p (e.g., the promoter of the tef2 gene), gpm1p (e.g., the promoter of the gpm1 gene) and/or tpi1p (e.g., the promoter of the tpi1 gene)). In a specific embodiment, the promoter is one or more of adh1p (e.g., the promoter of the adh1 gene), eno2p (e.g., the promoter of the eno2 gene), pgk1p (e.g., the promoter of the pgk1 gene), ydr524c-bp (e.g., the promoter of the ydr524c-b gene), tef1p (e.g., the promoter of the tef1 gene), tef2p (e.g., the promoter of the tef2 gene), tpi1p (e.g., the promoter of the tpi1 gene), gpm1p (e.g., the promoter of the gpm1 gene), rpl3p (e.g., the promoter of the rp/3 gene), cyc1p (e.g., the promoter of the cyc1p gene), tdh1p (e.g., the promoter of the tdh1 gene), qcr8p (e.g., the promoter of the qcr8 gene), tir1p (e.g., the promoter of the tir1 gene) or hor7p (e.g., the promoter of the hor7 gene).

In some embodiments, the promoters included in the first polynucleotide include, but are not limited a constitutive promoter (such as, for example, tef2p (e.g., the promoter of the TEF2 gene), cwp2p (e.g., the promoter of the CWP2 gene), ssa1p (e.g., the promoter of the SSA1 gene), eno1p (e.g., the promoter of the ENO1 gene), hxk1 (e.g., the promoter of the HXK1 gene), pgi1p (e.g., the promoter from the PGI1 gene), pfk1p (e.g., the promoter from the PFK1 gene), fba1p (e.g., the promoter from the FBA1 gene), gpm1p (e.g., the promoter from the GPM1 gene) and/or pgk1p (e.g., the promoter of the PGK1 gene). However, is some embodiments, it is preferable to limit the expression of the heterologous polypeptide. In some embodiments, the promoter or combination of promoters present on the first polynucleotide can include an inducible or modulated promoters such as, for example, a glucose-regulated promoter (e.g., the promoter of the HXT7 gene (referred to as hxt7p)), a pentose phosphate pathway promoter (e.g., the promoter of the ZWF1 gene (zwf1p)) or a sulfite-regulated promoter (e.g., the promoter of the GPD2 gene (referred to as gpd2p) or the promoter of the FZF1 gene (referred to as the fzf1p)), the promoter of the SSU1 gene (referred to as ssu1p), the promoter of the SSU1-r gene (referred to as ssur1-rp). In an embodiment, the promoter or combination of promoters include an anaerobic-regulated promoters, such as, for example tdh1p (e.g., the promoter of the TDH1 gene), pau5p (e.g., the promoter of the PAU5 gene), hor7p (e.g., the promoter of the HOR7 gene), adh1p (e.g., the promoter of the ADH1 gene), tdh2p (e.g., the promoter of the TDH2 gene), tdh3p (e.g., the promoter of the tdh3 gene), gpd1p (e.g., the promoter of the GPD1 gene), cdc19p (e.g., the promoter of the CDC19 gene), eno2p (e.g., the promoter of the ENO2 gene), pdc1p (e.g., the promoter of the PDC1 gene), hxt3p (e.g., the promoter of the HXT3 gene), dan1 (e.g., the promoter of the DAN1 gene) and tpi1p (e.g., the promoter of the TPI1 gene). In yet another embodiment, the promoter or combination of promoters can include a cytochrome c/mitochondrial electron transport chain promoter, such as, for example, the cyc1p (e.g., the promoter of the CYC1 gene) and/or the qcr8p (e.g., the promoter of the QCR8 gene). In an embodiment, the promoter or combination of promoters includes gpd1p, e.g., the promoter of the GPD1 gene. In another embodiment, the promoter or combination of promoters includes zwf1p, e.g., the promoter of the ZWF1 gene.

The second polynucleotide encodes a gapN polypeptide, a polypeptide encoded by a gapN gene ortholog and/or a polypeptide encoded by a gapN gene paralog. In some embodiments, the glyceraldehyde-3-phosphate dehydrogenase can be derived from a bacteria, for example, from the genus Streptococcus and, in some instances, from the species Streptococcus mutans. The glyceraldehyde-3-phosphate dehydrogenase can be encoded by the gapN gene from Streptococcus mutans, or a gapN gene ortholog, or a gapN gene paralog. In an embodiment, the gapN has the amino acid sequence of SEQ ID NO: 23, is a variant of the amino acid of SEQ ID NO: 23 having glyceraldehyde-3-phosphate dehydrogenase lacking phosphorylating activity or is a fragment of SEQ ID NO: 23 having glyceraldehyde-3-phosphate dehydrogenase lacking phosphorylating activity. In additional embodiments, the gapN is encoded by a nucleic acid molecule having the nucleic acid sequence of SEQ ID NO: 22 or comprising a degenerate sequence encoding SEQ ID NO: 23, a variant thereof or a fragment thereof.

In some embodiments, the glyceraldehyde-3-phosphate dehydrogenase can be derived from a bacteria, for example, from the genus Lactobacillus and, in some instances, from the species Lactobacillus delbrueckii. The glyceraldehyde-3-phosphate dehydrogenase can be encoded by the gapN gene from Lactobacillus delbrueckii, or a gapN gene ortholog, or a gapN gene paralog. In an embodiment, the gapN has the amino acid sequence of SEQ ID NO: 38, is a variant of the amino acid of SEQ ID NO: 38 having glyceraldehyde-3-phosphate dehydrogenase lacking phosphorylating activity or is a fragment of SEQ ID NO: 38 having glyceraldehyde-3-phosphate dehydrogenase lacking phosphorylating activity. In additional embodiments, the gapN is encoded by a nucleic acid molecule having the nucleic acid sequence of SEQ ID NO: 37 or comprising a degenerate sequence encoding SEQ ID NO: 38, a variant thereof or a fragment thereof.

In some embodiments, the glyceraldehyde-3-phosphate dehydrogenase can be derived from a bacteria, for example, from the genus Streptococcus and, in some instances, from the species Streptococcus thermophilus. The glyceraldehyde-3-phosphate dehydrogenase can be encoded by the gapN gene from Streptococcus thermophilus, or a gapN gene ortholog, or a gapN gene paralog. In an embodiment, the gapN has the amino acid sequence of SEQ ID NO: 40, is a variant of the amino acid of SEQ ID NO: 40 having glyceraldehyde-3-phosphate dehydrogenase lacking phosphorylating activity or is a fragment of SEQ ID NO: 40 having glyceraldehyde-3-phosphate dehydrogenase lacking phosphorylating activity. In additional embodiments, the gapN is encoded by a nucleic acid molecule having the nucleic acid sequence of SEQ ID NO: 39 or comprising a degenerate sequence encoding SEQ ID NO: 40, a variant thereof or a fragment thereof.

In some embodiments, the glyceraldehyde-3-phosphate dehydrogenase can be derived from a bacteria, for example, from the genus Streptococcus and, in some instances, from the species Streptococcus macacae. The glyceraldehyde-3-phosphate dehydrogenase can be encoded by the gapN gene from Streptococcus macacae, or a gapN gene ortholog, or a gapN gene paralog. In an embodiment, the gapN has the amino acid sequence of SEQ ID NO: 42, is a variant of the amino acid of SEQ ID NO: 42 having glyceraldehyde-3-phosphate dehydrogenase lacking phosphorylating activity or is a fragment of SEQ ID NO: 42 having glyceraldehyde-3-phosphate dehydrogenase lacking phosphorylating activity. In additional embodiments, the gapN is encoded by a nucleic acid molecule having the nucleic acid sequence of SEQ ID NO: 41 or comprising a degenerate sequence encoding SEQ ID NO: 42, a variant thereof or a fragment thereof.

In some embodiments, the glyceraldehyde-3-phosphate dehydrogenase can be derived from a bacteria, for example, from the genus Streptococcus and, in some instances, from the species Streptococcus hyointestinalis. The glyceraldehyde-3-phosphate dehydrogenase can be encoded by the gapN gene from Streptococcus hyointestinalis, or a gapN gene ortholog, or a gapN gene paralog. In an embodiment, the gapN has the amino acid sequence of SEQ ID NO: 44, is a variant of the amino acid of SEQ ID NO: 44 having glyceraldehyde-3-phosphate dehydrogenase lacking phosphorylating activity or is a fragment of SEQ ID NO: 44 having glyceraldehyde-3-phosphate dehydrogenase lacking phosphorylating activity. In additional embodiments, the gapN is encoded by a nucleic acid molecule having the nucleic acid sequence of SEQ ID NO: 43 or comprising a degenerate sequence encoding SEQ ID NO: 44, a variant thereof or a fragment thereof.

In some embodiments, the glyceraldehyde-3-phosphate dehydrogenase can be derived from a bacteria, for example, from the genus Streptococcus and, in some instances, from the species Streptococcus urinalis. The glyceraldehyde-3-phosphate dehydrogenase can be encoded by the gapN gene from Streptococcus urinalis, or a gapN gene ortholog, or a gapN gene paralog. In an embodiment, the gapN has the amino acid sequence of SEQ ID NO: 46, is a variant of the amino acid of SEQ ID NO: 46 having glyceraldehyde-3-phosphate dehydrogenase lacking phosphorylating activity or is a fragment of SEQ ID NO: 46 having glyceraldehyde-3-phosphate dehydrogenase lacking phosphorylating activity. In additional embodiments, the gapN is encoded by a nucleic acid molecule having the nucleic acid sequence of SEQ ID NO: 45 or comprising a degenerate sequence encoding SEQ ID NO: 46, a variant thereof or a fragment thereof.

In some embodiments, the glyceraldehyde-3-phosphate dehydrogenase can be derived from a bacteria, for example, from the genus Streptococcus and, in some instances, from the species Streptococcus canis. The glyceraldehyde-3-phosphate dehydrogenase can be encoded by the gapN gene from Streptococcus canis, or a gapN gene ortholog, or a gapN gene paralog. In an embodiment, the gapN has the amino acid sequence of SEQ ID NO: 48, is a variant of the amino acid of SEQ ID NO: 48 having glyceraldehyde-3-phosphate dehydrogenase lacking phosphorylating activity or is a fragment of SEQ ID NO: 48 having glyceraldehyde-3-phosphate dehydrogenase lacking phosphorylating activity. In additional embodiments, the gapN is encoded by a nucleic acid molecule having the nucleic acid sequence of SEQ ID NO: 47 or comprising a degenerate sequence encoding SEQ ID NO: 48, a variant thereof or a fragment thereof.

In some embodiments, the glyceraldehyde-3-phosphate dehydrogenase can be derived from a bacteria, for example, from the genus Streptococcus and, in some instances, from the species Streptococcus thoraltensis. The glyceraldehyde-3-phosphate dehydrogenase can be encoded by the gapN gene from Streptococcus thoraltensis, or a gapN gene ortholog, or a gapN gene paralog. In an embodiment, the gapN has the amino acid sequence of SEQ ID NO: 50, is a variant of the amino acid of SEQ ID NO: 50 having glyceraldehyde-3-phosphate dehydrogenase lacking phosphorylating activity or is a fragment of SEQ ID NO: 50 having glyceraldehyde-3-phosphate dehydrogenase lacking phosphorylating activity. In additional embodiments, the gapN is encoded by a nucleic acid molecule having the nucleic acid sequence of SEQ ID NO: 49 or comprising a degenerate sequence encoding SEQ ID NO: 50, a variant thereof or a fragment thereof.

In some embodiments, the glyceraldehyde-3-phosphate dehydrogenase can be derived from a bacteria, for example, from the genus Streptococcus and, in some instances, from the species Streptococcus dysgalactiae. The glyceraldehyde-3-phosphate dehydrogenase can be encoded by the gapN gene from Streptococcus dysgalactiae, or a gapN gene ortholog, or a gapN gene paralog. In an embodiment, the gapN has the amino acid sequence of SEQ ID NO: 52, is a variant of the amino acid of SEQ ID NO: 52 having glyceraldehyde-3-phosphate dehydrogenase lacking phosphorylating activity or is a fragment of SEQ ID NO: 52 having glyceraldehyde-3-phosphate dehydrogenase lacking phosphorylating activity. In additional embodiments, the gapN is encoded by a nucleic acid molecule having the nucleic acid sequence of SEQ ID NO: 51 or comprising a degenerate sequence encoding SEQ ID NO: 52, a variant thereof or a fragment thereof.

In some embodiments, the glyceraldehyde-3-phosphate dehydrogenase can be derived from a bacteria, for example, from the genus Streptococcus and, in some instances, from the species Streptococcus pyogenes. The glyceraldehyde-3-phosphate dehydrogenase can be encoded by the gapN gene from Streptococcus pyogenes, or a gapN gene ortholog, or a gapN gene paralog. In an embodiment, the gapN has the amino acid sequence of SEQ ID NO: 54, is a variant of the amino acid of SEQ ID NO: 54 having glyceraldehyde-3-phosphate dehydrogenase lacking phosphorylating activity or is a fragment of SEQ ID NO: 54 having glyceraldehyde-3-phosphate dehydrogenase lacking phosphorylating activity. In additional embodiments, the gapN is encoded by a nucleic acid molecule having the nucleic acid sequence of SEQ ID NO: 53 or comprising a degenerate sequence encoding SEQ ID NO: 54, a variant thereof or a fragment thereof.

In some embodiments, the glyceraldehyde-3-phosphate dehydrogenase can be derived from a bacteria, for example, from the genus Streptococcus and, in some instances, from the species Streptococcus ictaluri. The glyceraldehyde-3-phosphate dehydrogenase can be encoded by the gapN gene from Streptococcus ictaluri, or a gapN gene ortholog, or a gapN gene paralog. In an embodiment, the gapN has the amino acid sequence of SEQ ID NO: 56, is a variant of the amino acid of SEQ ID NO: 56 having glyceraldehyde-3-phosphate dehydrogenase lacking phosphorylating activity or is a fragment of SEQ ID NO: 56 having glyceraldehyde-3-phosphate dehydrogenase lacking phosphorylating activity. In additional embodiments, the gapN is encoded by a nucleic acid molecule having the nucleic acid sequence of SEQ ID NO: 55 or comprising a degenerate sequence encoding SEQ ID NO: 56, a variant thereof or a fragment thereof.

In some embodiments, the glyceraldehyde-3-phosphate dehydrogenase can be derived from a bacteria, for example, from the genus Clostridium and, in some instances, from the species Clostridium perfringens. The glyceraldehyde-3-phosphate dehydrogenase can be encoded by the gapN gene from Clostridium perfringens, or a gapN gene ortholog, or a gapN gene paralog. In an embodiment, the gapN has the amino acid sequence of SEQ ID NO: 58, is a variant of the amino acid of SEQ ID NO: 58 having glyceraldehyde-3-phosphate dehydrogenase lacking phosphorylating activity or is a fragment of SEQ ID NO: 58 having glyceraldehyde-3-phosphate dehydrogenase lacking phosphorylating activity. In additional embodiments, the gapN is encoded by a nucleic acid molecule having the nucleic acid sequence of SEQ ID NO: 57 or comprising a degenerate sequence encoding SEQ ID NO: 58, a variant thereof or a fragment thereof.

In some embodiments, the glyceraldehyde-3-phosphate dehydrogenase can be derived from a bacteria, for example, from the genus Clostridium and, in some instances, from the species Clostridium chromiireducens. The glyceraldehyde-3-phosphate dehydrogenase can be encoded by the gapN gene from Clostridium chromiireducens, or a gapN gene ortholog, or a gapN gene paralog. In an embodiment, the gapN has the amino acid sequence of SEQ ID NO: 60, is a variant of the amino acid of SEQ ID NO: 60 having glyceraldehyde-3-phosphate dehydrogenase lacking phosphorylating activity or is a fragment of SEQ ID NO: 60 having glyceraldehyde-3-phosphate dehydrogenase lacking phosphorylating activity. In additional embodiments, the gapN is encoded by a nucleic acid molecule having the nucleic acid sequence of SEQ ID NO: 59 or comprising a degenerate sequence encoding SEQ ID NO: 60, a variant thereof or a fragment thereof.

In some embodiments, the glyceraldehyde-3-phosphate dehydrogenase can be derived from a bacteria, for example, from the genus Clostridium and, in some instances, from the species Clostridium botulinum. The glyceraldehyde-3-phosphate dehydrogenase can be encoded by the gapN gene from Clostridium botulinum, or a gapN gene ortholog, or a gapN gene paralog. In an embodiment, the gapN has the amino acid sequence of SEQ ID NO: 62, is a variant of the amino acid of SEQ ID NO: 62 having glyceraldehyde-3-phosphate dehydrogenase lacking phosphorylating activity or is a fragment of SEQ ID NO: 62 having glyceraldehyde-3-phosphate dehydrogenase lacking phosphorylating activity. In additional embodiments, the gapN is encoded by a nucleic acid molecule having the nucleic acid sequence of SEQ ID NO: 61 or comprising a degenerate sequence encoding SEQ ID NO: 62, a variant thereof or a fragment thereof.

In some embodiments, the glyceraldehyde-3-phosphate dehydrogenase can be derived from a bacteria, for example, from the genus Bacillus and, in some instances, from the species Bacillus cereus. The glyceraldehyde-3-phosphate dehydrogenase can be encoded by the gapN gene from Bacillus cereus, or a gapN gene ortholog, or a gapN gene paralog. In an embodiment, the gapN has the amino acid sequence of SEQ ID NO: 64, is a variant of the amino acid of SEQ ID NO: 64 having glyceraldehyde-3-phosphate dehydrogenase lacking phosphorylating activity or is a fragment of SEQ ID NO: 64 having glyceraldehyde-3-phosphate dehydrogenase lacking phosphorylating activity. In additional embodiments, the gapN is encoded by a nucleic acid molecule having the nucleic acid sequence of SEQ ID NO: 63 or comprising a degenerate sequence encoding SEQ ID NO: 64, a variant thereof or a fragment thereof.

In some embodiments, the glyceraldehyde-3-phosphate dehydrogenase can be derived from a bacteria, for example, from the genus Bacillus and, in some instances, from the species Bacillus anthracis. The glyceraldehyde-3-phosphate dehydrogenase can be encoded by the gapN gene from Bacillus anthracis, or a gapN gene ortholog, or a gapN gene paralog. In an embodiment, the gapN has the amino acid sequence of SEQ ID NO: 66, is a variant of the amino acid of SEQ ID NO: 66 having glyceraldehyde-3-phosphate dehydrogenase lacking phosphorylating activity or is a fragment of SEQ ID NO: 66 having glyceraldehyde-3-phosphate dehydrogenase lacking phosphorylating activity. In additional embodiments, the gapN is encoded by a nucleic acid molecule having the nucleic acid sequence of SEQ ID NO: 65 or comprising a degenerate sequence encoding SEQ ID NO: 66, a variant thereof or a fragment thereof.

In some embodiments, the glyceraldehyde-3-phosphate dehydrogenase can be derived from a bacteria, for example, from the genus Bacillus and, in some instances, from the species Bacillus thuringiensis. The glyceraldehyde-3-phosphate dehydrogenase can be encoded by the gapN gene from Bacillus thuringiensis, or a gapN gene ortholog, or a gapN gene paralog. In an embodiment, the gapN has the amino acid sequence of SEQ ID NO: 68, is a variant of the amino acid of SEQ ID NO: 68 having glyceraldehyde-3-phosphate dehydrogenase lacking phosphorylating activity or is a fragment of SEQ ID NO: 68 having glyceraldehyde-3-phosphate dehydrogenase lacking phosphorylating activity. In additional embodiments, the gapN is encoded by a nucleic acid molecule having the nucleic acid sequence of SEQ ID NO: 67 or comprising a degenerate sequence encoding SEQ ID NO: 68, a variant thereof or a fragment thereof.

In some embodiments, the glyceraldehyde-3-phosphate dehydrogenase can be derived from a bacteria, for example, from the genus Pyrococcus and, in some instances, from the species Pyrococcus furiosus. The glyceraldehyde-3-phosphate dehydrogenase can be encoded by the gapN gene from Pyrococcus furiosus, or a gapN gene ortholog, or a gapN gene paralog. In an embodiment, the gapN has the amino acid sequence of SEQ ID NO: 32, is a variant of the amino acid of SEQ ID NO: 32 having glyceraldehyde-3-phosphate dehydrogenase lacking phosphorylating activity or is a fragment of SEQ ID NO: 32 having glyceraldehyde-3-phosphate dehydrogenase lacking phosphorylating activity. In additional embodiments, the gapN is encoded by a nucleic acid molecule having the nucleic acid sequence of SEQ ID NO: 31 or comprising a degenerate sequence encoding SEQ ID NO: 32, a variant thereof or a fragment thereof.

In some embodiments, the glyceraldehyde-3-phosphate dehydrogenase can be derived from a bacteria, for example, from the genus Populus and, in some instances, from the species Populus deltoides. The glyceraldehyde-3-phosphate dehydrogenase can be encoded by the gapN gene from Populus deltoides, or a gapN gene ortholog, or a gapN gene paralog.

Embodiments of glyceraldehyde-3-phosphate dehydrogenase can also be derived, without limitation, from the following (the number in brackets correspond to the Gene ID number): Triticum aestivum (543435); Streptococcus mutans (1028095); Streptococcus agalactiae (1013627); Streptococcus pyogenes (901445); Clostridioides difficile (4913365); Mycoplasma mycoides subsp. mycoides SC str. (2744894); Streptococcus pneumoniae (933338); Streptococcus sanguinis (4807521); Acinetobacter pittii (11638070); Clostridium botulinum A str. (5185508); [Bacillus thuringiensis] serovar konkukian str. (2857794); Bacillus anthracis str. Ames (1088724); Phaeodactylum tricornutum (7199937); Emiliania huxleyi (17251102); Zea mays (542583); Helianthus annuus (110928814); Streptomyces coelicolor (1101118); Burkholderia pseudomallei (U.S. Pat. Nos. 3,097,058, 3,095,849); variants thereof as well as fragments thereof.

Additional embodiments of glyceraldehyde-3-phosphate dehydrogenase can also be derived, without limitation, from the following (the number in brackets correspond to the Pubmed Accession number): Streptococcus macacae (WP_003081126.1), Streptococcus hyointestinalis (WP_115269374.1), Streptococcus urinalis (WP_006739074.1), Streptococcus canis (WP_003044111.1), Streptococcus pluranimalium (WP_104967491.1), Streptococcus equi (WP_012678132.1), Streptococcus thoraltensis (WP_018380938.1), Streptococcus dysgalactiae (WP_138125971.1), Streptococcus halotolerans (WP_062707672.1), Streptococcus pyogenes (WP_136058687.1), Streptococcus ictaluri (WP_008090774.1), Clostridium perfringens (WP_142691612.1), Clostridium chromiireducens (WP_079442081.1), Clostridium botulinum (WP_012422907.1), Bacillus cereus (WP_000213623.1), Bacillus anthracis (WP_098340670.1), Bacillus thuringiensis (WP_087951472.1), Pyrococcus furiosus (WP_011013013.1) as well as variants thereof and fragments thereof.

In some embodiments, the promoter-optized recombinant yeast cell does not include the one or more second genetic modifications described herein. In additional embodiments, the promoter-optized recombinant yeast cell can include the one or more second genetic modifications, the one or more third genetic modifications and/or the additional further genetic modifications described herein.

### Processes of Using the Recombinant Yeast Cell(s)

The processes described herein can be used for increasing ethanol yield. The processes described herein rely on the use of the recombinant yeast host cell described herein to increase ethanol yield. As indicated above, the recombinant yeast host cell of the present disclosure comprises one or more first genetic modification to increase ethanol yield (when compared to a parental yeast cell). In some embodiments, in processes of the present disclosure the ethanol yield obtained using the recombinant yeast host cell can be higher than the ethanol yield obtained using the parental yeast cell by at least 0.05, 0.06, 0.07, 0.08, 0.09, 0.1, 0.2, 0.3, 0.4, 0.5, 0.6, 0.7, 0.8, 0.9, 1.0, 2.0, 3.0, 4.0, 5.0 g/L or more (in comparable fermentation conditions). In some embodiments, the processes can include determining the ethanol yield obtained using the recombinant yeast cell and/or the parental yeast cell in fermentations conducted in comparable conditions. In some additional embodiments, the processes can include comparing the yield of ethanol obtained with the recombinant yeast cell with the yield in ethanol obtained with the parental yeast cell in fermentations conducted in comparable conditions. In some additional embodiments, the processes can include using a recombinant yeast cell which has been previously determined to exhibit an increase in ethanol yield with respect to the parental yeast cell and/or excluding a yeast cell which has been previously determined to exhibit an equal or a less ethanol yield with respect to the parental yeast cell.

The processes described herein can be used for improving at least one parameter of fermentation. The processes described herein rely on the use of the recombinant yeast host cell described herein to improve the at least one parameter of fermentation. In one embodiment, the at least one parameter of fermentation is fermentation kinetic. As used in the context of the present disclosure, the expression “fermentation kinetic” refers to the formation of biomass and ethanol during the growth phase of the fermentation. The growth phase refers to the period of time where cells are actively dividing and biomass concentrations are increasing (e.g., propagation). The ethanol production phase refers to a fermentation following a propagation. Fermentation kinetic can be assessed, for example, by determining specific growth rate, rate of ethanol accumulation, rate of glucose consumption and/or rate of CO₂ production. As indicated above, the recombinant yeast host cell of the present disclosure comprises the second genetic modification for increasing pyruvate decarboxylase activity providing it the ability to improve one or more fermentation parameters. In some embodiments, the processes can include determining the one or more fermentation parameters using the recombinant yeast cell, the intermediate yeast cell and/or the parental yeast cell during comparable fermentations. In some additional embodiments, the processes can include comparing the one or more fermentation parameters obtained with the recombinant yeast cell with the one or more fermentation parameters obtained with the intermediate yeast host and/or the parental yeast cell. In some additional embodiments, the processes can include using a recombinant yeast cell which has been previously determined to exhibit an improvement in at least one fermentation parameter with respect to the intermediate yeast cell and/or parental yeast cell and/or excluding a yeast cell which has been previously determined to lack an improvement in the one or more fermentation parameters with respect to the intermediate yeast cell and/or parental yeast cell. In an embodiment, the improvement in fermentation kinetic is observed in recombinant yeast cells having the second genetic modification as well as an inactivation in one or all copies of its native pdc genes.

The processes described herein can be used for decreasing glycerol production. The processes described herein rely on the use of the recombinant yeast host cell described herein to decrease glycerol production. In some embodiments, in the processes of the present disclosure, the amount of glycerol obtained using the recombinant yeast host cell can be lower than the amount of glycerol obtained using the parental yeast cell by at least 0.1, 0.2, 0.3, 0.4, 0.5, 0.6, 0.7, 0.8, 0.9, 1.0, 2.0, 3.0, 4.0, 5.0 g/L or more in comparable fermentations. In some embodiments, the processes can include determining the glycerol obtained using the recombinant yeast cell and/or the parental yeast cell in comparable fermentations. In some additional embodiments, the processes can include comparing the glycerol obtained with the recombinant yeast cell with the glycerol obtained with the parental yeast cell. In some additional embodiments, the processes can include using a recombinant yeast cell which has been previously determined to exhibit a decrease in glycerol production with respect to the parental yeast cell and/or excluding a yeast cell which has been previously determined to exhibit an equal or a higher glycerol production with respect to the parental yeast cell.

The processes described herein can be used for decreasing fusel alcohol production. The processes described herein rely on the use of the recombinant yeast host cell described herein to decrease fusel alcohol production. As used in the context of the present disclosure, the expression “fusel alcohol” refers the one or more higher alcohols (e.g., those with more than two carbons) which can be produced during the fermentation process by a yeast. Fusel alcohol include, without limitation, isoamyl alcohol, 2-methyl-1-butanol, isobutyl alcohol, 1-propanol, isopropanol, 1-butanol, 1-pentanol, 1-hexanol, 2-phenylethanol as well as mixtures thereof. Without being bound to theory, it is believed that fusel alcohols can be generated by the catabolism of amino acids. In some embodiments, in the processes of the present disclosure the amount of fusel alcohol obtained using the recombinant yeast host cell can be lower than the amount of fusel alcohol obtained using the parental yeast cell by at least 1% or more in comparable fermentations. In some embodiments, the processes can include determining the fusel alcohol obtained using the recombinant yeast cell and/or the parental yeast cell in comparable fermentations. In some additional embodiments, the processes can include comparing the fusel alcohol obtained with the recombinant yeast cell with the fusel alcohol obtained with the parental yeast cell. In some additional embodiments, the processes can include using a recombinant yeast cell which has been previously determined to exhibit a decrease in fusel alcohol production with respect to the parental yeast cell and/or excluding a yeast cell which has been previously determined to exhibit an equal or a higher fusel alcohol production with respect to the parental yeast cell.

The biomass that can be used in the processes to be converted to ethanol includes any type of biomass known in the art and described herein. For example, the biomass can include, but is not limited to, starch, sugar and lignocellulosic materials. Sugar material include, without limitation, cane and product derived from cane (cane juice or must for example). Starch materials can include, but are not limited to, mashes such as corn, wheat, rye, barley, rice, or milo. Sugar materials can include, but are not limited to, sugar beets, artichoke tubers, sweet sorghum, molasses or cane. The terms “lignocellulosic material”, “lignocellulosic substrate” and “cellulosic biomass” mean any type of substrate comprising cellulose, hemicellulose, lignin, or combinations thereof, such as but not limited to woody biomass, forage grasses, herbaceous energy crops, non-woody-plant biomass, agricultural wastes and/or agricultural residues, forestry residues and/or forestry wastes, paper-production sludge and/or waste paper sludge, waste-water-treatment sludge, municipal solid waste, corn fiber from wet and dry mill corn ethanol plants and sugar-processing residues. The terms “hemicellulosics”, “hemicellulosic portions” and “hemicellulosic fractions” mean the non-lignin, non-cellulose elements of lignocellulosic material, such as but not limited to hemicellulose (i.e., comprising xyloglucan, xylan, glucuronoxylan, arabinoxylan, mannan, glucomannan and galactoglucomannan), pectins (e.g., homogalacturonans, rhamnogalacturonan I and II, and xylogalacturonan) and proteoglycans (e.g., arabinogalactan-polypeptide, extensin, and pro line-rich polypeptides).

In a non-limiting example, the lignocellulosic material can include, but is not limited to, woody biomass, such as recycled wood pulp fiber, sawdust, hardwood, softwood, and combinations thereof; grasses, such as switch grass, cord grass, rye grass, reed canary grass, miscanthus, or a combination thereof; sugar-processing residues, such as but not limited to sugar cane bagasse; agricultural wastes, such as but not limited to rice straw, rice hulls, barley straw, corn cobs, cereal straw, wheat straw, canola straw, oat straw, oat hulls, and corn fiber; stover, such as but not limited to soybean stover, corn stover; succulents, such as but not limited to, agave; and forestry wastes, such as but not limited to, recycled wood pulp fiber, sawdust, hardwood (e.g., poplar, oak, maple, birch, willow), softwood, or any combination thereof. Lignocellulosic material may comprise one species of fiber; alternatively, lignocellulosic material may comprise a mixture of fibers that originate from different lignocellulosic materials. Other lignocellulosic materials are agricultural wastes, such as cereal straws, including wheat straw, barley straw, canola straw and oat straw; corn fiber; stovers, such as corn stover and soybean stover; grasses, such as switch grass, reed canary grass, cord grass, and miscanthus; or combinations thereof.

Substrates for cellulose activity assays can be divided into two categories, soluble and insoluble, based on their solubility in water. Soluble substrates include cellodextrins or derivatives, carboxymethyl cellulose (CMC), or hydroxyethyl cellulose (HEC). Insoluble substrates include crystalline cellulose, microcrystalline cellulose (Avicel), amorphous cellulose, such as phosphoric acid swollen cellulose (PASC), dyed or fluorescent cellulose, and pretreated lignocellulosic biomass. These substrates are generally highly ordered cellulosic material and thus only sparingly soluble.

It will be appreciated that suitable lignocellulosic material may be any feedstock that contains soluble and/or insoluble cellulose, where the insoluble cellulose may be in a crystalline or non-crystalline form. In various embodiments, the lignocellulosic biomass comprises, for example, wood, corn, corn stover, sawdust, bark, molasses, sugarcane, leaves, agricultural and forestry residues, grasses such as switchgrass, ruminant digestion products, municipal wastes, paper mill effluent, newspaper, cardboard or combinations thereof.

Paper sludge is also a viable biomass for lactate or acetate production. Paper sludge is solid residue arising from pulping and paper-making, and is typically removed from process wastewater in a primary clarifier. The cost of disposing of wet sludge is a significant incentive to convert the material for other uses, such as conversion to ethanol. Processes provided by the present disclosure are widely applicable. Moreover, the hydrolyzed biomass may be used to produce ethanol or higher value added chemicals, such as organic acids, aromatics, esters, acetone and polymer intermediates.

The biomass that can be used in the processes described herein is or comprise corn or a product derived from corn (also known as a corn derivative, which can be, for example, a corn mash (gelatinized or raw)). In some embodiments, the biomass includes starch, which can be raw, gelatinized or comprise a mixture or raw and gelatinized starch.

The process of the present disclosure comprise contacting the recombinant yeast cell of the present disclosure with the biomass so as to allow the hydrolysis of at least a part of the biomass and the conversion of the biomass (at least in part) into ethanol.

The fermentation process can be performed at temperatures of at least about 20° C., about 21° C., about 22° C., about 23° C., about 24° C., about 25° C., about 26° C., about 27° C., about 28° C., about 29° C., about 30° C., about 31° C., about 32° C., about 33°, about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C. In some embodiments, the fermentation process can be performed, at least in part, at high temperatures, for example at temperatures equal to or about 36° C., about 37° C., about 38° C., about 39° C., about 40° C. or higher.

In some embodiments, prior to fermentation, a step of liquefying starch can be included. The liquefaction of starch can be performed at a temperature of between about 70° C.-105° C. to allow for proper gelatinization and hydrolysis of the starch. In an embodiment, the liquefaction occurs at a temperature of at least about 70° C., 75° C., 80° C., 85° C., 90° C., 95° C., 100° C. or 105° C.

Alternatively or in combination, the liquefaction occurs at a temperate of no more than about 105° C., 100° C., 95° C., 90° C., 85° C., 80° C., 75° C. or 70° C. In yet another embodiment, the liquefaction occurs at a temperature between about 80° C. and 85° C. (which can include a thermal treatment spike at 105° C.).

The fermentation process can be include a batch fermentation, a continuous fermentation and/or the recycling of the recombinant yeast host cells during a plurality of fermentation cycles. For example, the recombinant yeast cell can be submitted to a plurality of fermentation cycles. In such embodiments, an initial fermenting population is inoculated in an fermentation medium which is then submitted to an initial fermentation. Once the initial fermentation has been completed (e.g., a fermentation product and a fermenting population have accumulated in the fermentation medium to provide a fermented fermentation medium), the resulting fermenting population is substantially isolated from the fermented fermentation medium. The isolating step can include, without limitation, centrifuging the fermented fermentation medium and/or acid washing the substantially isolated fermenting population. Once the initial fermentation cycle has been completed, the substantially isolated fermenting population is placed into contact (e.g., used to inoculate) a further fermentation medium and allowed to perform a further fermentation. Once the further fermentation has been completed (e.g., a fermentation product and a further fermenting population have accumulated in the further fermentation medium to provide a further fermented fermentation medium), the resulting fermenting population is substantially isolated from the fermented fermentation medium. The isolating step can include, without limitation, centrifuging the further fermented fermentation medium and/or acid washing the substantially isolated fermenting population. The substantially isolated fermenting population obtained can be submitted to yet a further fermentation cycle as described above. The plurality of fermentation cycles can include at least one continuous fermentation. The plurality of fermentation cycles can only include continuous fermentations. The plurality of fermentation cycles can include at least one batch fermentation. The plurality of fermentation cycles can only include batch fermentations. The processes of the present disclosure can include an initial fermentation cycle at least one, two, three, four, five, six, seven, eight, nine, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200 or more further fermentation cycles. In process comprising a plurality of fermentation cycles, the recombinant yeast cell is contacted with a substrate in at least one of the fermentation cycle (which can be an initial fermentation cycle or a further fermentation cycle). In additional embodiments, the recombinant yeast cell can ferment during one or more fermentation cycles.

In the recycling processes described herein, at the end of a fermentation cycle, the fermenting population is substantially isolated from the fermented fermentation medium. As used in the context of the present disclosure, the expression “substantially isolating” refers to the removal of the majority of the components of the fermented fermentation medium from the fermenting population. In some embodiments, “substantially isolating” refers to concentrating the fermenting population to at least 5, 10, 15, 20, 25, 30, 35, 45% or more when compared to the concentration of the fermenting population prior to the substantially isolation. In order to substantially isolate to fermenting population, the fermented fermentation medium can be centrifuged. Cell separation and recovery in the fuel ethanol process is carried out using stacked-disk, nozzle discharge type centrifuges, etc. In these machines, the feed-broth from the end of fermentation, often referred to in the process as “vinho bruto” or “beer” is introduced into the top of the machine, circulates to the bottom, and is then forced upward through a set of rotating disks. The rotation of these disks imparts a centrifugal force on the total feed, and particles. Yeast cells and other solids are forced downward and to the side of the machine. The cells then exit through nozzles at the outer edge of the machine creating a concentrated yeast cream. Clarified liquid, often called “vinho,”, “vinho delevurado” or “wine” exits the machine out the top.

Optionally the substantially isolated fermenting population can be washed. In a specific embodiment, the substantially isolated fermenting population can be submitted to an acid washing step. In the acid washing step, an acid or an acidic solution is put into contact with the fermenting population. In some embodiments, the acid or the acidic solution has a pH of between 2.0 and 2.2. In some embodiments, the contact between the substantially isolated fermenting population and the acid/acidic solution is maintained so as to reduce the contaminating bacterial population that may be present. For example, the contact between the substantially isolated fermenting population and the acid or the acidic solution can last at least 30, 40, 50, 60, 70, 80, 90, 100, 110, 120 minutes or more. In certain embodiments, the acid is sulphuric acid and/or the acidic solution comprises sulphuric acid. After the acid washing step, the pH of the acid washed fermenting population can be adjusted prior to the further fermentation cycle. In additional embodiments, the recombinant yeast cell can be recycled and even washed.

In some embodiments, the process can also include recuperating the fermentation product from the fermented fermentation medium or the further fermented fermentation medium. This can be used, for example, by distilling the fermented fermentation medium or the further fermented fermentation medium.

In some embodiments, the process can be used to produce ethanol at a particular rate. For example, in some embodiments, ethanol is produced at a rate of at least about 0.1 mg per hour per liter, at least about 0.25 mg per hour per liter, at least about 0.5 mg per hour per liter, at least about 0.75 mg per hour per liter, at least about 1.0 mg per hour per liter, at least about 2.0 mg per hour per liter, at least about 5.0 mg per hour per liter, at least about 10 mg per hour per liter, at least about 15 mg per hour per liter, at least about 20.0 mg per hour per liter, at least about 25 mg per hour per liter, at least about 30 mg per hour per liter, at least about 50 mg per hour per liter, at least about 100 mg per hour per liter, at least about 200 mg per hour per liter, at least about 300 mg per hour per liter, at least about 400 mg per hour per liter, at least about 500 mg per hour per liter, at least about 600 mg per hour per liter, at least about 700 mg per hour per liter, at least about 800 mg per hour per liter, at least about 900 mg per hour per liter, at least about 1 g per hour per liter, at least about 1.5 g per hour per liter, at least about 2 g per hour per liter, at least about 2.5 g per hour per liter, at least about 3 g per hour per liter, at least about 3.5 g per hour per liter, at least about 4 g per hour per liter, at least about 4.5 g per hour per liter, at least about 5 g per hour per liter, at least about 5.5 g per hour per liter, at least about 6 g per hour per liter, at least about 6.5 g per hour per liter, at least about 7 g per hour per liter, at least about 7.5 g per hour per liter, at least about 8 g per hour per liter, at least about 8.5 g per hour per liter, at least about 9 g per hour per liter, at least about 9.5 g per hour per liter, at least about 10 g per hour per liter, at least about 10.5 g per hour per liter, at least about 11 g per hour per liter, at least about 11.5 g per hour per liter, at least about 12 g per hour per liter, at least about 12.5 g per hour per liter, at least about 13 g per hour per liter, at least about 13.5 g per hour per liter, at least about 14 g per hour per liter, at least about 14.5 g per hour per liter or at least about 15 g per hour per liter.

During fermentation, the pH of the fermentation medium can be equal to or below 5.5, 5.4, 5.3, 5.2, 5.1, 5.0, 4.9, 4.8, 4.7., 4.6, 4.5, 4.4, 4.3, 4.2, 4.1, 4.0 or lower. In an embodiment, the pH of the fermentation medium (during fermentation) is between 4.0 and 5.5.

Ethanol production can be measured using any method known in the art. For example, the quantity of ethanol in fermentation samples can be assessed using HPLC analysis. Many ethanol assay kits are commercially available that use, for example, alcohol oxidase enzyme based assays.

In the process described herein, it is possible to add an exogenous source (e.g., to dose) of an enzyme to facilitate saccharification or improve fermentation yield. As such, the process can comprise including one or more dose of one or more exogenous enzyme during the saccharification and/or the fermentation step. The exogenous enzyme can be provided in a purified form or in combination with other enzymes (e.g., a cocktail). In the context of the present disclosure, the term “exogenous” refers to a characteristic of the enzyme, namely that it has not been produced during the saccharification or the fermentation step, but that it was produced prior to the saccharification or the fermentation step. The exogenous enzyme that can be used during the saccharification/fermentation process can include, without limitation, an alpha-amylase, a glucoamylase, a protease, a phytase, a pullulanase, a cellulase, a xylanase, a trehalase, or any combination thereof.

In some specific embodiments, it is possible to add a reduced amount of an exogenous source (e.g., to dose) of an enzyme when compared to a comparable (control) fermentation with the parental yeast cell. The amount of the exogenous enzyme is considered “reduced” with respect to amount of the exogenous enzyme used in the control fermentation because smaller doses or less doses are necessary. The amount of the exogenous enzyme used in the presence of the recombinant yeast cell allows achieving the same or a higher fermentation yield than the fermentation yield obtained with the control fermentation. In some specific embodiments, the recombinant yeast cell can reduce the amount of an exogenous protease needed to achieve at least the same fermentation yield as the control fermentation. In some specific embodiments, the recombinant yeast cell can reduce the amount of an exogenous glucoamylase needed to achieve at least the same fermentation yield as the control fermentation.

In the process described herein, it is possible to add a nitrogen source (usually urea or ammonia) to facilitate saccharification or improve fermentation yield. As such, the process can comprise including one or more amount of the nitrogen source prior to or during the saccharification and/or the fermentation step. In some embodiments, the process can comprise limiting the amount of the nitrogen source prior to or during the saccharification and/or the fermentation step. In other embodiments, the process can comprise omitting one or more amount of the nitrogen source prior to or during the saccharification and/or the fermentation step. The process of the present disclosure can be conducted, at least in part, under nitrogen scarcity conditions and, in further embodiments, without having detrimental consequences on the yield of ethanol, the fermentation parameter, the glycerol production and/or the fusel alcohol production.

For example, in an embodiment in which the nitrogen source is urea, the amount of the exogenous source of nitrogen required to complete the fermentation can be below 1000, 900, 800, 700, 600, 500, 400, 300, 200, 100, 90, 80, 70, 60, 50, 40, 30, 20, 10 ppm or less. In another embodiment in which the nitrogen source is urea, the amount of the exogenous source of nitrogen required to complete the fermentation can be between 10 and 100 ppm, 10 and 200 ppm, 10 and 300 ppm, 10 and 400 ppm, 10 and 500 ppm, 10 and 600 ppm, 10 and 700 ppm, 10 and 800 ppm, 10 and 900 ppm or 10 and 1000 ppm. In another embodiment in which the nitrogen source is urea, the amount of the exogenous source of nitrogen required to complete the fermentation can be between 20 and 100 ppm, 20 and 200 ppm, 20 and 300 ppm, 20 and 400 ppm, 20 and 500 ppm, 20 and 600 ppm, 20 and 700 ppm, 20 and 800 ppm, 20 and 900 ppm or 20 and 1000 ppm. In another embodiment in which the nitrogen source is urea, the amount of the exogenous source of nitrogen required to complete the fermentation can be between 30 and 100 ppm, 30 and 200 ppm, 30 and 300 ppm, 30 and 400 ppm, 30 and 500 ppm, 30 and 600 ppm, 30 and 700 ppm, 30 and 800 ppm, 30 and 900 ppm or 30 and 1000 ppm. In another embodiment in which the nitrogen source is urea, the amount of the exogenous source of nitrogen required to complete the fermentation can be between 40 and 100 ppm, 40 and 200 ppm, 40 and 300 ppm, 40 and 400 ppm, 40 and 500 ppm, 40 and 600 ppm, 40 and 700 ppm, 40 and 800 ppm, 40 and 900 ppm or 40 and 1000 ppm. In another embodiment in which the nitrogen source is urea, the amount of the exogenous source of nitrogen required to complete the fermentation can be between 50 and 100 ppm, 50 and 200 ppm, 50 and 300 ppm, 50 and 400 ppm, 50 and 500 ppm, 50 and 600 ppm, 50 and 700 ppm, 50 and 800 ppm, 50 and 900 ppm or 50 and 1000 ppm. In another embodiment in which the nitrogen source is urea, the amount of the exogenous source of nitrogen required to complete the fermentation can be between 60 and 100 ppm, 60 and 200 ppm, 60 and 300 ppm, 60 and 400 ppm, 60 and 500 ppm, 60 and 600 ppm, 60 and 700 ppm, 60 and 800 ppm, 60 and 900 ppm or 60 and 1000 ppm. In another embodiment in which the nitrogen source is urea, the amount of the exogenous source of nitrogen required to complete the fermentation can be between 70 and 100 ppm, 70 and 200 ppm, 70 and 300 ppm, 70 and 400 ppm, 70 and 500 ppm, 70 and 600 ppm, 70 and 700 ppm, 70 and 800 ppm, 70 and 900 ppm or 70 and 1000 ppm. In another embodiment in which the nitrogen source is urea, the amount of the exogenous source of nitrogen required to complete the fermentation can be between 80 and 100 ppm, 80 and 200 ppm, 80 and 300 ppm, 80 and 400 ppm, 80 and 500 ppm, 80 and 600 ppm, 80 and 700 ppm, 80 and 800 ppm, 80 and 900 ppm or 80 and 1000 ppm. In another embodiment in which the nitrogen source is urea, the amount of the exogenous source of nitrogen required to complete the fermentation can be between 90 and 100 ppm, 90 and 200 ppm, 90 and 300 ppm, 90 and 400 ppm, 90 and 500 ppm, 90 and 600 ppm, 90 and 700 ppm, 90 and 800 ppm, 90 and 900 ppm or 90 and 1000 ppm. In another embodiment in which the nitrogen source is urea, the amount of the exogenous source of nitrogen required to complete the fermentation can be between 100 and 200 ppm, 100 and 300 ppm, 100 and 400 ppm, 100 and 500 ppm, 100 and 600 ppm, 100 and 700 ppm, 100 and 800 ppm, 100 and 900 ppm or 100 and 1000 ppm. In another embodiment in which the nitrogen source is urea, the amount of the exogenous source of nitrogen required to complete the fermentation can be between 200 and 300 ppm, 200 and 400 ppm, 200 and 500 ppm, 200 and 600 ppm, 200 and 700 ppm, 200 and 800 ppm, 200 and 900 ppm or 200 and 1000 ppm. In another embodiment in which the nitrogen source is urea, the amount of the exogenous source of nitrogen required to complete the fermentation can be between 300 and 400 ppm, 300 and 500 ppm, 300 and 600 ppm, 300 and 700 ppm, 300 and 800 ppm, 300 and 900 ppm or 300 and 1000 ppm. In another embodiment in which the nitrogen source is urea, the amount of the exogenous source of nitrogen required to complete the fermentation can be between 400 and 500 ppm, 400 and 600 ppm, 400 and 700 ppm, 400 and 800 ppm, 400 and 900 ppm or 400 and 1000 ppm. In another embodiment in which the nitrogen source is urea, the amount of the exogenous source of nitrogen required to complete the fermentation can be between 500 and 600 ppm, 500 and 700 ppm, 500 and 800 ppm, 500 and 900 ppm or 500 and 1000 ppm. In another embodiment in which the nitrogen source is urea, the amount of the exogenous source of nitrogen required to complete the fermentation can be between 600 and 700 ppm, 600 and 800 ppm, 600 and 900 ppm or 600 and 1000 ppm. In another embodiment in which the nitrogen source is urea, the amount of the exogenous source of nitrogen required to complete the fermentation can be between 700 and 800 ppm, 700 and 900 ppm or 700 and 1000 ppm. In another embodiment in which the nitrogen source is urea, the amount of the exogenous source of nitrogen required to complete the fermentation can be between 800 and 900 ppm or 800 and 1000 ppm. In another embodiment in which the nitrogen source is urea, the amount of the exogenous source of nitrogen required to complete the fermentation can be between 900 and 1000 ppm. In another specific embodiment in which the nitrogen source is urea, the amount of the exogenous source of nitrogen required to complete the fermentation can be between 50 and 600 ppm. In another specific embodiment in which the nitrogen source is urea, the amount of the exogenous source of nitrogen required to complete the fermentation is equal to or below 600 ppm. The process can, in some embodiments, alleviate the need to supplement the hydrolyzed biomass with an exogenous source of nitrogen during the fermentation step.

The present invention will be more readily understood by referring to the following examples which are given to illustrate the invention rather than to limit its scope.

### Example I—Corn Mash Substrates

TABLE 1
Genotypes of the Saccharomyces cerevisiae
strains and isolates used in the example.
All the recombinant strains used directly or
indirectly strain M2390 as a parental strain.

| Desig- | Polypeptides | Native genes |
|---|---|---|
| nation | overexpressed | inactivated |
| M2390 | Not applicable, this is a wild-type train. | |
| M3744 | Glucoamylase (SEQ ID NO: 86) under the | fcy1 |
| | control of the tef2p | |
| M5301 | None | pdc1 |
| M5343 | None | pdc1 |
| M24914 | stl1 (SEQ ID NO: 8) under the control of the | stl1 |
| | adh1p | |
| M24032 | gapN (SEQ ID NO: 23) expressed under the | zwf1 |
| | control of the ZWF1p and the gpd1p | stl1 |
| | stl1 (SEQ ID NO: 8) expressed under the | |
| | control of the adh1p | |
| M26762 | pdc1 (SEQ ID NO: 11) expressed under the | None |
| | control of the adh1p | |
| M28045 | adhE (SEQ ID NO: 6) under the control of the | fdh1 |
| | gpd2p and the TPI1p | fdh2 |
| | pflA (SEQ ID NO: 2) under the control of the | stl1 |
| | adh1p | gpd2 |
| | pflB (SEQ ID NO: 4) under the control of the | ylr296w |
| | ENO1p | |
| | stl1 (SEQ ID NO: 8) under the control of the | |
| | TEF2p and the adh1p | |
| | pdc1 (SEQ ID NO: 11) expressed under the | |
| | control of the adh1p | |
| M28047 | gapN (SEQ ID NO: 23) expressed under the | zwf1 |
| | control of the ZWF1p and the gpd1p | stl1 |
| | stl1 (SEQ ID NO: 8) expressed under the | ylr296w |
| | control of the adh1p | |
| | pdc1 (SEQ ID NO: 11) expressed under the | |
| | control of the adh1p | |
| M28049 | adhE (SEQ ID NO: 6) under the control of the | fdh1 |
| | gpd2p and the TPI1p | fdh2 |
| | pflA (SEQ ID NO: 2) under the control of the | stl1 |
| | adh1p | gpd2 |
| | pflB (SEQ ID NO: 4) under the control of the | ylr296w |
| | ENO1p | |
| | stl1 (SEQ ID NO: 8) under the control of the | |
| | TEF2p and the adh1p | |
| | adhB (SEQ ID NO: 21) expressed under | |
| | control of the adh2p | |
| M28054 | gapN (SEQ ID NO: 23) expressed under the | zwf1 |
| | control of the ZWF1p and the gpd1p | stl1 |
| | stl1 (SEQ ID NO: 8) expressed under the | ylr296w |
| | control of the adh1p | |
| | adhB (SEQ ID NO: 21) expressed under the | |
| | control of the adh2p | |
| M28093 | adhE (SEQ ID NO: 6) under the control of the | fdh1 |
| | gpd2p and the TPI1p | fdh2 |
| | pflA (SEQ ID NO: 2) under the control of the | stl1 |
| | adh1p | gpd2 |
| | pflB (SEQ ID NO: 4) under the control of the | ylr296w |
| | ENO1p | |
| | stl1 (SEQ ID NO: 8) under the control of the | |
| | TEF2p and the adh1p | |
| | pdc1 (SEQ ID NO: 11) under the control of the | |
| | adh1p | |
| | adhB (SEQ ID NO: 21) expressed under the | |
| | control of the adh2p | |
| M28095 | gapN (SEQ ID NO: 23) expressed under the | zwf1 |
| | control of the ZWF1p and the gpd1p | stl1 |
| | stl1 expressed under the control of the adh1p | ylr296w |
| | adhB (SEQ ID NO: 21) expressed under the | |
| | control of the adh2p | |
| | pdc1 (SEQ ID NO: 11) expressed under the | |
| | control of the adh1p | |
| M28357 | pdc1 (SEQ ID NO: 11) under the control of the | pdc1 |
| | native pdc1p | |
| M28898 | stl1 (SEQ ID NO: 8) under the control of the | stl1 |
| | adh1p | pdc1 |
| | pdc1 (SEQ ID NO: 11) under the control of the | |
| | native pdc1p | |
| M29213 | pdc1 (SEQ ID NO: 69) under control of the | pdc1 |
| M29214 | native pdc1p | |
| T13869-1 | pdc1 (SEQ ID NO: 12) under the control of the | fur1 |
| | adh1p | |
| T13870-2 | pdc1 (SEQ ID NO: 69) under control of the | fur1 |
| | adh1p | |
| T13871-1 | stl1 (SEQ ID NO: 87) under the control of the | fur1 |
| | tef2p | |
| T13872-1 | stl1 (SEQ ID NO: 88) under the control of the | fur1 |
| | tef2p | |
| T13873-1 | stl1 (SEQ ID NO: 8) under the control of the | fur1 |
| | tef2p | |
| T13874-2 | stl1 (SEQ ID NO: 87) under the control of the | fur1 |
| | tef2p | |
| | pdc1 (SEQ ID NO: 12) under the control of the | |
| | adh1p | |
| T13875-1 | stl1 (SEQ ID NO: 88) under the control of the | fur1 |
| | tef2p | |
| | pdc1 (SEQ ID NO: 12) under the control of the | |
| | adh1p | |
| T13876-1 | stl1 (SEQ ID NO: 8) under the control of the | fur1 |
| | tef2p | |
| | pdc1 (SEQ ID NO: 12) under the control of the | |
| | adh1p | |
| T13877-1 | stl1 (SEQ ID NO: 87) under the control of the | fur1 |
| | tef2p | |
| | pdc1 (SEQ ID NO: 69) under control of the | |
| | adh1p | |
| T13878-2 | stl1 (SEQ ID NO: 88) under the control of the | fur1 |
| | tef2p | |
| | pdc1 (SEQ ID NO: 69) under control of the | |
| | adh1p | |
| T13879-1 | stl1 (SEQ ID NO: 8) under the control of the | fur1 |
| | tef2p | |
| | pdc1 (SEQ ID NO: 69) under control of the | |
| | adh1p | |
| T13880-1 | pdc1 (SEQ ID NO: 12) under the control of the | fur1 |
| | adh1p | fcy1 |
| | Glucoamylase (SEQ ID NO: 86) under the | |
| | control of the tef2p | |
| T13881-1 | pdc1 (SEQ ID NO: 69) under control of the | fur1 |
| | adh1p | fcy1 |
| | Glucoamylase (SEQ ID NO: 86) under the | |
| | control of the tef2p | |
| T13882-2 | stl1 (SEQ ID NO: 87) under the control of the | fur1 |
| | tef2p | fcy1 |
| | Glucoamylase (SEQ ID NO: 86) under the | |
| | control of the tef2p | |
| T13883-1 | stl1 (SEQ ID NO: 88) under the control of the | fur1 |
| | tef2p | fcy1 |
| | Glucoamylase (SEQ ID NO: 86) under the | |
| | control of the tef2p | |
| T13884-2 | stl1 (SEQ ID NO: 8) under the control of the | fur1 |
| | tef2p | fcy1 |
| | Glucoamylase (SEQ ID NO: 86) under the | |
| | control of the tef2p | |
| T13885-2 | stl1 (SEQ ID NO: 87) under the control of the | fur1 |
| | tef2p | fcy1 |
| | pdc1 (SEQ ID NO: 12) under the control of the | |
| | adh1p | |
| | Glucoamylase (SEQ ID NO: 86) under the | |
| | control of the tef2p | |
| T13886-1 | stl1 (SEQ ID NO: 88) under the control of the | fur1 |
| | tef2p | fcy1 |
| | pdc1 (SEQ ID NO: 12) under the control of the | |
| | adh1p | |
| | Glucoamylase (SEQ ID NO: 86) under the | |
| | control of the tef2p | |
| T13887-1 | stl1 (SEQ ID NO: 8) under the control of the | fur1 |
| | tef2p | fcy1 |
| | pdc1 (SEQ ID NO: 12) under the control of the | |
| | adh1p | |
| | Glucoamylase (SEQ ID NO: 86) under the | |
| | control of the tef2p | |
| T13888-1 | stl1 (SEQ ID NO: 87) under the control of the | fur1 |
| | tef2p | fcy1 |
| | pdc1 (SEQ ID NO: 69) under control of the | |
| | adh1p | |
| | Glucoamylase (SEQ ID NO: 86) under the | |
| | control of the tef2p | |
| T13889-1 | stl1 (SEQ ID NO: 88) under the control of the | fur1 |
| | tef2p | fcy1 |
| | pdc1 (SEQ ID NO: 69) under control of the | |
| | adh1p | |
| | Glucoamylase (SEQ ID NO: 86) under the | |
| | control of the tef2p | |
| T13890-1 | stl1 (SEQ ID NO: 8) under the control of the | fur1 |
| | tef2p | fcy1 |
| | pdc1 (SEQ ID NO: 69) under control of the | |
| | adh1p | |
| | Glucoamylase (SEQ ID NO: 86) under the | |
| | control of the tef2p | |

Effect of pdc1 knock-out, knock-in and heterologous expression on fermentation kinetic. Yeast strains M2390 (wild-type), M28357 (knock-out for native pdc1 and heterologous expression of pdc1), M26762 (knock-out for native pdc1 and heterologous expression of pdc1) and M5343 (knock-out for native pdc1) were cultivated in YPD 40 g/L glucose medium overnight prior to inoculation. Fermentations were conducted with 0.06 g/L of dry cell weight in 31.1% total solids liquefied corn mash containing 600 ppm urea and 0.69 AGU/gTS of exogenous glucoamylase (100% dose). Fermentations were incubated in a minivial (3 g) at 33.3° C. for 18 h, followed by 31.1° C. until 54 h. The results are shown in Table 2.

TABLE 2
Fermentation yield (in g/L) obtained with strains M2390,
M26762, M28357 and M5343. All results are provided at
drop, except for the metabolite “ethanol at 22 h”.

| | M2390 | M26762 | M28357 | M5343 |
|---|---|---|---|---|
| Pyruvate | 0.5 | 0.0 | 0.0 | 1.0 |
| Ethanol at 22 h | 101.2 | 104.9 | 105.9 | 96.7 |
| Ethanol | 134.1 | 134.4 | 135.6 | 131.8 |
| YP - glycerol | 8.2 | 7.5 | 6.9 | 8.5 |
| Glucose | 0.5 | 0.7 | 0.8 | 0.7 |
| Acetate | 0.8 | 0.9 | 0.9 | 0.7 |

The knockout pdc1 strain M5343 exhibited a slower kinetic than parental strain M2390 (Table 2). Strain M5343 was able to complete the fermentation, probably due to compensation from other Saccharomyces cerevisiae PDC enzymes. Strains M26762 and M28357 expressing heterologous pdc1 in place of the native pdc1 further improved kinetics relative to M2390 (Table 2). In addition, in strain M28357, an enhanced yield (1-1.5 g/L), less YP-glycerol (1 g/L), and slightly higher acetate is observed when compared to the wild-type strain M2390 (Table 2).

Yeast strains M2390, M26762 and M28357 were cultivated in YPD 40 g/L glucose medium overnight prior to inoculation. Fermentation was conducted with 0.06 g/L of dry cell weight in 33.3% total solids of a liquefied corn mash containing 518 ppm urea, exogenous glucoamylase and exogenous protease. Fermentations were incubated at 33.3° C. for the first 20 h, 32.2° C. (20 h-32 h), 31.6° C. (32 h-45 h) and 31.1° C. (45 h-72 h) in 125 mL bottles (25 g). The results are shown in Table 3.

TABLE 3
Fermentation yield (in g/L) obtained with strains M2390, M26762
and M28357. All results are provided at drop, except for the
metabolites “ethanol at 24 h” and “ethanol at 48 h”.

| | M2390 | M26762 | M28357 |
|---|---|---|---|
| Ethanol at 24 h | 114.8 | 119.3 | 120.6 |
| Ethanol at 48 h | 147.9 | 149.0 | 150.1 |
| Ethanol | 148.8 | 149.5 | 149.1 |
| Glucose | 0.5 | 0.4 | 0.5 |
| YP - glycerol | 8.9 | 8.5 | 7.9 |
| Pyruvate | 0.2 | 0.1 | 0.1 |

As shown in Table 3, both strains expressing the heterologous Zymonas mobilis pyruvate decarboxylase (M26762 and M28357) exhibited faster kinetics and improved yields when compared to the control strain M2390.

Effect of stl1, gapN, adhB, pflA, pflB and pdc1 expression on fermentation kinetic, yield and glycerol reduction. Yeast strains M2390, M28357 (knock-out for native pdc1 and heterologous expression of pdc1), M24914 (knock-out for native stl1 and heterologous expression of stl1) and M28898 (knock-out for natives pdc1 and stl1 and heterologous expression of pdc1 and stl1) were cultivated in in YPD 40 g/L glucose medium overnight prior to inoculation. Fermentations were conducted with 0.06 g/L of dry cell weight in 31.3% total solids of a liquefied corn mash containing 0 (no urea) or 459 ppm urea (plus urea), glucoamylase (100% dose). Fermentations were incubated at 33.3° C. for the first 18 h and 31.1° C. (18 h-54 h) in minivials (3 g). The results are shown in Table 4.

TABLE 4
Percentage in change in fermentation yield and glycerol reduction obtained
with strains M28357, M24914 and M28898 when compared to M2390.

| | | Yield | Glycerol Reduction |
|---|---|---|---|
| no urea | M28357 | 5.8% | −23.0% |
| no urea | M24914 | −0.7% | −8.6% |
| no urea | M28898 | 5.5% | −32.8% |
| plus urea | M28357 | 1.6% | −15.2% |
| plus urea | M24914 | 0.3% | −9.6% |
| plus urea | M28898 | 2.8% | −30.0% |

It was determined the effects of expressing stl1 and/or pdc1 had an effect on yield and glycerol reduction, especially in the absence of urea. Strain M28357 exhibited an improved yield and glycerol reduction when compared to strain M2390 (Table 4). Strain M28898 exhibited an improved yield and glycerol reduction when compared to strains M2390 and M24914 (Table 4).

Yeast strains M2390, M24032 (expressing both heterologous gapN and stl1) and M28047 (expressing heterologous gapN, stl1 and pdc1) were cultivated in YPD 40 g/L glucose medium overnight prior to inoculation. Fermentations were conducted with 0.06 g/L of dry cell weight in 33.3% total solids corn mash containing 518 ppm urea, 1.35×10⁻⁴ v/v of exogenous glucoamylase (100% dose) and 7.21×10⁻⁶ v/v of exogenous protease. Fermentations were incubated at 33.3° C. for the first 20 h, 32.2° C. (20 h-32 h), 31.6° C. (32 h-45 h) and 31.1° C. (45 h-72 h) in 125 mL bottles (25 g). The results are shown in Table 5.

TABLE 5
Fermentation yield (in g/L) obtained with
strains M2390, M24032 and M28047. All results
are provided at drop, except for the metabolites
“ethanol at 24 h” and “ethanol at 48 h”.

| | M2390 | M24032 | M28047 |
|---|---|---|---|
| Ethanol at 24 h | 114.8 | 108.3 | 112.7 |
| Ethanol at 48 h | 147.9 | 150.5 | 151.5 |
| Ethanol | 148.8 | 150.8 | 152.0 |
| Glucose | 0.5 | 0.3 | 0.3 |
| YP - glycerol | 8.9 | 4.2 | 3.9 |

As shown in Table 5, at 24 h into the fermentation, strain M24032 exhibited a lower ethanol yield than control strain M2390. The expression of pdc1 in strain M28407 (corresponding to strain M24032 in which pdc1 is overexpressed) increased the fermentation kinetic, as determined by the ethanol yield at 24 h.

Yeast strains M2390, M24032 (expressing both heterologous gapN and stl1), M28047 (expressing heterologous gapN, stl1 and pdc1), M28054 (expressing heterologous gapN, stl1 and adhB) and M28095 (expressing heterologous gapN, stl1, adhB and pdc1) were cultivated in YPD 40 g/L glucose medium overnight prior to inoculation. Fermentations were conducted with 0.06 g/L of dry cell weight in 34.7% total solids of a liquefied corn mash containing 165 ppm urea and exogenous glucoamylase (100% dose corresponding to 0.69 AGU/gTS). Fermentations were incubated at 33.8° C. for 48 h in minivials (3 g). The results are shown in Table 6.

TABLE 6
Fermentation yield (in g/L) obtained with strains M2390, M28032, M28047, M28054
and M28095. All results are provided at drop, except for the metabolite “ethanol at 22 h”.

| | M2390 | M24032 | M28047 | M28054 | M28095 |
|---|---|---|---|---|---|
| Ethanol at 22 h | 104.0 | 94.6 | 101.1 | 98.7 | 102.1 |
| Ethanol | 135.0 | 140.0 | 141.0 | 139.9 | 140.8 |
| Glucose | 2.5 | 1.8 | 1.6 | 1.9 | 1.8 |
| YP - glycerol | 10.4 | 4.9 | 4.7 | 4.9 | 4.6 |
| Acetate | 12 | 0.9 | 1.1 | 0.9 | 1.1 |

The expression of gapN, stl1 and adhB in strain M28054 reduced its fermentation kinetic when compared to control strain M2390 (Table 6). The expression of pdc1 in this genetic background (in strain M28095) increased ethanol titer at 22 h (when compared to strain M28054) and achieved a higher yield at drop (when compared to control strain M2390).

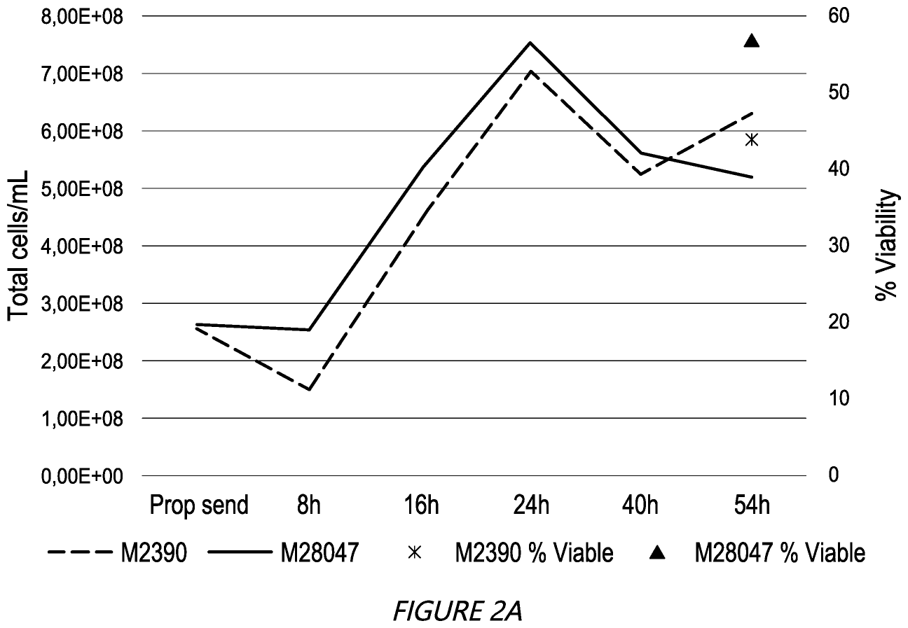

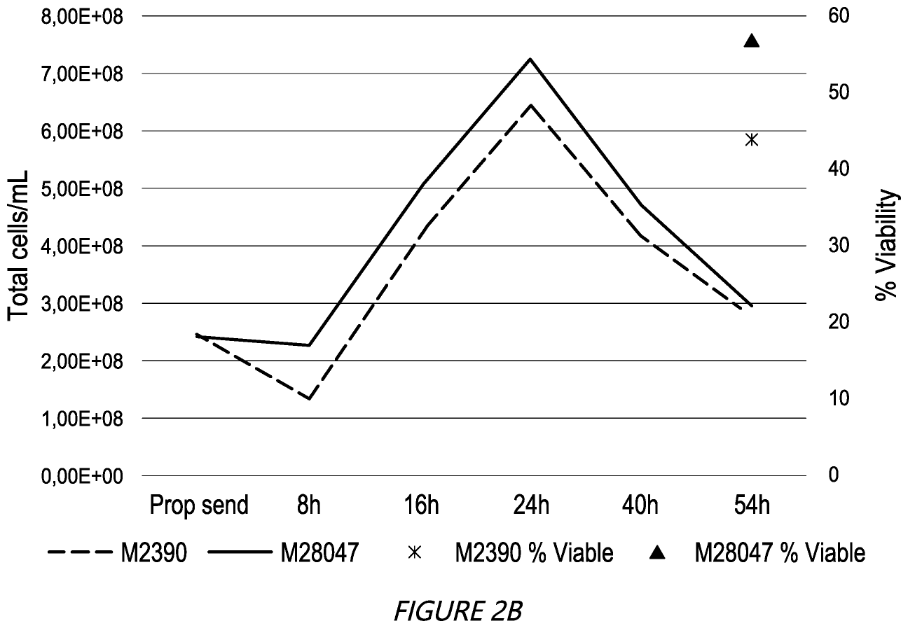

Effect of stl1, gapN, and pdc1 expression on biomass, fermentation kinetic, yield and fusel alcohol production. The specific growth and fusel alcohol production of yeast strains M2390 and M28047 (expressing heterologous gapN, stl1 and pdc1) were also determined during fermentation. More specifically, the strains were propagated prior to inoculation. Fermentation were conducted with 2.36% v/v from propagation in 31.1% total solids of a liquefied corn mash containing 527 ppm urea and exogenous glucoamylase (100% dose). Fermentations were incubated at 32.2° C. for 54 h in 2 L reactors. The results are shown in FIG. 2 and Table 7.

TABLE 7
Production of fusel alcohol (in mg/L) during fermentations with yeast strains M2390
and M28047. 1-prop: 1-propanol, isobut: isobutyl alcohol, 2-methyl-1 propanol, act-amyl: 2-
methyl-1-butanol, isoamyl : isoamyl alcohol, 3-methyl-1-butanol, isopentanol.

| Yeast strain | Time | 1-prop | isobut | act-amyl | isoamyl |
|---|---|---|---|---|---|
| M2390 | 40 h | 19 | 40 | 41 | 128 |
| M28047 | 40 h | 23 | 14 | 9 | 44 |
| M2390 | 54 h | 23 | 45 | 30 | 157 |
| M28047 | 54 h | 26 | 15 | 11 | 48 |

Yeast strain M28047 exhibited a higher total cell count (FIG. 2A) as well as a higher living cell count (FIG. 2B) throughout the fermentation when compared to yeast strain M2390.

Yeast strains M2390, M28045, M28049 and M28093 were cultivated in YPD 40 g/L glucose medium overnight prior to inoculation. Fermentations were conducted with 0.06 g/L of dry cell weight in 34.7% total solids of a liquefied corn mash containing 165 ppm urea and exogenous glucoamylase (100% dose corresponding to 0.69 AGU/gTS). Fermentations were incubated at 33.8° C. for 48 h in minivials (3 g). The results are shown in Table 8.

TABLE 8
Fermentation yield (in g/L) obtained with strains M2390, M28045, M28049 and
M28093. All results are provided at drop, except for the metabolite “ethanol at 22 h”.

| | M2390 | M28045 | M28049 | M28093 |
|---|---|---|---|---|
| Ethanol at 22 h | 104.0 | 94.3 | 93.2 | 91.5 |
| Ethanol | 135.0 | 141.1 | 139.6 | 141.6 |
| Glucose | 2.5 | 0.8 | 2.0 | 0.5 |
| YP - glycerol | 10.4 | 4.4 | 3.3 | 4.1 |
| Acetate | 1.2 | 0.8 | 0.4 | 0.7 |
| Formate | 0.0 | 0.0 | 0.2 | 0.1 |

Yeast strains M2390 (wild type), M24914 (expressing a heterologous stl1 polypeptide), M24032 (expressing heterologous stl1 and gapN polypeptides), M28357 (expressing a heterologous pdc1 polypeptide), M28898 (expressing heterologous stl1 and pdc1 polypeptides) and M28047 (expressing heterologous stl1, gapN and pdc1 polypeptides) were cultivated in YPD 40 g/L glucose medium overnight prior to inoculation. Fermentations were conducted with 0.06 g of dry cell weight/L in 32.2% total solids of a liquefied corn mash containing 236 ppm urea, and exogenous glucoamylase. Fermentations were incubated at 33.9° C. for 25 h, followed by 31.1° C. for the remainder of the fermentation in 25 g serum bottles attached to a CO₂ pressure monitoring system. Metabolites were determined by HPLC, except for acetaldehyde which was determined using GS-FIP. The data is provided on FIG. 3 and Table 9.

TABLE 9
Acetaldehyde (in g/L) obtained with strains
M2390, M28357, M24914, M28898,
M24032, and M28047 at 18 h or 64 h.

| Strain | 18 h | 64 h |
|---|---|---|
| M2390 | 0.08 | 0.15 |
| M28357 | 0.10 | 0.11 |
| M24914 | 0.03 | 0.13 |
| M28898 | 0.05 | 0.09 |
| M24032 | 0.05 | 0.14 |
| M28047 | 0.06 | 0.07 |

Strains expressing the heterologous stl1 polypeptides, without co-expressing the heterologous pdc1 polypeptide, exhibited a lower ethanol peak than the parental yeast strain M2390 (FIGS. 3A to 3D). Strains expressing the heterologous pdc1 polypeptide exhibited an increase in the ethanol peak and finished fermentation faster when compared to the parental yeast strain M2390 (FIGS. 3A to 3D). In addition, strains expressing the heterologous pdc1 polypeptide accumulated less acetaldehyde than the parent yeast strain M2390 (Table 9).

Effect of various pdc1 heterologous expression on fermentation kinetic. Yeast strains M2390, M28357, M29213, M29214, and M5301 were cultivated in YPD 40 g/L glucose medium overnight prior to inoculation. Fermentations were conducted with 0.06 g of dry cell weight/L in 33.3% total solids of a liquefied corn mash containing 518 ppm urea, exogenous glucoamylase and exogenous protease. Fermentations were incubated at 33.3° C. for the first 20 h, 32.2° C. (20 h-32 h), 31.6° C. (32 h-45 h) and 31.1° C. (45 h-72 h) in minivials (3 g). The results are provided in Table 10.

TABLE 10
Fermentation yield (in g/L) obtained with strains M2390, M28357, M29213, M29214.
and M5301. All results are provided at drop, except for the metabolite “ethanol at 24 h”.

| Strain | Ethanol at 24 h | Ethanol at 72 h | Glucose | Glycerol | Pyruvate |
|---|---|---|---|---|---|
| M2390 | 111.6 | 148.7 | 0.7 | 8.9 | 0.1 |
| M28357 | 120.0 | 150.5 | 0.4 | 7.7 | 0.0 |
| M29213 | 121.5 | 148.1 | 0.7 | 8.6 | 0.0 |
| M29214 | 121.0 | 149.2 | 0.7 | 8.6 | 0.0 |
| M5301 | 111.0 | 146.4 | 1.3 | 8.8 | 1.0 |

Effect of stl1, glucoamylase and pdc1 expression on yield and glycerol reduction. Yeast strains M2390 and M3744 as well as yeast isolates T13869-1, T13870-2, T13871-1, T13872-1, T13873-1, T13874-2, T13875-1, T13876-1, T13877-1, T13878-2, T13879-1, T13880-1, T13881-1, T13882-2, T13883-1, T13884-2, T13885-2, T13886-1, T13887-1, T13888-1, T13889-1 and T13890-1 were cultivated in YPD 40 g/L glucose medium overnight prior to inoculation. Fermentations were conducted with 0.06 g of dry cell weight/L in 31.8% total solids of a liquefied corn mash containing 236 ppm urea, and exogenous glucoamylase (0.65 AGU/gTS enzyme dose=100% for yeasts strain and isolates not expressing a glucoamylase; 80% dose for yeast isolates expressing a glucoamylase). Fermentations were incubated at 33.9° C. for the first 25 h, and 33.1° C. for the remaining of the fermentation (25 h-64 h) in minivials (4 g). The results are provided in Table 11.

TABLE 11
Fermentation yield (in g/L) obtained with yeast strain M2390 as well
as yeast isolates (to be completed). All results are provided at drop.

| Strain or isolate | Glucose | YP-Glycerol | Acetate | Ethanol |
|---|---|---|---|---|
| M2390 | 1.7 | 10.0 | 1.6 | 143.3 |
| T13869-1 | 1.4 | 10.2 | 1.8 | 142.2 |
| T13870-2 | 1.7 | 10.4 | 1.7 | 141.6 |
| T13871-1 | 1.3 | 7.3 | 1.3 | 143.8 |
| T13872-1 | 1.4 | 8.4 | 1.5 | 143.3 |
| T13873-1 | 0.9 | 8.0 | 1.2 | 143.3 |
| T13874-2 | 0.9 | 6.3 | 1.4 | 145.9 |
| T13875-1 | 1.0 | 7.2 | 1.4 | 146.1 |
| T13876-1 | 1.1 | 6.8 | 1.4 | 144.9 |
| T13877-1 | 0.9 | 6.7 | 1.3 | 145.2 |
| T13878-2 | 1.0 | 7.6 | 1.5 | 143.9 |
| T13879-1 | 1.1 | 7.2 | 1.4 | 144.8 |
| M3744 | 0.7 | 8.2 | 1.4 | 144.2 |
| T13880-1 | 0.7 | 8.0 | 1.3 | 146.7 |
| T13881-1 | 0.6 | 8.3 | 1.4 | 144.5 |
| T13882-2 | 0.7 | 6.8 | 1.2 | 144.0 |
| T13883-1 | 0.8 | 7.3 | 1.3 | 144.0 |
| T13885-2 | 0.9 | 6.4 | 1.4 | 145.8 |
| T13886-1 | 0.9 | 7.3 | 1.5 | 145.0 |
| T13888-1 | 1.0 | 6.7 | 1.3 | 144.6 |
| T13889-1 | 1.0 | 7.9 | 1.5 | 144.0 |

### Example II—Sugar Cane Substrates

TABLE 12
Genotypes of the Saccharomyces cerevisiae strains used
in the example. All the recombinant strains used directly
or indirectly have strain M17328 as a parental strain.

| | | |
|---|---|---|
| | | Native genes |
| Designation | Polypeptides overexpressed | inactivated |

Strains derived from M17328 - a wild-type train

| | | |
|---|---|---|
| M18447 | stl1 (SEQ ID NO: 8) encoded by | fcy1 |
| | the nucleic acid molecule having | ime1 |
| | the sequence of SEQ ID NO: 71 | |
| M27892 | gpd2 (SEQ ID NO: 90) encloded by | gpd1 |
| | the nucleic acid molecule having | |
| | the sequence of SEQ ID NO: 91 | |
| | stl1 (SEQ ID NO: 8) encoded by | |
| | encoded by the nucleic acid molecule | |
| | having the sequence of SEQ ID NO: 71 | |
| M30719 | pdc1 (SEQ ID NO: 12) encoded by | pdc1 |
| | the nucleic acid molecule having | |
| | the sequence of SEQ ID NO: 70 | |
| M32292 | stl1 (SEQ ID NO: 8) encoded by | fcy1 |
| | the nucleic acid molecule having | ime1 |
| | the sequence of SEQ ID NO: 71 | pdc1 |
| | pdc1 (SEQ ID NO: 12) encoded | |
| | by the nucleic acid molecule | |
| | having the sequence of SEQ ID NO: 70 | |
| M30743 | gpd2 (SEQ ID NO: 90) encloded by | gpd1 |
| | the nucleic acid molecule having | pdc1 |
| | the sequence of SEQ ID NO: 91 | |
| | stl1 (SEQ ID NO: 8) encoded by | |
| | the nucleic acid molecule having | |
| | the sequence of SEQ ID NO: 71 | |
| | pdc1 (SEQ ID NO: 12) encoded by | |
| | the nucleic acid molecule having the | |
| | sequence of SEQ ID NO: 70 | |

Strains derived from M2390 - a wild-type train

| | | |
|---|---|---|
| T13869-1 | pdc1 (SEQ ID NO: 12) under | fur1 |
| | the control of the adh1p | |
| T13870-1 | pdc1 (SEQ ID NO: 69) under | fur1 |
| | control of the adh1p | |
| T13872-1 | stl1 (SEQ ID NO: 88) under | fur1 |
| | the control of the tef2p | |
| T13875-1 | stl1 (SEQ ID NO: 88) under | fur1 |
| | the control of the tef2p | |
| | pdc1 (SEQ ID NO: 12) under | |
| | the control of the adh1p | |
| T13878-2 | stl1 (SEQ ID NO: 88) under | fur1 |
| | the control of the tef2p | |
| | pdc1 (SEQ ID NO: 69) under | |
| | control of the adh1p | |
| T13871-1 | stl1 (SEQ ID NO: 87) under | fur1 |
| | the control of the tef2p | |
| T13874-2 | stl1 (SEQ ID NO: 87) under | fur1 |
| | the control of the tef2p | |
| | pdc1 (SEQ ID NO: 12) under | |
| | the control of the adh1p | |
| T13877-1 | stl1 (SEQ ID NO: 87) under | fur1 |
| | the control of the tef2p | |
| | pdc1 (SEQ ID NO: 69) under | |
| | control of the adh1p | |
| T13873-1 | stl1 (SEQ ID NO: 8) under | fur1 |
| | the control of the tef2p | |
| T13876-1 | stl1 (SEQ ID NO: 8) under | fur1 |
| | the control of the tef2p | |
| | pdc1 (SEQ ID NO: 12) under | |
| | the control of the adh1p | |
| T13879-1 | stl1 (SEQ ID NO: 8) under | fur1 |
| | the control of the tef2p | |
| | pdc1 (SEQ ID NO: 69) under | |
| | control of the adh1p | |

Strains were propagated aerobically in 5 mL of YP 40 g/L glucose overnight, washed with water and resuspended in 1 mL of sterile water. Forty microliters of the washed strain was inoculated into 40 mL of commercial sugarcane substrate sourced from Brazilian sugarcane ethanol mills. The fermentation was incubated at 3300 with shaking at 150 rpm with pressure monitoring to determine when the fermentation was complete. The fermentation was considered to be completed once it was recorded that no more 002 is being produced as determined by a mass flow meter. Samples were taken for HPLC at the end of the fermentation to determine the ethanol and glycerol production in fermentation.

The performances of strains M17328 (wild-type), M 0.8447 (expressing stl1 only), M30719 (expressing heterologous pdc only) and M32292 (expressing both −t and heterologous pdc1) on a single fermentation cycle on a Brazilian must was compared. The results are provided in Table 12.

TABLE 12
Ethanol, glycerol and fermentation time are
expressed as the % change versus the
control yeast M17328 in order to
compare between fermentation substrates.

| Strain | Ethanol | Glycerol | Fermentation time |
|---|---|---|---|
| M18447 | 1.4% | −10.3% | 1.6% |
| M30719 | 2.3% | −12.9% | −5.4% |
| M32292 | 3.9% | −23.1% | −3.9% |

Strain M18447 achieved a higher ethanol and a lower glycerol yield but fermented more slowly, when compared to the control yeast strain M17328. Strains M30719 and M32292 (both expressing a heterologous pdc1) achieved a higher ethanol and a lower glycerol yield and fermented more quickly, when compared to the control yeast strain M17328.

The performance of control strain M17328 and engineered strains M18447, M30719 and M32292 were monitored in a fed-batch high cell density (10% v/v yeast) fermentation with pH 2 acid treatment for 16 cycles of fermentation. Each cycle started with a 1 hour acid treatment of the cells from the previous cycle. The fermentations were then fed commercially sourced sugarcane must from Brazilian mills for 4 hours at 33° C. and 150 rpm shaking. Fermentations were monitored by off-gas analysis to determine when strains had completed fermentation. At the start and end of each cycle, HPLC and GC-FID were completed on samples. A mass balance was completed on each cycle to determine the amount of metabolite produced in each cycle per gram of sugar fed. The percent change relative to the control strain M17328 was determined for each metabolite over each cycle. The average percent difference from M17328 over all cycles is shown in Table 13.

TABLE 13
Ethanol (g/g), glycerol (g/g), fermentation time (h),
pyruvate (g/g), iso-butanol (g/g), amyl-alcohol (g/g), and
iso-amyl-alcohol (g/g) are expressed as the % change versus the control
yeast M17328 in order to compare between fermentation substrates.

| Strain | M18447 | M30719 | M32292 |
|---|---|---|---|
| Ethanol | +0.9 | +1.2 | +2.1 |
| Glycerol | −5 | −10 | −17 |
| Fermentation time | +6% | −2% | +2% |
| Pyruvate | −21 | −38 | −80 |
| Acetaldehyde | +12 | −34 | −22 |
| Iso-butanol | +15 | −62 | −64 |
| Amyl-alcohol | +12 | −97 | −98 |
| Iso-amyl-alcohol | +5 | −77 | −73 |

Over the 16 cycles, strains M30719 and M32292 were faster to finish fermentation compared to the control yeast strain M17328. Strains M30719 and M32292 also showed higher ethanol production and lower glycerol compared to parent strain M17328 and strain M18447. Strains M30719 and M32292 exhibited decreased pyruvate and acetaldehyde levels compared to control strain M17328. Strains M30719 and M32292 also exhibited lower fusel alcohol (iso-butanol, active amyl alcohol and iso-amyl alcohol) levels compared to control yeast strain M17328.

The performances of strains M17328 (wild-type), M27892 (including a glycerol reduction technology and expressing stl1), M30719 (expressing a heterologous pdc1 only) and M30743 (including a glycerol reduction technology and expressing stl1) on a single cycle of fermentation on Brazilian must was compared. The results are provided in Table 14.

TABLE 14
Ethanol (g/g), glycerol (g/g), and fermentation time are expressed as the % change
versus the control yeast M17328 in order to compare between fermentation substrates.

| Strain | M27892 | M30719 | M30743 |
|---|---|---|---|
| Ethanol | 0.8% | 2.5% | 3.5% |
| Glycerol | −27% | −16% | −38% |
| Fermentation time | 18% | −7% | 11% |

Strains M27892, M30719, and M30743 all produced more ethanol and less glycerol than the wild-type strain M17328. Strain M27892 was the slowest of the strains to complete the fermentation. The presence of pdc1 in strain M30743 did improve the fermentation kinetic.

The performances of strains M2390 as well as isolates T13869-1, T13870-1, T13872-1, T13875-1, T13878-2, T13871-1, T13874-2, T13877-1, T13873-1, T13876-1 and T13879-1 (expressing various heterologous stl1 and/or pdc1) on a single cycle of fermentation on Brazilian must was compared. The results are provided in Table 15.

TABLE 15
Ethanol (g/g), glycerol (g/g), and fermentation
time are expressed as the % change
versus the control yeast M2390 in order
to compare between fermentation substrates.

| Strain | Ethanol | Glycerol | Fermentation time |
|---|---|---|---|
| M2390 | 0.0% | 0% | 0% |
| T13869-1 | 2.3% | −8% | −4% |
| T13870-1 | 0.8% | −7% | −1% |
| T13872-1 | 1.1% | −6% | −3% |
| T13875-1 | 2.9% | −15% | −8% |
| T13878-2 | 0.5% | −13% | −3% |
| T13871-1 | 1.7% | −13% | −8% |
| T13874-2 | 3.9% | −24% | −12% |
| T13877-1 | 2.4% | −21% | −9% |
| T13873-1 | −0.8% | −3% | 7% |
| T13876-1 | 1.7% | −15% | −7% |
| T13879-1 | 1.8% | −11% | −4% |

Strains co-expressing both heterologous stl1 and pdc1 (T13875-1, T13878-2, T13874-2, T13877-1, T13876-1, and T13879-1) had better ethanol yield, higher glycerol reduction and/or shorter fermentations than corresponding strains expressing only heterologous stl1 (T13872-1, T113871-1, and T13873-1).

### Example III—Promoters

TABLE 16
Genotypes of the Saccharomyces cerevisiae strains
used in the example. All the recombinant strains used
directly or indirectly strain M2390 as a parental strain.

| Designation | Polypeptides overexpressed | Native genes inactivated |
|---|---|---|
| M2390 | Not applicable, this is a wild-type train. | |
| M24032 | stl1 (SEQ ID NO: 8) under | stl1 |
| | the control of the adh1p | |
| M25489 | stl1 (SEQ ID NO: 8) under | stl1 |
| | the control of the eno2p | |
| M25484 | stl1 (SEQ ID NO: 8) under | stl1 |
| | the control of the pgk1p | |
| M25507 | stl1 (SEQ ID NO: 8) under | stl1 |
| | the control of the ydr524c-bp | |
| M25494 | stl1 (SEQ ID NO: 8) under | stl1 |
| | the control of the tef1p | |
| M25480 | stl1 (SEQ ID NO: 8) under | stl1 |
| | the control of the tef2p | |
| M25499 | stl1 (SEQ ID NO: 8) under | stl1 |
| | the control of the gpm1p | |
| M25478 | stl1 (SEQ ID NO: 8) under | stl1 |
| | the control of the tpi1p | |
| M25503 | stl1 (SEQ ID NO: 8) under | stl1 |
| | the control of the rpl3p | |
| M25481 | stl1 (SEQ ID NO: 8) under | stl1 |
| | the control of the cyc1p | |
| M25472 | stl1 (SEQ ID NO: 8) under | stl1 |
| | the control of the tdh1p | |
| M25487 | stl1 (SEQ ID NO: 8) under | stl1 |
| | the control of the qcr8p and hxt3p | |
| M25486 | stl1 (SEQ ID NO: 8) under | stl1 |
| | the control of the tir1p | |
| M25475 | stl1 (SEQ ID NO: 8) under | stl1 |
| | the control of the hor7p | |

The different yeast strains were cultivated in YPD 40 g/L glucose medium at 3500 with aeration overnight prior to inoculation at a final concentration of 0.06 g/L of dry cell weight in a liquefied corn mash. More specifically, the yeast strains were inoculated in a 32.2%-33.2% total solids corn mash containing 165 ppm urea and exogenous glucoamylase (100% dose corresponding to 0.6 AGU/gTS). Permissive fermentations were conducted in minivials (volume of 3 g) incubated at 33.300 for 48 h. The results are shown in Table 17.

TABLE 17
Fermentation yield (in g/L) obtained with the various strains. All results
are provided at drop, except for the metabolite “ethanol at 22 h”.

| | | | | |
|---|---|---|---|---|
| Strain | Ethanol - 22 h | Ethanol | Glucose | Glycerol |

Substrate comprising 32.2% total solids

| | | | | |
|---|---|---|---|---|
| M2390 | 109.4 | 144.1 | 2.9 | 10.8 |
| M24032 | 100.6 | 147.7 | 1.4 | 5.1 |
| M25494 | 102.9 | 147.6 | 1.2 | 5.7 |
| M25499 | 102.9 | 148.0 | 1.2 | 5.6 |
| M25507 | 102.4 | 147.9 | 1.2 | 5.8 |
| M25480 | 103.6 | 147.2 | 1.5 | 6.4 |
| M25484 | 101.7 | 147.7 | 1.8 | 5.3 |
| M25503 | 103.5 | 147.12 | 1.2 | 6.2 |
| M25478 | 102.7 | 147.6 | 1.7 | 5.8 |

Substrate comprising 33.2% total solids

| | | | | |
|---|---|---|---|---|
| M2390 | N.A. | 142.2 | 1.1 | 10.3 |
| M24032 | N.A. | 144.0 | 2.9 | 5.0 |
| M25486 | N.A. | 142.6 | 0.9 | 7.6 |
| M25487 | N.A. | 141.3 | 1.6 | 8.2 |
| M25489 | N.A. | 143.3 | 1.7 | 5.1 |
| M25472 | N.A. | 143.4 | 1.2 | 7.1 |
| M25475 | N.A. | 143.4 | 2.1 | 5.9 |
| M25481 | N.A. | 142.4 | 1.0 | 8.2 |

While the invention has been described in connection with specific embodiments thereof, it will be understood that the scope of the claims should not be limited by the preferred embodiments set forth in the examples, but should be given the broadest interpretation consistent with the description as a whole.

1. A recombinant yeast cell for making ethanol, the recombinant yeast cell comprising:
a) one or more first genetic modifications to increase a yield of ethanol in the recombinant yeast cell as compared to a parental yeast cell; and
b) a second genetic modification to increase pyruvate decarboxylase activity in the recombinant yeast cell when compared to the parental yeast cell;
wherein the parental yeast cell lacks the first genetic modification and the second genetic modification.

2. The recombinant yeast cell of claim 1, wherein the one or more first genetic modification is:
capable of causing a reduction in a specific cell growth rate in an intermediate yeast cell as compared to the parental strain, and/or
capable of causing a reduction in an ethanol production rate in the intermediate yeast cell as compared to the parental cell,
wherein the intermediate yeast cell comprises the one or more first genetic modifications and lacks the second genetic modification.

3. The recombinant yeast cell of claim 1, wherein the one or more first genetic modification is for, when compared to the parental yeast cell:
(i) reducing the production of glycerol;
(ii) downregulating glycerol synthesis;
(iii) decreasing the activity or production of one or more enzymes that facilitate glycerol synthesis; and/or
(iv) facilitating glycerol transport.

4. The recombinant yeast cell of claim 3, wherein the one or more first genetic modification comprises;
a genetic modification for reducing the expression or inactivating one or more of the following native genes: gpd1, gpd2, gpp1 and/or gpp2, when compared to the parental yeast cell;
a genetic modification for overexpressing a native polypeptide having glycerol proton symporter activity, and/or expressing a heterologous polypeptide having glycerol proton symporter activity;
a genetic modification for increasing formate/acetyl-CoA production, when compared to the parental yeast cell;
a genetic modification for overexpressing a native polypeptide having pyruvate formate lyase activity and/or expressing a heterologous polypeptide having pyruvate formate lyase activity;
a genetic modification for increasing acetaldehyde/alcohol dehydrogenase activity, when compared to the parental yeast cell; and/or
a genetic modification for expressing a heterologous polypeptide having acetaldehyde/alcohol dehydrogenase activity.

5. (canceled)

6. The recombinant yeast cell of claim 4, wherein the native or the heterologous polypeptide having glycerol proton symporter activity is stl1.

7. (canceled)

8. (canceled)

9. The recombinant yeast cell of claim 4, wherein the native or the heterologous polypeptide having pyruvate formate lyase activity comprises pflA and/or pflB.

10. (canceled)

11. (canceled)

12. The recombinant yeast cell of claim 4, wherein the heterologous polypeptide having acetaldehyde/alcohol dehydrogenase activity comprises an acetaldehyde/alcohol dehydrogenase.

13. The recombinant yeast cell of claim 12, wherein the acetaldehyde/alcohol dehydrogenase is adhE.

14. The recombinant yeast cell of claim 1, wherein the second genetic modification is for expressing a heterologous polypeptide having pyruvate decarboxylase activity.

15. The recombinant yeast cell of claim 14, wherein the heterologous polypeptide having pyruvate decarboxylase activity:
has a lower Km than a native polypeptide having pyruvate decarboxylase; and/or
has the amino acid sequence of SEQ ID NO: 12, 14, 16, 17, 34, 35, 36 or 69, is a variant of the amino acid sequence of SEQ ID NO: 12, 14, 16, 17, 34, 35, 36 or 69 having pyruvate decarboxylase activity or is a fragment of the amino acid sequence of SEQ ID NO: 12, 14, 16, 17, 34, 35, 36 or 69 having pyruvate decarboxylase activity.

16. (canceled)

17. The recombinant yeast cell of claim 1 having at least one inactivated copy of a native gene encoding a native polypeptide having pyruvate decarboxylase activity.

18. The recombinant yeast cell of claim 1 comprising at least one of a third genetic modification:
for overexpressing a native enzyme belonging to EC 1.2.1.9 or 1.2.1.90 and/or expressing a heterologous enzyme belonging to EC 1.2.1.9 or 1.2.1.90; or
for overexpressing a native polypeptide having alcohol dehydrogenase activity and/or expressing a heterologous polypeptide having alcohol dehydrogenase activity.

19. The recombinant yeast cell of claim 18, wherein the heterologous polypeptide belonging to EC 1.2.1.9 or 1.2.1.90 is gapN.

20. The recombinant yeast cell of claim 18 having at least one inactivated copy of a native gene encoding:
a native polypeptide having glucose-6-phosphate dehydrogenase activity; and/or
a native gene encoding a native polypeptide having butanediol dehydrogenase activity.

21. The recombinant yeast cell of claim 20, wherein the native or the heterologous polypeptide having alcohol dehydrogenase activity is adhB and/or adhA.

22. (canceled)

23. (canceled)

24. The recombinant yeast cell of claim 1 being from the genus Saccharomyces sp or from the species of Saccharomyces cerevisiae.

25. (canceled)

26. A method of making a recombinant yeast cell for producing ethanol, the method comprising introducing, in a parental yeast cell, one or more first genetic modification and a second genetic modification to obtain the recombinant yeast cell, wherein:
a) the first genetic modification is for increasing a yield of ethanol in the recombinant yeast cell when compared to the parental yeast cell; and
b) the second genetic modification is for increasing pyruvate decarboxylase activity in the recombinant yeast cell when compared to the parental yeast cell;
wherein the parental yeast cell lacks the one or more first genetic modification and the second genetic modification.

27.-38. (canceled)

39. A process for making ethanol, the process comprising contacting the recombinant yeast cell of claim 1 with a substrate under a condition allowing the conversion of at least part of the substrate into ethanol.

40.-51. (canceled)


SEQUENCE LISTING (88 sequences)

SEQ ID NO: 1 - DNA, 879 bp, Bifidobacterium adolescentis
  source 1..879: mol_type = genomic DNA
atgtccgaacacatcttcagatccactactagacacatgttgagagattccaaggactacgttaatcaaactttgatgggtggtttgtctggtttcgaatctccaattggtttggatagattggacagaatcaaggctttgaagtctggtgatatcggttttgttcattcctgggatattaacacctctgttgatggtccaggtactagaatgactgtttttatgtctggttgcccattgagatgtcaatactgtcaaaatccagacacctggaaaatgagagatggtaaaccagtttactacgaagccatggtcaaaaagattgaaagatacgccgatttgttcaaagctactggtggtggtattactttttctggtggtgaatctatgatgcaaccagcttttgtttccagagtttttcatgctgctaagcaaatgggtgttcatacttgtttggatacctctggttttttgggtgcttcttacactgatgatatggttgatgatatcgacttgtgcttgttggatgttaagtcaggtgatgaagaaacctaccataaggttaccggtggtattttacaacctaccattgatttcggtcaaagattggctaaagccggtaaaaagatctgggttagattcgttttggtcccaggtttgacttcttctgaagaaaatgttgaaaacgtcgccaagatttgtgaaacttttggtgatgccttggaacacattgatgttttgccatttcaccaattgggtagaccaaaatggcacatgttgaatattccatacccattggaagatcaaaagggtccatctgctgctatgaagcaaagagttgttgaacaattccaatcccatggtttcaccgtttactaa

SEQ ID NO: 2 - protein, 292 aa, Bifidobacterium adolescentis
MSEHIFRSTTRHMLRDSKDYVNQTLMGGLSGFESPIGLDRLDRIKALKSGDIGFVHSWDINTSVDGPGTRMTVFMSGCPLRCQYCQNPDTWKMRDGKPVYYEAMVKKIERYADLFKATGGGITFSGGESMMQPAFVSRVFHAAKQMGVHTCLDTSGFLGASYTDDMVDDIDLCLLDVKSGDEETYHKVTGGILQPTIDFGQRLAKAGKKIWVRFVLVPGLTSSEENVENVAKICETFGDALEHIDVLPFHQLGRPKWHMLNIPYPLEDQKGPSAAMKQRVVEQFQSHGFTVY

SEQ ID NO: 3 - DNA, 2376 bp, Bifidobacterium adolescentis
  source 1..2376: mol_type = genomic DNA
atggctgctgttgatgctaccgctgtttctcaagaagaattggaagctaaagcttgggaaggttttactgaaggtaactggcaaaaggatatcgatgttagagacttcatccaaaagaactacactccatacgaaggtgatgaatcttttttggctgatgctaccgataagaccaaacatttgtggaaatacttggacgacaactacttgtccgtcgaaagaaaacaaagagtttacgacgttgatactcatactccagctggtattgatgcttttccagctggttatattgattccccagaagttgataacgtcatcgttggtttacaaaccgatgttccatgtaagagggctatgatgccaaatggtggttggagaatggttgaacaagctatcaaagaagccggtaaagaaccagatccagaaatcaagaagatcttcaccaagtacagaaagacccataacgatggtgtttttggtgtttacaccaagcaaatcaaggttgctagacacaacaagattttgactggtttgccagatgcttatggtagaggtagaattatcggtgattatagaagagttgccttgtacggtgttaacgctttgattaagttcaagcaaagagacaaggactccattccatacagaaacgatttcaccgaaccagaaatcgaacattggatcagattcagagaagaacacgacgaacaaatcaaggctttgaagcaattgatcaacttgggtaacgaatacggtttggatttgtctagaccagctcaaactgctcaagaagctgttcaatggacttatatgggttatttggcttccgttaagtctcaagatggtgctgctatgtcttttggtagagtttctaccttcttcgacgtctacttcgaaagagatttgaaggctggtaagattactgaaaccgatgcccaagaaatcatcgataacttggtcatgaagttgagaatcgtcagattcttgagaactaaggattacgatgccattttctctggtgatccatattgggctacttggtctgatgctggttttggtgatgatggtagaactatggttaccaagacctccttcagattattgaacactttgaccttggaacatttgggtccaggtccagaacctaacattactattttttgggacccaaagttgccagaagcttacaaaagattctgcgccagaatttctattgatacctccgctattcaatacgaatccgacaaagaaatcagatctcattggggtgatgatgctgctattgcttgttgtgtttctccaatgagagtcggtaagcaaatgcaatttttcgctgctagagtcaactctgctaaggctttgttgtacgctattaacggtggtagagacgaaatgactggtatgcaagtcatcgataagggtgttatcgatccaatcaaacctgaagctgatggtactttggactacgaaaaggttaaggctaattacgaaaaggccttggaatggttgtctgaaacttatgttatggccttgaacatcatccattacatgcatgataagtacgcctacgaatctattgaaatggccttgcatgacaaagaagtctatagaactttgggttgtggtatgtctggtttgtctattgctgctgattctttgtctgcttgtaagtacgctaaggtttacccaatctacaacaaggatgctaaaactactccaggtcacgaaaacgaatatgttgaaggtgctgatgatgatttgatcgttggttatagaaccgaaggtgactttccattatacggtaacgatgatgatagagctgatgatattgccaagtgggttgtttctactgttatgggtcaagttaagagattgccagtttacagagatgctgttccaacccaatccattttgactattacctccaacgtcgaatacggtaaagctactggtgcttttccatcaggtcataagaaaggtactccatatgctccaggtgctaatccagaaaatggtatggattctcatggtatgttgccatctatgttctccgttggtaagatcgattacaacgatgctttggatggtatttctttgaccaacactattaccccagatggtttgggtagagacgaagaagaaagaatcggtaacttggttggtattttggatgctggtaatggtcatggtctataccatgctaacatcaacgtcttgagaaaagaacaattggaagatgccgttgaacacccagaaaagtatccacatttgaccgttagagtttctggttacgctgttaacttcgtcaagttgaccaaagaacaacaattggatgtcatctccagaacttttcatcaaggtgctgttgttgattaa

SEQ ID NO: 4 - protein, 791 aa, Bifidobacterium adolescentis
MAAVDATAVSQEELEAKAWEGFTEGNWQKDIDVRDFIQKNYTPYEGDESFLADATDKTKHLWKYLDDNYLSVERKQRVYDVDTHTPAGIDAFPAGYIDSPEVDNVIVGLQTDVPCKRAMMPNGGWRMVEQAIKEAGKEPDPEIKKIFTKYRKTHNDGVFGVYTKQIKVARHNKILTGLPDAYGRGRIIGDYRRVALYGVNALIKFKQRDKDSIPYRNDFTEPEIEHWIRFREEHDEQIKALKQLINLGNEYGLDLSRPAQTAQEAVQWTYMGYLASVKSQDGAAMSFGRVSTFFDVYFERDLKAGKITETDAQEIIDNLVMKLRIVRFLRTKDYDAIFSGDPYWATWSDAGFGDDGRTMVTKTSFRLLNTLTLEHLGPGPEPNITIFWDPKLPEAYKRFCARISIDTSAIQYESDKEIRSHWGDDAAIACCVSPMRVGKQMQFFAARVNSAKALLYAINGGRDEMTGMQVIDKGVIDPIKPEADGTLDYEKVKANYEKALEWLSETYVMALNIIHYMHDKYAYESIEMALHDKEVYRTLGCGMSGLSIAADSLSACKYAKVYPIYNKDAKTTPGHENEYVEGADDDLIVGYRTEGDFPLYGNDDDRADDIAKWVVSTVMGQVKRLPVYRDAVPTQSILTITSNVEYGKATGAFPSGHKKGTPYAPGANPENGMDSHGMLPSMFSVGKIDYNDALDGISLTNTITPDGLGRDEEERIGNLVGILDAGNGHGLYHANINVLRKEQLEDAVEHPEKYPHLTVRVSGYAVNFVKLTKEQQLDVISRTFHQGAVVD

SEQ ID NO: 5 - DNA, 2733 bp, Bifidobacterium adolescentis
  source 1..2733: mol_type = genomic DNA
atggccgacgccaagaagaaagaagaacctactaagccaaccccagaagaaaaattggctgctgctgaagctgaagttgatgctttggttaagaaaggtttgaaggccttggacgaattcgaaaaattggatcaaaagcaagtcgatcacatcgttgctaaagcttcagttgctgctttgaacaaacatttggttttggctaagatggccgttgaagaaactcatagaggtttggttgaagataaggccaccaagaatattttcgcttgtgaacatgtcaccaactatttggctggtcaaaagaccgttggtatcattagagaagatgatgttttgggtatcgacgaaattgctgaaccagttggtgttgttgctggtgttactccagttactaatccaacttctaccgctattttcaagtccttgattgccttgaaaaccagatgcccaattatctttggttttcatccaggtgctcaaaactgttctgttgctgctgctaaaatcgttagagatgctgctattgctgctggtgctccagaaaactgtattcaatggattgaacacccatccattgaagctactggtgctttgatgaagcacgatggtgttgctactattttggctactggtggtccaggtatggttaaggctgcttattcttctggtaaaccagctttgggtgttggtgctggtaatgctccagcttatgttgataagaacgttgatgttgttagagctgccaacgatttgattttgtctaagcacttcgactacggtatgatttgtgctactgaacaagctattatcgccgataaggatatctatgctccattggtcaaagaattgaagagaagaaaggcctacttcgttaatgctgacgaaaaagctaagttggaacagtatatgttcggttgtaccgcttactctggtcaaactccaaagttgaattctgttgttccaggtaagtccccacagtatattgctaaagctgccggtttcgaaattccagaagatgctacaattttggccgctgaatgtaaagaagtcggagaaaacgaaccattgaccatggaaaaattggcaccagttcaagctgttttgaagtccgataacaaagaacaagccttcgaaatgtgcgaagccatgttgaaacatggtgctggtcatactgctgctattcatacaaacgatagagacttggtcagagaatacggtcaaagaatgcatgcctgcagaattatttggaactctccatcttctttgggtggtgttggtgatatctacaatgctattgctccatctttgactttgggttgtggttcttatggtggtaattctgtttccggtaatgttcaagccgtcaacttgattaacatcaagagaatcgctagaagaaacaacaacatgcaatggttcaagattccagctaagacttactttgaacctaacgccatcaagtacctaagagatatgtacggtatcgaaaaggctgttatcgtttgcgataaggtcatggaacaattgggtatcgttgataagatcatcgatcaattgagagccagatctaacagagttaccttcagaatcatcgattacgttgaaccagaaccatctgttgaaacagttgaaaggggtgctgctatgatgagagaagaatttgaacctgataccattattgctgttggtggtggttctccaatggatgcttctaagattatgtggttgttgtacgaacacccagaaatttcattctccgatgtcagagaaaagttcttcgacattagaaagagagcctttaagattccaccattgggtaaaaaggccaagttggtatgtattccaacctcttcaggtactggttctgaagttactccattcgctgttattaccgatcataagactggttacaagtacccaattaccgattatgctttgactccatctgttgctatcgttgatccagttttggctagaactcaacctagaaaattggcttctgatgctggttttgatgctttgacacatgcttttgaagcctacgtttctgtttacgctaacgatttcactgatggtatggctttacatgctgctaaattggtttgggataacttggctgaatccgttaatggtgaaccaggtgaagaaaaaactagagcccaagaaaagatgcataacgctgctactatggctggtatggcatttggttctgcttttttgggtatgtgtcatggtatggctcatacaattggtgctttgtgtcatgttgctcatggtagaactaactccattttgttgccatacgtcatcagatacaacggttctgttcctgaagaacctacatcttggccaaagtacaacaagtatattgccccagaaagataccaagaaatcgctaagaacttgggtgttaatccaggtaaaactcctgaagaaggtgttgaaaatttggctaaggctgtcgaagattacagagataacaagttgggtatgaacaagtccttccaagaatgtggtgttgacgaagattactactggtccattatcgatcaaattggtatgagagcctacgaagatcaatgtgctccagctaatccaagaattccacaaatcgaagatatgaaggatattgctattgccgcttactacggtgtttctcaagctgaaggtcataagttgagagttcaaagacaaggtgaagctgctacagaagaagcttctgaaagagcttaa

SEQ ID NO: 6 - protein, 910 aa, Bifidobacterium adolescentis
MADAKKKEEPTKPTPEEKLAAAEAEVDALVKKGLKALDEFEKLDQKQVDHIVAKASVAALNKHLVLAKMAVEETHRGLVEDKATKNIFACEHVTNYLAGQKTVGIIREDDVLGIDEIAEPVGVVAGVTPVTNPTSTAIFKSLIALKTRCPIIFGFHPGAQNCSVAAAKIVRDAAIAAGAPENCIQWIEHPSIEATGALMKHDGVATILATGGPGMVKAAYSSGKPALGVGAGNAPAYVDKNVDVVRAANDLILSKHFDYGMICATEQAIIADKDIYAPLVKELKRRKAYFVNADEKAKLEQYMFGCTAYSGQTPKLNSVVPGKSPQYIAKAAGFEIPEDATILAAECKEVGENEPLTMEKLAPVQAVLKSDNKEQAFEMCEAMLKHGAGHTAAIHTNDRDLVREYGQRMHACRIIWNSPSSLGGVGDIYNAIAPSLTLGCGSYGGNSVSGNVQAVNLINIKRIARRNNNMQWFKIPAKTYFEPNAIKYLRDMYGIEKAVIVCDKVMEQLGIVDKIIDQLRARSNRVTFRIIDYVEPEPSVETVERGAAMMREEFEPDTIIAVGGGSPMDASKIMWLLYEHPEISFSDVREKFFDIRKRAFKIPPLGKKAKLVCIPTSSGTGSEVTPFAVITDHKTGYKYPITDYALTPSVAIVDPVLARTQPRKLASDAGFDALTHAFEAYVSVYANDFTDGMALHAAKLVWDNLAESVNGEPGEEKTRAQEKMHNAATMAGMAFGSAFLGMCHGMAHTIGALCHVAHGRTNSILLPYVIRYNGSVPEEPTSWPKYNKYIAPERYQEIAKNLGVNPGKTPEEGVENLAKAVEDYRDNKLGMNKSFQECGVDEDYYWSIIDQIGMRAYEDQCAPANPRIPQIEDMKDIAIAAYYGVSQAEGHKLRVQRQGEAATEEASERA

SEQ ID NO: 7 - DNA, 1710 bp, Saccharomyces cerevisiae
  source 1..1710: mol_type = genomic DNA
atgaaggatttaaaattatcgaatttcaaaggcaaatttataagcagaaccagtcactggggacttacgggtaagaagttgcggtatttcatcactatcgcatctatgacgggcttctccctgtttggatacgaccaagggttgatggcaagtctaattactggtaaacagttcaactatgaatttccagcaaccaaagaaaatggcgatcatgacagacacgcaactgtagtgcagggcgctacaacctcctgttatgaattaggttgtttcgcaggttctctattcgttatgttctgcggtgaaagaattggtagaaaaccattaatcctgatgggttccgtaataaccatcattggtgccgttatttctacatgcgcatttcgtggttactgggcattaggccagtttatcatcggaagagtcgtcactggtgttggaacagggttgaatacatctactattcccgtttggcaatcagaaatgtcaaaagctgaaaatagagggttgctggtcaatttagaaggttccacaattgcttttggtactatgattgcttattggattgattttgggttgtcttataccaacagttctgttcagtggagattccccgtgtcaatgcaaatcgtttttgctctcttcctgcttgctttcatgattaaactacctgaatcgccacgttggctgatttctcaaagtcgaacagaagaagctcgctacttggtaggaacactagacgacgcggatccaaatgatgaggaagttataacagaagttgctatgcttcacgatgctgttaacaggaccaaacacgagaaacattcactgtcaagtttgttctccagaggcaggtcccaaaatcttcagagggctttgattgcagcttcaacgcaatttttccagcaatttactggttgtaacgctgccatatactactctactgtattattcaacaaaacaattaaattagactatagattatcaatgatcataggtggggtcttcgcaacaatctacgccttatctactattggttcattttttctaattgaaaagctaggtagacgtaagctgtttttattaggtgccacaggtcaagcagtttcattcacaattacatttgcatgcttggtcaaagaaaataaagaaaacgcaagaggtgctgccgtcggcttatttttgttcattacattctttggtttgtctttgctatcattaccatggatatacccaccagaaattgcatcaatgaaagttcgtgcatcaacaaacgctttctccacatgtactaattggttgtgtaactttgcggttgtcatgttcaccccaatatttattggacagtccggttggggttgctacttattttttgctgttatgaattatttatacattccagttatcttctttttctaccctgaaaccgccggaagaagtttggaggaaatcgacatcatctttgctaaagcatacgaggatggcactcaaccatggagagttgctaaccatttgcccaagttatccctacaagaagtcgaagatcatgccaatgcattgggctcttatgacgacgaaatggaaaaagaggactttggtgaagatagagtagaagacacctataaccaaattaacggcgataattcgtctagttcttcaaacatcaaaaatgaagatacagtgaacgataaagcaaattttgagggttga

SEQ ID NO: 8 - protein, 569 aa, Saccharomyces cerevisiae
MKDLKLSNFKGKFISRTSHWGLTGKKLRYFITIASMTGFSLFGYDQGLMASLITGKQFNYEFPATKENGDHDRHATVVQGATTSCYELGCFAGSLFVMFCGERIGRKPLILMGSVITIIGAVISTCAFRGYWALGQFIIGRVVTGVGTGLNTSTIPVWQSEMSKAENRGLLVNLEGSTIAFGTMIAYWIDFGLSYTNSSVQWRFPVSMQIVFALFLLAFMIKLPESPRWLISQSRTEEARYLVGTLDDADPNDEEVITEVAMLHDAVNRTKHEKHSLSSLFSRGRSQNLQRALIAASTQFFQQFTGCNAAIYYSTVLFNKTIKLDYRLSMIIGGVFATIYALSTIGSFFLIEKLGRRKLFLLGATGQAVSFTITFACLVKENKENARGAAVGLFLFITFFGLSLLSLPWIYPPEIASMKVRASTNAFSTCTNWLCNFAVVMFTPIFIGQSGWGCYLFFAVMNYLYIPVIFFFYPETAGRSLEEIDIIFAKAYEDGTQPWRVANHLPKLSLQEVEDHANALGSYDDEMEKEDFGEDRVEDTYNQINGDNSSSSSNIKNEDTVNDKANFEG

SEQ ID NO: 9 - DNA, 1548 bp, Saccharomycopsis fibuligera
  source 1..1548: mol_type = genomic DNA
atgatcagattgaccgttttcttgaccgctgtttttgctgctgttgcttcttgtgttccagttgaattggataagagaaacaccggtcatttccaagcttattctggttataccgttaacagatctaacttcacccaatggattcatgaacaaccagctgtttcttggtactacttgttgcaaaacatcgattacccagaaggtcaattcaaatctgctaaaccaggtgttgttgttgcttctccatctacatctgaaccagattacttctaccaatggactagagataccgctattaccttcttgtccttgattgctgaagttgaagatcattctttctccaacactaccttggctaaggttgtcgaatattacatttccaacacctacaccttgcaaagagtttctaatccatccggtaacttcgattctccaaatcatgatggtttgggtgaacctaagttcaacgttgatgatactgcttatacagcttcttggggtagaccacaaaatgatggtccagctttgagagcttacgctatttctagatacttgaacgctgttgctaagcacaacaacggtaaattattattggccggtcaaaacggtattccttattcttctgcttccgatatctactggaagattattaagccagacttgcaacatgtttctactcattggtctacctctggttttgatttgtgggaagaaaatcaaggtactcatttcttcaccgctttggttcaattgaaggctttgtcttacggtattccattgtctaagacctacaatgatccaggtttcacttcttggttggaaaaacaaaaggatgccttgaactcctacattaactcttccggtttcgttaactctggtaaaaagcacatcgttgaatctccacaattgtcatctagaggtggtttggattctgctacttatattgctgccttgatcacccatgatatcggtgatgatgatacttacaccccattcaatgttgataactcctacgttttgaactccttgtattacctattggtcgacaacaagaacagatacaagatcaacggtaactacaaagctggtgctgctgttggtagatatcctgaagatgtttacaacggtgttggtacttctgaaggtaatccatggcaattggctactgcttatgctggtcaaactttttacaccttggcctacaattccttgaagaacaagaagaacttggtcatcgaaaagttgaactacgacttgtacaactccttcattgctgatttgtccaagattgattcttcctacgcttctaaggattctttgactttgacctacggttccgataactacaagaacgttatcaagtccttgttgcaattcggtgactcattcttgaaggttttgttggatcacatcgatgacaacggtcaattgactgaagaaatcaacagatacaccggttttcaagctggtgcagtttctttgacttggtcatctggttctttgttgtctgctaatagagccagaaacaagttgatcgaattattgtga

SEQ ID NO: 10 - protein, 515 aa, Saccharomycopsis fibuligera
  SIGNAL 1..26
MIRLTVFLTAVFAAVASCVPVELDKRNTGHFQAYSGYTVNRSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 11 - DNA, 1707 bp, Zymomonas mobilis
  source 1..1707: mol_type = genomic DNA
atgtcttacaccgttggtacttacttggctgaaagattggttcagatcggtttgaaacatcattttgctgttgctggtgattacaacttggttttgttggacaacctgctgttgaacaagaacatggaacaagtttactgctgcaacgaattgaactgtggtttttctgctgaaggttacgctagagctaaaggtgctgctgctgcagttgttacttattctgttggtgctttgtctgctttcgatgctattggtggtgcttatgctgaaaacttgccagttattttgatttccggtgctccaaacaacaatgatcatgctgctggtcatgttttacatcatgctttgggtaagaccgattaccactatcaattggaaatggccaagaatattactgcagctgctgaagctatctacactccagaagaagctccagctaaaatcgatcatgttattaagaccgccttgagggaaaaaaagccagtttatttggaaatcgcttgcaacattgcttctatgccatgtgctgctccaggtccagcttctgctttgtttaatgatgaagcttctgatgaggcttctttgaatgctgccgttgaagaaactttgaagttcattgctaacagagataaggttgccgttttggttggttctaaattgagagctgctggtgctgaagaagctgctgttaagtttgctgatgccttaggtggtgctgttgcaactatggctgctgctaaatcatttttcccagaagaaaacccacactacattggtacatcttggggtgaagtttcttatccaggtgttgaaaagactatgaaggaagctgacgctgttattgctttggctccagtttttaacgattactctactactggttggaccgatattccagatccaaaaaagttggttttggccgaacctagatctgttgttgttaacggtattagattcccatccgttcacttgaaggattacttgactagattggcccaaaaggtgtctaaaaaaacaggtgctttggacttcttcaagtctttgaacgctggtgaattgaaaaaagctgctcctgctgatccatctgctccattggttaatgctgaaattgctagacaagttgaagctttgttgactccaaacactaccgttattgctgaaactggtgattcttggtttaacgcccaaagaatgaagttgccaaatggtgctagagtcgaatacgaaatgcaatggggtcatattggttggtctgttccagctgcttttggttatgcagttggtgctcctgagagaagaaacattttgatggttggtgatggttctttccagttgactgctcaagaggtagctcaaatggttagattgaaattgcccgtgatcatcttcctgattaacaactacggttacaccatcgaagttatgattcatgatggtccctacaacaacatcaagaattgggattatgctggcctgatggaagtttttaatggtaatggtggttacgattctggtgctggtaaaggtttgaaagctaaaactggtggtgaattagctgaagccattaaggttgctttagctaatactgatggtccaaccttgattgaatgcttcattggtagagaagattgcaccgaagaattggttaagtggggtaaaagagttgctgctgccaattctagaaaaccagttaacaagctgttgtaa

SEQ ID NO: 12 - protein, 568 aa, Zymomonas mobilis
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIANRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGIRFPSVHLKDYLTRLAQKVSKKTGALDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 13 - DNA, 1671 bp, Zymobacter palmae
  source 1..1671: mol_type = genomic DNA
atgtacaccgttggtatgtacttggctgaaagattggctcaaatcggtttgaaacatcattttgctgttgccggtgattacaacttggttttgttggatcaactgctgttgaacaaggatatggaacaagtttactgctgcaacgaattgaactgtggtttttctgctgaaggttatgctagagctagaggtgctgctgctgcaatagttactttttctgttggtgctatttccgccatgaatgctattggtggtgcttatgctgaaaacttgccagttattttgatctccggttctccaaacactaacgattatggtactggtcatatcttgcatcataccattggtactactgactacaactaccaattggaaatggttaagcacgttacctgtgctagagaatctattgtctcagctgaagaagctccagctaagattgatcatgttattagaaccgccttgagggaaagaaaaccagcttatttggaaattgcttgtaacgttgctggtgctgaatgtgttagaccaggtccaatcaattctttgttgagagaattggaagtcgaccaaacttctgttactgcagctgttgatgctgcagttgaatggttgcaagatagacaaaacgttgttatgttggtcggttctaaattgagagctgctgcagctgaaaaacaagctgttgctttagctgatagattgggttgtgctgttactattatggctgctgaaaaaggattcttcccagaagatcatccaaatttcagaggtctatattggggtgaagtttcttcagaaggtgctcaagaattggttgaaaacgctgatgctattttgtgtttggctccagtttttaacgattacgctactgttggttggaattcttggccaaaaggtgataatgttatggttatggataccgacagagttactttcgctggtcaatcttttgaaggcttgtctttgtctacattcgctgctgctttggcagaaaaagctccatctagaccagctactactcaaggtactcaagctcctgttttgggtattgaagctgcagaacctaatgctccattgactaatgacgaaatgaccagacaaatccagtccttgattacttctgataccactttgactgctgaaactggtgattcttggtttaacgcttctagaatgccaattccaggtggtgctagagttgaattagaaatgcaatggggtcatatcggttggtctgttccatctgcttttggtaatgctgttggttctccagaaagacgtcatattatgatggttggtgatggttcctttcaattgactgctcaagaggttgctcaaatgatcagatacgaaatcccagtgatcatcttcttgatcaacaacagaggttacgttatcgaaatcgccattcatgatggtccatacaactacattaagaactggaattacgccggtttgatcgatgttttcaatgatgaagatggtcacggtttgggtttgaaagcttctactggtgcagaattagaaggtgccattaagaaggctttggataatagaagaggtccaaccttgatcgaatgcaacattgctcaagatgactgtaccgaaactttgattgcttggggtaaaagagttgctgctaccaattctagaaagccacaagcttaa

SEQ ID NO: 14 - protein, 556 aa, Zymobacter palmae
MYTVGMYLAERLAQIGLKHHFAVAGDYNLVLLDQLLLNKDMEQVYCCNELNCGFSAEGYARARGAAAAIVTFSVGAISAMNAIGGAYAENLPVILISGSPNTNDYGTGHILHHTIGTTDYNYQLEMVKHVTCARESIVSAEEAPAKIDHVIRTALRERKPAYLEIACNVAGAECVRPGPINSLLRELEVDQTSVTAAVDAAVEWLQDRQNVVMLVGSKLRAAAAEKQAVALADRLGCAVTIMAAEKGFFPEDHPNFRGLYWGEVSSEGAQELVENADAILCLAPVFNDYATVGWNSWPKGDNVMVMDTDRVTFAGQSFEGLSLSTFAAALAEKAPSRPATTQGTQAPVLGIEAAEPNAPLTNDEMTRQIQSLITSDTTLTAETGDSWFNASRMPIPGGARVELEMQWGHIGWSVPSAFGNAVGSPERRHIMMVGDGSFQLTAQEVAQMIRYEIPVIIFLINNRGYVIEIAIHDGPYNYIKNWNYAGLIDVFNDEDGHGLGLKASTGAELEGAIKKALDNRRGPTLIECNIAQDDCTETLIAWGKRVAATNSRKPQA

SEQ ID NO: 15 - DNA, 1782 bp, Pisum sativum
  source 1..1782: mol_type = genomic DNA
atggaaaccgaaactgaaactccaaatggttctactccatgtccaacatctgctccatctgctattccattaagaccatcttcatgtgatggtactatgggtagacatttggctagaagattggttgaaatcggtgtcagagatgttttctctgttccaggtgatttcaacttgaccttgttggatcatttgattgccgaaccagaattgaacttggttggttgttgtaacgaattgaatgctggttatgctgctgatggttatggtagagctaaaggtgttggtgcttgtgttgttacttttactgttggtggtctgtccattttgaatgctattgctggtgcttactctgaaaacttgccagttatatgtattgtcggtggtccaaactctaacgattacggtactaatagaatcttgcatcacaccattggtttgccagatttctctcaagaattgcaatgcttccaaaccattacctgttttcaagctgttgtcaacaacttggatgatgcccatgaattgattgataccgctatttctaccgccttgaaagaatctaagccagtctacatttctatcggttgtaatttgccagctattccacatccaacttttgctagagatccagttccatttttcttggctccaagagtttctaatcaagctggtttagaagctgctgttgaagaagctgcagcatttttgaacaaagctgttaagccagttatcgttggtggaccaaaattgagagttgctaaagctcaaaaggccttcatggaatttgctgaagcttctggttatccaattgctgttatgccatctggtaaaggtttggttccagaaaatcatccacacttcattggtacttattggggtgctgtttctacttcatattgcggtgaaatagttgaatccgctgatgcttatgttttcgttggtccaattttcaacgactactcttctgttggttactccctgttgatcaagaaagagaagtctttgatcgttcagccaaacagagttactattggtaacggtttgtctctaggttgggtttttatggctgatttcttgactgctttggccaagaaagttaagactaacactacagccgtcgaaaactacagaagaatctacgttccaccaggtattccattgaaaagagaaaaagacgaaccattgagagtcaacgttttgttcaagcacattcaagccttgatttctggtgatactgctgttattgctgaaactggtgattcttggtttaactgccaaaagttgagactgccagaaaactgtggttacgaattccaaatgcaatacggttctattggttggtcagttggtgctactttgggttatgctcaagctgctactgataagagagttattgcttgtattggtgacggctctttccaagttactgctcaagatatttccaccatgattagatgcggtcagagatccattattttcttgattaacaacggtggctacaccatcgaagtcgaaattcatgatggtccatacaacgttatcaagaactgggattacactggtttcgtttccgctattcataacggtcaaggtaaatgttggaccgctaaagttagaaccgaagaagatttgacagaagctattgctactgctactggtgctgaaaaagattccttgtgcttcattgaagttttcgcccataaggatgacacctccaaagaattattggaatggggttctagagttgctgctgctaattctagaccacctaatccacaatga

SEQ ID NO: 16 - protein, 593 aa, Pisum sativum
METETETPNGSTPCPTSAPSAIPLRPSSCDGTMGRHLARRLVEIGVRDVFSVPGDFNLTLLDHLIAEPELNLVGCCNELNAGYAADGYGRAKGVGACVVTFTVGGLSILNAIAGAYSENLPVICIVGGPNSNDYGTNRILHHTIGLPDFSQELQCFQTITCFQAVVNNLDDAHELIDTAISTALKESKPVYISIGCNLPAIPHPTFARDPVPFFLAPRVSNQAGLEAAVEEAAAFLNKAVKPVIVGGPKLRVAKAQKAFMEFAEASGYPIAVMPSGKGLVPENHPHFIGTYWGAVSTSYCGEIVESADAYVFVGPIFNDYSSVGYSLLIKKEKSLIVQPNRVTIGNGLSLGWVFMADFLTALAKKVKTNTTAVENYRRIYVPPGIPLKREKDEPLRVNVLFKHIQALISGDTAVIAETGDSWFNCQKLRLPENCGYEFQMQYGSIGWSVGATLGYAQAATDKRVIACIGDGSFQVTAQDISTMIRCGQRSIIFLINNGGYTIEVEIHDGPYNVIKNWDYTGFVSAIHNGQGKCWTAKVRTEEDLTEAIATATGAEKDSLCFIEVFAHKDDTSKELLEWGSRVAAANSRPPNPQ

SEQ ID NO: 17 - protein, 405 aa, Pisum sativum
PVYISIGCNLPAIPHPTFSRDPVPFSLAPKLSNQMGLEAAVEAAAEFLNKAVKPVLVGGPKLRVAKASDAFVELADASGYALAVMPSAKGMVPEHHPHFIGTYWGAVSTAFCAEIVESADAYLFAGPIFNDYSSVGYSLLLKKEKAIIVMPDRVVIANGPAFGCVLMNDFLKALAKRLKHNNVAYENYHRIFVPDGTPLKSASKEPLRVNVMFQHIQKMLSSETAVIAETGDSWFNCQKLKLPEGCGYEFQMQYGSIGWSVGATLGYAQAVPEKRVIACIGDGSFQVTAQDVSTMLRCGQKTIIFLINNGGYTIEVEIHDGPYNVIKNWNYTGLVDAIHNGEGKCWTTKVFCEEELVEAIAKATGPKKDSLCFIEVIVHKDDTSKELLEWGSRVSAANSRPPNPQ

SEQ ID NO: 18 - DNA, 1014 bp, Zymomonas mobilis
  source 1..1014: mol_type = genomic DNA
atgaaggctgctgttattaccaaggatcataccattgaagttaaggacaccaaattgaggccattgaaatatggtgaagccttgttggaaatggaatactgtggtgtttgtcatactgacttgcatgttaagaatggtgatttcggtgacgaaactggtagaattactggtcatgaaggtatcggtatcgttaagcaagttggtgaaggtgttacttctttgaaggttggtgatagagcttctgttgcctggtttttcaaaggttgtggtcattgtgaatactgcgtttctggtaacgaaaccttgtgtagaaatgttgaaaacgctggttacactgttgatggtgctatggctgaagaatgtatagttgttgctgattactccgttaaggttccagatggtttggatccagctgttgcttcttctattacttgtgctggtgttactacttacaaggctgttaaggtttcccaaattcaaccaggtcaatggttggctatctatggtttaggtggtttgggtaatttggcattgcaatacgctaagaatgttttcaacgctaaggttatcgccatcgatgttaacgatgaacaattggctttcgccaaagaattgggtgctgatatggttattaacccaaagaatgaagatgccgccaagatcattcaagaaaaagttggtggtgctcatgctactgttgttactgctgttgctaagtctgcttttaactctgctgttgaagctattagagctggtggtagagttgttgcagttggtttgccaccagaaaagatggatttgtctattccaagattggtcttggatggtatcgaagttttgggttctttggttggtactagagaggatttgaaagaagcttttcaattcgctgctgaaggtaaggttaagccaaaagttactaagagaaaggtcgaagagatcaaccagattttcgacgaaatggaacatggtaaattcaccggtagaatggttgttgatttcacccatcattaa

SEQ ID NO: 19 - protein, 337 aa, Zymomonas mobilis
MKAAVITKDHTIEVKDTKLRPLKYGEALLEMEYCGVCHTDLHVKNGDFGDETGRITGHEGIGIVKQVGEGVTSLKVGDRASVAWFFKGCGHCEYCVSGNETLCRNVENAGYTVDGAMAEECIVVADYSVKVPDGLDPAVASSITCAGVTTYKAVKVSQIQPGQWLAIYGLGGLGNLALQYAKNVFNAKVIAIDVNDEQLAFAKELGADMVINPKNEDAAKIIQEKVGGAHATVVTAVAKSAFNSAVEAIRAGGRVVAVGLPPEKMDLSIPRLVLDGIEVLGSLVGTREDLKEAFQFAAEGKVKPKVTKRKVEEINQIFDEMEHGKFTGRMVVDFTHH

SEQ ID NO: 20 - DNA, 1149 bp, Zymomonas mobilis
  source 1..1149: mol_type = genomic DNA
atggcttcttccactttctacatcccatttgttaatgagatgggtgaaggttctttggaaaaggccattaaggatttgaatggttccggttttaagaacgccttgatagtttctgatgccttcatgaacaaatccggtgttgttaagcaagttgccgatttgttgaaagcccaaggtattaactctgctgtttacgatggtgttatgccaaatccaactgttactgctgttttggaaggtctgaaaatcttgaaggataacaactccgatttcgtcatctctttaggtggtggttctccacatgattgtgctaaagctattgctttggttgctactaatggtggtgaagttaaggattacgaaggtatcgataagtctaagaaaccagctttgccactgatgtctattaacactactgctggtactgcttctgaaatgaccagattctgtattatcaccgatgaagttagacacgtcaagatggctatagttgatagacatgttaccccaatggtttccgttaatgatcctttgttgatggttggtatgcctaaaggtttgactgctgctactggtatggatgctttgactcatgcttttgaagcttactcttctacagctgctactccaattactgatgcttgtgctttgaaagctgcttccatgattgctaagaacttgaaaactgcttgcgataacggtaaagatatgccagctagagaagctatggcttacgctcaatttttggctggtatggcttttaacaatgcctctttgggttatgttcatgctatggctcatcaattaggtggctattacaatttgccacatggtgtctgtaatgccgttttgttaccacatgttttggcttacaatgcttctgttgttgctggtagattgaaggatgttggtgttgctatgggtttagatattgctaacttgggtgacaaagaaggtgctgaagctactattcaagctgttagagatttggctgcctctattggtattccagctaatttgactgaattgggtgccaagaaagaagatgttccattattggctgatcatgccttgaaagatgcatgcgctttgactaatccaagacaaggtgatcaaaaagaggtcgaagagttgttcttgtctgcttga

SEQ ID NO: 21 - protein, 382 aa, Zymomonas mobilis
MASSTFYIPFVNEMGEGSLEKAIKDLNGSGFKNALIVSDAFMNKSGVVKQVADLLKAQGINSAVYDGVMPNPTVTAVLEGLKILKDNNSDFVISLGGGSPHDCAKAIALVATNGGEVKDYEGIDKSKKPALPLMSINTTAGTASEMTRFCIITDEVRHVKMAIVDRHVTPMVSVNDPLLMVGMPKGLTAATGMDALTHAFEAYSSTAATPITDACALKAASMIAKNLKTACDNGKDMPAREAMAYAQFLAGMAFNNASLGYVHAMAHQLGGYYNLPHGVCNAVLLPHVLAYNASVVAGRLKDVGVAMGLDIANLGDKEGAEATIQAVRDLAASIGIPANLTELGAKKEDVPLLADHALKDACALTNPRQGDQKEVEELFLSA

SEQ ID NO: 22 - DNA, 1428 bp, Streptococcus mutans
  source 1..1428: mol_type = genomic DNA
atgacaaaacaatataaaaattatgtcaatggcgagtggaagctttcagaaaatgaaattaaaatctacgaaccggccagtggagctgaattgggttcagttccagcaatgagtactgaagaagtagattatgtttatgcttcagccaagaaagctcaaccagcttggcgatcactttcatacatagaacgtgctgcctaccttcataaggtagcagatattttgatgcgtgataaagaaaaaataggtgctgttctttccaaagaggttgctaaaggttataaatcagcagtcagcgaagttgttcgtactgcagaaatcattaattatgcagctgaagaaggccttcgtatggaaggtgaagtccttgaaggcggcagttttgaagcagccagcaagaaaaaaattgccgttgttcgtcgtgaaccagtaggtcttgtattagctatttcaccatttaactaccctgttaacttggcaggttcgaaaattgcaccggctcttattgcgggaaatgttattgcttttaaaccaccgacgcaaggatcaatctcagggctcttacttgctgaagcatttgctgaagctggacttcctgcaggtgtctttaataccattacaggtcgtggttctgaaattggagactatattgtagaacatcaagccgttaactttatcaatttcactggttcaacaggaattggggaacgtattggcaaaatggctggtatgcgtccgattatgcttgaactcggtggaaaagattcagccatcgttcttgaagatgcagaccttgaattgactgctaaaaatattattgcaggtgcttttggttattcaggtcaacgctgtacagcagttaaacgtgttcttgtgatggaaagtgttgctgatgaactggtcgaaaaaatccgtgaaaaagttcttgcattaacaattggtaatccagaagacgatgcagatattacaccgttgattgatacaaaatcagctgattatgtagaaggtcttattaatgatgccaatgataaaggagccgctgcccttactgaaatcaaacgtgaaggtaatcttatctgtccaatcctctttgataaggtaacgacagatatgcgtcttgcttgggaagaaccatttggtcctgttcttccgatcattcgtgtgacatctgtagaagaagccattgaaatttctaacaaatcggaatatggacttcaggcttctatctttacaaatgatttcccacgcgcttttggtattgctgagcagcttgaagttggtacagttcatatcaataataagacacagcgcggtacggacaacttcccattcttaggggctaaaaaatcaggtgcaggtattcaaggggtaaaatattctattgaagctatgacaactgttaaatccgtcgtatttgatatcaaataa

SEQ ID NO: 23 - protein, 475 aa, Streptococcus mutans
MTKQYKNYVNGEWKLSENEIKIYEPASGAELGSVPAMSTEEVDYVYASAKKAQPAWRSLSYIERAAYLHKVADILMRDKEKIGAVLSKEVAKGYKSAVSEVVRTAEIINYAAEEGLRMEGEVLEGGSFEAASKKKIAVVRREPVGLVLAISPFNYPVNLAGSKIAPALIAGNVIAFKPPTQGSISGLLLAEAFAEAGLPAGVFNTITGRGSEIGDYIVEHQAVNFINFTGSTGIGERIGKMAGMRPIMLELGGKDSAIVLEDADLELTAKNIIAGAFGYSGQRCTAVKRVLVMESVADELVEKIREKVLALTIGNPEDDADITPLIDTKSADYVEGLINDANDKGAAALTEIKREGNLICPILFDKVTTDMRLAWEEPFGPVLPIIRVTSVEEAIEISNKSEYGLQASIFTNDFPRAFGIAEQLEVGTVHINNKTQRGTDNFPFLGAKKSGAGIQGVKYSIEAMTTVKSVVFDIK

SEQ ID NO: 24 - protein, 348 aa, Saccharomyces cerevisiae
MSIPETQKGVIFYESHGKLEYKDIPVPKPKANELLINVKYSGVCHTDLHAWHGDWPLPVKLPLVGGHEGAGVVVGMGENVKGWKIGDYAGIKWLNGSCMACEYCELGNESNCPHADLSGYTHDGSFQQYATADAVQAAHIPQGTDLAQVAPILCAGITVYKALKSANLMAGHWVAISGAAGGLGSLAVQYAKAMGYRVLGIDGGEGKEELFRSIGGEVFIDFTKEKDIVGAVLKATDGGAHGVINVSVSEAAIEASTRYVRANGTTVLVGMPAGAKCCSDVFNQVVKSISIVGSYVGNRADTREALDFFARGLVKSPIKVVGLSTLPEIYEKMEKGQIVGRYVVDTSK

SEQ ID NO: 25 - DNA, 1545 bp, Saccharomycopsis fibuligera
  source 1..1545: mol_type = genomic DNA
atgatcagattgaccgttttcttgaccgctgtttttgctgctgttgcttcttgtgttccagttgaattggataagagaaacaccggtcatttccaagcttattctggttataccgttgctagatctaacttcacccaatggattcatgaacaaccagctgtttcttggtactacttgttgcaaaacatcgattacccagaaggtcaattcaaatctgctaaaccaggtgttgttgttgcttctccatctacatctgaaccagattacttctaccaatggactagagataccgctattaccttcttgtccttgattgctgaagttgaagatcattctttctccaacactaccttggctaaggttgtcgaatattacatttccaacacctacaccttgcaaagagtttctaatccatccggtaacttcgattctccaaatcatgatggtttgggtgaacctaagttcaacgttgatgatactgcttatacagcttcttggggtagaccacaaaatgatggtccagctttgagagcttacgctatttctagatacttgaacgctgttgctaagcacaacaacggtaaattattattggccggtcaaaacggtattccttattcttctgcttccgatatctactggaagattattaagccagacttgcaacatgtttctactcattggtctacctctggttttgatttgtgggaagaaaatcaaggtactcatttcttcaccgctttggttcaattgaaggctttgtcttacggtattccattgtctaagacctacaatgatccaggtttcacttcttggttggaaaaacaaaaggatgccttgaactcctacattaactcttccggtttcgttaactctggtaaaaagcacatcgttgaatctccacaattgtcatctagaggtggtttggattctgctacttatattgctgccttgatcacccatgatatcggtgatgatgatacttacaccccattcaatgttgataactcctacgttttgaactccttgtattacctattggtcgacaacaagaacagatacaagatcaacggtaactacaaagctggtgctgctgttggtagatatcctgaagatgtttacaacggtgttggtacttctgaaggtaatccatggcaattggctactgcttatgctggtcaaactttttacaccttggcctacaattccttgaagaacaagaagaacttggtcatcgaaaagttgaactacgacttgtacaactccttcattgctgatttgtccaagattgattcttcctacgcttctaaggattctttgactttgacctacggttccgataactacaagaacgttatcaagtccttgttgcaattcggtgactcattcttgaaggttttgttggatcacatcgatgacaacggtcaattgactgaagaaatcaacagatacaccggttttcaagctggtgcagtttctttgacttggtcatctggttctttgttgtctgctaatagagccagaaacaagttgatcgaattattg

SEQ ID NO: 26 - protein, 515 aa, Saccharomycopsis fibuligera
  SIGNAL 1..26
MIRLTVFLTAVFAAVASCVPVELDKRNTGHFQAYSGYTVARSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 27 - DNA, 1527 bp, synthetic construct
  source 1..1527: mol_type = other DNA
atgaggttcccatctattttcaccgctgttttgtttgctgcttcttctgctttggctaacaccggtcatttccaagcttattctggttataccgttaacagagctaacttcacccaatggattcatgaacaaccagctgtttcttggtactacttgttgcaaaacatcgattacccagaaggtcaattcaaagctgctaaaccaggtgttgttgttgcttctccatctacatctgaaccagattacttctaccaatggactagagataccgctattaccttcttgtccttgattgctgaagttgaagatcattctttctccaacactaccttggctaaggttgtcgaatattacatttccaacacctacaccttgcaaagagtttctaatccatccggtaacttcgattctccaaatcatgatggtttgggtgaacctaagttcaacgttgatgatactgcttatacagcttcttggggtagaccacaaaatgatggtccagctttgagagcttacgctatttctagatacttgaacgctgttgctaagcacaacaacggtaaattattattggccggtcaaaacggtattccttattcttctgcttccgatatctactggaagattattaagccagacttgcaacatgtttctactcattggtctacctctggttttgatttgtgggaagaaaatcaaggtactcatttcttcaccgctttggttcaattgaaggctttgtcttacggtattccattgtctaagacctacaatgatccaggtttcacttcttggttggaaaaacaaaaggatgccttgaactcctacattaactcttccggtttcgttaactctggtaaaaagcacatcgttgaatctccacaattgtcatctagaggtggtttggattctgctacttatattgctgccttgatcacccatgatatcggtgatgatgatacttacaccccattcaatgttgataactcctacgttttgaactccttgtattacctattggtcgacaacaagaacagatacaagatcaacggtaactacaaagctggtgctgctgttggtagatatcctgaagatgtttacaacggtgttggtacttctgaaggtaatccatggcaattggctactgcttatgctggtcaaactttttacaccttggcctacaattccttgaagaacaagaagaacttggtcatcgaaaagttgaactacgacttgtacaactccttcattgctgatttgtccaagattgattcttcctacgcttctaaggattctttgactttgacctacggttccgataactacaagaacgttatcaagtccttgttgcaattcggtgactcattcttgaaggttttgttggatcacatcgatgacaacggtcaattgactgaagaaatcaacagatacaccggttttcaagctggtgcagtttctttgacttggtcatctggttctttgttgtctgctaatagagccagaaacaagttgatcgaattattgtga

SEQ ID NO: 28 - protein, 508 aa, synthetic construct
  SIGNAL 1..19
MRFPSIFTAVLFAASSALANTGHFQAYSGYTVNRANFTQWIHEQPAVSWYYLLQNIDYPEGQFKAAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 29 - DNA, 3008 bp, Kluyveromyces lactis
  source 1..3008: mol_type = genomic DNA
aagttcatttcaatgtctttcggaaaacctatagttctatcctgtccctaatagtgttcatattgggcagtggattctttttatttatgttaagcattccctatgtacctcactaggaaaagggcacttggacatcataattcttccttttatagcaatacttttctttctagcttccgtcctacctttttcataccggatttcaccttgtcagttcataataatatcaatactcacctattaagagccacatgacgtgcgtgcgattgagtacttaaaaactactgttaccttttgtccatcgtgtaactttgcattttctttgtcacattttcctgtacaagaactaacttgacaaaattgattttccttgaagagaagtcgaattaaaagtttaacaggaatatactgggacggacaaaaagatggaaaccaagaccctgatacactcaggggctgctaaggagatgagctatactgagaggtataatgtcgcaccgctaatacctttgcctgagtatctttttcatagattatttcagctcaactgccgtactgtgtttggagttgccaattattcaactgcgaaactatatcaagccatagcagttagcgggatacatgggatccaaacgataaatcaattgaacacatcatttgcagtagatgcatacgggagagctatcggagtgagctgctacgtcactagcgaatctgcagaattaggccatgttaatggtttttttggatcattttgtgaatatgttcccatcttacaggtagtcgtcttggaacagtctcatgatcttgagaggttgattggagatgtttcaatctttcatgacgtagtggatgatccttctgagatagacagttgtgtacgaacgcttttttgggggaaacgccctgtttatatgggcctccgatcgaaggatgccacgaaacttgtccccagtagttctctaaacggaaacatagcagataagatgggcataaagaacactttctttcaaacggacacaataaagagggttatagacaaaatcattgccgaagtttacgcttcatcaaggccattgatcgtggttgatgcattaattgatagatataattacaacagcacaatacaaaatttcctaacggaaacaggaataccatttgtgacaaccttaatgtcaaaaggttctattgacgaaagcttacccaactttgttgggactttcttaggtaccatgtcacagcccattgttcgagaatatatgaataatgcagattgtacgttaattttaggatgtatgatcgaaaacttcaaaaactcgtactgtagattcaactacaagagtaaaaaccaaatcttactttggaatgatagagttaaaattgaaaataatataatacctgatattttacttcatgaactactcccacagctaatagcgtcattagatactaccaaaatagtaaactctcgtccagtaacaataccgaatatgatacccagagtagaaccgcaaccggtgacatttttgcgacaggagtacctttggtttaagatgtctacatggctaaaacaaggtgacgttattatttctgaatctggtacctcagctattggtctcctacaacaaaaattcccagataatactaggctagtatcccaagcaatttggaattcatctggttattctatcggtgcatgccttggaattttggcagcatatcgcgatatggggacattggataagcataggattatattaatggtgggtgatgggtctttacaatttactttccaagaactaagcaccatattgacgcacggtttcaaaccttacatttttgtaataaataatcaaggttataccgttgacagaaccttgaatagagaaaagacccatctgaacgctacatacttcgatatccaaccgtgggaactattaaaactcccgtcacttttctattcccaagagtatttcaagagaagatgtatgtcggttggggaattgaatagtttattaagcgataaagagttcaataaatccgatcagttgaaaattgtagaactaatccttccatccatggatgttccagtactactcgatccacgggatgacagtagtgatgatgaatcttctccgcagcataaaagacccagaacgtgaaaggagaagggtgaaacagatatcagagaactttcacgcttcaaatatatatattatttaataagcactgaacacacaagtacctgcttggtgatcagtctcaatgttaaccgcattaccattaacaacacggcacgcttttcgtatcggcactaacgaagagcacaccaaaagtcacgtgtgcattataccgatattcacgcaaagaacagaatgaagtaaataaatactctattgatgtcaaatacagataatatgagcgaaaaaatatcatcttctcagggtaacactgatcagagatattccagaaccattaccattaagttaattgtcacgtgccggtcagtacgtatcacgtgactgtaacatacatcagccaaccaatcagatcgcttccgttcacagtgacgtccaacctctgacaagaattgcttttccccacgctttaatccccattaagattaccctgcagcaaaacgcacaacgggaaaccaatgagaaaaaagtctcagaggcttcacagaatttggctcatctcgtgtcttgaccgatgttccccaaatgatagacttgtttcgaagcattttttcggtttaagtagttaaaaacaggtaggagaaaaattttagtatgcgcactatggtttgttgataatttcatatataactaacatattaagatttgatattcgattgaatttgatgtttctttagcaagtctttttagcgttttcttttttctcctttctatcttaattaaagtttcccaaactatcagtattaaggattgattcgtcaaatcaaataactgttacacaaccaacacaactaatataaagtcataatg

SEQ ID NO: 30 - protein, 578 aa, Kluyveromyces lactis
METKTLIHSGAAKEMSYTERYNVAPLIPLPEYLFHRLFQLNCRTVFGVANYSTAKLYQAIAVSGIHGIQTINQLNTSFAVDAYGRAIGVSCYVTSESAELGHVNGFFGSFCEYVPILQVVVLEQSHDLERLIGDVSIFHDVVDDPSEIDSCVRTLFWGKRPVYMGLRSKDATKLVPSSSLNGNIADKMGIKNTFFQTDTIKRVIDKIIAEVYASSRPLIVVDALIDRYNYNSTIQNFLTETGIPFVTTLMSKGSIDESLPNFVGTFLGTMSQPIVREYMNNADCTLILGCMIENFKNSYCRFNYKSKNQILLWNDRVKIENNIIPDILLHELLPQLIASLDTTKIVNSRPVTIPNMIPRVEPQPVTFLRQEYLWFKMSTWLKQGDVIISESGTSAIGLLQQKFPDNTRLVSQAIWNSSGYSIGACLGILAAYRDMGTLDKHRIILMVGDGSLQFTFQELSTILTHGFKPYIFVINNQGYTVDRTLNREKTHLNATYFDIQPWELLKLPSLFYSQEYFKRRCMSVGELNSLLSDKEFNKSDQLKIVELILPSMDVPVLLDPRDDSSDDESSPQHKRPRT

SEQ ID NO: 31 - DNA, 1005 bp, Pyrococcus furiosus
  source 1..1005: mol_type = genomic DNA
atgaagatcaaggttggtatcaacggttacggtactattggtaaaagagttgcttacgctgttaccaagcaagatgatatggaattgatcggtgttactaagaccaagccagattttgaagcttacagagctaaagaattgggtattccagtttacgctgcttctgaagaatttttgccaagatttgaaaaggccggtttcgaagttgaaggtactttgaatgatttgttggagaaggttgatatcatcgttgatgctactccaggtggtatgggtgaaaaaaacaagcagttgtacgaaaaggctggtgttaaggctatttttcaaggtggtgaaaaagctgaagttgcccaagtttcttttgttgctcaagctaattatgaagccgccttgggtaaagattacgttagagttgtttcttgtaacaccaccggtttggttagaacattgaacgctattaaggattacgtcgattacgtttacgccgttatgattagaagggctgctgatccaaatgatattaagagaggtcctattaacgccatcaagccatctgttactattccatctcatcatggtccagatgttcaaaccgttattccaatcaacattgaaacctccgctttcgttgttccaactaccattatgcatgttcactccatcatggtggaattgaaaaagccattgaccagagaagatgttatcgacatcttcgaaaacaccaccagagttttgttgttcgaaaaagaaaagggtttcgaatccaccgctcaattgattgaatttgctagagacttgcaccgtgaatggaacaacttatacgaaattgccgtctggaaagagtccattaacgtaaagggtaaccgtttgttctacatccaagctgttcatcaagaatccgatgttatcccagaaaacattgatgctattagggccatgttcgaaattgctgaaaaatgggagtctatcaaaaagaccaacaagtccttgggtatcctgaagtaa

SEQ ID NO: 32 - protein, 334 aa, Pyrococcus furiosus
MKIKVGINGYGTIGKRVAYAVTKQDDMELIGVTKTKPDFEAYRAKELGIPVYAASEEFLPRFEKAGFEVEGTLNDLLEKVDIIVDATPGGMGEKNKQLYEKAGVKAIFQGGEKAEVAQVSFVAQANYEAALGKDYVRVVSCNTTGLVRTLNAIKDYVDYVYAVMIRRAADPNDIKRGPINAIKPSVTIPSHHGPDVQTVIPINIETSAFVVPTTIMHVHSIMVELKKPLTREDVIDIFENTTRVLLFEKEKGFESTAQLIEFARDLHREWNNLYEIAVWKESINVKGNRLFYIQAVHQESDVIPENIDAIRAMFEIAEKWESIKKTNKSLGILK

SEQ ID NO: 33 - DNA, 1692 bp, Saccharomyces cerevisiae
  source 1..1692: mol_type = genomic DNA
atgtctgaaattactttgggtaaatatttgttcgaaagattaaagcaagtcaacgttaacaccgttttcggtttgccaggtgacttcaacttgtccttgttggacaagatctacgaagttgaaggtatgagatgggctggtaacaccaacgaattgaacgctgcttacgccgctgatggttacgctcgtatcaagggtatgtcttgtatcatcaccaccttcggtgtcggtgaattgtctgctttgaacggtattgccggttcttacgctgaacacgtcggtgttttgcacgttgttggtgtcccatccatctctgctcaagctaagcaattgttgttgcaccacaccttgggtaacggtgacttcactgttttccacagaatgtctgccaacatttctgaaaccactgctatgatcactgacattgctaccgccccagctgaaattgacagatgtatcagaaccacttacgtcacccaaagaccagtctacttaggtttgccagctaacttgttcgacttgaacgtcccagctaagttgttgcaaactccaattgacatgtctttgaagccaaacgatgctgaatccgaaaaggaagtcattgacaccatcttggctttggtcaaggatgctaagaacccaattatcttggctgatgcttgttgttccagacacgacgtcaaggctgaaactaagaagttgattgacttgactcaattcccagctttcgtcaccccaatgggtaagggttccattgacgaacaacacccaagatacggtggtgtttacgtcggtaccttgtccaagccagaagttaaggaagccgttgaatctgctgacttgattttgtctgtcggtgctttgttgtctgatttcaacaccggttctttctcttactcttacaagaccaagaacattgtcgaattccactccgaccacatgaagatcagaaacgccactttcccaggtgtccaaatgaaattcgttttgcaaaagttgttgaccactattgctgacgccgctaagggttacaagccagttgctgtcccagctagaactccagctaacgctgctgtcccagcttctaccccattgaagcaagaatggatgtggaaccaattgggtaacttcttgcaagaaggtgatattgtcattgctgaaaccggtacctccgctttcggtatcaaccaaaccactttcccaaacaacacctacggtatctctcaagtcttatggggttccattggtttcaccactggtgctaccttgggtgctgctttcgctgctgaagaaattgatccaaagaagagagttatcttattcattggtgacggttctttgcaattgactgttcaagaaatctccaccatgatcagatggggcttgaagccatacttgttcgtcttgaacaacgatggttacaccattgaaaagttgattcacggtccaaaggctcaatacaacgaaattcaaggttgggaccacctatccttgttgccaactttcggtgctaaggactacgaaacccacagagtcgctaccaccggtgaatgggacaagttgacccaagacaagtctttcaacgacaactctaagatcagaatgattgaaatcatgttgccagtcttcgatgctccacaaaacttggttgaacaagctaagttgactgctgctaccaacgctaagcaataa

SEQ ID NO: 34 - protein, 563 aa, Saccharomyces cerevisiae
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 35 - protein, 563 aa, Saccharomyces cerevisiae
MSEITLGKYLFERLSQVNCNTVFGLPGDFNLSLLDKLYEVKGMRWAGNANELNAAYAADGYARIKGMSCIITTFGVGELSALNGIAGSYAEHVGVLHVVGVPSISSQAKQLLLHHTLGNGDFTVFHRMSANISETTAMITDIANAPAEIDRCIRTTYTTQRPVYLGLPANLVDLNVPAKLLETPIDLSLKPNDAEAEAEVVRTVVELIKDAKNPVILADACASRHDVKAETKKLMDLTQFPVYVTPMGKGAIDEQHPRYGGVYVGTLSRPEVKKAVESADLILSIGALLSDFNTGSFSYSYKTKNIVEFHSDHIKIRNATFPGVQMKFALQKLLDAIPEVVKDYKPVAVPARVPITKSTPANTPMKQEWMWNHLGNFLREGDIVIAETGTSAFGINQTTFPTDVYAIVQVLWGSIGFTVGALLGATMAAEELDPKKRVILFIGDGSLQLTVQEISTMIRWGLKPYIFVLNNNGYTIEKLIHGPHAEYNEIQGWDHLALLPTFGARNYETHRVATTGEWEKLTQDKDFQDNSKIRMIEVMLPVFDAPQNLVKQAQLTAATNAKQ

SEQ ID NO: 36 - protein, 563 aa, Saccharomyces cerevisiae
MSEITLGKYLFERLKQVNVNTIFGLPGDFNLSLLDKIYEVDGLRWAGNANELNAAYAADGYARIKGLSVLVTTFGVGELSALNGIAGSYAEHVGVLHVVGVPSISAQAKQLLLHHTLGNGDFTVFHRMSANISETTSMITDIATAPSEIDRLIRTTFITQRPSYLGLPANLVDLKVPGSLLEKPIDLSLKPNDPEAEKEVIDTVLELIQNSKNPVILSDACASRHNVKKETQKLIDLTQFPAFVTPLGKGSIDEQHPRYGGVYVGTLSKQDVKQAVESADLILSVGALLSDFNTGSFSYSYKTKNVVEFHSDYVKVKNATFLGVQMKFALQNLLKVIPDVVKGYKSVPVPTKTPANKGVPASTPLKQEWLWNELSKFLQEGDVIISETGTSAFGINQTIFPKDAYGISQVLWGSIGFTTGATLGAAFAAEEIDPNKRVILFIGDGSLQLTVQEISTMIRWGLKPYLFVLNNDGYTIEKLIHGPHAEYNEIQTWDHLALLPAFGAKKYENHKIATTGEWDALTTDSEFQKNSVIRLIELKLPVFDAPESLIKQAQLTAATNAKQ

SEQ ID NO: 37 - DNA, 1431 bp, Lactobacillus delbrueckii
  source 1..1431: mol_type = genomic DNA
atgaccgaacactacttgaactacgttaatggtgaatggcgtgattctgctgatgccattgaaatttttgaaccagctactggtaagtccttgggtactgttccagctatgtctcatgaagatgttgattacgttatgaactccgctaaaaaagctttgccagcttggagagctttgtcttatgttgaaagagctgcttacttgcaaaaggctgctgatatcttgtatagagatgccgaaaagattggctccaccttgtctaaagaaattgccaagggtttgaagtcctccattggtgaagttactagaactgctgaaatcgttgagtacactgctaaagttggtgttactttggatggtgaagtaatggaaggtggtaattttgaagctgcctctaaaaacaagttggccgttgttagaagagaaccagttggtttggttttggctatttctccattcaactacccagttaatttggccggttctaaaattgctccagctttgatgggtggtaatgttgttgcttttaaaccaccaactcagggttctatttctggtttgttgttagctaaggcttttgctgaagctggtttgcctgctggtgtttttaacactattactggtagaggtagagtcatcggtgattacatcgttgaacatccagctgttaacttcatcaactttactggttcttctgccgttggtaagaatattggtaaattggctggtatgaggccaatcatgttggaattaggtggtaaagatgctgccatcgttttggaagatgctgatttggatttgaccgctaagaatatcgttgctggtgcttttggttattctggtcaaagatgtactgctgttaagcgtgttttggtcatggattctgttgctgatgaattggttgaaaaggttactgctttggccaaggatttgactgttggtattcctgaagaagatgccgatattactccattgattgataccaagtctgccgattatgttcagggtttgattgaagaggctgcagaaaaaggtgctaaacctttgtttgacttcaagagagaaggcaacttgatctacccaatggttatggatcaagttaccaccgatatgagattggcttgggaagaaccatttggtccagttttgcctttcatcagagttaagtcagctgatgaagctgttatgattgccaacgaatctgaatatggcttgcagtcctctgttttctctagaaattttgaaaaggctttcgccattgccggtaagttggaagttggtacagttcatattaacaacaagacccaaagaggtccagataactttccatttttgggtgttaagtcatctggtgccggtgttcaaggtgtcaaatattctattcaagctatgaccagagtcaagtccgttgttttcaacatcgaagattaa

SEQ ID NO: 38 - protein, 476 aa, Lactobacillus delbrueckii
MTEHYLNYVNGEWRDSADAIEIFEPATGKSLGTVPAMSHEDVDYVMNSAKKALPAWRALSYVERAAYLQKAADILYRDAEKIGSTLSKEIAKGLKSSIGEVTRTAEIVEYTAKVGVTLDGEVMEGGNFEAASKNKLAVVRREPVGLVLAISPFNYPVNLAGSKIAPALMGGNVVAFKPPTQGSISGLLLAKAFAEAGLPAGVFNTITGRGRVIGDYIVEHPAVNFINFTGSSAVGKNIGKLAGMRPIMLELGGKDAAIVLEDADLDLTAKNIVAGAFGYSGQRCTAVKRVLVMDSVADELVEKVTALAKDLTVGIPEEDADITPLIDTKSADYVQGLIEEAAEKGAKPLFDFKREGNLIYPMVMDQVTTDMRLAWEEPFGPVLPFIRVKSADEAVMIANESEYGLQSSVFSRNFEKAFAIAGKLEVGTVHINNKTQRGPDNFPFLGVKSSGAGVQGVKYSIQAMTRVKSVVFNIED

SEQ ID NO: 39 - DNA, 1434 bp, Streptococcus thermophilus
  source 1..1434: mol_type = genomic DNA
atggctaagcagtacaagaactacgttaacggtgaatggaaaacctccgaaaactctattactatctacgctccagctaatggtgaagaattgggttctgttccagctatgtctcaagctgaagttgatgaagtttatgctgctgctaaagctgctttgccagcttggagagctttgtcttatgctgaaagagctgcttacttgcataaggctgctgatattttggaaagagatgccgaaaagatcggtcaggttttgtctaaagaaatctccaagggtttgaagtccgctattggtgaagttgttagaaccgccgaaattattcattacgctgctgaagaaggtttgaggttggaaggtgaagtattagaaggtggtgcttttgatgctggttccaaaaaaaagattgccgtcgttagaagagaaccagttggtttggttttggctatttctccattcaactacccagttaatttggccggttcaaaaattgctccagctttgattgctggtgatgttgttgcttttaaaccaccaactcaaggttccatttctggtttgttgttggttgaagcttttgtcgaagctggtattccagctggtgttttgaattctattactggtagaggttccgttatcggtgattatatcgttgaacacaaggccgttgatttcattaacttcactggttctactccagtcggtgaaaacattggtagattggctgctatgaggccagttatgttggaattaggtggtaaagatgctgccatcgttttggaagatgctgatttggatttgaccgctaagaatatcgttgctggtgctttcgattattctggtcaaagatgtactgccattaagcgtgttttggttatggattctgttgccgatgaattggttgaaaaggttactgctttggttggtaacattactgttggtatgccagaagaatctgcttctgttactccattgattgataccaaagctgccgattttgttcaaggtttgattgatgatgctgttgaacaaggtgctactgctaaaactgaattgaagagagaaggcaacttgatctacccagctgtttttgatcatgttaccaccgatatgagattggcttgggaagaaccatttggtccagttttgccttttatcagagtttcctctgttgaagaagccatcaagatctctaacgaatctgaattcggtttacaaggtgccgttttcactcaagattatccaagagcttttgccattgccgaacaattggaagttggtactgttcacattaacaacaagacccaaagaggtactgataactttccattcttgggtgtaaaaggttctggtgctggtactcaaggtgttaagtattctattgaagctatgaccagagtcaagtccaccgtttttgatatctctgactactaa

SEQ ID NO: 40 - protein, 477 aa, Streptococcus thermophilus
MAKQYKNYVNGEWKTSENSITIYAPANGEELGSVPAMSQAEVDEVYAAAKAALPAWRALSYAERAAYLHKAADILERDAEKIGQVLSKEISKGLKSAIGEVVRTAEIIHYAAEEGLRLEGEVLEGGAFDAGSKKKIAVVRREPVGLVLAISPFNYPVNLAGSKIAPALIAGDVVAFKPPTQGSISGLLLVEAFVEAGIPAGVLNSITGRGSVIGDYIVEHKAVDFINFTGSTPVGENIGRLAAMRPVMLELGGKDAAIVLEDADLDLTAKNIVAGAFDYSGQRCTAIKRVLVMDSVADELVEKVTALVGNITVGMPEESASVTPLIDTKAADFVQGLIDDAVEQGATAKTELKREGNLIYPAVFDHVTTDMRLAWEEPFGPVLPFIRVSSVEEAIKISNESEFGLQGAVFTQDYPRAFAIAEQLEVGTVHINNKTQRGTDNFPFLGVKGSGAGTQGVKYSIEAMTRVKSTVFDISDY

SEQ ID NO: 41 - DNA, 1428 bp, Streptococcus macacae
  source 1..1428: mol_type = genomic DNA
atgaccaagcagtacaaggattacgttaatggtgaatggaagctgtccaagaacgatatcaaaatctatgaaccagcttccggtgctgaattgggtttagttccagctatgtctactgaagaggttgattatgtttatgcttccgctcataaggctttgaaagaatggcgtgctttgtcttatgttgaaagagctgcttacttgcataaggttgccgatattttggaaagagatgccgaaaaaattggtgccgtcttgtctaaagaagttgctaaaggttacaagtccgccgtttctgaagttattagaaccgctgaaattatcaactacgctgctgaagagggtttgagaatggaaggtgaagttttggaaggtggttcttttgaagctgcttccaaaaagaagattgccgttgttagaagagaaccagttggtttggttttggctatttctccattcaactacccagttaatttggccggttctaaaattgctccagctttgattgctggtaacgttgttgcttttaaaccaccaactcaaggttccatttctggtttgttgttggctgaagcttttgctgaagcaggtttgccagctggtgtttttaacactattactggtagaggttccgaaatcggtgattacatcgttgaacatccagctgttaacttcatcaacttcactggttctactccaatcggtgaaagaattggtagaatggctggtatgaggccaatcatgttggaattaggtggtaaagattccgccattgtcttggaagatgctgatttggaattgaccgctaagaatatcgttgctggtgcttttggttattctggtcaaagatgtactgctgttaagcgtgttttagttatggaaggcgttgctgataagttggtcgaaaagattagagaaaaggttttggccttgaccattggtaatccagaaaacgatgctgatattaccccattgattgataccaaggctgctgattttgttgaaggtttgattaacgacgccaaagaaaagggtgctgataacttgactgaaatcaagagagaaggtaacttgatctgcccagttttgttcgataaggttactaccgatatgagattggcttgggaagaaccatttggtccagttttgccaattatcagagttaagtctgtcgaagaagccattgccatctctaatcaatctgaatacggtctgcaagcctctattttcactaatgattttccaagagctttcggtatcgccgaacaattggaagttggtactgttcatttgaacaacaagacccaaagaggtacagataactttccatttttgggcgctaaaaaatcaggtgctggtattcaaggtgtcaagtactctattgaagctatgactaccgtcaagtccgttgttttcgatatcaagtga

SEQ ID NO: 42 - protein, 475 aa, Streptococcus macacae
MTKQYKDYVNGEWKLSKNDIKIYEPASGAELGLVPAMSTEEVDYVYASAHKALKEWRALSYVERAAYLHKVADILERDAEKIGAVLSKEVAKGYKSAVSEVIRTAEIINYAAEEGLRMEGEVLEGGSFEAASKKKIAVVRREPVGLVLAISPFNYPVNLAGSKIAPALIAGNVVAFKPPTQGSISGLLLAEAFAEAGLPAGVFNTITGRGSEIGDYIVEHPAVNFINFTGSTPIGERIGRMAGMRPIMLELGGKDSAIVLEDADLELTAKNIVAGAFGYSGQRCTAVKRVLVMEGVADKLVEKIREKVLALTIGNPENDADITPLIDTKAADFVEGLINDAKEKGADNLTEIKREGNLICPVLFDKVTTDMRLAWEEPFGPVLPIIRVKSVEEAIAISNQSEYGLQASIFTNDFPRAFGIAEQLEVGTVHLNNKTQRGTDNFPFLGAKKSGAGIQGVKYSIEAMTTVKSVVFDIK

SEQ ID NO: 43 - DNA, 1431 bp, Streptococcus hyointestinalis
  source 1..1431: mol_type = genomic DNA
atgaccaaggcttacaagaactacgttaatggtgaatggaagctgtccgaagaatccattgaaatttttgctccagctaccggtgaatctttgggtactgttccagctatgactactgctgaagttgatgaagtttacgctaaagctaaagctgctcaaccagcttggagagctttgtcttatgttgaaagagctgcttacttgcataaggttgccgatattttggttagagatgccgaaaaaattggtgccgtcttgtctaaagaaattgctaagggttacaagtccgccgtttctgaagttattagaaccgctgaaattatcaactacgctgctgaagagggtttgagaatggaaggtgaagttttggaaggtggttcttttgaagctgcttccaaaaacaagattgccatcgttagaagagaaccagttggtttggttttggctatttctccattcaactacccaatcaatttggccggttctaaaattgctcctgctttgatttctggtaacgttgttgctttgaaaccaccaactcaaggttctatttctggtttgttgttggctgaagcttttgctgaagctggtttgccagctggtgtttttaacactattactggtagaggttccgttatcggtgattacatcgttgaacatgaagccgtcaatttcattaacttcactggttctactccaatcggtgaaagaattggtaaattggctggtatgaggccaatcatgttggaattaggtggtaaagattccgccattgtcttggaagatgctgatttggatttgaccgccaagaacattattgctggtgcttttggttattccggtcaaagatgtactgctgttaagagggtattagttatggattccgttgccgatgaattggtcgaaaagattagacaacaagtcttggacttgaccattggtaatcctgaagatgatgctgatattaccccattgattgataagaatgctgccgattttgtcgaaggtttgattaacgatgcttctgataagggtgcagaagctttgactgaaatcaagagagaaggtaacttgatctgcccagttttgttcgataaggttactaccgatatgagattggcttgggaagaaccatttggtccagttttgccaattatcagagttaagtctgttgaagaagccatcgagatctctaacaaatccgaatatggtctgcaagcttctgttttcactaacaattttccactggccttcaagatcgcttctcaattggaagttggtactgtccatattaacaacaagacccaaagaggtactgacaactttccatttttgggtgctaaaaaatcaggtgctggtgttcaaggtgttaagtactctattgaagctatgacctctgtcaagtccgttgtttttgatattgccaagtaa

SEQ ID NO: 44 - protein, 476 aa, Streptococcus hyointestinalis
MTKAYKNYVNGEWKLSEESIEIFAPATGESLGTVPAMTTAEVDEVYAKAKAAQPAWRALSYVERAAYLHKVADILVRDAEKIGAVLSKEIAKGYKSAVSEVIRTAEIINYAAEEGLRMEGEVLEGGSFEAASKNKIAIVRREPVGLVLAISPFNYPINLAGSKIAPALISGNVVALKPPTQGSISGLLLAEAFAEAGLPAGVFNTITGRGSVIGDYIVEHEAVNFINFTGSTPIGERIGKLAGMRPIMLELGGKDSAIVLEDADLDLTAKNIIAGAFGYSGQRCTAVKRVLVMDSVADELVEKIRQQVLDLTIGNPEDDADITPLIDKNAADFVEGLINDASDKGAEALTEIKREGNLICPVLFDKVTTDMRLAWEEPFGPVLPIIRVKSVEEAIEISNKSEYGLQASVFTNNFPLAFKIASQLEVGTVHINNKTQRGTDNFPFLGAKKSGAGVQGVKYSIEAMTSVKSVVFDIAK

SEQ ID NO: 45 - DNA, 1428 bp, Streptococcus urinalis
  source 1..1428: mol_type = genomic DNA
atgaccaagcagtacaagaactacgttaatggtgaatggaagttgtccgaaaacgagattaagatatatgctccagcttccggtgaagaattgggttctgttccagctatgactcaagctgaagttgatgatgtttacgcttctgctaaagctgctttgccagcttggagagctttgtcttatgttgaaagagctaactacttgcataaggccgctgatattttggttagagatgctgaaaagatcggctccgttttgtctcaagaagttgctaaaggtcataagtccgctgtttccgaagttattagaaccgctgaaattatcaactacgctgctgaagagggtttgagaatggaaggtgaagttttggaaggtggttcttttgaagctgcttccaaaaagaagattgccatcgttagaagagaaccagttggtttggttttggctatttctccattcaactacccagttaatttggccggttctaaaattgctccagcattgattgctggtaacgttgttgctttgaaaccaccaactcaaggttccatttctggtattttgttggctcaagcttttgctgaagctggtattccagctggtgttttcaacactattactggtagaggttccgttatcggtgattacatcgttgaacatgaagccgttaacttcatcaatttcactggttctactccagtcggtgaaagaataggtaaattggctggtatgaggccaatcatgttggaattaggtggtaaagattccgccattgtcttggaagatgctgatttggatgttgctgctaagaatatcgttgctggtgcttttggttattctggtcaaagatgtactgctgttaagcgtgttttggtcatggattctgttgctgatgcattggttgaaaaggtgtctcaaaaggtttctgctttgactattggtaaccctgaagatgatgctgatattaccccattgattgataccaaggctgctgattttgttgaaggtttgattaacgacgccaaagaaaaaggtgcacaaccattgcacgaaatcaagagagaaggtaatttggtttgcccattggttttcgataaggttactaccgatatgagattggcttgggaagaaccatttggtccagttttgcctttcatcagagttaagtctgttgaagaagccatcaagatctccaacgaatctgaatatggtctgcaagcttctgttttcactaacaattttccaagagctttcgccattgccgaacaattggaagttggtactgttcacattaacaacaagacccaaagaggtactgataactttccatttttgggcgctaaaaaatctggtgctggtgttcaaggtgttaagtactctattgaagctatgacctctgtcaagtccgttgtttttgatatcgagtaa

SEQ ID NO: 46 - protein, 475 aa, Streptococcus urinalis
MTKQYKNYVNGEWKLSENEIKIYAPASGEELGSVPAMTQAEVDDVYASAKAALPAWRALSYVERANYLHKAADILVRDAEKIGSVLSQEVAKGHKSAVSEVIRTAEIINYAAEEGLRMEGEVLEGGSFEAASKKKIAIVRREPVGLVLAISPFNYPVNLAGSKIAPALIAGNVVALKPPTQGSISGILLAQAFAEAGIPAGVFNTITGRGSVIGDYIVEHEAVNFINFTGSTPVGERIGKLAGMRPIMLELGGKDSAIVLEDADLDVAAKNIVAGAFGYSGQRCTAVKRVLVMDSVADALVEKVSQKVSALTIGNPEDDADITPLIDTKAADFVEGLINDAKEKGAQPLHEIKREGNLVCPLVFDKVTTDMRLAWEEPFGPVLPFIRVKSVEEAIKISNESEYGLQASVFTNNFPRAFAIAEQLEVGTVHINNKTQRGTDNFPFLGAKKSGAGVQGVKYSIEAMTSVKSVVFDIE

SEQ ID NO: 47 - DNA, 1428 bp, Streptococcus canis
  source 1..1428: mol_type = genomic DNA
atgactacccagtacaagaacttggttaatggtgaatggaagttgtccgaaaacgagattaagatatatgctccagctaccggtgaagaattgggttctgttccagctatgtctagagaagaggttgatgctgtttatggtgctgctagacaagctttggctggttggagagctttgtcttatgttgaaagagctgctttcttgcataaggctgctgatattttggttagagatgccgaaaagattggtgccatcttgtctaaagaagttgctaaaggtcacaaagctgccgtttctgaagttattagaaccgccgaaattatcaactatgctgctgaagagggtttgagaatggaaggtgaagttttggaaggtggttcttttgaagctgcttccaaaaaaaagatcgccatcgttagaagagaaccagttggtttggttttggctatttctccattcaattacccagttaatttggccggttcaaaaattgctccagcattgattgctggtaacgttgttgctttgaaaccaccaactcaaggttctatttctggtttgttgttggctgaagcttttgctgaagctggtattccagctggtgttttcaacactattactggtagaggttccgttatcggtgattacatcgttgaacatgaagccgttaacttcatcaacttcactggttctactccaatcggtgaaagaataggtaaattggctggtatgaggccaatcatgttggaattaggtggtaaagattccgccattgtcttggaagatgctgatttggatttggctgctaagaatatcgttgctggtgcttttggttattctggtcaaagatgtactgctgttaagcgtgttttggtcatggaatctgttgctgatgatttggtcgaaaagatcagagataaggtcttgcaattgaccattggtaaccctgaagataacgctgatattacccctttgattgatacttctgctgccgattttgttgagggcttgattaaggatgctgttgataagggtgctactgctcatactgatattaagagagaaggtaacttgatctgcccaatcttgttcgatcatgttactaccgatatgagattggcttgggaagaaccatttggtccagttttgccaattattagagttgcctctgttgaagaagccatcaagatttctaacgaatctgaatacggtctgcaagcctctatttttactaccaattttccacaagctttcggtatcgctgaacaattggaagttggtactgttcacattaacaacaagacccaaagaggtactgataactttccatttttgggcgctaaaaaatctggtgctggtgttcaaggtgttaagtactctattgaagctatgacctccgttaagtccgttgttttcgatatccaatga

SEQ ID NO: 48 - protein, 475 aa, Streptococcus canis
MTTQYKNLVNGEWKLSENEIKIYAPATGEELGSVPAMSREEVDAVYGAARQALAGWRALSYVERAAFLHKAADILVRDAEKIGAILSKEVAKGHKAAVSEVIRTAEIINYAAEEGLRMEGEVLEGGSFEAASKKKIAIVRREPVGLVLAISPFNYPVNLAGSKIAPALIAGNVVALKPPTQGSISGLLLAEAFAEAGIPAGVFNTITGRGSVIGDYIVEHEAVNFINFTGSTPIGERIGKLAGMRPIMLELGGKDSAIVLEDADLDLAAKNIVAGAFGYSGQRCTAVKRVLVMESVADDLVEKIRDKVLQLTIGNPEDNADITPLIDTSAADFVEGLIKDAVDKGATAHTDIKREGNLICPILFDHVTTDMRLAWEEPFGPVLPIIRVASVEEAIKISNESEYGLQASIFTTNFPQAFGIAEQLEVGTVHINNKTQRGTDNFPFLGAKKSGAGVQGVKYSIEAMTSVKSVVFDIQ

SEQ ID NO: 49 - DNA, 1428 bp, Streptococcus thoraltensis
  source 1..1428: mol_type = genomic DNA
atgtccaagcagtacaagaacttggttaatggtgaatggaagttgtccgacaacgaaatcaaaatctatgctccagctactggtgaagaattgggttctgttccagctatgtctcaagaagaggttgattacgtttacgaaactgctaaagctgctcaaccagcttggagagctttgtcttatgttgaaagagctgcttacttgcataaggttgccgatattttggatagagatgccgaaaagattggtgaggtcttgtctaaagaaattgccaaaggttacaaggctgccgtttctgaagttactagaactgctgatattatcagatacgctgctgaagagggtgttagaatgcaaggtgaagttttggaaggtggttcttttgatgctgcctccaaaaaaaagattgccatggttagaagagagccattgggtttagttttggctatttctccattcaactacccagttaatttggccggttctaaaattgctccagcattgatttctggtaacgttgttgctttgaaaccaccaactcaaggttctatttctggtttggttttagctgaagttttcgctgaagctggtattccagctggtgttttttctactattactggtagaggttccgttatcggtgattacatcgttgaacatgaagccgttaacttcatcaacttcactggttctactccagttggtgaaagaataggtaaaatggctggtatgaggccaatcatgttggaattaggtggtaaagattccgccattgtcttggaagatgctgatttggaagttgctgctaagaatatcgttgatggtgcttttggttactctggtcaaagatgtactgctgttaagcgtgttttggttatggattctgttgccgatgaattggtcgaaatgttgagagaaaaggtcttgaagttgactgttggtaaccctgaagataacgcagatattaccccattgattgatactgctgctgccgattttgttgaaggtttagttaatgatgccgttgaaaaaggtgctgatgctaagactgatatcttgagagaaggtaacttgatctacccaatcttgttcgataacgttactaccgatatgaagttggcttgggaagaaccatttggtccagttttgccagttatcagagtttcctctgttgaagaagccatcgaaatctctaacaaatctgaatacggtctgcaagcttccgttttcactaatgattttccattggctttctctatcgccgaacaattagaagttggtactgttcacattaacaacaagacccaaagaggtactgataactttccatttttgggcgctaaaaaatctggtgctggtactcaaggtgttaagtactctattgaagctatgaccaccgttaagtccgttgttttcgatatcaagtaa

SEQ ID NO: 50 - protein, 475 aa, Streptococcus thoraltensis
MSKQYKNLVNGEWKLSDNEIKIYAPATGEELGSVPAMSQEEVDYVYETAKAAQPAWRALSYVERAAYLHKVADILDRDAEKIGEVLSKEIAKGYKAAVSEVTRTADIIRYAAEEGVRMQGEVLEGGSFDAASKKKIAMVRREPLGLVLAISPFNYPVNLAGSKIAPALISGNVVALKPPTQGSISGLVLAEVFAEAGIPAGVFSTITGRGSVIGDYIVEHEAVNFINFTGSTPVGERIGKMAGMRPIMLELGGKDSAIVLEDADLEVAAKNIVDGAFGYSGQRCTAVKRVLVMDSVADELVEMLREKVLKLTVGNPEDNADITPLIDTAAADFVEGLVNDAVEKGADAKTDILREGNLIYPILFDNVTTDMKLAWEEPFGPVLPVIRVSSVEEAIEISNKSEYGLQASVFTNDFPLAFSIAEQLEVGTVHINNKTQRGTDNFPFLGAKKSGAGTQGVKYSIEAMTTVKSVVFDIK

SEQ ID NO: 51 - DNA, 1428 bp, Streptococcus dysgalactiae
  source 1..1428: mol_type = genomic DNA
atgactacccagtacaagaacttggttaatggtgattggaagttgtccgaatccgatattaagatatatgctccagctaccggtgaagaattgggttctgttccagctatgactcaagctgaagttgatgctgtttatgcttctgctaaaaaagctttgccagcttggagagctttgtcttatgttgaaagagctgcttacttgcataaggctgctgatattttggttagagatgccgaaaaaattggtgccgtcttgtctaaagaagttgctaaaggtcacaaagctgccgtttctgaagttattagaaccgccgaaattatcaactatgctgctgaagagggtttgagaatggaaggtgaagttttggaaggtggttcttttgaagctgcttccaaaaaaaagatcgccatcgttagaagagaaccagttggtttggttttggctatttctccattcaattacccagttaacttggccggttctaaaattgctccagcattgattgctggtaacgttgttgctttgaaaccaccaactcaaggttctatttctggtttgttgttggctgaagcttttgctgaagctggtattccagctggtgttttcaacactattactggtagaggttccgttatcggtgattacatcgttgaacatgaagccgttaacttcatcaatttcactggttctactccaatcggtgaaggtattggtaaattggctggtatgaggccaatcatgttggaattaggtggtaaagattccgccattgtcttggaagatgctgatttggctttggctgctaagaatatcgttgctggtgcttttggttattctggtcaaagatgtactgctgttaagcgtgttttggtcatggataaggttgcagatcaattggctgctgaaatcaagactttggtcgaaaaattgtctgtcggtatgcctgaagatgatgcagatattactccattgattgataccaaggctgccgattttgttgaaggtttgattaaggatgctgcagataagggtgctactgctttgactacttttaacagagaaggcaacttgatctccccagttttgtttgatcatgttaccaccgatatgagattggcttgggaagaaccatttggtccagttttgccaattatcagagttacctctgttgaagaagccatcgaaatttctaacgcttccgaatatggtctgcaagcttctattttcactaacaactttccaaaggctttcggtattgccgaacaattagaagttggtactgttcacttgaacaacaagactcaaagaggtacagataacttcccatttttgggtgctaaaaagtctggtgctggtgttcaaggtgttaagtattctattgaagctatgaccaccgttaagtccgttgttttcgatatccaatga

SEQ ID NO: 52 - protein, 475 aa, Streptococcus dysgalactiae
MTTQYKNLVNGDWKLSESDIKIYAPATGEELGSVPAMTQAEVDAVYASAKKALPAWRALSYVERAAYLHKAADILVRDAEKIGAVLSKEVAKGHKAAVSEVIRTAEIINYAAEEGLRMEGEVLEGGSFEAASKKKIAIVRREPVGLVLAISPFNYPVNLAGSKIAPALIAGNVVALKPPTQGSISGLLLAEAFAEAGIPAGVFNTITGRGSVIGDYIVEHEAVNFINFTGSTPIGEGIGKLAGMRPIMLELGGKDSAIVLEDADLALAAKNIVAGAFGYSGQRCTAVKRVLVMDKVADQLAAEIKTLVEKLSVGMPEDDADITPLIDTKAADFVEGLIKDAADKGATALTTFNREGNLISPVLFDHVTTDMRLAWEEPFGPVLPIIRVTSVEEAIEISNASEYGLQASIFTNNFPKAFGIAEQLEVGTVHLNNKTQRGTDNFPFLGAKKSGAGVQGVKYSIEAMTTVKSVVFDIQ

SEQ ID NO: 53 - DNA, 1428 bp, Streptococcus pyogenes
  source 1..1428: mol_type = genomic DNA
atggctaagcagtacaagaacttggttaatggtgaatggaagttgtccgaaaacgaaattactatctatgctccagctaccggtgaagaattgggttctgttccagctatgactcaagctgaagttgatgctgtttatgcttctgctaaaaaagctttgccagcttggagagctttgtcttatgttgaaagagctgcttacttgcataaggctgctgatattttggttagagatgccgaaaaaattggtgccgtcttgtctaaagaagttgctaaaggtcacaaagctgccgtttctgaagttattagaaccgccgaaattatcaactatgctgctgaagagggtttgagaatggaaggtgaagttttggaaggtggttcttttgaagctgcttccaaaaaaaagatcgccatcgttagaagagaaccagttggtttggttttggctatttctccattcaactacccagttaatttggccggttcaaaaattgctccagcattgattgctggtaacgttgttgctttgaaaccaccaactcaaggttctatttctggtttgttgttggctgaagcttttgctgaagctggtattccagctggtgttttcaacactattactggtagaggttccgttatcggtgattacatcgttgaacatgaagccgttaacttcatcaatttcactggttctactccaatcggtgaaggtattggtaaattggctggtatgaggccaatcatgttggaattaggtggtaaagattccgccattgtcttggaagatgctgatttggctttggctgctaagaatatcgttgctggtgcttttggttattctggtcaaagatgtactgctgttaagcgtgttttggtcatggataaggttgcagatcaattggctgctgaaatcaagactttggtcgaaaaattgtctgtcggtatgcctgaagatgatgcagatattactccattgattgataccaaggctgccgattttgttgaaggtttgattaaggatgctaccgataagggtgctactgctttgactgcttttaatagagaaggcaacttgatctccccagttttgtttgatcatgttaccaccgatatgagattggcttgggaagaaccatttggtccagttttgccaattattagagttactactgtcgaagaggccatcaagatttctaacgaatctgaatacggtctgcaagcctctatttttactaccaattttccaaaggctttcggtatcgctgaacaattggaagttggtactgttcacttgaacaacaagactcaaagaggtacagataacttcccatttttgggtgctaaaaagtctggtgctggtgttcaaggtgttaagtattctattgaagctatgaccaccgttaagtccgttgttttcgatatccaatga

SEQ ID NO: 54 - protein, 475 aa, Streptococcus pyogenes
MAKQYKNLVNGEWKLSENEITIYAPATGEELGSVPAMTQAEVDAVYASAKKALPAWRALSYVERAAYLHKAADILVRDAEKIGAVLSKEVAKGHKAAVSEVIRTAEIINYAAEEGLRMEGEVLEGGSFEAASKKKIAIVRREPVGLVLAISPFNYPVNLAGSKIAPALIAGNVVALKPPTQGSISGLLLAEAFAEAGIPAGVFNTITGRGSVIGDYIVEHEAVNFINFTGSTPIGEGIGKLAGMRPIMLELGGKDSAIVLEDADLALAAKNIVAGAFGYSGQRCTAVKRVLVMDKVADQLAAEIKTLVEKLSVGMPEDDADITPLIDTKAADFVEGLIKDATDKGATALTAFNREGNLISPVLFDHVTTDMRLAWEEPFGPVLPIIRVTTVEEAIKISNESEYGLQASIFTTNFPKAFGIAEQLEVGTVHLNNKTQRGTDNFPFLGAKKSGAGVQGVKYSIEAMTTVKSVVFDIQ

SEQ ID NO: 55 - DNA, 1428 bp, Streptococcus ictaluri
  source 1..1428: mol_type = genomic DNA
atgaccaaagagtacaagaacttggttaacggtgaatggaagttgtccgataacaacattactatctacgaaccagctactggtaaagctttgggttctgttccagctatgtctcaagaagaggttgattacgtttacgcttctgctaaacaagctttgccaaaatggcgtgctttgtcttatgttgaaagagctgcttacttgcataaggctgctgatattttggttagagatgccgaaaagattggtgccatcttgtctaaagaagttgctaagggttttaaggctgccgtttctgaagttgttagaaccgctgaaattatcaactatgctgctgaagagggtttgagaatgcaaggtgaagttttggaaggtggttcttttgaagctgcttccaaaaaaaagatcgccatcgttagaagagaaccagttggtttggttttggctatttctccattcaactacccagttaatttggccggttctaaaattgctccagctttgattgctggtaacgttgttgctttgaaaccaccaactcaaggttctatttctggtttgttgttggctgaagcttttgctgaagctggtattccagctggtgttttcaacactattactggtagaggttccgttatcggtgattacatcgttgaacatgaagccgttaacttcatcaacttcactggttctactgctattggtgaaggtattggtaaattggctggtatgaggccaatcatgttggaattaggtggtaaagattccgccattgtcttggaagatgctgatttggttttagctgctaagaacatagtttctggtgcttttggttactctggtcaaagatgtactgccgttaagagaatcttggttatggattctgttgctgatcaattggcctccgaaatcaagattttggtcgaacaattgtccgttggtatccctgaagaagatgcagatattactccattgattgataccaaggccgctgattttgttgaaggtttgattgatgatgctaaggctaaaggtgctttggctttgactgaatgtaagagagataacaacttgatctccccagttttgttcgatagagttactaccgatatgagattggcttgggaagaaccatttggtccagttttgcctttgatcagagtttcctctgttgaagaagccatcgaaatttctaacgcttccgaatatggtctgcaagcttctatttttaccaacaactttccacaagctttcgctatcgctgaacaattggaagttggtactgttcacttgaacaacaagactcaaagaggtacagataacttcccatttttgggtgctaaaaaatctggtgctggtgttcaaggtgttaagtactctattgaagctatgaccaacttgaagtccgttgttttcgatatcaagtaa

SEQ ID NO: 56 - protein, 475 aa, Streptococcus ictaluri
MTKEYKNLVNGEWKLSDNNITIYEPATGKALGSVPAMSQEEVDYVYASAKQALPKWRALSYVERAAYLHKAADILVRDAEKIGAILSKEVAKGFKAAVSEVVRTAEIINYAAEEGLRMQGEVLEGGSFEAASKKKIAIVRREPVGLVLAISPFNYPVNLAGSKIAPALIAGNVVALKPPTQGSISGLLLAEAFAEAGIPAGVFNTITGRGSVIGDYIVEHEAVNFINFTGSTAIGEGIGKLAGMRPIMLELGGKDSAIVLEDADLVLAAKNIVSGAFGYSGQRCTAVKRILVMDSVADQLASEIKILVEQLSVGIPEEDADITPLIDTKAADFVEGLIDDAKAKGALALTECKRDNNLISPVLFDRVTTDMRLAWEEPFGPVLPLIRVSSVEEAIEISNASEYGLQASIFTNNFPQAFAIAEQLEVGTVHLNNKTQRGTDNFPFLGAKKSGAGVQGVKYSIEAMTNLKSVVFDIK

SEQ ID NO: 57 - DNA, 1461 bp, Clostridium perfringens
  source 1..1461: mol_type = genomic DNA
atgttctcctgcatcaagggtaaagaaagaaccttcagaaacttgatcaacggtgaatggatcaactcctcatccgataagttcatcgatatctattctccagttggtaactgcttggttggtaaagttccagctatgactactgatgaagttgatttggctatcaagtccgctaaagaagctcaaaaggtttggagaaatgttccagttaacaagagagccgagatcttgtacaaagctgccgatattttgatcgaaaaggttgaagatatcgccgagatcatgatgagagaaattggtaaggataagaagtccgccgaatccgaaattttgagatctgctgattacattaagttcactgctgataccgctaagaacttgtctggtgaatctattccaggtgattcatttccaggtttcaagaggaacaagatttccttggttactagagaaccattgggtgttgttttggcaatttctccattcaactacccaatcaatttggccgcttctaaaattgctccagctttggttgctggtaattccgttgttttgaaaccagctactcaaggttcattgtgtggtctgtatttggctaaggtttttgaacaagctggtgttcctgctggtgttttgaatactgttactggtagaggttccgaaatcggtgattatatcgttacccatccagagatcgatttcattaactttactggttctaccgaagttggcaccagaatttctagaattactaccatggttcccttgttgatggaattaggtggtaaagatgctgctatcgttttggctgatgctgatttggatttggctgcttcaaatatcgttgctggtgcttattcttactctggtcaaagatgtactgccgtcaagagaattttggttgttaatgaagttgccgacaagttggtggaaaaggtcaaagaaaaggtccagaacttgaagattggtaacccattggaagaagatgttgatatcgttccattgatcgattctaaggctgctgattttgtttgggaattgattgatgatgccagagaaaaaggtgcccatttgttggttggtggtacaagagaagaaaacatgatctacccaactttgttcgataacgttaccaccgatatgagattggcttgggaagaaccatttggtccagttttgccaattatcagagttaaggacaaggatgaagccattgaaatcgctaacaaatctgaatacggcctgcaatcttctgttttcaccgaaaacattaacgaagctttctacgttgccgatagattggaagttggtactgttcaagtcaacaacaagactgaaagaggtcctgatcattttccattcttgggtgttaaggcttctggtattggtacacaaggtatcagatactccatcgaatctatgtctagaccaaaggctaccgttatcaacttggttagatga

SEQ ID NO: 58 - protein, 486 aa, Clostridium perfringens
MFSCIKGKERTFRNLINGEWINSSSDKFIDIYSPVGNCLVGKVPAMTTDEVDLAIKSAKEAQKVWRNVPVNKRAEILYKAADILIEKVEDIAEIMMREIGKDKKSAESEILRSADYIKFTADTAKNLSGESIPGDSFPGFKRNKISLVTREPLGVVLAISPFNYPINLAASKIAPALVAGNSVVLKPATQGSLCGLYLAKVFEQAGVPAGVLNTVTGRGSEIGDYIVTHPEIDFINFTGSTEVGTRISRITTMVPLLMELGGKDAAIVLADADLDLAASNIVAGAYSYSGQRCTAVKRILVVNEVADKLVEKVKEKVQNLKIGNPLEEDVDIVPLIDSKAADFVWELIDDAREKGAHLLVGGTREENMIYPTLFDNVTTDMRLAWEEPFGPVLPIIRVKDKDEAIEIANKSEYGLQSSVFTENINEAFYVADRLEVGTVQVNNKTERGPDHFPFLGVKASGIGTQGIRYSIESMSRPKATVINLVR

SEQ ID NO: 59 - DNA, 1461 bp, Clostridium chromiireducens
  source 1..1461: mol_type = genomic DNA
atgttcaactgcatcaagtgcgagaacaacaacttcaagaacttgattaacggtgaatgggttggtaacaaggataacaaggttatcgaaatctactccccattggacaattctttggttggtactgttccagctatgacccaagaagatattgatcacgttattcaagttgccaaggacggtcaaagagaatggtctaaagttccaatgaacgaaagagccgagatcttgtacaaagctgctgatattttggttgaaaacgccaacgaattggtcgacattatgattagagaaatcggcaaggaccgtaagagttccaaatctgaaattcatagaaccgccgacttcattagattcactgctgatactgctaagaacatggctggtgaatctattccaggtgatacttttccaggtttcaagaggaacaagatttccgttgttaacagagaaccattgggtgttgttttggctatttctccattcaactaccccattaacttgtccgcttctaaaattgctccagccattatcgttggtaactccgttgttttgaaaccagctactcaaggttctttgtgtggtctgtatttggctaaggttttccaagaggctggtgttccaaatggtgttttgaacactattactggtaagggttccgaaattggtgattatgctgttactcataagggcgtcaacttcattaactttactggttctactgaggtcggtgtcaagatttctaagattacttctatggtccccttgttgatggaattaggtggtaaagatgctgccatcgttttgaaagatgccgatttggatttggctgctaacaatatcgttgctggtggttattcttactctggtcaaagatgtactgccgtcaagagaattttggttttggaaaaggttgccgatgagttggtcaaaaaggtcaaagaaaagatgtccaacttgactgttggtaacccattggataaggatgttgatatcgttccattgatctctaccaagtctgctgatttcgttgaagagttgattaaggatgccattgataagggtgcagatttggttgttggtggtaaaagagatggtaacttgatctacccaaccttgttcgataatgttaccggtgatatgagaattgcttgggaagaaccatttggtccagttttgccaattatgagagttaaggacaaggatgaagccatcgaaattgctaacaagtccgaatatggtttacaaggtgctgttttcaccgaaaacattgaagatgctttctacgttgccgatagattggaagttggtacagttcaagttaacaacaagactgaaagaggtccagatcattttccattcttgggtgttaaggcttctggtattggtacacaaggtatcagatactccatcgaatctatgtctagaccaaaggctaccgttatcaacttggttagatga

SEQ ID NO: 60 - protein, 486 aa, Clostridium chromiireducens
MFNCIKCENNNFKNLINGEWVGNKDNKVIEIYSPLDNSLVGTVPAMTQEDIDHVIQVAKDGQREWSKVPMNERAEILYKAADILVENANELVDIMIREIGKDRKSSKSEIHRTADFIRFTADTAKNMAGESIPGDTFPGFKRNKISVVNREPLGVVLAISPFNYPINLSASKIAPAIIVGNSVVLKPATQGSLCGLYLAKVFQEAGVPNGVLNTITGKGSEIGDYAVTHKGVNFINFTGSTEVGVKISKITSMVPLLMELGGKDAAIVLKDADLDLAANNIVAGGYSYSGQRCTAVKRILVLEKVADELVKKVKEKMSNLTVGNPLDKDVDIVPLISTKSADFVEELIKDAIDKGADLVVGGKRDGNLIYPTLFDNVTGDMRIAWEEPFGPVLPIMRVKDKDEAIEIANKSEYGLQGAVFTENIEDAFYVADRLEVGTVQVNNKTERGPDHFPFLGVKASGIGTQGIRYSIESMSRPKATVINLVR

SEQ ID NO: 61 - DNA, 1461 bp, Clostridium botulinum
  source 1..1461: mol_type = genomic DNA
atgttcaaccacatcaaggacgaaaacaacaccttcaagaacttgattaacggtgagtgggtttcctctagatctttcgttgaaatcaagtcccctctgtctaattctttgttgggtagagttccagctatgaccaaagaagaagttgatattgctgttcagaccgctaaagaagctcaaaaaaagtggaacaagatcaccattaacgaaagggctgagatcttgtacaaagcctctgatattttgttggagaacatcgacgaactgtccgaattgatgatgatggaaattgccaaggatagaaagtcctgcagatctgaagtttctagaacctccgatttcattaagttcactgctgatactgccaagaatttgtccggtgaatctattccaggtgattctttcccaggtttcaaaaacaacaaggtgtccattgtcaaaagggaaccattgggtgttgtattggctatttctccattcaactaccccattaacttgtccgcttctaaaattgctccaggtttgatggctggtaactctgttgttttgaagccagctactcaaggttctttgtgtggtctatatttggccagaatttttgaaaaggctggtgttccagctggtgttttgaacactattactggtaagggttctgaaatcggtgattacattactacccataagggcattaacttcatcaacttcactggttctactgaagttggtgctagaatttctaagatgacctctatggttcccctgttgatggaattaggtggtaaagatgctgctatcgttttggaagatgctgatttggaattgactgcctctaatatcgttgctggtggttattcttattccggtcaaagatgtactgccgtcaagagaattttggttgttgataaggttgccgacaagctgttggaaaagatcaaagaaaagatgaagaaactgaccgtcggtaacccattggaaaaagatgttgatatcgtccccttgatttcttctaaggctgctgatttcgttatcgaattgattgaagatgccaagtccaaaggtgcagatttgatagttggtggtaatagagaaggcaacttgatctatccaaccttgtttgataacgttaccaccgatatgagattggcttgggaagaaccatttggtccagttttgccaattatcagagttaaggataaggacgaagccattgaaatcgctaacaaatccgaatatggtctgcaatctgctgttttcaccaagaacattaacgatgctttttacgtcgccgataagttggaagttggtactgttcaaatcaacaacaagactgaaagaggtccagataactttccttttatgggtgtaaaagcttccggtattggtacacaaggtatcaagtactccatcgaatctatgtctagaccaaaggccaccattatcaacttgtccattcataactaa

SEQ ID NO: 62 - protein, 486 aa, Clostridium botulinum
MFNHIKDENNTFKNLINGEWVSSRSFVEIKSPLSNSLLGRVPAMTKEEVDIAVQTAKEAQKKWNKITINERAEILYKASDILLENIDELSELMMMEIAKDRKSCRSEVSRTSDFIKFTADTAKNLSGESIPGDSFPGFKNNKVSIVKREPLGVVLAISPFNYPINLSASKIAPGLMAGNSVVLKPATQGSLCGLYLARIFEKAGVPAGVLNTITGKGSEIGDYITTHKGINFINFTGSTEVGARISKMTSMVPLLMELGGKDAAIVLEDADLELTASNIVAGGYSYSGQRCTAVKRILVVDKVADKLLEKIKEKMKKLTVGNPLEKDVDIVPLISSKAADFVIELIEDAKSKGADLIVGGNREGNLIYPTLFDNVTTDMRLAWEEPFGPVLPIIRVKDKDEAIEIANKSEYGLQSAVFTKNINDAFYVADKLEVGTVQINNKTERGPDNFPFMGVKASGIGTQGIKYSIESMSRPKATIINLSIHN

SEQ ID NO: 63 - DNA, 1440 bp, Bacillus cereus
  source 1..1440: mol_type = genomic DNA
atgactacctctaacacctacaagttctacttgaatggtgaatggcgtgaatcttcttctggtgaaactattgaaatcccctctccatacttgcatgaagttattggtcaagttcaagccattaccagaggtgaagttgatgaagctattgcttctgctaaagaagctcaaaaatcttgggcagaagcttccttgcaagatagagctaaatacttgtacaaatgggccgatgaattggtcaatatgcaagacgaaattgccgacatcatcatgaaggaagttggtaaaggttacaaggacgccaagaaagaagttgttagaaccgctgatttcatcaggtacactattgaagaggctttacacatgcatggtgaatctatgatgggtgattcttttccaggtggtactaagtctaagttggccattattcaaagggctccattgggtgttgttttggctattgctccattcaattacccagttaatttgtccgctgctaaattggctccagctttgattatgggtaacgccgttattttcaaaccagctactcaaggtgctatctccggtattaagatggttgaagccttgcataaggctggtttgccaaaaggtttggttaatgttgctactggtagaggttctgttatcggtgattatttggttgaacacgagggtatcaacatggtttctttcactggtggtacaaacaccggtaaacatttggctaaaaaggctgctatgatcccattggttttggaattaggtggtaaagatccaggtatcgttagagaagatgctgacttacaagatgctgctaaccatatagtttctggtgctttttcttactccggtcaaagatgtactgctatcaaaagagttttggtccacgaaaacgttgctgacgaattggttggtttgttgcaagaacaagttgccaaattgtctgttggttctcctgaacaagattctactatcgttccattgatcgatgataagtctgccgattttgttcaaggcttggttgatgatgctgttgaaaaaggtgctaccatcgttattggtaacaagagggaaagaaacttgatctacccaaccttgattgatcacgttaccgaagatatgaaggttgcttgggaagaaccatttggtccaattttgccaattatcagagtctcctctgatgaacaagccattgaaattgctaacaagtctgaattcggtctgcaagcttctgttttcaccaaggatattaacaaggctttcgctattgccaacaagattgaaactggttccgttcaaatcaacggtagaactgaaagaggtccagatcattttcctttcattggtgtaaaaggttctggtatgggtgctcaaggtattagaaaatccttggaatccatgaccagagaaaaggttactgttttgaacctggtctaa

SEQ ID NO: 64 - protein, 479 aa, Bacillus cereus
MTTSNTYKFYLNGEWRESSSGETIEIPSPYLHEVIGQVQAITRGEVDEAIASAKEAQKSWAEASLQDRAKYLYKWADELVNMQDEIADIIMKEVGKGYKDAKKEVVRTADFIRYTIEEALHMHGESMMGDSFPGGTKSKLAIIQRAPLGVVLAIAPFNYPVNLSAAKLAPALIMGNAVIFKPATQGAISGIKMVEALHKAGLPKGLVNVATGRGSVIGDYLVEHEGINMVSFTGGTNTGKHLAKKAAMIPLVLELGGKDPGIVREDADLQDAANHIVSGAFSYSGQRCTAIKRVLVHENVADELVGLLQEQVAKLSVGSPEQDSTIVPLIDDKSADFVQGLVDDAVEKGATIVIGNKRERNLIYPTLIDHVTEDMKVAWEEPFGPILPIIRVSSDEQAIEIANKSEFGLQASVFTKDINKAFAIANKIETGSVQINGRTERGPDHFPFIGVKGSGMGAQGIRKSLESMTREKVTVLNLV

SEQ ID NO: 65 - DNA, 1440 bp, Bacillus anthracis
  source 1..1440: mol_type = genomic DNA
atgactacctctaacacctacaagttctacttgaatggtgaatggcgtgaatcttcttctggtgaaactattgaaatcccctctccatacttgcatgaagttattggtcaagttcaagccattaccagaggtgaagttgatgaagctattgcttctgctaaagaagctcaaaaatcttgggcagaagcttccttgcaagatagagctaaatacttgtacaaatgggccgatgaattggtcaatatgcaagacgaaattgccgacatcatcatgaaggaagttggtaaaggttacaaggacgccaagaaagaagttgttagaaccgctgatttcatcaggtacactattgaagaggctttacacatgcatggtgaatctatgatgggtgattcttttccaggtggtactaagtctaagttggccattattcaaagggctccattgggtgttgttttggctattgctccattcaattacccagttaatttgtccgctgctaaattggctccagctttgattatgggtaacgccgttattttcaaaccagctactcaaggtgctatctccggtattaagatggttgaagccttgcataaggctggtttgccaaaaggtttggttaatgttgctactggtagaggttctgttatcggtgattatttggttgaacacgagggtatcaacatggtttctttcactggtggtacaaacaccggtaaacatttggctaaaaaggctgctatgatcccattggttttggaattaggtggtaaagatccaggtatcgttagagaagatgctgacttacaagatgctgctaaccatatagtttctggtgctttttcttactccggtcaaagatgtactgctatcaaaagagttttggtccacgaaaacgttgctgacgaattggttggtttgttgaaagaacaagttgccaagttgtctgttggttctcctgaacaagattctactatcgttccattgatcgatgataagtctgccgattttgttcaaggcttggttgatgatgctgttgaaaaaggtgctaccatcgttattggtaacaacagggaaagaaacttgatctacccaaccttgattgatcacgttaccgaagaaatgaaggttgcttgggaagaaccatttggtccaattttgccaattatcagagtctcctctgatgaacaagccattgaaattgctaacaagtctgaattcggtctgcaagcttctgttttcaccaaggatattaacaaggctttcgctattgccaacaagattgaaactggttccgttcaaatcaacggtagaactgaaagaggtccagatcattttcctttcattggtgtaaaaggttctggtatgggtgctcaaggtattagaaaatccttggaatccatgaccagagaaaaggttactgttttgaacctggtctaa

SEQ ID NO: 66 - protein, 479 aa, Bacillus anthracis
MTTSNTYKFYLNGEWRESSSGETIEIPSPYLHEVIGQVQAITRGEVDEAIASAKEAQKSWAEASLQDRAKYLYKWADELVNMQDEIADIIMKEVGKGYKDAKKEVVRTADFIRYTIEEALHMHGESMMGDSFPGGTKSKLAIIQRAPLGVVLAIAPFNYPVNLSAAKLAPALIMGNAVIFKPATQGAISGIKMVEALHKAGLPKGLVNVATGRGSVIGDYLVEHEGINMVSFTGGTNTGKHLAKKAAMIPLVLELGGKDPGIVREDADLQDAANHIVSGAFSYSGQRCTAIKRVLVHENVADELVGLLKEQVAKLSVGSPEQDSTIVPLIDDKSADFVQGLVDDAVEKGATIVIGNNRERNLIYPTLIDHVTEEMKVAWEEPFGPILPIIRVSSDEQAIEIANKSEFGLQASVFTKDINKAFAIANKIETGSVQINGRTERGPDHFPFIGVKGSGMGAQGIRKSLESMTREKVTVLNLV

SEQ ID NO: 67 - DNA, 1440 bp, Bacillus thuringiensis
  source 1..1440: mol_type = genomic DNA
atgactacctctaacacctacaagttctacttgaatggtgaatggcgtgaatcttcttctggtgaaactattgaaatcccctctccatacttgcatgaagttattggtcaagttcaagccattaccagaggtgaagttgatgaagctattgcttctgctaaagaagctcaaaaatcttgggcagaagcttccttgcaagatagagctaaatacttgtacaaatgggccgatgaattggtcaatatgcaagacgaaattgccaacatcatcatgaaggaagttggtaagggttacaaggatgccaagaaagaagttgttagaaccgccgatttcatcagatacactattgaagaggctttacacatgcacggtgaatctatgatgggtgattcttttccaggtggtactaagtctaagttggccattattcaaagggctccattgggtgttgttttggctattgctccattcaattacccagttaatttgtccgctgctaaattggctccagctttgattatgggtaacgccgttattttcaaaccagctactcaaggtgctatctccggtattaagatggttgaagccttgcataaggctggtttgccaaaaggtttggttaatgttgctactggtagaggttctgttatcggtgattatttggttgaacacgagggtatcaacatggtttctttcactggtggtacaaacaccggtaaacatttggctaaaaaggctgctatgatcccattggttttggaattaggtggtaaagatccaggtatcgttagagaagatgctgacttacaagatgctgctaaccatatagtttctggtgctttttcttactccggtcaaagatgtactgctatcaaaagagttttggtccacgaaaacgttgctgacgaattggttggtttgttgaaagaacaagttgccaagttgtctgttggttctcctgaacaagattctactatcgttccattgatcgatgataagtccgctgattttgttcaaggcttggttgatgatgctgttgaaaaaggtgctaccatcgttattggtaacaagagggaaagaaacttgatctacccaaccttgattgatcacgttaccgaagaaatgaaggttgcttgggaagaaccatttggtccaattttgccaattatcagagtctcctctgatgaacaagccattgaaattgctaacaagtctgaattcggtctgcaagcttctgttttcaccaaggatattaacaaggctttcgctatcgccaacaagattgaaactggttctgttcaaatcaacggtagaactgaaagaggtccagatcattttcctttcattggtgtaaaaggttctggtatgggtgctcaaggtattagaaaatccttggaatccatgaccagagaaaaggttactgttttgaacctggtctaa

SEQ ID NO: 68 - protein, 479 aa, Bacillus thuringiensis
MTTSNTYKFYLNGEWRESSSGETIEIPSPYLHEVIGQVQAITRGEVDEAIASAKEAQKSWAEASLQDRAKYLYKWADELVNMQDEIANIIMKEVGKGYKDAKKEVVRTADFIRYTIEEALHMHGESMMGDSFPGGTKSKLAIIQRAPLGVVLAIAPFNYPVNLSAAKLAPALIMGNAVIFKPATQGAISGIKMVEALHKAGLPKGLVNVATGRGSVIGDYLVEHEGINMVSFTGGTNTGKHLAKKAAMIPLVLELGGKDPGIVREDADLQDAANHIVSGAFSYSGQRCTAIKRVLVHENVADELVGLLKEQVAKLSVGSPEQDSTIVPLIDDKSADFVQGLVDDAVEKGATIVIGNKRERNLIYPTLIDHVTEEMKVAWEEPFGPILPIIRVSSDEQAIEIANKSEFGLQASVFTKDINKAFAIANKIETGSVQINGRTERGPDHFPFIGVKGSGMGAQGIRKSLESMTREKVTVLNLV

SEQ ID NO: 69 - protein, 558 aa, Gluconacetobacter diazotrophicus
MTYTVGRYLADRLAQIGLKHHFAVAGDYNLVLLDQLLLNTDMQQIYCSNELNCGFSAEGYARANGAAAAIVTFSVGALSAFNALGGAYAENLPVILISGAPNANDHGTGHILHHTLGTTDYGYQLEMARHITCAAESIVAAEDAPAKIDHVIRTALREKKPAYLEIACNVAGAPCVRPGGIDALLSPPAPDEASLKAAVDAALAFIEQRGSVTMLVGSRIRAAGAQAQAVALADALGCAVTTMAAAKSFFPEDHPGYRGHYWGEVSSPGAQQAVEGADGVICLAPVFNDYATVGWSAWPKGDNVMLVERHAVTVGGVAYAGIDMRDFLTRLAAHTVRRDATARGGAYVTPQTPAAAPTAPLNNAEMARQIGALLTPRTTLTAETGDSWFNAVRMKLPHGARVELEMQWGHIGWSVPAAFGNALAAPERQHVLMVGDGSFQLTAQEVAQMIRHDLPVIIFLINNHGYTIEVMIHDGPYNNVKNWDYAGLMEVFNAGEGNGLGLRARTGGELAAAIEQARANRNGPTLIECTLDRDDCTQELVTWGKRVAAANARPPRAG

SEQ ID NO: 70 - DNA, 1707 bp, synthetic construct; identical to SEQ ID NO: 11
  source 1..1707: mol_type = other DNA

SEQ ID NO: 71 - DNA, 1710 bp, synthetic construct; identical to SEQ ID NO: 7
  source 1..1710: mol_type = other DNA

SEQ ID NO: 72 - DNA, 1071 bp, Sporotrichum pulverulentum
  source 1..1071: mol_type = genomic DNA
atgttggcctacagattcattccaggtgaacctaatccagttccacaacaagttccaattccaactccagctgctgaagaagttttggttaaggttttggctgctggtgtttgtcatactgatgttggtattttgactccaggtgacatgttgaatttgtggtgtgcttctaagtctttcaccttgggtcatgaaggtgctggtattattgctgaaattccagcttctgttgcttctgcttttccaaatttgaaggttggtgattacgttgctatgtggtgtggtgatgcttgtaaaaagccagcttgttctgtttgtgcttacggttttactgatttgtgcaccttcgatagattgcatggtgttggtttggatggtacttgggctgagtatattactttacatgcttcttgcgttgttccagttcctgcttctccagaaagaattccaccagctgttatttctgctgctactgatgctactttgtctccatatcatgctatgaaaacctgttgtggtgttagaccagaacataccgttttgtgtatgggtactggtggtttgggtttgaatggtgttgctattgctaagaagtgtttgagagctagatgtgttattgcttgcgatacaagacaatccgctagagaagatgctttagcagctggtgctgattatgctgttggtccagaagaattggctggtttggttgaagaaaagaagttggttgttgatttcgccttcgatttcgttggtattcaagctactttcgattcttgcttcgctgctattagaccaggtggtactattcatgttttaggtttgggtgctaacgcccaacaatatcaacaattggttgccatgtctaagaacctgactttgaaaacatctttctggggtgctagatctgatttggctgaagtattgcaagctattgcagaaggtacattgcaacctaaagttgataccagaccaatgtccgaagttgttgaagttttagacgaaatgagagccggtagattacaagctagagttgctttgattccagaaggtgcagcttcagcttctgcataa

SEQ ID NO: 73 - protein, 356 aa, Sporotrichum pulverulentum
MLAYRFIPGEPNPVPQQVPIPTPAAEEVLVKVLAAGVCHTDVGILTPGDMLNLWCASKSFTLGHEGAGIIAEIPASVASAFPNLKVGDYVAMWCGDACKKPACSVCAYGFTDLCTFDRLHGVGLDGTWAEYITLHASCVVPVPASPERIPPAVISAATDATLSPYHAMKTCCGVRPEHTVLCMGTGGLGLNGVAIAKKCLRARCVIACDTRQSAREDALAAGADYAVGPEELAGLVEEKKLVVDFAFDFVGIQATFDSCFAAIRPGGTIHVLGLGANAQQYQQLVAMSKNLTLKTSFWGARSDLAEVLQAIAEGTLQPKVDTRPMSEVVEVLDEMRAGRLQARVALIPEGAASASA

SEQ ID NO: 74 - DNA, 1053 bp, Aspergillus nidulans
  source 1..1053: mol_type = genomic DNA
atgtccattccaactatgcaatgggctcaagttgctgaaaaagttggtggtccattggtttacaagcaaattccagttccaaaaccaggtccagatcaaattttggtcaagatcagatactctggtgtttgtcatactgacttgcatgctatgatgggtcattggcctattccagttaagatgccattggtcggtggtcatgaaggtgctggtatagttgttgctaaaggtgaattggttcacgaattcgaaattggtgatcaagctggtattaagtggttgaatggttcttgtggtgaatgcgaattctgcagacaatctgatgatccattgtgtgctagagcacaattgtctggttatactgttgatggtactttccaacaatacgctttgggtaaagcttctcatgcttctaaaattccagctggtgttccattagatgctgctgctccagttttgtgtgcaggtattactgtttacaagggtttgaaagaagctggcgttagaccaggtcaaactgttgctatagttggtgctggtggtggtttgggttctttggctcaacaatatgctaaagccatgggtattagagttgttgcagttgatggtggtgatgagaaaagggctatgtgtgaatctttgggtactgaaacttacgttgacttcaccaagtctaaggatttggttgctgatgttaaggctgctactccagatggtttaggtgctcatgctgttattttgttggctgtttctgaaaagccatttcaacaagctaccgaatacgttagatctagaggtactatagttgctattggtttgccaccagatgcttatttgaaagccccagttattaacaccgttgtcagaatgattaccatcaagggttcttacgttggtaacagacaagatggtgttgaagctttggatttctttgctagaggtttgattaaggctccattcaaaactgctccattgaaggatttgccaaagatctacgaattgatggaacaaggtagaattgccggtagatacgttttggaaatgccagaatga

SEQ ID NO: 75 - protein, 350 aa, Aspergillus nidulans
MSIPTMQWAQVAEKVGGPLVYKQIPVPKPGPDQILVKIRYSGVCHTDLHAMMGHWPIPVKMPLVGGHEGAGIVVAKGELVHEFEIGDQAGIKWLNGSCGECEFCRQSDDPLCARAQLSGYTVDGTFQQYALGKASHASKIPAGVPLDAAAPVLCAGITVYKGLKEAGVRPGQTVAIVGAGGGLGSLAQQYAKAMGIRVVAVDGGDEKRAMCESLGTETYVDFTKSKDLVADVKAATPDGLGAHAVILLAVSEKPFQQATEYVRSRGTIVAIGLPPDAYLKAPVINTVVRMITIKGSYVGNRQDGVEALDFFARGLIKAPFKTAPLKDLPKIYELMEQGRIAGRYVLEMPE

SEQ ID NO: 76 - DNA, 1068 bp, Natronomonas pharaonis
  source 1..1068: mol_type = genomic DNA
atgagagccgttgtttttgaagaggctggtgaacctatggaagttcaagaagttgatagacctgaagctgatgctgacgaaatagttgttgaaactgaagcttgtggtgtctgtagatctgattggcatgcttggcaaggtgattgggattggattggtgttgctccaactccaggtttggtttttggtcatgaaccagttggtactgtcaaagaagttggagaggacgtttctgaattgtctgttggtgatagagtttgcgttccattcaatttgtctgatggtacttgtagatactgcaagcaaggtagagctaacatttgtgaaagggttgttccaatgggttttgttccatttcaaccaggtgcttttgctgaagaatttccagttagaaacgctgatcataacgccattaagattccagatggtgttgatccagttgatgttgctggtttgggttgtagatttgctactgcttttcatggtttggttcacagagttgatgtcacaccaggtgactgggttgcagttcatggttgcggtggtgttggtttgtctgctattcatgttgctgatgctttgggtgctaatgttataggtgttgacttgggtgaagataagttggataaggctgttgaattaggtgctgatgccactgttaatgttaccgaagttgaagatgttccacaagctgttaaggctcatactccatcttctagaggtgttgaagtttctgttgatgcattaggtgttgctgaaacctgtagaaactctgttaactctttggctaaaggtggtcaaaacttgcaaattggtttgactacttctgaagaggaaggtgaagtttcattgccagttgataacatcgtcatggaagaaagagaattcgttggttcttttggtatgccaccacatgaatacgaagagatcttttctatgatggcccaagataagattgacccaggtaaaattgtctccgaaaccgtttctttggaagaagttcctgaagttattgcctccatggatgattatggtactgttggtattccagtttgcgacgaattctga

SEQ ID NO: 77 - protein, 355 aa, Natronomonas pharaonis
MRAVVFEEAGEPMEVQEVDRPEADADEIVVETEACGVCRSDWHAWQGDWDWIGVAPTPGLVFGHEPVGTVKEVGEDVSELSVGDRVCVPFNLSDGTCRYCKQGRANICERVVPMGFVPFQPGAFAEEFPVRNADHNAIKIPDGVDPVDVAGLGCRFATAFHGLVHRVDVTPGDWVAVHGCGGVGLSAIHVADALGANVIGVDLGEDKLDKAVELGADATVNVTEVEDVPQAVKAHTPSSRGVEVSVDALGVAETCRNSVNSLAKGGQNLQIGLTTSEEEGEVSLPVDNIVMEEREFVGSFGMPPHEYEEIFSMMAQDKIDPGKIVSETVSLEEVPEVIASMDDYGTVGIPVCDEF

SEQ ID NO: 78 - DNA, 1128 bp, Homo sapiens
  source 1..1128: mol_type = genomic DNA
atgtctactgctggtaaggttattaagtgtaaggctgctgttttgtgggaagttaagaagccattctccatcgaagatgttgaagttgctccaccaaaagcttacgaagttagaatcaaaatggttgccgttggtatctgtagaaccgatgatcatgttgtttctggtaacttggttactccattgccagttattttgggtcatgaagctgctggtatagttgaatctgttggtgaaggtgttactactgttaagccaggtgacaaagttattcctttgttcactccacaatgtggtaagtgtagagtctgtaagaatccagaatctaactactgcttgaagaacgatttgggtaatccaagaggtacattgcaagatggtactagaagattcacttgtagaggtaagccaatccatcatttcttgggtacttctactttctctcagtacactgttgttgacgaaaatgctgttgctaagattgatgctgcttctccattggaaaaggtttgtttgattggttgtggtttctctactggttatggttctgctgttaacgttgctaaagttactccaggttctacttgtgctgtttttggtttaggtggtgttggtttgtcagctgttatgggttgtaaagcagctggtgctgctagaattattgccgttgatattaacaaggacaagttcgctaaggccaaagaattgggtgctactgaatgtattaacccacaagattacaagaagcccattcaagaggtcttgaaagaaatgactgatggtggtgtcgatttctcattcgaagttattggtcgtctggatacaatgatggcatctttgttgtgttgccatgaagcttgtggtacatctgttatagttggtgttccaccagcttctcaaaacttgtctatcaatccaatgttgctgttgactggtagaacttggaaaggtgctgtttacggtggtttcaaatccaaagaaggtatcccaaaattggtcgctgatttcatggccaaaaagttttcattggatgccttgatcactcacgttttgccattcgaaaagatcaacgaaggtttcgatttgttgcactccggtaagtctattagaactgttttgaccttctaa

SEQ ID NO: 79 - protein, 375 aa, Homo sapiens [sequence omitted]

SEQ ID NO: 80 - DNA, 1125 bp, Saimiri sciureus
  source 1..1125: mol_type = genomic DNA
atgggtactgctggtaaggttattaagtgtaaagctgctgttttgtgggaccaaaagaagccattttccatcgaagaaattgaagttgctccaccaaagaccaaagaagtcagaatcaagattttggctaccggtatctgtagaaccgatgatcatgttatcaagggcaagatggtttctaagttcccagttatagttggtcatgaagctactggtatcgttgaatctattggtgaaggtgttactactgttaagccaggtgacaaagttatccctttgtttttgccacaatgcagagaatgtaacgcttgttgtaatccagatggtaacttgtgcattagatccgatattactggtagaggtgttttggctgatggtactactagattcacttgtaaaggtaagccagtctaccatttcatgaacacttctactttcactgagtacaccgttgttgatgaatcttccgttgctaagattgatgatacagctccaccagaaaaggtttgtttgattggttgtggtttctctactggttatggtgctgctgttaagactggtaaagttaagcctggttctacctgtgttgtttttggtttaggtggtgttggtttgtccgttattatgggttgtaaatctgctggtgcctccagaattattggtatcgatttgaacaaggacaagttcgaaaaggctatgactgttggtgctaccgaatgtatttctccaaaggattctaccaagccaatctccgaagttttgtctgaaatgactggtaacaatgtcggttacaccttcgaagttattggtagattggataccatgattgatgctttggcttcttgccatatgaactacggtacatctgttgttgttggtgccccaccatctgctaaaatgttgacttatgatcccatgctgttgttcactggtagaacttggaaaggttgtgttttcggtggtttgaagtctagagatgatgttccaaagttggtcaccgaatttttggctaagaagttcgatttggaccaattgatcactcacgttttgccattcaagcaaatcaatgaaggtttcgagttgttgaactccggtcaatctattagaactgtcttgaccttctaa

SEQ ID NO: 81 - protein, 374 aa, Saimiri sciureus
MGTAGKVIKCKAAVLWDQKKPFSIEEIEVAPPKTKEVRIKILATGICRTDDHVIKGKMVSKFPVIVGHEATGIVESIGEGVTTVKPGDKVIPLFLPQCRECNACCNPDGNLCIRSDITGRGVLADGTTRFTCKGKPVYHFMNTSTFTEYTVVDESSVAKIDDTAPPEKVCLIGCGFSTGYGAAVKTGKVKPGSTCVVFGLGGVGLSVIMGCKSAGASRIIGIDLNKDKFEKAMTVGATECISPKDSTKPISEVLSEMTGNNVGYTFEVIGRLDTMIDALASCHMNYGTSVVVGAPPSAKMLTYDPMLLFTGRTWKGCVFGGLKSRDDVPKLVTEFLAKKFDLDQLITHVLPFKQINEGFELLNSGQSIRTVLTF

SEQ ID NO: 82 - DNA, 771 bp, Meyerozyma guilliermondii
  source 1..771: mol_type = genomic DNA
atgctgaaaacctccattagaaacattgccagacaacaattgagaaccgctccaatgttgagagctgctccagtttttagagctaatgctggtgttagaattgctagaactttggcctcttacaccattccagaaaaacaaaaggccgtcatctttgaaactaacggtggtgaattgaagtacaccgatattccagttccaaagccaaagcctaacgagttgttgatcaatgttaagtactctggtgtttgccatactgacttgcatgcttggaaaggtgattggccattggatacaaaattgccattggttggtggtcatgaaggtgccggtgttgttgttggtatgggtgaaaacgttgttggttggaaagttggtgatttggctggtattaagtggttgaatggttcttgtcaagcttgtgaatattgtgatggtggtcacgaatctaattgtgctcaagctgatttgtctggttacactcatgatggttctttccaagaatatgctaccgccgattatattcaagctgctagaattccagaaggtactgatttggcacaagttgctccaattttgtgtgcaggtattactgtttacaaggctttgaaaactgccaacttgactcaaggtcaatgggttgctatttctggtgctggtggtggtttgggtactttggctgttcaatatgctaaagctatgggttacttggttgtcggtatagatggtggtgctgataaggctcaagttagacaagttttcggttgctga

SEQ ID NO: 83 - protein, 256 aa, Meyerozyma guilliermondii
MLKTSIRNIARQQLRTAPMLRAAPVFRANAGVRIARTLASYTIPEKQKAVIFETNGGELKYTDIPVPKPKPNELLINVKYSGVCHTDLHAWKGDWPLDTKLPLVGGHEGAGVVVGMGENVVGWKVGDLAGIKWLNGSCQACEYCDGGHESNCAQADLSGYTHDGSFQEYATADYIQAARIPEGTDLAQVAPILCAGITVYKALKTANLTQGQWVAISGAGGGLGTLAVQYAKAMGYLVVGIDGGADKAQVRQVFGC

SEQ ID NO: 84 - DNA, 771 bp, Rattus norvegicus; identical to SEQ ID NO: 82
  source 1..771: mol_type = genomic DNA

SEQ ID NO: 85 - protein, 374 aa, Rattus norvegicus
MANQVIRCKAAVAWEAGKPLSIEEIEVAPPQAHEVRIKIIATAVCHTDAYTLSGADPEGCFPVILGHEGAGIVESVGEGVTKLKAGDTVIPLYIPQCGECKFCLNPKTNLCQKIRVTQGKGLMPDGTSRFTCKGKPILHFMGTSTFSEYTVVADISVAKIDPSAPLDKVCLLGCGISTGYGAAVNTAKVEPGSTCAVFGLGGVGLAVIMGCKVAGASRIIGIDINKDKFAKAKEFGATECINPQDFSKSIQEVLIEMTDGGVDFSFECIGNVKVMRSALEAAHKGWGVSVVVGVAASGEEISTRPFQLVTGRTWKGTAFGGWKSVESVPKLVSEYMSKKIKVDEFVTGNLSFDQINKAFDLMHSGNSIRTVLKM

SEQ ID NO: 86 - protein, 515 aa, synthetic construct; identical to SEQ ID NO: 26
  SIGNAL 1..26

SEQ ID NO: 87 - protein, 571 aa, Millerozyma farinosa
MGFELWGRTNTGGLRGRPLRVAITAVATTGFSLFGYDQGLMSGIITGTEFNEEFPPTWSKPHYNASEKRHATVVQGAVTACYEIGCFFGALFALVRGDRIGRRPLVIVGAVLIIIGTVISTAAFGEHWGLGQFVIGRVITGIGNGMNTATIPVWQSEISRPENRGKLVNLEGSVIAIGTFVAYWIDFGLSYVNSSVQWRFPVAFQIVFAAGLLGGILFMPESPRWLLAHGKKEQAHIVLGALNDLDPNDDHVLAESTVITDAINRFSRSQLGFKELMSGGKNQHFARMVIGSSTQFFQQFTGCNAAIYYSTVLFEETIFVGDRRLSLVMGGVFASVYALATIPSFFLVDKLGRRNLFLIGATGQALSFTITFACLINPTKQNAKGAAVGIFLFITFFAFTILPLPWIYPPEINPLRTRTVASAVSTCTNWLTNFAVVMFTPIFINDAQWGCYLFFACLNYAFIPVIFWFYPETAGRSLEEIDIIFAKAYTDGRPPWRVAATMPHLSLKEQEEQGMQLGLYDNEAEKQKFEQTENLMSSSSSAKLPEEGSNVNENENENTNEKDQTPKPTDV

SEQ ID NO: 88 - protein, 569 aa, Saccharomyces paradoxus
MKDSKLSKFKGRFMSRTSHWGLTGQKLRYFITIASMTGFSLFGYDQGLMASLITGKQFNYEFPATKENGDHDRHATVVQGATTSCYELGCFAGSLFVMFYGERIGRKPLILMGSIITIIGAVISTCAFRDYWALGQFIVGRVVTGVGTGLNTSTIPVWQSEMSKAENRGLLVNLEGSTIAFGTMIAYWIDFGFSYTNSSVQWRFPVSMQIVFALFLLAFMIKLPESPRWLISQSRTEEARYLVGTLDDTDPNDEEVITEVAMLHDAVNRTKHEKHSLSSLFSRGKSQNLQRALIAASTQFFQQFTGCNAAIYYSTVLFNKTIKLDHRLSMIIGGVFATIYALSTIGSFFLIEKLGRRKLFLLGATGQAVSFTITFACLVKENKENARGAAVGLFLFITFFGLSLLSLPWIYPPEIASMKVRASTNAFSTCTNWLCNFAVVMFTPIFIGQSGWGCYLFFAVMNYLYIPVIFFFYPETAGRSLEEIDIIFAKAYEDGTQPWRVANHLPKLSLQEVEDHANALGSYDDEMEKDDFAEDRVEDTYNQINGDNSSSSSNIKNEDTVNDKANSES